(19) United States
(12) Patent Application Publication
(10) Pub. No.: US 2004/0241748 A1
(43) Pub. Date: Dec. 2, 2004

(54) Self-assembling arrays and uses thereof

(76) Inventors: Dana Ault-Riche, Los Gatos, CA (US); Krishnanand D. Kumble, Los Altos, CA (US); Rainer Schulz, Los Altos, CA (US); Kenneth Schulz, Los Altos, CA (US)

Correspondence Address: Stephanie L. Seidman, Fish & Richardson P.C., 12390 El Camino Real, San Diego, CA, 92130-2081, US

(21) Appl. No.: 10/699,113

(22) Filed: Oct. 30, 2003

Related U.S. Application Data
(60) Provisional application No. 60/446,687, filed on Feb. 10, 2003.

(51) Int. Cl. G01N 33/53; G01N 33/543

(52) U.S. Cl. 435/7.1; 436/518

(57) ABSTRACT

Provided herein are methods, combinations, kits and systems for self-assembly of a self-assembling array to produce self-assembled arrays. The self-assembling arrays contain addressed collections of capture agents and are used with binding partners and reagents for conjugation of binding partners to molecules and/or biological particles for display in self-assembled arrays. The capture agents at each locus in a self-assembling array are specific to one of a set of binding partner molecules. The binding partners are conjugated to molecules and/or biological particles for display. Following conjugation, the resulting conjugates are sorted on the array based on the specific interaction of the binding partner and the capture agent to produce a self-assembled array.

### RELATED APPLICATIONS

Benefit of priority under 35 U.S.C. §119(e) to U.S. provisional application Serial No. 60/446,687, filed Feb. 10, 2003, to Dana Ault-Riche, Krishnanand D. Kumble, Rainer Schulz and Kenneth Schulz, entitled “SELF-ASSEMBLING ARRAYS AND USES THEREOF” is claimed.

This application also is related to U.S. provisional application Serial No. 60/422,923, filed Oct. 30, 2002, to Dana Ault-Riche and Bruce Atkinson, entitled “METHODS FOR PRODUCING POLYPEPTIDE-TAGGED COLLECTIONS AND CAPTURE SYSTEMS CONTAINING THE TAGGED POLYPEPTIDES” and to U.S. provisional application Serial No. 60/423,018, filed Oct. 30, 2002, to Dana Ault-Riche, Bruce Atkinson, Lynne Jesaitis, Krishnanand D. Kumble and Gizette Sperinde, entitled “SYSTEMS FOR CAPTURE AND ANALYSIS OF BIOLOGICAL PARTICLES AND METHODS USING THE SYSTEMS.”

This application also is related to U.S. application Ser. No. 09/910,120, filed Jul. 18, 2001, to Dana Ault-Riche and Paul D. Kassner, entitled “COLLECTIONS OF BINDING PROTEINS AND TAGS AND USES THEREOF FOR NESTED SORTING AND HIGH THROUGHPUT SCREENING,” published as U.S. application Serial No. 20020137053, and to U.S. provisional application Serial No. 60/219,183, filed Jul. 19, 2000, to Dana Ault-Riche entitled “COLLECTIONS OF ANTIBODIES FOR NESTED SORTING AND HIGH THROUGHPUT SCREENING.” This application is related to International PCT application No. WO 02/06834. This application also is related to U.S. provisional application Serial No. 60/352,011, filed Jan. 24, 2002, to Dana Ault-Riche and Paul D. Kassner, entitled “USE OF COLLECTIONS OF BINDING PROTEINS AND TAGS FOR SAMPLE PROFILING” and to U.S. patent application Ser. No. 10/351,891 filed Jan. 24, 2003, to Dana Ault-Riche and Paul D. Kassner, entitled “USE OF COLLECTIONS OF BINDING PROTEINS AND TAGS FOR SAMPLE PROFILING AND OTHER APPLICATIONS,” and to International PCT application No. WO03/062402.

This application also is related to U.S. provisional application Serial No. 60/422,923, filed Oct. 30, 2002, to Dana Ault-Riche and Bruce Atkinson, entitled “METHODS FOR PRODUCING POLYPEPTIDE-TAGGED COLLECTIONS AND CAPTURE SYSTEMS CONTAINING THE TAGGED POLYPEPTIDES” and to U.S. provisional application Serial No. 60/423,018, filed Oct. 30, 2002, to Dana Ault-Riche, Bruce Atkinson, Lynne Jesaitis, Krishnanand D. Kumble and Gizette Sperinde, entitled “SYSTEMS FOR CAPTURE AND ANALYSIS OF BIOLOGICAL PARTICLES AND METHODS USING THE SYSTEMS”

This application also is related to U.S. application Ser. No. attorney dkt no. 25885-1754 and 25885-1754PC, entitled “METHODS FOR PRODUCING POLYPEPTIDE-TAGGED COLLECTIONS AND CAPTURE SYSTEMS CONTAINING THE TAGGED POLYPEPTIDES,” to U.S. application Serial No. attorney dkt. nos. 25885-1759 and 25885-1759PC, each entitled “SYSTEMS FOR CAPTURE AND ANALYSIS OF BIOLOGICAL PARTICLES AND METHODS USING THE SYSTEMS”, and to U.S. application Ser. No. attorney dkt. nos. 25885-1755PC, each entitled, “SELF-ASSEMBLING ARRAYS AND USES THEREOF”, filed the same day herewith.

The subject matter of each of the above-noted applications, provisional applications, published applications and internaional applications is incorporated in its entirety by reference thereto.

### FIELD OF INVENTION

Self-assembling arrays that contain collections of binding proteins, called capture agents herein, and binding partners, and, particularly to methods for preparation and use of the self-assembling array are provided. The self-assembling arrays methods and collection technology integrate high throughput screening, addressable arrays and related products and methods.

### BACKGROUND

Genomics and proteomics have delivered massive amounts of information and data about life's molecular components, moving the bottle neck of drug discovery downstream by providing targets and leads to companies and laboratories focused on drug discovery and improved diagnostics. For example, the sequencing of the human genome has led to the identification of approximately 30,000 genes. These 30,000 genes, in turn, can generate many-fold greater diversity in message RNA transcripts through alternate splicing reactions. Even more diversity is created through processing of the message RNA into proteins and further post-translational modifications. The combination of these chemical processes (alternative RNA splicing, protein processing and post-translational modifications) increase the diversity of chemical entities into the millions. Further, the chemical environment of a cell is largely controlled by the proteins in the cell. Therefore, information about the abundance, modification state, and activity of the proteins in a cellular sample is extremely valuable in understanding cellular biology. All of this genotypic and phenotypic information is vital to the development of new pharmaceuticals and better diagnostic tests for the treatment of disease, and therefore, must be examined.

To this end, a multitude of technologies available are designed to gather biological information on a faster and faster scale. For example, robotics and miniaturization technologies lead to advances in the rate at which information on complex samples is generated. High-throughput screening technologies permit routine analysis of tens of thousands of samples; microfluidics and DNA array technologies permit information from a single sample to be gathered in a massively parallel manner. DNA array chips can simultaneously measure the quantity of more than 10,000 different RNA molecules in a sample in a single experiment. Continuing changes in analytical innovations and increasing costs of analytical technologies have made it difficult for companies and researchers focused on drug discovery and diagnostics to identify the most cost-effective, efficient and flexible methods and equipment for their specific needs. The challenge for pharmaceutical and biotechnology companies and researchers is three-fold: reduce product development costs, decrease time to market and increase the probability of success for the most promising leads. Achievement of these goals requires efficient, flexible and low cost analytical technologies for the investigation of targets identified by genomic and proteomic methods.

Factors, such as an aging population and a need for new pharmaceuticals create enormous pressures for new cost-effective and more rapid technologies to discover new and better pharmaceutical and diagnostic products. Improved methods for the separation and detection of components of complex mixtures and the effect of perturbations, such as therapeutic compounds and molecules and alterations in experimental and physiological conditions can provide improved diagnostic tests. For example, DNA and proteome array and microarray technologies makes it possible to, for example, efficiently analyze gene expression and function; validate and optimize drug targets; evaluate a potential drug's mode of action or potential toxic side effects; monitor the genetic stability of cell lines used in research; identify previously undetected phenotypes resulting from genetic changes in cell lines; and compare normal and diseased cells for drug discovery, diagnostics and toxicogenomics, in a single or high-throughput format, thereby decreasing the time required to identify or validate a particular diagnostic technique or therapeutic compound. These tools are only available at high cost and low experimental flexibility (i.e., only available with certain addressable molecules or compounds), prompting a high percentage of companies and researchers to prepare their own need-specific arrays and microarrays in-house, resulting in an inefficient use of laboratory materials and time, thereby decreasing productivity and increasing the cost required for the development of new diagnostics and therapeutic compounds.

Therefore, there remains a need for new methods and technologies to provide tools and methods that can allow companies and researchers to increase productivity and decrease their cost burden. Therefore, among the objects herein, it is an object to provide such tools and methods.

### SUMMARY OF THE INVENTION

Provided herein are methods, combinations, kits and systems for self-assembly of a self-assembling array to produce self-assembled arrays. The self-assembling arrays contain addressed collections of capture agents and are used with binding partners and reagents for conjugation of binding partners to molecules and/or biological particles for display in self-assembled arrays. The capture agents at each locus in a self-assembling array are specific to one of a set of binding partner molecules. The binding partners are conjugated to molecules and/or biological particles for display. Following conjugation, the resulting conjugates are sorted on the array based on the specific interaction of the binding partner and the capture agent to produce a self-assembled array.

Provided herein are self-assembling arrays and methods of use thereof to produce self-assembled arrays. The self-assembling arrays provided herein allow the company or researcher to have a flexible experimental surface or an addressable array that can be adapted for use with virtually any analytical system, while unloading the time and cost burden of preparing the arrays. Provided are binding partner and complementary capture agent sets for use to array any selected compounds and/or biological molecules. The resulting self-assembled arrays can be used in any desired application, including but are not limited to, diagnostic assays of biological materials. In such applications, for example, a binding partner is conjugated to any molecule, compound or biological particle of choice using standard conjugation methods and then immobilized via a specific interaction with a second addressably arrayed molecule, such as a capture agent attached to a support. Tools and methods of this type are advantageous for diagnostically assaying one or more biological sample(s), having one or more target(s) per sample, on a single array. Tools and methods of this type also are advantageous for investigating the effect of perturbations, such as drug molecules and conditions, on systems, molecules or biological particles of interest to the researcher. In addition, the specificity and/or affinity between the binding partners and their complementary capture agents is designed to be relatively specific so to avoid or minimize cross-contamination within an array. Therefore, provided herein are combinations, collections, kits and methods for developing lower cost and user specific analytical techniques and technologies for improving diagnostics and drug discovery. These techniques can then be used by a company or researcher to investigate a specific molecule or biological particle for drug discovery, genomic or proteomic investigations in a single or high-throughput format.

The self-assembling arrays provided herein are universal arrays. Arrays of capture agent are provided, particularly as positionally addressable arrays. Tags (binding partners) that specifically bind to each capture agent also are provided. Then the tags are linked by a user, directly or indirectly via a linker(s) to the molecules or biological particles to be arrayed. The tags are linked to the molecules and or biological particles to be arrayed such that they retain their specificity and ability to bind to the capture agents. Hence, any desired moieties can be arrayed by linking them to the tags, which specifically bind to the capture agents. The resulting arrays of capture agents linked to tags linked to the arrayed moieties (molecules, biological particles) can be used for any purpose for which an array of the moieties are arrayed.

Thus, provided herein are combinations, kits, methods and systems for preparing and using self-assembling arrays for developing diagnostics and pharmaceuticals. Methods for discovering compounds that have pharmaceutical and diagnostic applications are provided. The combinations, kits, methods and systems provided herein are tools that provide a way to discover a broad and diverse range of candidate therapeutics and to provide diagnostic tests.

The combinations and kits provided herein contain addressable collections of capture agents and a plurality of sets of binding partners, which specifically bind to a unique capture agent, and optionally conjugating agents for effecting covalent linkage of a binding partner to a displayed molecule and/or displayed biological particle. The combinations and kits optionally contain instructions for use of the addressable collection of capture agents and binding partners to prepare self-assembled arrays and/or software for analysis of assays using the resulting arrays. The capture agents at each locus within the array bind to one set of binding partners. In one embodiment, the number of sets of binding partners is equal to the number of unique capture agents.

The combinations and kits also are provided herein that contain addressable collections of capture agents and a list setting forth the amino acid sequences that comprise the binding portion of polypeptide binding partners for each member of the collection of capture agents or setting forth sequences of nucleotides that encode the sequences of amino acids of binding partners that specifically bind to each member of the collection of capture agents. The combination can further contain one or more conjugation reagents, wherein the reagent effects covalent linkage of a binding partner to a displayed molecule or displayed biological particle.

Capture agents are molecules that have a specificity for other molecules or biological particles, such as a ligand or anything that includes a defined sequence of amino acids. Capture agents can be naturally-occurring or synthetic molecules, and include any molecule, including nucleic acids, small organics, proteins and complexes that specifically bind to other molecules or specific sequences of amino acids. For example, capture agents can be polypeptides such as antibodies, including monoclonal and polyclonal antibodies, single chain antibodies (scFvs) and fragments thereof that retain the ability to specifically bind to a binding partner.

Exemplary antibodies include, but are not limited to, anti-peptide antibodies such as an anti-E-tag antibody, an anti-FLAG M2 antibody, an anti-Glu-Glu antibody, an anti-HA.11 antibody, an anti-HSV-tag antibody, an anti-c-myc antibody, an anti-T7 tag antibody, an anti-VSV G antibody, an anti-V5 antibody, an anti-AB2 antibody, an anti-AB4 antibody, an anti-B34 antibody, an anti-P5D4 A antibody, an anti-P5D4 B antibody, an anti-4C10 antibody, an anti-AB3 antibody, an anti-AB6 antibody, an anti-KT3 A antibody, an anti-KT3 B antibody, an anti-KT3 C antibody, an anti-7.23 antibody, an anti-HOPC1 antibody, an anti-S1 antibody, an anti-E2 antibody, an anti-His tag antibody, an anti-AU1 antibody, an anti-AU5 antibody, an anti-IRS antibody, an anti-NusA antibody, an anti-MBP antibody, an anti-TBP antibody and an anti-TRX antibody.

Binding partners for use in the combinations and kits provided herein include polypeptide tags which bind to capture agents. Binding partners can be antibodies, including monoclonal antibodies, single chain antibodies (scFvs) and fragments thereof. Exemplary polypeptide tags include, but are not limited to, an E-tag polypeptide (SEQ ID No.1), a FLAG polypeptide (SEQ ID No.2), a Glu-Glu polypeptide (SEQ ID No.3), a HA.11 polypeptide (SEQ ID No.4), a HSV-tag polypeptide (SEQ ID No. 5), a c-myc polypeptide (SEQ ID No.6), a T7 tag polypeptide (SEQ ID No. 7), a VSV-G polypeptide (SEQ ID No.8), a V5 polypeptide (SEQ ID No. 9), an AB2 polypeptide (SEQ ID No.10), an AB4 polypeptide (SEQ ID No. 11), a B34 polypeptide (SEQ ID No.12), a P5D4-A polypeptide (SEQ ID No. 13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a S1 polypeptide (SEQ ID No.23), an E2 polypeptide (SEQ ID No.24), a His tag polypeptide (SEQ ID No.25), an AU1 polypeptide (SEQ ID No. 26), an AU5 polypeptide (SEQ ID No.27), an IRS polypeptide (SEQ ID No. 28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32). Combinations can include the polypeptide tags or sequences of nucleotides that encode the polypeptide tags.

The combinations can be positionally addressed, for example as spots on a solid support or an array. Examples of solid supports include, but are not limited to, silicon, cellulose, metal, polymeric surfaces, radiation grafted supports, gold, nitrocellulose, polyvinylidene difluoride (PVDF), radiation grafted polytetrafluoroethylene, polystyrene, glass and activated glass. The solid support can contain a well or pit or plurality thereof in a surface of the solid support. The solid support can also be chosen from plates, beads, microbeads, whiskers, combs, hybridization chips, membranes, single crystals, ceramics and self-assembling monolayers. In one exemplary embodiment, the collection is addressed as loci on a solid support and each locus on the solid support contains polyclonal antibodies specific for one binding partner. Such polyclonal antibodies can have an avidity for the binding partner about 10⁸-10¹². The collection of capture agents also are addressably tagged by linking them to electronic, chemical, optical or color-coded labels.

The capture agents can be attached to the solid support by a variety of means. For example, capture agents are attached to the solid support by a covalent bond, an electrostatic bond, a hydrogen bond or a combination thereof. Attachment can also include a linker between the collections of capture agents and the solid support. Linkers include but are not limited to, oligopeptides, oligonucleotides, oligopolyamides, oligoethyleneglycerol, oligoacrylamides, alkyl chains of between about 6 to about 20 carbon atoms, and combinations thereof. In one embodiment, the attachment is a cleavable attachment, such as cleavable by an enzyme, a chemical agent or electromagnetic radiation, such as visible, ultraviolet and infrared radiation. A chemical agent for cleavage can be chosen from, but not limited to, reducing agents, oxidizing agents, hydrolyzing agents and combinations thereof. In one exemplary embodiment, the collections of capture agents are conjugated with biotin or a biotin derivative and the solid support is conjugated with avidin, streptavidin or a derivative thereof, so that the capture agents are linked to the support.

The combinations and kits provided herein contain one or more conjugation reagents for effecting covalent linkage of a binding partner to a displayed molecule and/or displayed biological particle. The binding partners can be either linked to a particular molecule or biological particle directly through a chemical conjugation, by a linker between the binding partner and the molecule and/or biological particle or can be linked by producing fusion proteins from nucleic acid encoding the binding partner linked directly or indirectly to nucleic acid encoding the molecule. Linkers include, but are not limited to, a peptide linker, a chemical linker, or a cleavable linker, such as acid-cleavable, heat labile and photocleavable linkers. The linkage between the binding partner and the molecule and/or biological particle can also be a linkage through an intermediate molecule, such as a bead, including an electronic, chemical, optical, or color-coded labeled bead.

Conjugation reagents effecting linkage between a binding partner and a molecule and/or biological particle and between capture agents and solid supports include, but are not limited to, the covalent linkages such as thiol-thiol, thiol-amine, amine-amine, amine-carboxylic acid, thiol-carboxylic acid, thiol-carbohydrate and amine-non selective linkages. Conjugation reagents for use in the combinations and kits provided herein include, but are not limited to, ethylene glycol bis[succinimidylsuccinate](EGS); Ethylene glycol bis[sulfosuccinimidylsuccinate](Sulfo-EGS); Bis[2-(Sulfosuccinimidooxycarbonyloxy)ethyl]sulfone (Sulfo-BSOCOES); Bis[2-(succinimidooxycarbonyloxy)ethyl]sulfone (BSOCOES); Dithiobis[succinimidylpropionate](DSP); 3,3′-Dithiobis[sulfosuccin-imidylpropionate (DTSSP); Dimethyl 3,3′-dithiobispropionimidate.2HCl (DTBP); Disuccinimidyl suberate (DSS); Bis[sulfosuccinimidyl]suberate (BS3); Dimethyl Suberimidate.2HCl (DMS); Dimethyl pimelimidate.2HCl (DMP); Dimethyl adipimidate.2HCl (DMA); Disuccinimidyl glutarate (DSG); Methyl N-succinimidyl adipate (MSA); Disuccinimidyl tartarate (DST); Disulfosuccinimidyl tartarate (Sulfo-DST); 1,5-Difluoro-2,4-dinitrobenzene (DFDNB); (4-Succinimidyloxycarbonyl-methyl-a-[2-pyridyidithioltoluene (SMPT); 4-Sulfosuccinimidyl-6-methyl-a-(2-pyridyidithio)toluamido]hexanoate) (Sulfo-LC-SMPT); N-[k-Maleimidoundecanoyloxy]sulfosuccinimide ester (Sulfo-KMUS); Succinimidyl-4-(N-Maleimidomethyl)cyclohexane-1-carboxy-(6-amidocaproate) (LC-SMCC); N-k-Maleimidoundecanoic acid (KMUA); Sulfosuccinimidyl 6-(3-[2-pyridyldithio]-propionamido)hexanoate (Sulfo-LC-SPDP); Succinimidyl 6-(3-[2-pyridyidithio]-propionamido)hexanoate (LC-SPDP); Succinimidyl 4-[p-maleimidophenyl]butyrate (SMPB); Sulfosuccinimidyl-4-(P-Maleimidophenyl)Butyrate (Sulfo-SMPB); Succinimidyl-6-[β-maleimidopropionamidolhexanoate (SMPH); Sulfosuccinimidyl 4-[N-maleimidomethyl]cyclohexane-1-carboxylate (Sulfo-SMCC); Succinimidyl 4-[N-maleimidomethyl]cyclohexane-1-carboxylate (SMCC); N-Succinimidyl [4-iodoacetyl]aminobenzoate (SIAB); N-Sulfosuccinimidyl [4-iodoacetyl]aminobenzoate (Sulfo-SIAB); N-[g-Maleimidobutyryloxy]sulfosuccinimide ester (Sulfo-GMBS); N-[g-Maleimidobutyryloxy]succinimide ester (GMBS); m-Maleimidobenzoyl-N-hydroxysuccinimide ester (MBS); m-Maleimidobenzoyl-N-hydroxysulfosuccinimide ester (Sulfo-MBS); [N-e-Maleimidocaproyloxy]sulfosuccinimide ester (Sulfo-EMCS); N-e-Maleimidocaproic acid (EMCA); [N-e-Maleimidocaproyloxy]succinimide ester (EMCS); N-Succinimidyl-[4-vinylsulfonyl]benzoate (SVSB); N-[β-Maleimidopropyloxy]succinimide ester (BMPS); N-Succinimidyl 3-[2-pyridyidithio]-propionamido (SPDP); Succinimidyl 3-[bromoacetamido]propionate (SBAP); N-[β-Maleimidopropionic acid (BMPA); N-[α-Maleimidoacetoxy]succinimide ester (AMAS); N-Succinimidyl-S-acetylthiopropionate (SATP); N-Succinimidyl iodoacetate (SIA); Sulfosuccinimidyl 2-[7-amino-4-methylcoumarin-3-acetamido]ethyl-1,3′dithiopropionate (SAED); Sulfosuccinimidyl-2-[p-azidosalicylamido]ethyl-1,3′-dithiopropionate (SASD); Sulfosuccinimidyl 2[m-azido-o-nitrobenzamido]-ethyl-1,3′-dithiopropionate (SAND); N-Succinimidyl-6-[4′-azido-2′-nitrophenylamino]hexanoate (SANPAH); N-Sulfosuccinimidyl-6-[4′-azido-2′-nitrophenylamino]hexanoate (Sulfo-SANPAH); Sulfosuccinimidyl [4-azidosalicylamido]-hexanoate (Sulfo-NHS-LC-ASA); Sulfosuccinimidyl-[perfluoroazidobenzamido]ethyl-1,3′-dithiopropionate (SFAD); N-Sulfosuccinimidyl (4-azidophenyl)-1,3′-dithiopropionate (Sulfo-SADP); N-Succinimidyl(4-azidophenyl)-1,3′-dithiopropionate (SADP); N-Hydroxysulfosuccinimidyl-4-azidobenzoate (Sulfo-HSAB); N-Hydroxysuccinimidyl-4-azidosalicylic acid (NHS-ASA); N-5-Azido-2-nitrobenzoyloxysuccinimide (ANB-NOS); N-[e-Trifluoroacetylcaproyloxyl-succinimide ester (TFCS); Succinimidyl-[4-(psoralen-8-yloxy)]butyrate (SPB(NHS-Psoralen)); Sulfosuccinimidyl [2-6-(biotinamido)-2-(p-azidobenzamido)-hexanoamido]-ethyl-1,3′-dithiopropionate (Sulfo-SBED); 1,4-Di-[3′-(2′-pyridyldithio)-propionamido]butane (DPDPB); 1,11-bis-Maleimidotetraethyleneglycol (BM[PEO]₄); Bis-Maleimidohexane (BMH); 1,8-bis-Maleimidotriethyleneglycol (BM[PEO]₃); 1,6-Hexane-bis-vinylsulfone (HBVS); Dithio-bis-maleimidoethane (DTME); 1,4-bis-Maleimidobutane (BMB); 1,4 bis-Maleimidyl-2,3-dihydroxybutane (BMDB); Bis-Maleimidoethane (BMOE); N-[k-Maleimidoundecanoic acid]hydrazide (KMUH); 4-(4-N-Maleimidophenyl)butyric acid hydrazide hydrochloride (MPBH); 4-(N-Maleimidomethyl)cyclohexane-1-carboxyl hydrazide hydrochloride (M₂C₂H); [N-e-Maleimidocaproic acid]hydrazide (EMCH); 3-(2-Pyridyldithio)propionyl hydrazide (PDPH); 3-Maleimidophenyl boronic acid (MPBA); N-[fl-Maleimidopropionic acid]hydrazide.TFA (BMPH); N-[4-(p-Azidosalicylamido) butyl]-3′-(2′-pyridyidithio) propionamide (APDP); N-[p-Maleimidophenyl]isocyanate (PMPI); p-Azidobenzoyl hydrazide (ABH); p-Azidophenyl glyoxal monohydrate (APG); Bis-[b-(4-Azidosalicylamido)ethyl]disulfide (BASED); 4-[p-Azidosalicylamido]butylamine (ASBA); 3-[(2-Aminoethyl) dithio]propionic acid.HCl (AEDP); and 1-Ethyl-3-[3-dimethylaminopropyl]carbodiimide Hydrochloride (EDC).

Combinations provided herein can also contain information on using the combination, tutorials or technical support information. Kits containing the combinations described herein and packaging material or instructions for using the kit for preparation, use and/or analysis of self-assembled arrays are provided. Packaging material includes, but is not limited to, ice, dry ice, styrofoam, foam, plastic, cellophane, shrink wrap, bubble wrap, paper, cardboard, starch peanuts, twist ties, metal clips, metal cans, drierite, glass and rubber.

Also provided herein are methods for preparing a self-assembled array by providing an addressable array of capture agents that have predetermined binding partners; preparing a plurality of sets of conjugates, where each set of conjugates comprises a biological particle and/or molecule linked to a binding partner or plurality thereof, and the binding partner binds to one of the capture agents in the array. The addressable array of capture agents is contacted with the sets of conjugates to produce the self-assembled array. Also provided is a method for preparing a self-assembled array, by providing an addressable array of capture agents that have predetermined binding partners; preparing sets of binding partners, where a binding partner is a polypeptide encoded by a nucleic acid; and preparing a plurality of sets of conjugates, where each set of conjugates comprises a biological particle and/or molecule linked to a binding partner or plurality thereof from one set of binding partners, and the binding partner binds to one of the capture agents in the array. The addressable array of capture agents is contacted with the sets of conjugates to produce the self-assembled array.

Also provided herein are self-assembled arrays containing an addressable array of capture agents that have predetermined binding partners; and a plurality of sets of conjugates, where each set of conjugates comprises a biological particle and/or molecule linked to a binding partner or plurality thereof, and the binding partners are specifically bound to their capture agents. The self-assembled arrays and methods for preparing self-assembled arrays use capture agents, collections and arrays of capture agents, binding partners and reagents for effecting conjugation such as described herein.

The conjugate complexes, including the binding partner and the molecule and/or biological particle, are contacted with the addressed collections of capture agents, in which the agents at each loci specifically bind to the same tag, under conditions which allow the binding partners to bind to the loci containing the capture agents to produce the self-assembled array. The self-assembled arrays provided herein can be used to assess an effect of the interaction between an exogenous molecule and/or biological particle with a the self-assembled array. The self-assembled arrays can also be used to assess the effect of a candidate compound or condition on the interaction between an exogenous molecule and/or biological particle with a the self-assembled array. The resulting self-assembled array can be used in a variety of methods including methods in which the arrayed conjugated molecules or biological particles are assessed and identified, and methods in which the self-assembled arrays are used to bind to additional exogenous molecules and/or biological particles in order to assess interactions of the conjugated molecule and/or biological particles displayed by the self-assembled array with test and/or known candidate compounds and/or conditions, such as pH, temperature, ionic strength, pressure and other parameters.

Examples of exogenous molecules and test compounds for use with self-assembled arrays include, but are not limited to, an organic compound, an inorganic compound, a metal complex, a receptor, a ligand, an enzyme, an antibody, a protein, a nucleic acid, a peptide nucleic acid, DNA, RNA, a polynucleotide, an oligonucleotide, an oligosaccharide, a lipid, a lipoprotein, an amino acid, a peptide, a cyclic peptide, a polypeptide, a peptidomimetic, a carbohydrate, a cofactor, a drug, a prodrug, a lectin, a sugar, a glycoprotein, a biomolecule, a macromolecule, a biopolymer, a polymer, a sub-cellular structure, a sub-cellular compartment, a virus, a phage, a cell, a liposome, and a micellar agent. Test conditions can also be selected from, but not limited to, a variation in buffer or solution components, pH, temperature, exposure to light, aerobic or anaerobic conditions, concentration of components, duration of experimental detection, ionic strength, pressure, agitation, and organic or aqueous interaction medium.

Assessment of an effect includes, but is not limited to, a change in structure, function, a physical change, a chemical or a morphological change, signal transduction, protein trafficking, gene expression, translation, the pattern (profile) of captured molecules, degradation of a biopolymer in or on the biological particle, proliferation, cell death, apoptosis, morphological changes, gene expression, transcription, translation, receptor internalization, receptor shedding, receptor-mediated activation of the biological particle or a receptor thereon or therein, differentiation, dedifferentiation, interactions among biological particles, endocytosis, phagocytosis, exocytosis, phosphorylation, dephosphorylation and change in kinetics of an intra-particle reaction.

Exemplary methods of use for self-assembled arrays include identifying molecules that modulate trafficking in biological particles, and identifying a molecule that modulates activity or functional or structural property in or of molecules and/or biological particles. A method is provided for identifying a molecule that modulates trafficking in biological particles by preparing a self-assembled array such as described herein; contacting an exogenous biological particle to the self-assembled array; monitoring trafficking in the exogenous biological particle, to thereby identifying the conjugated molecule(s) from among the self-assembled array that modulate the trafficking in the exogenous biological particle. A method also is provided for identifying an exogenous molecule that modulates trafficking in biological particles by preparing a self-assembled array such as described herein; adding a candidate compound or exposing the self-assembled array to a condition before, during or after contacting the self-assembled array with an exogenous biological particle; and monitoring trafficking in the exogenous biological particle, to thereby identify the candidate compound(s) and/or condition(s) that modulate trafficking in the exogenous biological particle. Examples of conjugated molecules or candidate compounds that can be used to modulate trafficking include, but are not limited to, oligonucleotides, oligonucleosides, polypeptides, such as enzymes, proteins, receptors, cellular adhesion molecules, antibodies and fragments thereof, amino acids, nucleotides, nucleosides, peptide nucleic acids, oligosaccharides, monosaccharides, organic compounds, inorganic compounds, metal complexes, metal ions, lipids, lipoproteins, peptidomimetics, carbohydrates, cofactors, drugs, prodrugs, lectins, sugars, glycoproteins, biomolecules, macromolecules, biopolymers, polymers, sub-cellular structures, sub-cellular compartments or any combination, portion, salt, or derivative thereof.

Provided herein also is a method for identifying a molecule that modulates activity or functional or structural property in or of molecules and/or biological particles, by preparing a self-assembled array such as described herein; contacting the self-assembled array with exogenous molecules; monitoring the activity, function or structural property in or of the conjugated molecules and/or biological particles in the self-assembled array, to thereby identify the exogenous molecule(s) that modulate the activity, function or structural property in or of the conjugated molecules and/or biological particles in the self-assembled array. Also provided is a method for identifying a molecule that modulates an activity or functional or structural property in or of a conjugated molecule and/or biological particle in a self-assembled array, by preparing a self-assembled array such as described herein; adding a candidate compound or exposing the self-assembled array to a condition before, during or after contacting the self-assembled array with exogenous molecules; and monitoring the activity, function or structural property in or of the conjugated molecules and/or biological particles, to thereby identify the candidate compound(s) and/or condition(s) that modulate the activity, function or structural property in or of the conjugated molecules and/or biological particles in the self-assembled array. The activity, function or structural property modulated can include, but is not limited to, proliferation, apoptosis, morphology, transcription, translation, receptor internalization, receptor shedding, signal transduction, receptor-mediated activation of a biological particle, receptor-activated signaling in a biological particle, differentiation, dedifferentiation, interactions among constituent proteins and/or protein complexes and components thereof, interactions among biological particles, endocytosis, phagocytosis, exocytosis, phosphorylation, dephosphorylation and change in kinetics of an intra-particle reaction.

Also provided herein are methods of analyzing and processing arrays, program products for use with arrays, apparatus for processing array data, and systems for analysis of a collection of arrays. Such methods, program products and systems can be used with any of the methods, combinations, kits and systems for self-assembled arrays provided herein.

A method is provided herein for analyzing and/or processing a signal or plurality thereof at loci on an exposed positionally addressable array, by receiving image data corresponding to pixel luminosity information at the exposed locus within the positionally addressable array, wherein the locus comprises capture agents; specifying pre-determined input parameters for processing the received image data that include parameters that specify a predetermined array locus or plurality thereof on a surface of the array; determining an actual location of a locus or plurality thereof on the array; and processing the image data for each exposed locus in accord with the determined actual locus and the input parameters. A plurality of arrays can be processed by this method. The method can include processing by determining or detecting luminosity at the actual locus on the array. The actual locus can be determined by processing image data that corresponds to pixels located a distance from a pre-selected locus such that luminosity information for the pixels indicates increasing values; and recording an array location that corresponds to a local maximum of intensity values as the actual location of the locus. The pre-determined input parameters for processing the received image data include parameters that specify all loci on the surface of the array.

Also provided herein is a method for processing a signal or plurality thereof at loci that display a detectable signal in or on an array, including a self-assembled array, by receiving image data corresponding to pixel luminosity information for locations for a plurality of the loci of the array; for a particular locus of the array, determining neighbor luminosity effects on the image data for the particular locus that are produced from adjacent neighbor loci; and compensating for the neighbor luminosity effects of the adjacent neighbor loci from the image data for the particular locus. This method can process a plurality of arrays, including addressable arrays and self-assembled arrays. The method includes determining neighbor luminosity effects by determining a luminosity value corresponding to an illumination value for a location at each neighbor locus of the particular locus; determining an actual location on the top surface of the canvas for each locus; determining an array distance from the particular locus to each of the associated neighbor loci; and compensating for the luminosity effects from each of the neighbor loci in accord with their respective distance to the particular locus. The actual location for each particular locus is determined by processing image data that corresponds to pixels located a distance from each particular locus of interest such that luminosity information for the pixels indicates increasing values; and recording an array location that corresponds to a local maximum of intensity values as the actual location of the particular locus of interest. The array distance is determined by subtracting the determined actual locations of the particular locus to determine distances between the respective locations of the particular locus and the associated neighbor loci.

Also provided is a program product for use in a computer device that executes program instructions recorded in a computer-readable medium to perform a method for analyzing arrays that have loci that display a detectable signal in or on the array, the program product containing a recordable medium and a plurality of computer-readable instructions executable by the computer device. The computer-readable instructions executable by the computer device perform a method of receiving image data corresponding to pixel luminosity information of the loci; specifying input parameters for processing the received image data that include parameters that specify a predicted array location for each locus of the array, determining an actual location for each locus; and processing the image data for each location in accord with the determined actual location and the input parameters. The actual location is determined by processing image data that corresponds to pixels located a distance from a locus such that luminosity information for the pixels indicates increasing values; and recording an array location that corresponds to a local maximum of intensity values as the actual locus.

Also provided herein is a program product for use in a computer device that executes program instructions recorded in a computer-readable medium to perform a method for processing an array with loci that display a detectable signal in or on the array, containing a recordable medium and a plurality of computer-readable instructions executable by the computer device. The computer-readable instructions executable by the computer device perform the method of receiving image data corresponding to pixel luminosity information of the loci for a plurality of the locations of the array, for a particular locus in the array, determining neighbor luminosity effects on the image data for the particular location that are produced from adjacent neighbor locations, and compensating for the neighbor luminosity effects of the adjacent neighbor locations from the image data for the particular location. The neighbor luminosity effects are determined by determining a luminosity value corresponding to an illumination value for a biological material location at each neighbor location of the particular location; determining an actual location on the top surface of the canvas for each biological material location; determining an array distance from the location of the particular biological material to each of the associated neighbor locations; compensating for the luminosity effects from each of the neighbor locations in accord with their respective distance to the location of the particular biological material. The actual locus is determined by processing image data that corresponds to pixels located a distance from a locus such that luminosity information for the pixels indicates increasing values; and recording the array location that corresponds to a local maximum of intensity values as the actual location of the biological material of interest. The array distance is determined by subtracting the determined actual locations of the biological locations to determine distances between the respective locations of the biological material and the neighbor locations.

Also provided herein is an apparatus that processes data produced from imaging detectable loci on an array, containing a computer processor that executes program instructions; an input processor that receives input parameters for processing received image data, including input parameters that specify a predicted array location for each locus on the array, wherein the image data corresponds to pixel luminosity information for loci in the array; an image analysis processor that determines an actual location on the array for each locus, and processes the image data for each location in accord with the determined actual location and the input parameters. The image analysis processor determines the actual location by processing image data that corresponds to pixels located a distance from a particular locus such that luminosity information for the pixels indicates increasing values, and recording an array location that corresponds to a local maximum of intensity values as the actual location of the biological material of interest.

Also provided herein is an apparatus that processes data produced from imaging detectable loci in an array, the apparatus containing a computer processor that executes program instructions; an input processor that receives image data comprising pixel luminosity information for loci in the array; and an image analysis processor that determines neighbor luminosity effects on image data for a particular locus produced from adjacent neighbor locations, and that compensates for the neighbor luminosity effects of the adjacent neighbor locations from the image data for the particular location. The image analysis processor determines neighbor luminosity effects by determining a luminosity value corresponding to an illumination value for a biological material location at each neighbor location of the particular locus, determining an actual location on a surface of the array for each detectable locus, determining an array distance from the location of the particular biological material to each of the associated neighbor locations, and compensating for the luminosity effects from each of the neighbor locations in accord with their respective distance to the location of the detectable locus. The image analysis processor determines the actual location for each detectable locus by processing image data that corresponds to pixels located a distance from each detectable locus such that luminosity information for the pixels indicates increasing values, and recording an array location that corresponds to a local-maximum of intensity values as the actual location of the biological material of interest. The image analysis processor determines the array distance by subtracting the determined actual locations of the biological locations to determine distances between the respective locations.

Also provided herein is a system for analysis of a collection of self-assembled arrays, containing an addressable collection of capture agents and binding partners, comprising sets of capture agents and binding partners, wherein a set of capture agents is selected to specifically bind to a set of binding partners with sufficiently high affinity to produce collections of self-assembled arrays; a conjugation reagent, comprising a compound or molecule sufficient for the conjugation of the sets of binding partners to sets of molecules or biological particles; a computer programmed with instructions for controlling and directing production of an image of the conjugated sets of molecules or biological particles displayed on the collections of self-assembled arrays; and software for processing of image data produced by the collections of self-assembled arrays. The system can be an automated system and can also include a microplate reader and/or a charge coupled device (CCD) camera.

Particular exemplary embodiments and methods include the embodiments described below, including in the claims, and any embodiments apparent therefrom.

### DESCRIPTION OF THE DRAWINGS

FIGS. 1A-1B schematically depict exemplary self-assembled arrays; FIG. 1A depicts the components of the self-assembled array and FIG. 1B depicts an exemplary use of a self-assembled array to detect an interaction between a displayed molecule or biological particle and a target molecule or biological particle.

FIGS. 2A-2B depict exemplary methods for isolating capture agent/binding partner pairs; FIG. 2A shows a panning method and FIG. 2B shows an immunization method.

FIG. 3 depicts an exemplary collection of self-assembled arrays containing capture agents with bound binding partner tagged-molecules.

FIGS. 4A and 4B depict screening for candidate compounds or conditions that modulate interactions and screening for candidate compounds or conditions that modulate the effect of interactions, respectively. The figures depict different exemplary screening methods using capture systems to capture cells in the presence and absence of candidate compounds and conditions. The figures depict displayed antibodies, but it is to be understood that the self-assembled arrays as provided here are designed to display any molecule and/or biological particle of interest, such as, but not limited to, small molecules, libraries of small molecules, peptides, libraries of peptides, cyclic peptides, libraries of cyclic peptides, polypeptides, libraries of polypeptides. Such small molecules, peptides and polypeptides and mimetics and libraries thereof are known to those of skill in the art.

FIG. 5 depicts the binding of anti-peptide antibody capture agents to peptide binding partners displayed on the self-assembling array followed by detection with a goat anti-mouse IgG antibody-HRP conjugate.

FIG. 6 depicts the binding of peptide binding partners conjugated to Neutravidin-HRP to antibody capture agents displayed on the self-assembled array followed by detection of the Neutravidin-HRP conjugate.

FIG. 7 depicts the binding of human IgG-peptide conjugated to antibody capture agents displayed on the self-assembled array followed by detection with an anti-human IgG antibody-HRP conjugate.

FIG. 8 depicts the binding of anti-human IFNy primary antibody-peptide conjugate to anti-peptide antibody capture agents displayed on the self-assembled array followed by binding to IFNy and detection with a secondary anti-human IFNy antibody-HRP conjugate.

FIG. 9 depicts a block diagram of an exemplary system 900 that can perform data processing.

FIG. 10 depicts a flow diagram that illustrates exemplary processing that is controlled by a computer 908.

FIG. 11 depicts a flow diagram that represents exemplary operations executed by the computer system for each spot to determine actual spot location on the slide.

FIG. 12 depicts a representation of an exemplary display window 1202 from which a user can designate the input features that can be invoked.

FIG. 13 depicts an exemplary window 1302 that can be produced when the user selects the “Settings” display button from the FIG. 12 window and then selects the “Plate Settings” tab from the resulting “Settings” window.

FIG. 14 depicts an exemplary window 1402 that can be produced when the “Array Settings” tab of the “Settings” display is selected.

FIG. 15 depicts an exemplary display window 1502 that can result from choosing the “Select from Image” button of FIG. 13.

FIG. 16 depicts an example of a color map, which is a color-coded representation of the canvas image.

FIG. 17 depicts an exemplary graph output 1702 that can be produced after image analysis by the software.

FIG. 18 depicts an exemplary graph output window 1802 that can result from selecting the “Graph” button on the image analysis main window in FIG. 12.

FIG. 19 depicts an example of a suitable computer system 1900 that can implement the functionality described herein.

FIG. 20 depicts an exemplary process for designing polypeptide binding partners.

For clarity of disclosure, and not by way of limitation, the detailed description is divided into the subsections that follow.

### DETAILED DESCRIPTION OF EXEMPLARY EMBODIMENTS

A. Definitions

B. Components of the Self-Assembling Array

1. The Capture Agent Array

a. Printing the Array

b. Support Materials for Immobilizing Capture Agents

(1) Natural Support Materials

(2) Synthetic Supports

C. Immobilization and Activation

2. Capture Agents

3. Binding partners and Preparation Thereof

4. Identification of Capture Agents—Binding partner Pairs

a. Panning Phage Displayed Peptide Libraries

b. Analysis of Complementarity-determining Regions (CDRs) of the Antibody

c. Theoretical Molecular Modelling of Three-Dimensional Antibody Structure

d. Raising Antibodies in vivo

5. Interactions between Capture Agents and Binding Partners

6. Molecules and Biological Particles for Displaying

a. Exemplary Displayed Molecules and Biological Particles

b. Identification of Displayed Molecules and Biological Particles

(1) Empirical

(2) Data-Mining

7. Conjugation of a Binding Partner to a Displayed Molecule or Biological Particle

a. Fusion Proteins

b. Chemical Conjugation

(1) Thiol-Thiol and Thiol-Amine Conjugates

(2) Amine-Amine Conjugates

(3) Conjugates Involving Other Functional Groups

c. Linkers

(1) Acid cleavable, photocleavable and heat sensitive linkers

(2) Peptide Linkers

(3) Other Linkers

d. Indirect Linkages

8. Imaging and Analytical Software

C. Combinations and Kits

1. Reagents

2. Types of Kits

D. Computer Systems

E. Uses for Combinations, Kits and Systems

1. Identifying Perturbations that Modulatean Interaction or Secondary Effect of an Interaction between a Self-Assembled Array and a Target Molecule and/or Biological particle

a. Perturbations and Screening Methods

b. Use of Perturbations to Identify Interactions

2. Cell Surface Profiling

3. Receptor Agonist/Antagonist Discovery

4. Protein-protein Interactions Including Association-dissociation Assays and Changes in Protein Confirmation

5. Biopolymer Degradation Assays

6. Protein Trafficking Assays

7. Analysis of Modulation of Subcellular Conditions and Processes

8. Assays for assessing Cell Growth and Proliferation

9. Assays for assessing Apoptosis

10. Assays to Assess Changes in Cell Morphology

11. Receptor Internalization Assays

12. Receptor-mediated Cell Activation Assays

13. Receptor Activated Cell Signaling

14. Epitope Mapping

15. Expression of Secreted Polypeptides by Tumor Cells

16. Differentiation/Dedifferentiation Assays

17. Discovery of Molecules that Block Binding, Cleavage and/or Post-translational Modifications

18. Discovery of Antibodies to Apically-localized Cell-surface Proteins, Carbohydrates and Lipids

19. Detection of Phosphorylation and Dephosphorylation Activities

20. Determination and Monitoring of Chemical or Enzymatic Kinetics

21. Screening and Identification of Cyclic Peptides with Antibiotic Activity

F. Identification of binding partner polypeptides

1. Overview of the methods

2. Description of the methods

a. Use of non-naturally occurring amino acids for polypeptide design and generation

b. Generation of polypeptides

G. Identification of binding proteins for polypeptide binding partner pairs

1. Raising antibodies

2. Phage display

3. Generation of Binding protein-binding partner pairs

H. EXAMPLES

### A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases.

Reference thereto evidences the availability and public dissemination of such information.

As used herein, a displayed molecule or a displayed biological particle refers to a molecule or biological particle that is conjugated to a binding partner and addressably displayed by contacting the molecule or biological particle—binding partner conjugate with a plurality of addressable capture agents.

As used herein, a target molecule or a target biological particle refers to a molecule or biological particle that is exposed to or contacted with a self-assembled array.

As used herein, nested sorting refers to the process of decreasing diversity using the addressable collections of capture agents provided herein.

As used herein, profiling refers to detection and/or identification of a plurality of components, generally 3 or more, such as 4, 5, 6, 7, 8, 10, 50, 100, 500, 1000, 10⁴, 10⁵, 10⁶, 10⁷ or more, in a sample. A profile refers to the identified loci to which components of a sample detectably bind. The profile can be detected as a pattern on a solid surface, such as in embodiments when the addressable collection includes an array of capture agents on a solid support, in which case the profile can be presented as an visual image. In embodiments, such as those in which the capture agents and bound tagged molecules are on color-coded beads or are otherwise detectably labeled, a profile refers to the identified binding partners and/or capture agents to which component(s) is(are) detectably bound, which can be in the form of a list or database or other such compendium.

As used herein, an image refers to a collection of datapoints representative of a profile. An image can be a visual, graphical, tabular, matrix or any other depiction of such data. It can be stored as a database or in any other suitable form.

As used herein, a database refers to a collection of data items.

As used herein, a relational database is a collection of data items organized as a set of formally-described tables from which data can be accessed or reassembled in many different ways without having to reorganize the database tables. Such databases are readily available commercially, for example, from ORACLE, IBM, MICROSOFT, SYBASE, COMPUTER ASSOCIATES, SAP, or multiple other vendors. Databases can be stored on computer-readable media, such floppy disks, compact disks, digital video disks, computer hard drives and other such media.

As used herein, an address refers to a unique identifier whereby an addressed entity can be identified. An addressed moiety is one that can be identified by virtue of its address. Addressing can be effected by position on a surface, such as the locus or loci, or by other identifier, such as a tag encoded with a bar code or other symbology, a chemical tag, an electronic, such RF tag, a color-coded tag or other such identifier.

As used herein, a capture system refers to an addressable collection of capture agents and polypeptide-tagged molecules bound thereto, where each different binding partner specifically binds to a different capture agent.

A self-assembling array is an addressable collection of capture agents, where the capture agents specifically bind to predetermined binding partners.

As used herein, a self-assembled array is an array that results when a self-assembling array is combined with molecules or biological particles that are conjugated to binding partners specific for the capture agents in a self-assembling array.

As used herein, the components of a self-assembled array include a self assembling array, and binding partners specific therefor or nucleic acids encoding the binding partners or sequence information for synthesis of the binding partners or nucleic acids encoded thereby, and optionally conjugation reagents.

As used herein, a molecule, such as capture agent, that specifically binds to a polypeptide, such as a polypeptide tagged molecule provided herein, typically has a binding affinity (Kₐ) of at least about 10⁶ I/mol, 10⁷ I/mol, 10⁸ I/mol, 10⁹ I/mol, 10¹⁰ I/mol or greater (generally 10⁸ or greater) and binds generally with greater affinity (typically at least 10-fold, generally 100-fold) to the molecules and biological particles that are to be detected or assessed in the methods that employ the self-assembling arrays. Such a molecule also is referred to herein as a molecule that “specifically binds to” and/or “is specific for” another molecule. For example, a binding partner that specifically binds to a capture agent has an affinity for that capture agent as described above. Thus, affinity refers to the strength of interaction between a capture agent and a binding partner.

As used herein, avidity refers to the total binding strength of a polyvalent antibody with an antigen.

As used herein, specificity (or selectively) with respect to a binding partner tag and a capture agent refers to the greater affinity the tag and capture agent exhibit for each other compared to other molecules and biological particles that are to be arrayed by the self-assembling arrays (capture systems).

As used herein, used to “bind” to a self-assembling array (capture system) means to interact with sufficient affinity to addressably immobilize the bound moiety (biological particle) temporarily under the conditions of a particular experiment. For purposes herein, it is an interaction that permits molecules, such as scFvs, or biological particles, such as cells, to be retained at a locus when the molecules or biological particles are tagged with a binding partner and are then contacted with the self-assembling array (capture systems) so that they no longer move by Brownian motion or other microcurrents in a composition.

As used herein, conjugation refers to the formation of a complex between a binding partner and a molecule and/or biological particle. The binding partner is conjugated to the molecule and/or biological particle with a sufficient K_{d} so that interaction is stable upon binding of the binding partner to the capture agents in the array. Further, the conjugates are such that the binding partners are conjugated to the molecules or biological particles such that the binding partners retain their specificity for their capture agent.

As used herein, a landscape is the information produced or presented on a canvas or array.

As used herein, an addressable collection of anti-tag capture agents (also referred to herein as an addressable collection of capture agents) is a collection of reagents, such as antibodies, enzymes and other such molecules and biological particles, that specifically bind to pre-selected binding partners that contain sequences of amino acids, such as epitopes in antigens, in which each member of the collection is labeled and/or is positionally located to permit identification of the capture agent, such as the antibody, and tag. The addressable collection is typically an array or other encoded collection in which each locus contains capture agents, such as antibodies, of a single specificity and is identifiable. The collection can be in the liquid phase if other discrete identifiers, such as chemical, electronic, colored, fluorescent or other tags are included. Capture agents, include antibodies and other anti-tag receptors. Any moiety, such as a protein, nucleic acid or other such moiety, that specifically binds to a pre-determined sequence of amino acids, such as an epitope, is contemplated for use as a capture agent.

As used herein, an addressable collection of binding sites refers to the resulting sites produced upon binding of the capture agents provided herein to polypeptide-tagged reagents. Each capture agent sorts reagents (such as molecules and biological particles) by virtue of their tags, each tag is linked to a plurality of different molecules, generally polypeptides. As a result, upon sorting,-the capture agent and binding partner reagent form a complex and the resulting complex can bind to further molecules. Since the tagged reagents specific for each capture agent can contain a plurality of different molecules that share the same tag, when bound to a plurality of different capture agents the resulting collection presents a highly diverse collection of binding sites. The collection is addressable because the identity of the tags is known or can be ascertained.

As used herein, binding partner (epitope tag) refers to any molecule that can be conjugated to a displayed molecule and/or biological particle and specifically binds to a capture agent.

As used herein, a polypeptide tag generally refers to a binding partner that contains includes a sequence of amino acids, herein also referred to as an epitope, to which a capture agent, such as an antibody, specifically binds. The epitope can be other than a polypeptide, as long as at least a portion of it specifically binds to a capture agent. Furthermore, the tags (or encoding nucleic acid molecules) can include a plurality of domains, including, but are not limited to a tag-specific amplification sequence (herein referred to as an R-tag) and nucleic acid encoding a ligand-binding domain.

For binding partners, such as polypeptide tags, the specific sequence of amino acids to which each capture agent binds is referred to herein generically as an epitope. Any sequence of amino acids that binds to a receptor (capture agent) therefor is contemplated. For purposes herein the sequence of amino acids of the tag, such as epitope portion of the polypeptide (epitope) tag, that specifically binds to a capture agent is designated “E,” and each unique epitope is an Eₘ.

As used herein, a fusion protein refers to a polypeptide that contains at least two components, such as a biomolecular component of a displayed molecule and a binding partner, such as a polypeptide tag, and is produced by expression of nucleic acid in a host cell.

As used herein, an array refers to a collection of elements, such as antibodies, containing three, four, five, six or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interaction of the molecules or biological particles), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface. Thus, for example, positionally addressable arrays can be arrayed on a substrate, such as glass, including microscope slides, paper, nylon or any other type of membrane, filter, chip, glass slide, or any other suitable solid support. If needed the substrate surface is functionalized, derivatized or otherwise rendered capable of binding to a binding partner. In some instances, those of skill in the art refer to microarrays. A microarray is a positionally addressable array, such as an array on a solid support, in which the loci of the array are at high density. For example, a typical array formed on a surface the size of a standard 96 well microtiter plate (128 mm×86 mm) with 96 loci, 384, or 1536 are not microarrays. Arrays (typically, although not necessarily, on microtiter plate-sized supports) at higher densities such as greater than 2000, 3000, 4000 and more loci per plate (or support) are considered microarrays. Thus, microarrays are high density arrays such that the number of loci per mm² is greater than 0.2 loci/mm², 0.3 loci/mm², 0.35 loci/mm², 0.4 loci/mm² or greater. Any array containing three or more loci in which the loci are at such densities is a microarray.

As used herein, a canvas is a collection of arrays, such as those provided herein. The size of each array and number in a canvas can vary and is at least two arrays per canvas.

As used herein, a support (also referred to as a matrix support, a matrix, an insoluble support or solid support) refers to any solid or semisolid or insoluble support to which a capture agent, typically a molecule, biological particle or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. A support can be of any geometry, including particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads,” are often, but not necessarily, spherical. Such reference, however, does not constrain the geometry of the matrix, which can be any shape, including random shapes, needles, fibers, and elongated. Roughly spherical “beads,” particularly microspheres that can be used in the liquid phase, also are contemplated. The “beads” can include additional components, such as magnetic or paramagnetic particles (see, e.g., Dyna beads® (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be order of cubic microns. Such particles are collectively called “beads.”

As used herein, a capture agent refers to a molecule that has a specificity for other molecules or biological particles, such as a ligand or anything that includes a defined sequence of amino acids. Capture agents can be naturally-occurring or synthetic molecules, and include any molecule, including nucleic acids, small organics, proteins and complexes that specifically bind to other molecules or specific sequences of amino acids. Capture agents also are referred to as receptors and also are referred to in the art as anti-ligands. As used herein, the terms, capture agent, receptor and anti-ligand are interchangeable. Capture agents can be used in their unaltered state or as aggregates with other species. They can be attached or in physical contact with, covalently or noncovalently, a binding member, either directly or indirectly via a specific binding substance or linker. Examples of capture agents, include, but are not limited to: antibodies, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, and organelles. For example, the capture agents can specifically bind to DNA binding proteins, such as zinc fingers, leucine zippers and modified restriction enzymes.

Examples of capture agents, include but are not limited to:

a) enzymes and other catalytic polypeptides, including, but not limited to, portions thereof to which substrates specifically bind, enzymes modified to retain binding activity but lacking catalytic activity;

b) antibodies and portions thereof that specifically bind to antigens or sequences of amino acids;

c) nucleic acids;

d) cell surface receptors, opiate receptors and hormone receptors and other receptors that specifically bind to ligands, such as hormones. For the collections herein, the other binding partner, referred to herein as a binding partner for each refers the substrate, antigenic sequence, nucleic acid binding protein, receptor ligand, or binding portion thereof.

As noted, contemplated herein, are pairs of molecules, generally proteins that specifically bind to each other. One member of the pair can be a molecule, such as polypeptide, which can be conjugated to a displayed molecule and/or biological particle, that is used as a tag and the other member is any molecule that specifically binds thereto. The collections of capture agents, such as antibodies or enzymes or portions thereof and mixtures thereof, that specifically bind to a target molecule or a known or knowable defined sequence of amino acids that is typically at least about 3 to 10 amino acids in length. Other examples of capture agents are set forth throughout the disclosure.

As used herein, a molecule refers to any compound found in nature, or derivatives thereof, including but not limited to, biopolymers, biomolecules, macromolecules or components or precursors thereof, such as peptides, proteins, organic compounds, oligonucleotides or monomeric units of the peptides, organics, nucleic acids and other macromolecules. A monomeric unit refers to one of the constituents from which the resulting compound is built. Thus, monomeric units include, nucleotides, amino acids, and pharmacophores from which small organic molecules are synthesized.

As used herein, a conjugate or cross-linked complex refers to a complex between a binding partner and a molecule and/or biological particle. The binding partner is conjugated to the molecule and/or biological particle with a sufficient K_{d} so that interaction is stable upon binding of the binding partner to the capture agents in the array. Further, the conjugates are such that the binding partners are conjugated to the molecules or biological particles such that the binding partners retain their specificity for their capture agent.

As used herein, a conjugation reagent, such as a bifunctional or trifunctional reagent, refers to any chemical or biological compound or molecule that assists in the conjugation of two or more molecules. The conjugation reagent, such as a binfunctional or trifunctional reagent, can be part of the linkage between the two or more molecules or facilitate the conjugation without becoming a physical part of the linkage.

As used herein, a heterobifunctional reagent facilitates the conjugation of two different functional groups, such as a thiol and an amine. As used herein, a homobifunctional reagent facilitates the conjugation of two identical functional groups, such as two amines or two thiols. As used herein, a trifunctional reagent facilitates the conjugation of three or more identical or different functional groups.

As used herein, printing refers to immobilization of capture agents onto a solid support, such as, but not limited to, an array.

As used herein, self-sorting refers to separation of various epitope-tagged molecule(s) based on the affinity of the epitope for a specific capture agent.

As used herein, the total display refers to the total diversity of molecules being displayed on the arrays.

As used herein, a B cell refers to a lymphocyte that develops from hemopoietic stem cells in the bone marrow of adults and the liver of fetuses and is responsible for the production of circulating antibodies.

As used herein, a T cell refers to a lymphocyte that develops in thymus from precursor cells that migrate .there from the hemopoietic tissues via the blood. T cells fall into two main classes, cytotoxic T cells and helper T cells. Cytotoxic T cells kill infected cells, whereas helper T cells help to activate macrophages, B cells and cytotoxic T cells.

As used herein, antibody refers to an immunoglobulin, whether natural or partially or wholly synthetically, such as recombinantly, produced, including any derivative thereof that retains the specific binding ability of the antibody. Hence, antibody includes any protein having a binding domain that is homologous or substantially homologous to an immunoglobulin binding domain. For purposes herein, antibody includes antibody fragments, such as Fab fragments, which are composed of a light chain and the variable region of a heavy chain. Antibodies include members of any immunoglobulin class, including IgG, IgM, IgA, IgD and IgE. Furthermore, for purposes herein, the capture agents, such as antibodies, can be binding portions thereof.

Hence for purposes herein, any set of pairs of binding members, referred to generically herein as a capture agent/binding partner, can be used instead of antibodies and epitopes per se. The methods, combinations and kits herein include sets of capture agent/binding partner pairs, such as an antibody/binding partner pair, and rely on their specific interactions for the immobilization and sorting of various displayed molecules or biological particles. As such, any combination of capture agents and binding partners (e.g., receptors/ligands) can be used. Also contemplated herein are receptors that specifically binding to a sequence of amino acids.

As used herein, a monoclonal antibody refers to an antibody secreted by a hybridoma clone. Because each such clone is derived from a single B cell, all of the antibody molecules are identical. Monoclonal antibodies can be prepared using standard methods known to those with skill in the art (see, e.g., Kohler et al. Nature 256:495 (1975) and Kohler etal. Eur. J. Immunol. 6: 511 (1976)). For example, an animal is immunized by standard methods to produce antibody-secreting somatic cells. These cells are then removed from the immunized animal for fusion to myeloma cells.

Somatic cells with the potential to produce antibodies, particularly B cells, are suitable for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511 (1976); Shulman etal. Nature 276: 269 (1978); Volk et al. J. Virol. 42:220 (1982)). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumor cells to produce their own antibodies. The purpose of using monoclonal techniques is to obtain fused hybrid cell lines with unlimited life spans that produce the desired single antibody under the genetic control of the somatic cell component of the hybridoma. To eliminate the production of tumor cell antibodies by the hybridomas, myeloma cell lines incapable of producing endogenous light or heavy immunoglobulin chains are used. A third reason for selection of these cell lines is for their suitability and efficiency for fusion. Other methods for producing hybridomas and monoclonal antibodies are well known to those of skill in the art.

As used herein, antibody fragment refers to any derivative of an antibody that is less than full length, retaining at least a portion of the full-length antibody's specific binding ability. Examples of antibody fragments include, but are not limited to, Fab, Fab′, F(ab)₂, single-chain Fvs (scFv), Fv, dsFv, diabody and Fd fragments. The fragment can include multiple chains linked together, such as by disulfide bridges. An antibody fragment generally contains at least about 50 amino acids and typically at least 200 amino acids.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, a F(ab)₂ fragment is an antibody fragment that results from digestion of an immunoglobulin with pepsin at pH 4.0-4.5; it can be recombinantly produced.

As used herein, a Fab fragment is an antibody fragment that results from digestion of an immunoglobulin with papain; it can be recombinantly produced.

As used herein, scFvs refer to antibody fragments that contain a variable light chain (V_{L}) and variable heavy chain (V_{H}) covalently connected by a polypeptide linker in any order. The linker is of a length such that the two variable domains are bridged without substantial interference. Exemplary linkers are (Gly-Ser)n residues with some Glu or Lys residues dispersed throughout to increase solubility.

As used herein, hsFv refers to antibody fragments in which the constant domains normally present in an Fab fragment have been substituted with a heterodimeric coiled-coil domain (see, e.g., Arndt et al. (2001) J Mol Biol. 7:312:221-228).

As used herein, diabodies are dimeric scFv; diabodies typically have shorter peptide linkers than scFvs, and they preferentially dimerize.

As used herein, humanized antibodies refer to antibodies that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, idiotype refers to a set of one or more antigenic determinants specific to the variable region of an immunoglobulin molecule.

As used herein, anti-idiotype antibody refers to an antibody directed against the antigen-specific part of the sequence of an antibody or T cell receptor. In principle, an anti-idiotype antibody inhibits a specific immune response.

As used herein, phage display refers to the expression of proteins or peptides on the surface of filamentous bacteriophage.

As used herein, panning refers to an affinity-based selection procedure for the isolation of phage displaying a molecule with a specificity for a desired capture molecule or epitope.

As used herein, a candidate compound refers to any compound identified by the screening methods provided herein and refers to any compound that modulates molecular interactions and can be a candidate for use as a therapeutic or as a lead compound for the design of a therapeutic. Such compounds, include but are not limited to, small molecules, including small organic molecules, peptides, peptide mimetics, antisense molecules or dsRNA, such as RNAi, antibodies, fragments of antibodies, recombinant antibodies and other such compounds that can serve as drug candidates or lead compounds.

As used herein, high-throughput screening (HTS) refers to processes that test a large number of samples, such as samples of candidate compounds that are candidate modulators of interactions between nucleic acids and proteins. HTS operations are amenable to automation and are typically computerized to handle sample preparation, assay procedures and the subsequent processing of large volumes of data.

As used herein, a condition refers to any variable experimental or environmental parameter including, but not limited to, buffer or solution components, pH, temperature, exposure to light, aerobic or anaerobic conditions, concentration of components, duration of experimental detection, ionic strength, pressure, agitation, and organic or aqueous interaction medium.

As used herein, a perturbation refers to any input that modulates an interaction between or among molecular and/or biological particle components of a target interaction. Perturbations include, but are not limited to, compounds that modulate such interactions, such as small effector molecules, including, for example, small organics, other effector molecules, antisense oligonucleotides, double-stranded RNA, nucleic acid molecules, polypeptides, and conditions and alterations thereof, such as pH, ionic strength, temperature, conductivity, anaerobic conditions, aerobic conditions, concentration, time, pressure and light or the absence thereof and other conditions.

As used herein, staining refers to the visualization of molecules bound to the self-assembled self-assembling array. Staining can be non-specific, semi-specific or specific depending on what is labelled in a sample and when it is detected. Non-specific staining refers to the labelling of non-fractionated or all components in a particular sample generally, although not necessarily, prior to exposure to the self-assembling array (capture system). Semi-specific staining as used herein refers to labelling of a portion of a sample, such as, but not limited to, the proteins located on the cell surface or on cellular membranes, either before, during or after exposure to the self-assembling array (capture system). Specific staining as used herein refers to the labelling of a specific component of a sample, typically after the exposure of the sample to the self-assembled array (capture system). The stain can be any molecule that associates with that permits visualization or detection of bound molecules.

As used herein, attachment refers to the attachment of a label to a molecule and/or biological particle. The attachment can include, but is not limited to, covalent attachment, an affinity interaction, hybridization, electrostatic interaction and an operably linked macromolecule, such as a fusion protein.

As used herein, a label is a detectable marker that can be attached or linked directly or indirectly or associated with a molecule and/or biological particle. The detection method can be any method known in the art.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for example, the biological activity of a luciferase is its oxygenase activity whereby, upon oxidation of a substrate, light is produced.

As used herein, functional activity refers to a polypeptide or portion thereof or to a compound that displays one or more activities associated with a full-length protein or compound. Functional activities include, but are not limited to, biological activity, catalytic or enzymatic activity, antigenicity (ability to bind to or compete with a polypeptide for binding to an anti-polypeptide antibody), immunogenicity, ability to form multimers, and the ability to specifically bind to a receptor or ligand for a polypeptide.

As used herein, a modulator is any molecule or condition that alters an interaction or reaction between or among molecules

As used herein, an inhibitor is any molecule or condition that inhibits an interaction or reaction between or among molecules.

As used herein, an enhancer is any molecule or condition that enhances an interaction or reaction between or among molecules.

As used herein, a subcellular compartment or an organelle is a membrane-enclosed compartment in a eukaryotic cell that has a distinct structure, macromolecular composition, and function. Organelles include, but are not limited to, the nucleus, mitochondria, chloroplast, and Golgi apparatus.

As used herein, screening refers to a process for analyzing molecules and/or biological particles, such as sets of molecules and library compounds, by methods that include, but are not limited to, ultraviolet-visible (UV-VIS) spectroscopy, infra-Red (IR) spectroscopy, fluorescence spectroscopy, fluorescence resonance energy transfer (FRET), NMR spectroscopy, circular dichroism (CD), mass spectrometry, other analytical methods, high throughput screening, combinatorial screening, enzymatic assays, antibody assays and other biological and/or chemical screening methods or any combination thereof.

As used herein, in silico refers to research and experiments performed using a computer. In silico methods include, but are not limited to, molecular modelling studies, biomolecular docking experiments, and virtual representations of molecular structures and/or processes, such as molecular interactions.

As used herein, cell capture refers to the immobilization of a cell by a self-assembled array (capture system) provided herein.

As used herein, biological sample refers to any sample obtained from a living or viral source and includes any cell type or tissue of a subject from which nucleic acid or protein or other macromolecule can be obtained. The biological sample can be a sample obtained directly from a biological source or processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants. Also included are soil and water samples and other environmental samples, viruses, bacteria, fungi algae, protozoa and components thereof. Hence bacterial and viral and other contamination of food products and environments can be assessed. The methods herein are practiced using biological samples and in some embodiments, such as for profiling, also can be used for testing any sample.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, the term “biopolymer” is a biological molecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein, respectively. Biopolymer include, but are not limited to, nucleic acid, proteins, polysaccharides, lipids and other macromolecules. Nucleic acids include DNA, RNA, and fragments thereof. Nucleic acids can be derived from genomic DNA, RNA, mitochondrial nucleic acid, chloroplast nucleic acid and other organelles with separate genetic material.

As used herein, a biomolecule is any compound found in nature, or derivatives thereof. Biomolecules include but are not limited to: oligonucleotides, oligonucleosides, proteins, peptides, amino acids, peptide nucleic acids (PNAs), oligosaccharides and monosaccharides.

As used herein, a biological particle refers to any portion of a living organism or a virus or other such agent and includes, but is not limited to, a virus, such as a viral vector or viral capsid with or without packaged nucleic acid, phage, including a phage vector or phage capsid, with or without encapsulated nucleic acid, a single cell, including eukaryotic and prokaryotic cells or fragments thereof, a liposome or micellar agent or other packaging particle, a prion and other such biological materials.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well known methods (see, for example, Weiler et al. Nucleic acids Res. 25: 2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, production by recombinant means by using recombinant DNA methods means the use of the well known methods of molecular biology for expressing proteins encoded by cloned DNA.

As used herein, substantially identical to a product means sufficiently similar so that the property of interest is sufficiently unchanged so that the substantially identical product can be used in place of the product.

As used herein, equivalent, when referring to two sequences of nucleic acids, means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 1, below) that do not substantially alter the activity or function of the protein or peptide. When “equivalent” refers to a property, the property does not need to be present to the same extent but the activities are generally substantially the same. “Complementary,” when referring to two nucleotide sequences, means that the two sequences of nucleotides are capable of hybridizing, generally with less than 25%, with less than 15%, and even with less than 5% or with no mismatches between opposed nucleotides. Generally to be considered complementary herein are two molecules which hybridize under conditions of high stringency.

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1×SSPE or SSC, 0.1% SDS, 65° C.

2) medium stringency: 0.2×SSPE or SSC, 0.1% SDS, 50° C.

3) low stringency: 1.0×SSPE or SSC, 0.1% SDS, 50° C.

Equivalent conditions refer to conditions that select for substantially the same percentage of mismatch in the resulting hybrids. Additions of ingredients, such as formamide, Ficoll, and Denhardt's solution affect parameters such as the temperature under which the hybridization is conducted and the rate of the reaction. Thus, hybridization in 5×SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al., vol. 3, p. B.13, see, also, numerous catalogs that describe commonly used laboratory solutions). It is understood that equivalent stringencies can be achieved using alternative buffers, salts and temperatures.

The term “substantially” identical or homologous or similar varies with the context as understood by those skilled in the relevant art and generally means at least 70%, at least 80%, at least 90%, and at least 95% identity.

As used herein, a reporter gene construct is a nucleic acid molecule that includes a nucleic acid encoding a reporter operatively linked to a transcriptional control sequences. Transcription of the reporter gene is controlled by these sequences. The activity of at least one or more of these control sequences is directly or indirectly regulated by a cell surface protein or other protein that interacts with tagged molecules or other molecules in the self-assembled array (capture system). The transcriptional control sequences include the promoter and other regulatory regions, such as enhancer sequences, that modulate the activity of the promoter, or control sequences that modulate the activity or efficiency of the RNA polymerase that recognizes the promoter, or control sequences are recognized by effector molecules, including those that are specifically induced by interaction of an extracellular signal with a cell surface protein. For example, modulation of the activity of the promoter can be effected by altering the RNA polymerase binding to the promoter region, or, alternatively, by interfering with initiation of transcription or elongation of the mRNA. Such sequences are herein collectively referred to as transcriptional control elements or sequences. In addition, the construct can include sequences of nucleotides that alter translation of the resulting mRNA, thereby altering the amount of reporter gene product.

As used herein, staining or labeling refers to moieties used to visualize or detect biological particles or molecules.

As used herein, “reporter” or “reporter moiety” refers to any moiety that allows for the detection of a molecule of interest, such as a protein expressed by a cell, or a biological particle. Typical reporter moieties include, include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene”, can be expressed as a fusion protein with a protein of interest or under to the control of a promoter of interest.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous, rather two or more components are juxtaposed so that the components are in a relationship permitting them to function in their intended manner. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases. Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune-modulation. Regulatory regions typically bind one or more trans-acting proteins which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to an including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, regulatory molecule refers to a polymer of deoxyribonucleic acid (DNA) or ribonucleic acid (RNA), or an oligonucleotide mimetic, or a polypeptide or other molecule that is capable of enhancing or inhibiting expression of a gene.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. Typically, the items of a combination can be used together in the steps of a method, associated together in a kit to practice a method or represent some associated function or use.

As used herein, a kit refers to a packaged combination. A packaged combination, optionally including a label or labels, instructions and/or reagents for their use or for use of the combination.

As used herein, packaging material means any material known to those of skill in the art that can be used for packaging pharmaceutical products and/or reagents for use in research and manufacture, such as reagents for chemical reactions, biological reactions, high throughput screening. Exemplary packaging material includes, but is not limited to, containers, vials, blister packs, bottles, tubes, inhalers, pumps, bags, tubes and any container means and wrapping for holding or containing the components of a kit or combination. As used herein, packaging material is material that can be used to package the kits and combinations described herein. Such packaging material can include, but is not limited to, ice, dry ice, styrofoam, foam, plastic, cellophane, shrink wrap, bubble wrap, paper, cardboard, starch peanuts, twist ties, metal clips, metal cans, drierite, glass, and rubber. (see products listed at www.papermart.com. for examples of packaging material).

As used herein, “a computer-based system” refers to the hardware, software, and data storage media and methods used to analyze array image data. The minimum hardware of the computer-based systems provided herein include a central processing unit (CPU), input mean, output means and data storage means. A skilled artisan can select a suitable computer-based systems for use in the methods and systems provided herein.

As used herein, “recorded” refers to a process for storing information on computer readable medium. A skilled artisan can readily adopt any of the presently known methods for recording information on computer readable medium to array image data. The choice of the data storage structure can generally be based on the media and platforms chosen to access the stored information. In addition, a variety of data processor programs and formats can be used to store the array image information on computer readable medium. The image information can be represented in a word processing text file, formatted in commercially-available software such as MICROSOFT Word®, graphics files or represented in the form of an ASCII file, stored in a database application, such as DB2®, Sybase® and Oracle®. A skilled artisan can adapt any number of data processor structuring formats (e.g., text file or database) in order to obtain computer readable medium having recorded thereon the information as described herein.

As used herein, fluid refers to any composition that can flow. Fluids thus encompass compositions that are in the form of semi-solids, pastes, solutions, aqueous mixtures, gels, lotions, creams and other such compositions.

As used herein, antigenic means that a polypeptide induce an immune response. Highly antigenic polypeptides are those that reproducibly and predictably induce an immune response.

As used herein, antigenic ranking refers to a statistical probability that an amino acid or set thereof occurs in an antigenic polypeptide, including epitopes in naturally occurring polypeptides.

As used herein, highly antigenic, highly specific polypeptides (HAHS) mean polypeptides that specifically bind to a capture agent and that are antigenic such that specifically binding capture agents are readily designed or prepared. For example, the polypeptides that result from application of the methods raise or produce high titer antiserum in rodents, such as mice. Hence methods readily produce pairs of polypeptides (the highly antigenic highly specific polypeptides) and capture agents.

As used herein, a similarity ranking refers to a comparison among amino acids and is represented or determined as a probability or fraction that two amino acids are structurally and/or functionally similar. For example, two identical amino acids have a similarity ranking of 100; two very dissimilar amino acids, such as proline and tyrosine have a ranking of 0.

As used herein, a subset of a set contains at least one less member than the set.

As used herein, a critical residue or amino acid in an HAHS polypeptide is one that influences the affinity or specificity of binding to the binding protein (capture agent). Critical residues taken from the set of naturally occurring amino acids can only be replaced by a subset of amino acids (usually 1 or 2 amino acids) or in some cases, can not be replaced by any other amino acid from this set.

As used herein, a non-critical residue or amino acid in an HAHS polypeptide is one that does not influence the affinity or specificity of binding to the binding protein (capture agent). Noncritical residues can be replaced by a larger subset of amino acids (for example, when taken from the set of naturally occurring amino acids, they can be replaced usually 10 or more amino acids or in some cases, by any other amino acid from this set) without affecting the affinity or specificity of binding. In some cases, non-critical residues are used to confer additional functionalities or properties on polypeptides. In this case, they can typically only be replaced by a limited number of amino acids to retain the functionality or property.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions can be made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, an amino acid is an organic compound containing an amino group and a carboxylic acid group. A polypeptide comprises two or more amino acids. For purposes herein, amino acids include the twenty naturally-occurring amino acids non-natural amino acids, and amino acid analogs. These include amino acids wherein a-carbon has a side chain.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

As used herein, neighbor luminosity refers to the illumination contribution from neighboring loci in arrays that enters the imaging device field of view of a particular locus. The arrays are labeled or processed arrays in which certain loci are detectable labeled.

As used herein, an exposed self-assembled array is a self-assembled array that as been used for testing or an experiment or screening or diagnosing/prognosticating. Thus, the self assembled array, has for example, been reacted with molecule(s), biological particle(s) or subjected to a perturbation, such as a candidate compound or a condition, such that a detectable reaction occurs, whereby loci can be detected.

As used herein, an exposed locus is a locus on a self-assembled array one that is detectably labeled, such as with a luminescent label, and is detected following exposure of the self-assembled array to a target molecule(s), biological particle(s) or a perturbation, such as a candidate compound or a condition.

The methods, combinations, kits and systems herein are described and exemplified with particular reference to antibodies capture agents, and binding partners that include sequences of amino acids to which the antibodies bind, but is it to be understood that the methods herein can be practiced with any capture agent and any binding partner therefor. It also to be understood that combinations of collections of any capture agents and binding partners therefor are contemplated for use in any of the embodiments described herein. It also is to be understood that reference to an array is intended to encompass any addressable collection, whether it is in the form of a physical (solid phase) array or a labeled collection, such as capture agents bound to colored beads.

### B. Components of Self-Assembling Arrays

Provided herein are methods, combinations, kits and systems for preparing and using self-assembling arrays. The self-assembling arrays include arrays of capture agents to which known and available binding partners specifically bind, and binding partners that specifically bind to each capture agent. The arrays of capture agents can include loci that bind to controls and/or that serve as control loci.

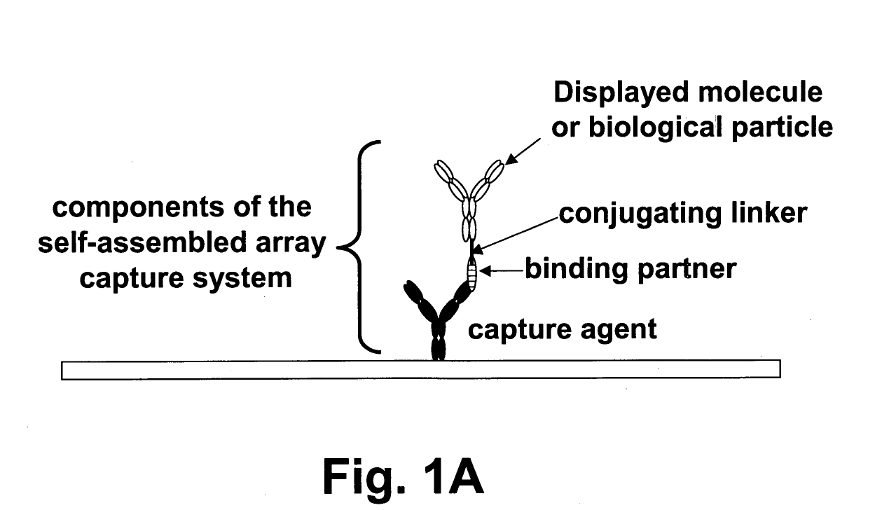

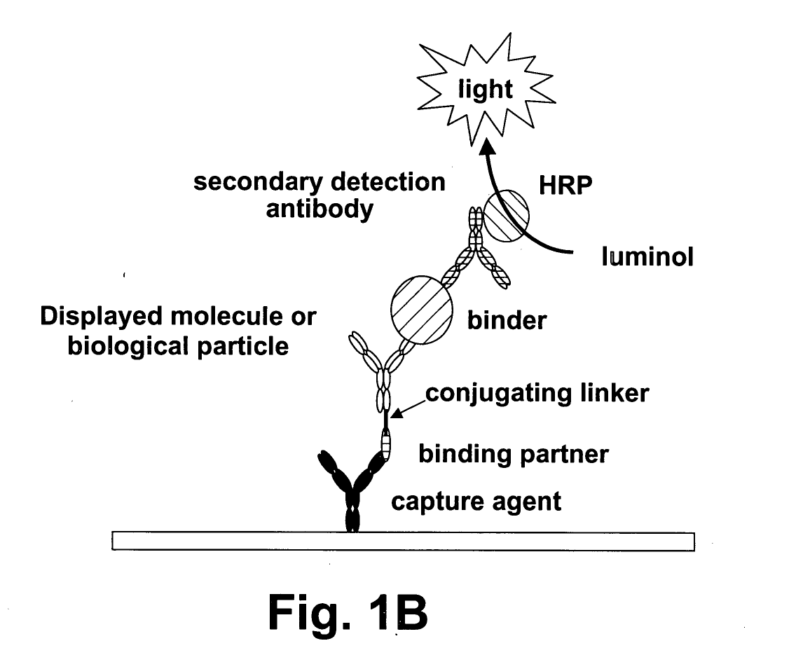

The binding partners are linked, such as cross-linked, to molecules andor biological particles that are to be displayed. After linking the molecule and/or biological particle to each binding partner to produce conjugates, the resulting conjugates are contacted with the collection of capture agents to produce self-assembled arrays that display the molecules and/or biological particles. An exemplary self-assembling array is shown in FIG. 1.

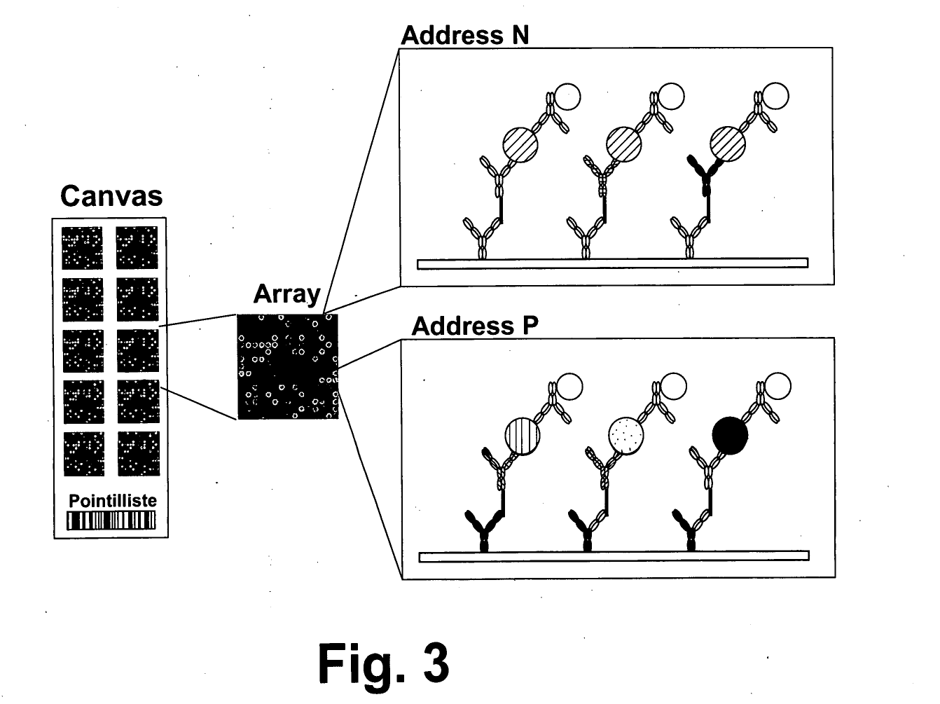

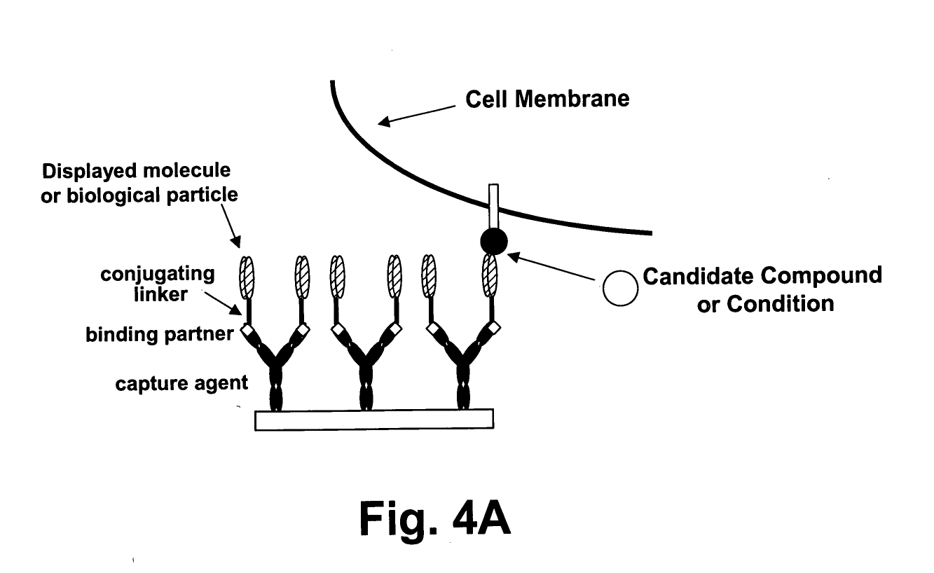

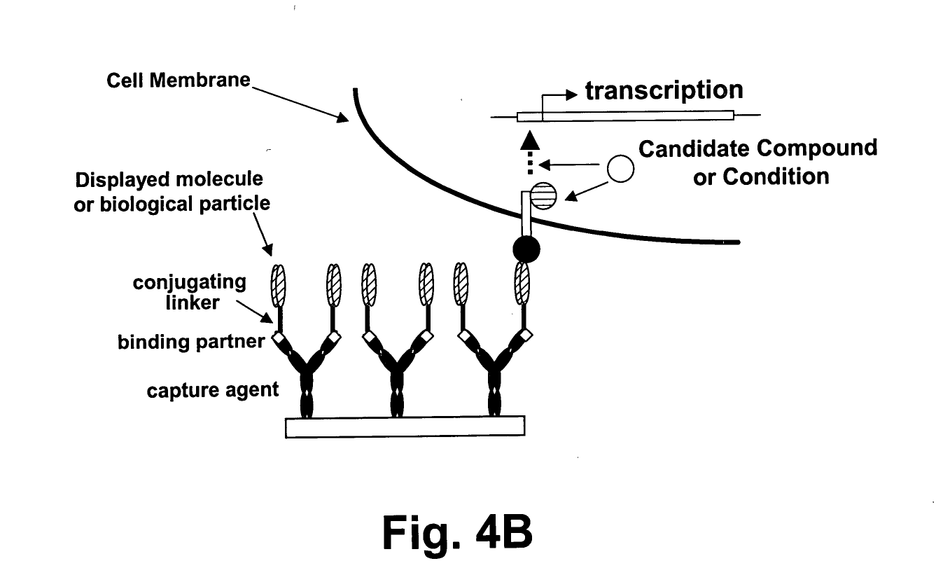

The self-assembled arrays have a variety uses and applications, including, but not limited to, detecting interactions, such as biological and chemical interactions, and/or activities and other events, which can occur simultaneously or sequentially, among a plurality of molecules and/or biological particles. Hence, also provided are methods, combinations, kits and systems for preparing and using a self-assembling array to produce self-assembled arrays for monitoring interactions among a plurality of molecules and/or biological particles in which one or more different events, such as reactivity to a drug compound or binding interaction, that occur at various addressable loci within the array, simultaneously or sequentially are monitored and/or distinguished (see, for example, FIGS. 1 and 3). In addition, methods, combinations, kits and systems to produce self-assembled arrays that permit identification of perturbations, such as candidate compounds and conditions, that modulate such interactions are provided (see, for example, FIGS. 4A and 4B).

For example, the methods, combinations, kits and systems that include self-assembled arrays provided herein can be used to elucidate biochemical pathways, diseases processes and many other processes and reactions, definition of the function of an inhibitor or an enhancer within a molecular system, understanding of receptor-signal recognition and interactions, and study of antibody-antigen recognition, hybridization of nucleic acids and heterogeneous or homogeneous complex formation from multiple components.

Practice of the methods provided herein involves some or all of the following steps: (1) identifying and obtaining a plurality of molecules and/or biological particles for display; (2) conjugating each of the molecules or biological particles to different binding partners to produce conjugates; (3) contacting the conjugates with an array containing addressable capture agents to which the binding partners bind, thereby arraying the conjugates by virtue of interaction between the capture agents located at defined loci within the array and the binding partner in the conjugates to display the biological particles and/or molecules. Each type of capture agent interacts specifically with a particular binding partner to produce a self-assembled array. The resulting self-assembled arrays can be used in any experiment or for any purpose in which an array is employed. For example, the methods can include contacting the arrayed displayed molecules and/or biological particles with an identical or different target molecule and/or biological particle; and (4) detecting the resulting interaction (or lack of interaction) or the effect of the interaction. Optionally, the some or all of following additional steps can be performed: (5) identifying a perturbation, such as a candidate compound and/or a condition, of the interaction or effect of the interaction; (6) exposing the interaction to a perturbation; and (7) detecting and/or monitoring the interaction or effect of the interaction in the presence of the perturbation. These optional steps can be performed before, after or during any of the steps, such as after step (3) or step (4), or after any other steps in such method. Other optional additional steps include labelling one or more of the displayed molecules or biological particles, the target molecule and/or biological particle and the candidate compound. Screening can also be performed before or after any of steps of the methods. Further, the steps of the methods of detecting and/or monitoring an interaction or the effect of an interaction provided herein can be used iteratively. An interaction, an effect of an interaction or a perturbation identified by the methods herein can be again subjected to some or all of the above noted steps to further identify interactions, effects of interactions or perturbations.

In practice, to begin the method, molecules and/or biological particles (i.e., moieties of interest) that are to be displayed or arrayed are identified or otherwise selected. Displayed molecules and/or biological particles also include complexes of molecules and/or biological particles with other moieties. Selection of molecules and/or biological particles depends upon the application of the resulting self-assembled arrays, which can be used, for example, for assessing and/or detecting one or more known or unknown events, which can occur simultaneously or sequentially, and for a variety of other applications. The molecules and/or biological particles of interest can be identified by any method known to those of skill in the art, including from sources described herein, other methods described herein and by methods apparent to those skilled in the art based upon the description herein. For example, databases of literature, molecules and biological particles can be mined randomly for target interactions of interest. Empirical methods can also be employed for the identification of displayed molecules or biological particles. A displayed molecule and/or biological particle can be selected based on a variety of criteria, including, but not limited to, availability, cost, improving the understanding of the problem to be solved and applicability to a larger system. Other criteria for the selection of a displayed molecule and/or biological particle is described herein, and apparent to those skilled in the art based description herein. Generally the selection and/or identification of molecules and/or biological particles is up to the user of the self-assembling arrays and components thereof.

Following identification or selection, chosen displayed molecules and/or biological particles are obtained. The number of molecules and biological particles selected can vary depending on the size of the array and the number of capture agent/binding partner pairs available. A displayed molecule and/or biological particle can be identified and obtained by a variety of methods, including, but not limited to, isolation from complex mixtures, commercial sources, other methods described herein and by methods apparent to those with skill in the art based upon the description herein. For example, databases of biomolecules can be mined for displayed molecules, such as, but not limited to a specific protein, nucleic acid, antibody, virus, cell, and enzyme.

Once sets of displayed molecules or biological particle of interest are obtained, each set is conjugated to a binding partner, including, but not limited to, a peptide, a protein or an antibody or other such moiety. Generally, although not necessarily, each set of molecules is conjugated to a different binding partner. The molecule and/or biological particle is conjugated such that the aspect that makes them of interest, such as their 3-D structure or biological activity, is not altered Further, the molecule and/or biological particle is conjugated with a binding partner that is specific for a capture agent that is addressable within the array. Optionally, the molecule and/or biological particle of interest can be labelled with a detectable label, such as a luminescent label, to permit or provide for detection of the displayed molecule and/or biological particle within an addressable array.

The conjugated displayed molecules or biological particles are then contacted with the addressably arrayed capture agents, such as capture agents printed on a solid support, that interact with the binding partner. Contact of the conjugated molecule and/or biological particle with the array can be performed, for example, individually or as a batch sample under conditions whereby the binding partners bind to capture agents. Particular conditions for capture depend upon the type of capture agents, binding partners and molecules or biological particles conjugated thereto. Such conditions are standard, such as those for forming complexes between antibodies and antigens, and/or can be empirically determined. Within the array, the conjugated molecule and/or biological particles typically, although not necessarily, are different at each locus. Optionally, a perturbation, such as a candidate compound or a condition, can be added to the array prior to, simultaneously with or following contacting the conjugated molecule and/or biological particle with the capture agents.

Once the conjugated displayed molecule and/or biological particle is sorted onto the array, one or more additional molecules or biological particles, including, but not limited to, a drug, an antibody or a protein co-factor, can be added and its interaction with the conjugated displayed molecules or biological particles assessed. This interaction can be assessed by any method known to those skilled in the art, including, but not limited to, detection of a secondary antibody; a conformational change; a binding interaction; complexation; hybridization; transfection; hydrophobic interaction; signal transduction; membrane translocation; electron transfer; conversion of a reactant to a product via a catalytic mechanism; chaperoning of compounds inter- and intracellularly; fusion of liposomes to membranes; infection of a foreign pathogen into a host cell or organism, such as a virus (HIV, influenza virus, polio virus, adenovirus, etc.) or bacteria (Escherichia coli, Pseudomonas aeruginosa, Salmonella enteritidis, etc.); initiation of a regulatory cascade, detoxification of cells and organisms; and cell replication and division.

Methods provided herein can be used for a variety of purposes including, but not limited to, drug screening and interaction assessment. In methods provided herein directed to drug screening, the displayed interaction is known and perturbations are screened to identify candidate compounds and/or conditions that modulate the interaction among components of the target interaction. In methods herein directed to assessment of unknown molecular and/or biological particle interactions, the effect of a perturbation on a specific interaction or specific events is predetermined or preidentified, and any effect of the perturbation on unknown interactions or events can be used to identify the interaction or events in question. Thus, in an optional step of the methods provided herein, candidate compounds or reaction conditions are altered to identify perturbations that modulate the interaction or activity of the displayed molecule and/or biological particle with a target molecule and/or biological particle.

Candidate compounds and conditions can be identified from any source, including, but not limited to, commercial databases, literature research, empirical methods, other methods described herein and by methods apparent to those with skill in the art based upon the description herein. Candidate compounds and conditions can have a known or unknown effect on the interaction or activity. Further, the candidate compounds and conditions can be added prior to, simultaneously with, or after contacting the sorted conjugated displayed molecule and/or biological particle with a target molecule and/or biological particle. Additionally, the candidate compound can be labeled with a detectable label, including, but not limited to, a luminescent label and a secondary antibody. A label on the candidate compound can be of the same class (e.g., all luminescent labels) as those on the displayed molecule and/or biological particle or can of a different class (e.g., luminescent labels on the displayed molecule and/or biological particle and a secondary antibody on the candidate compound). Further, a candidate compound can be labeled with a detectable label either in addition to or in the place of the labelled displayed molecule and/or biological particle of interest. Reactions can be performed singly, such as sequentially or simultaneously or in parallel in high throughput formats.

A detailed description of the exemplary components used in the combinations, kits, methods and systems is set forth below and is exemplified with reference to protein/protein interactions. It is understood that the scope of the disclosure is not limited to the exemplified embodiments, that steps can be optional and performed sequentially or simultaneously or in a different order, that components described with reference to one step can be used in other steps of the methods and that other techniques for performing aspects of the methods can be substituted.

1. The Capture Agent Array

The self-assembling arrays provided herein contain a collection different capture agents, such as antibodies that bind to pre-selected and/or pre-designed binding partners, such as a peptide, with high affinity and specificity (see, for example, FIG. 3). A typical collection contains at least about 10, 20, 30, 50, 100, 500, 1000 or more capture agents that are addressable, such as by occupying a unique locus on a solid support, or by virtue of linkage to a detectable label or tag, such as linkage to a bar-coded support or RF-tag labeled support, a color-coded support or other such addressable format. Each locus contains a single type of capture agent that binds to a single specific binding partner. Prior to formation of the self-assembled array, each binding partner is conjugated to sets of displayed molecules and/or biological particles (displayed moieties; i.e., the moieties for whom arrays is desired). Generally each binding partner is conjugated to a different moiety. The resulting conjugate is contacted with the arrayed capture agents, such as capture agents printed on a solid support, under conditions whereby the binding partners in the conjugates bind to their cognate capture argent, thereby arraying the conjugated moiety.

For positionally addressable arrays, supports for use in the self-assembled arrays as described herein are such that the capture agents can be placed on the support material in discrete, addressable loci. Each locus contains a one or a multiplicity of capture agents with a single specificity. Each locus is of a size suitable for detection, such as on the. order of 0.5 to 500, 0.5 to 200, 0.5 to 100, 0.5 to 50, 0.5 to 10 microns, 1 to 5 microns, typically, about 50-300 microns, 100 to 150 microns, such as 1.30 or 280 microns. The particular size of the locus depends upon the intended application or use. In some instances, smaller loci are desired, in other cases larger loci are desirable. If needed, such size can be empirically determined.

In preparing the arrays, a sufficient amount of capture agents is delivered to the surface to functionally cover it. Generally the volume of capture agent-containing mixture delivered for preparation of the arrays depends upon the size of the loci. For example, where loci are about 100-200 microns, about 1 nanoliter is delivered. In particular, delivery of about 1 nanoliter results in a locus that has a diameter of about 1 30 microns, delivery of 4 nanoliters results in locus that has a diameter of about 280-300 microns. The exact size that results depends upon a variety of factors, including but limited to properties of the solution, such as viscosity, and properties of the surface. Volumes smaller than a nanoliter, such as about 50 to about 200 picoliters also can be employed. This amount can be very roughly about 10 million to 100,000 molecules per locus, where each locus has a plurality of capture agents that recognize a single binding partner. The size of the array and each locus should be such that positive reactions in the screening step can be imaged, generally by imaging an entire array or a plurality thereof, such as at least 5, 10, 12, 15, 20, 24, 50, 96, 100, 150, 200, 250 and more arrays, at the same time.

a. Printing the Array

A support (see below for exemplary supports), such as KODAK paper plus gelatin or other suitable matrix can be used, and then ink jet and stamping technology can be used to print the arrays reproducibly. The arrays are printed with, for example, a piezo or inkjet printer, a pintool, or other such nanoliter or lower dispensing device. For example, arrays with 1000 spots can be printed. A plurality of replicate arrays, such as 24 or 48 or more can be placed on a sheet the size of a conventional 96 well plate.

Among the embodiments contemplated herein are sheets of arrays, such as functionalized or derivatized plastic, each with replicates of the capture agent array. These are prepared using piezo or inkjet dispensing system. A large number, for example, 1000 can be printed at a time using, for example, a print.head with 1000 different holes (such as a stamp with 500 μM holes). It can be fabricated from, for example, molded plastic with a lot of holes, such as 1000 holes each filled with 1000 different antibodies. Each hole can be linked to reservoirs with are linked to conduits of decreasing size and ultimately into the head. Each array on the sheet can be spatially separated, and/or separated by a physical barrier, such as a plastic ridge, or a chemical barrier, such a hydrophobic barrier. The sheets with the arrays can be conveniently the size of a 96 well plate or smaller. Each array contains a plurality of addressable capture agents specific for the pre-selected set of binding partners. For example, 33×33 arrays contain roughly 1000 capture agents, each locus on each array containing capture agents that specifically bind to a single pre-selected binding partner. For dispensing of the capture agents onto the surface, functional surface coverage is desired, such that a screened molecule and/or biological particle is detectable. To achieve this, for example, about 500 picoliters per capture agent of a 1 to 2 mg/ml stock solution from the starting collection can be deposited per spot on the array. The exact amount(s) can be empirically determined and can depend upon variables, such as the surface of the array and the sensitivity of the detection methods. The capture agents can be non-covalently or covalently linked, such as by sulfhydryl linkages to amides on the surface. Generally, linkages is through chemical interaction, such as physisorption, hydrophobic interactions, and other interactions typical of surface chemistries.

Dispensing and immobilizing systems are widely available and well known (see, e.g., systems available from Cartesian Systems, Irvine, CA, which has a system for printing on flat surfaces; from Illumina, which employs the tips of fiber optic cables as supports; from TEXAS INSTRUMENTS, which has a surface plasmon resonance chip (i.e., protein derivatized gold); inkjet systems, such as those from Microfab Technologies (Plano Tex.), Incyte (Palo Alto, Calif.), Protogene (Mountain View, Calif.), Packard BioSciences (Meriden Conn.) and other such systems for dispensing and immobilizing proteins to suitable support surfaces). Other systems such as blunt and quill pins, solenoid and piezo nanoliter dispensers, pintools and others also are contemplated.

b. Support for Immobilizing Capture Agents

Supports for immobilizing the capture agents are any of the insoluble materials known for immobilization of ligands and other molecules, used in many chemical syntheses and separations, such as in affinity chromatography, in the immobilization of biologically active materials, and during chemical syntheses of biomolecules, including proteins, amino acids and other organic molecules and polymers. Suitable supports include any material, including biocompatible polymers, that can act as a support matrix for attachment of the antibody material. The support material is selected so that it does not interfere with the chemistry or biological screening reaction.

Supports that also are contemplated for use herein include fluorophore-containing or fluorophore-impregnated supports, such as microplates and beads (commercially available, for example, from Amersham, Arlington Heights, Ill.; plastic scintillation beads from Nuclear Technology, Inc., San Carlos, Calif. and Packard, Meriden, Conn., and colored bead-based supports (fluorescent particles encapsulated in microspheres) from Luminex Corporation, Austin, TX (see, International PCT application No. WO/0114589, which is based on U.S. application Ser. No. 09/147,710; see International PCT application No. WO/0113119, which is U.S. application Ser. No. 09/022,537). The microspheres from Luminex, for example, are internally color-coded by virtue of the encapsulation of fluorescent particles and can be provided as a liquid array. The capture agents, such as antibodies, are linked directly or indirectly by any suitable method and linkage or interaction to the surface of the bead and bound proteins can be identified by virtue of the color of the bead to which they are linked. Detection can be effected by any method, and can be combined with chromogenic or fluorescent detectors or reporters that result in a detectable change in the color of the microsphere (bead) by virtue of the colored reaction and color of the bead. Detection methods include, but are not limited to, methods including, ultraviolet-visible (UV-VIS) spectroscopy, infra-Red (IR) spectroscopy, fluorescence spectroscopy, fluorescence resonance energy transfer (FRET), NMR spectroscopy, circular dichroism (CD), mass spectrometry, other analytical methods, enzymatic assays for detection, antibody assays and other biological and/or chemical detection methods or any combination thereof.

For the bead-based arrays, the capture agents, such as antibodies, are attached to the color-coded beads in separate reactions. The code of the bead identifies the capture agent, such as an antibody, attached to it. The beads then can be mixed and subsequent binding steps performed in solution. They then can be arrayed, for example, by packing them into a microfabricated flow chamber, with a transparent lid, that permits only a single layer of beads to form resulting in a two-dimensional array. The beads to which a protein is bound are identified, thereby identifying the capture agent and the binding partner. The beads are imaged, for example, with a CCD camera to identify beads that have reacted. The codes of such beads are identified, thereby identifying the capture agent, which in turn identifies the polypeptide tag and, ultimately, the displayed protein.

The support can also be a relatively inert polymer, which can be grafted by ionizing radiation to permit attachment of a coating of polystyrene or other such polymer that can be derivatized and used as a support. Radiation grafting of monomers allows a diversity of surface characteristics to be generated on supports (see, e.g., Maeji et al. (1994) Reactive Polymers 22:203-212; and Berg et al. (1989) J. Am. Chem. Soc. 111:8024-8026). For example, radiolytic grafting of monomers, such as vinyl monomers, or mixtures of monomers, to polymers, such as polyethylene and polypropylene, produce composites that have a wide variety of surface characteristics. These methods have been used to graft polymers to insoluble supports for synthesis of peptides and other molecules.

Support materials are typically insoluble substrates that are solid, porous, deformable, or hard, and have any required structure and geometry, including, but not limited to: beads, pellets, disks, capillaries, hollow fibers, needles, solid fibers, random shapes, thin films and membranes, and most generally, form solid surfaces with addressable loci. The supports can also include an inert strip, such as polytetrafluoroethylene strip (marketed under the trademark TEFLON® (Trademark, E. I. DuPont)) or other material to which the capture agents antibodies and other molecules do not adhere, to aid in handling the supports, and can include an identifying symbology.

The preparation of and use of such supports are well known to those of skill in this art; there are many such materials and preparations thereof known. For example, naturally-occurring materials, such as agarose and cellulose, can be isolated from their respective sources, and processed according to known protocols, and synthetic materials can be prepared in accord with known protocols. These materials include, but are not limited to, inorganics, natural polymers, and synthetic polymers, including, but are not limited to: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (see, Merrifield (1964) Biochemistry 3:1385-1390), polyacrylamides, latex gels, polystyrene, dextran, polyacrylamides, rubber, silicon, plastics, nitrocellulose, celluloses, natural sponges, polystyrene, radiation grafted polymers, polyvinylidene fluoride (PVDF), and many others. Selection of the supports is governed, at least in part, by their physical and chemical properties, such as solubility, functional groups, mechanical stability, surface area swelling propensity, hydrophobic or hydrophilic properties and intended use.

(1) Natural Support Materials

Naturally-occurring supports include, but are not limited to agarose, other polysaccharides, collagen, celluloses and derivatives thereof, glass, silica, and alumina. Methods for isolation, modification and treatment to render them suitable for use as supports is well known to those of skill in this art (see, e.g., Hermanson et al. (1992) Immobilized Affinity Ligand Techniques, Academic Press, Inc., San Diego). Gels, such as agarose, can be readily adapted for use herein. Natural polymers such as polypeptides, proteins and carbohydrates; metalloids, such as silicon and germanium, that have semiconductive properties, also can be adapted for use herein. Also, metals such as platinum, gold, nickel, copper, zinc, tin, palladium, silver can be adapted for use herein. Other supports of interest include oxides of the metal and metalloids such as Pt-PtO, Si-SiO, Au-AuO, TiO₂, Cu-CuO, and the like. Also compound semiconductors, such as lithium niobate, gallium arsenide and indium-phosphide, and nickel-coated mica surfaces, as used in preparation of molecules for observation in an atomic force microscope (see, e.g., III et al. (1993) Biophys J. 64:919) can be used as supports. Methods for preparation of such matrix materials are well known.

For example, U.S. Pat. No. 4,175,183 describes a water insoluble hydroxyalkylated cross-linked regenerated cellulose and a method for its preparation. A method of preparing the product using near stoichiometric proportions of reagents is described. Use of the product directly in gel chromatography and as an intermediate in the preparation of ion exchangers also is described.

(2) Synthetic Supports

There are innumerable synthetic supports and methods for their preparation known to those of skill in the art. Synthetic supports are typically produced by polymerization of functional matrices, or copolymerization from two or more monomers from a synthetic monomer and naturally occurring matrix monomer or polymer, such as agarose.

Synthetic matrices include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield (1964) Biochemistry 3:1385-1390; Berg et al. (1990) in Innovation Perspect. Solid Phase Synth. Collect. Pap., Int. Symp., 1st, Epton, Roger (Ed), pp. 453-459; Berg et al. (1989) in Pept., Proc. Eur. Pept. Symp., 20th, Jung, G. et al. (Eds), pp. 196-198; Berg et al. (1989) J. Am. Chem. Soc. 111:8024-8026; Kent et al. (1979) Isr. J. Chem. 17:243-247; Kent et al. (1978) J. Org. Chem. 43:2845-2852; Mitchell et al. (1976) Tetrahedron Lett. 42:3795-3798; U.S. Pat. No. 4,507,230; U.S. Pat. No. 4,006,117; and U.S. Pat. No. 5,389,449). Methods for preparation of such support matrices are well-known to those of skill in this art.

Synthetic support matrices include those made from polymers and co-polymers such as polyvinylalcohols, acrylates and acrylic acids such as polyethylene-co-acrylic acid, polyethylene-co-methacrylic acid, polyethylene-co-ethylacrylate, polyethylene-co-methyl acrylate, polypropylene-co-acrylic acid, polypropylene-co-methyl-acrylic acid, polypropylene-co-ethyl-acrylate, polypropylene-co-methyl acrylate, polyethylene-co-vinyl acetate, polypropylene-co-vinyl acetate, and those containing acid anhydride groups such as polyethylene-co-maleic anhydride, polypropylene-co-maleic anhydride and the like. Liposomes have also been used as solid supports for affinity purifications (Powell et al. (1989) Biotechnol. Bioeng. 33:173).

For example, U.S. Pat. No. 5,403,750, describes the preparation of polyurethane-based polymers. U.S. Pat. No. 4,241,537 describes a plant growth medium containing a hydrophilic polyurethane gel composition prepared from chain-extended polyols; random copolymerization can be performed with up to 50% propylene oxide units so that the prepolymer is a liquid at room temperature. U.S. Pat. No. 3,939,123 describes lightly crosslinked polyurethane polymers of isocyanate terminated prepolymers containing poly(ethyleneoxy) glycols with up to 35% of a poly(propyleneoxy) glycol or a poly(butyleneoxy) glycol. In producing these polymers, an organic polyamine is used as a crosslinking agent. Other supports and preparation thereof are described in U.S. Pat. Nos. 4,177,038, 4,175,183, 4,439,585, 4,485,227, 4,569,981, 5,092,992, 5,334,640, 5,328,603.

U.S. Pat. No. 4,162,355 describes a polymer suitable for use in affinity chromatography, which is a polymer of an aminimide and a vinyl compound having at least one pendant halo-methyl group. An amine ligand, which affords sites for binding in affinity chromatography is coupled to the polymer by reaction with a portion of the pendant halo-methyl groups and the remainder of the pendant halo-methyl groups are reacted with an amine containing a pendant hydrophilic group. A method of coating a substrate with this polymer also is described. An exemplary aminimide is 1,1-dimethyl-1-(2-hydroxyoctyl)amine methacrylimide and vinyl compound is a chloromethyl styrene.

U.S. Pat. No. 4,171,412 describes specific supports based on hydrophilic polymeric gels, generally of a macroporous character, which carry covalently bonded D-amino acids or peptides that contain D-amino acid units. The basic support is prepared by copolymerization of hydroxyalkyl esters or hydroxyalkylamides of acrylic and methacrylic acid with crosslinking acrylate or methacrylate comonomers are modified by the reaction with diamines, amino acids or dicarboxylic acids and the resulting carboxy terminal or amino terminal groups are condensed with D-analogs of amino acids or peptides. The peptide containing D-amino-acids also can be synthesized stepwise on the surface of the carrier.

U.S. Pat. No. 4,178,439 describes a cationic ion exchanger and a method for preparation thereof. U.S. Pat. No. 4,180,524 describes chemical syntheses on a silica support.

Immobilized Artificial Membranes (IAMs; see, e.g., U.S. Pat. Nos. 4,931,498 and 4,927,879) also can be used. IAMs mimic cell membrane environments and can be used to bind molecules that preferentially associate with cell membranes (see, e.g., Pidgeon et al. (1990) Enzyme Microb. Technol. 12:149).

Among the supports contemplated herein are those described in International PCT application Nos WO 00/04389, WO 00/04382 and WO 00/04390; KODAK film supports coated with a matrix material; see also, U.S. Pat. Nos. 5,744,305 and 5,556,752 for other supports of interest. Also of interest are colored “beads,” such as those from Luminex (Austin, Tex.).

C. Immobilization and Activation

Numerous methods have been developed for the immobilization of proteins and other biomolecules onto solid or liquid supports (see, e.g., Mosbach (1976) Methods in Enzymology 44; Weetall (1975) Immobilized Enzymes, Antigens, Antibodies, and Peptides; and Kennedy et al. (1983) Solid Phase Biochemistry, Analytical and Synthetic Aspects, Scouten, ed., pp. 253-391; see, generally, Affinity Techniques. Enzyme Purification: Part B. Methods in Enzymology, Vol. 34, ed. W. B. Jakoby, M. Wilchek, Acad. Press, N.Y. (1974); Immobilized Biochemicals and Affinity Chromatography, Advances in Experimental Medicine and Biology, vol. 42, ed. R. Dunlap, Plenum Press, N.Y. (1974)).

Among the most commonly used methods are absorption and ad-sorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, ImmunoTechnology Catalog & Handbook, 1992-1993, which describes the preparation of and use of such reagents and provides a commercial source for such reagents; and Wong (1993) Chemistry of Protein Conjugation and Cross Linking, CRC Press; see, also DeWitt et al. (1993) Proc. Natl. Acad. Sci. U.S.A. 90: 6909; Zuckermann et al. (1992) J Am. Chem. Soc. 114:10646; Kurth et al. (1994) J Am. Chem. Soc. 116:2661; Ellman et al. (1994) Proc. Natl. Acad. Sci. U.S.A. 91:4708; Sucholeiki (1994) Tetrahedron Lttrs. 35:7307; and Su-Sun Wang (1976) J. Org. Chem. 41:3258; Padwa et al. (1971) J. Org. Chem. 41:3550 and Vedejs et al. (1984) J. Org. Chem. 49:575, which describe photo-sensitive linkers).

To effect immobilization, a solution of the capture agent, such as an antibody or other biological particle, is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO/86 03840)

A large variety of methods are known for attaching biological molecules, including proteins and nucleic acids, to solid supports (see. e.g., U.S. Pat. No. 5451683). For example, U.S. Pat. No. 4,681,870 describes a method for introducing free amino or carboxyl groups onto a silica support. These groups can subsequently be covalently linked to other groups, such as a protein or other anti-ligand, in the presence of a carbodiimide. Alternatively, a silica matrix can be activated by treatment with a cyanogen halide under alkaline conditions. The anti-ligand is covalently attached to the surface upon addition to the activated surface. Another method involves modification of a polymer surface through the successive application of multiple layers of biotin, avidin and extenders (see, e.g., U.S. Pat. No. 4,282,287); other methods involve photoactivation in which a polypeptide chain is attached to a solid substrate by incorporating a light-sensitive unnatural amino acid group into the polypeptide chain and exposing the product to low-energy ultraviolet light (see, e.g., U.S. Pat. No. 4,762,881). Oligonucleotides have also been attached using photochemically active reagents, such as a psoralen compound, and a coupling agent, which attaches the photoreagent to the substrate (see, e.g., U.S. Pat. No. 4,542,102 and U.S. Pat. No. 4,562,157). Photoactivation of the photoreagent binds a nucleic acid molecule to the substrate to give a surface-bound probe.

Covalent binding of the protein or other biomolecule or organic molecule and/or biological particle to chemically activated solid matrix supports such as glass, synthetic polymers, and cross-linked polysaccharides is a more frequently used immobilization technique. The molecule and/or biological particle can be directly linked to the matrix support or linked via a linker, such as a metal (see, e.g., U.S. Pat. No. 4,179,402; and Smith et al. (1992) Methods: A Companion to Methods in Enz. 4:73-78). An example of this method is the cyanogen bromide activation of polysaccharide supports, such as agarose. The use of perfluorocarbon polymer-based supports for enzyme immobilization and affinity chromatography is described in U.S. Pat. No. 4,885,250). In this method the biomolecule is first modified by reaction with a perfluoroalkylating agent such as perfluorooctylpropylisocyanate described in U.S. Pat. No. 4,954,444. Then, the modified protein is adsorbed onto the fluorocarbon support to effect immobilization.

The activation and use of supports are well known and can be effected by any such known methods (see, e.g., Hermanson et al. (1992) Immobilized Affinity Ligand Techniques, Academic Press, Inc., San Diego). For example, the coupling of the amino acids can be accomplished by techniques familiar to those in the art and provided, for example, in Stewart and Young, 1984, Solid Phase Synthesis, Second Edition, PIERCE Chemical Co., Rockford.

Molecules also can be attached to supports through kinetically inert metal ion linkages, such as Co(III), using, for example, native metal binding sites on the molecules, such as IgG binding sequences, or genetically modified proteins that bind metal ions (see, e.g., Smith et al. (1992) Methods: A Companion to Methods in Enzymology 4, 73 (1992); III et al. (1993) Biophys J. 64:919; Loetscher et al. (1992) J. Chromatography 595:113-199; U.S. Pat. No. 5,443,816; Hale (1995) Analytical Biochem. 231:46-49).

Other suitable methods for linking molecules and biological particles to solid supports are well known to those of skill in the art (see, e.g., U.S. Pat. No. 5,416,193). These linkers include linkers that are suitable for chemically linking molecules, such as proteins and nucleic acid, to supports include, but are not limited to, disulfide bonds, thioether bonds, hindered disulfide bonds, and covalent bonds between free reactive groups, such as amine and thiol groups. These bonds can be produced using heterobifunctional reagents to produce reactive thiol groups on one or both of the moieties and then reacting the thiol groups on one moiety with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid diihydrazide, that are cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30: 379-386).

Exemplary linkages include direct linkages effected by adsorbing the molecule and/or biological particle to the surface of the support. Other exemplary linkages are photocleavable linkages that can be activated by exposure to light (see, e.g., Baldwin et al. (1995) J. Am. Chem. Soc. 117:5588; Goldmacher et al. (1992) Bioconj. Chem. 3:104-107, which linkers are herein incorporated by reference). The photocleavable linker is selected such that the cleaving wavelength that does not damage linked moieties. Photocleavable linkers are linkers that are cleaved upon exposure to light (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages). Other linkers include fluoride labile linkers (see, e.g., Rodolph et al. (1995) J. Am. Chem. Soc. 117:5712), and acid labile linkers (see, e.g., Kick et al. (1995) J. Med. Chem. 38:1427)). The selected linker depends upon the particular application and, if needed, can be empirically selected.

2. Capture Agents

As noted, capture agents are molecules and/or biological particles that have specificity or affinity for another molecule, biological particle or other moiety, such as for a defined sequence of amino acids or a binding site on another molecule, such as a ligand, or for purposes herein, a binding partner. For purposes herein, the term capture agent, receptor and anti-ligand are interchangeable. Capture agents include any agent that specifically binds with sufficient affinity (for further use of the resulting self-assembling arrays) to binding partners, which can be conjugated to molecule and/or biological particles.

Any molecule that specifically binds to another is a capture agent as defined herein, including, but not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; sub-cellular structure; sub-cellular compartment or any combination, portion, salt, or derivative thereof; a virus, such as a viral vector or viral capsid with or without packaged nucleic acid; phage, including a phage vector or phage capsid, with or without encapsulated nucleotide acid; a cell, including eukaryotic and prokaryotic cells or fragments thereof; a liposome or micellar agent or other packaging particle; and other such biological materials.

Examples of capture agents are described throughout the disclosure herein. Capture agents can be naturally-occurring or synthetic molecules, and include any molecule, such as nucleic acids, small organics, proteins and complexes that specifically bind to specific sequences of amino acids or other molecules. Capture agents can be used in their unaltered state or as aggregates with other species. They can be attached, covalently or noncovalently, or in physical contact with a binding member, either directly or indirectly via a specific binding substance or linker. As contemplated herein, capture agents are one of a pair of molecules that specifically bind to each other. One member of the pair is a binding partner, such as a polypeptide, that is used as a conjugation tag that can be linked to a molecule and/or biological particle; the other member, the. capture agent, is anything that specifically binds thereto. Examples of capture agents, include, but are not limited to: antibodies and binding fragments thereof, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, organic compounds, and organelles.

A particular capture agent can be selected based on numerous factors including, but not limited to, the ease by which it can be obtained, the ease of its experimental manipulation, and the breadth of experimental data previously known or unknown in the literature or through personal experience.

Capture agents provided herein can be obtained by any method known to those of skill in the art. Such methods include, but are not limited to, purchase from commercial sources, synthetic preparation, isolation from complex mixtures and academic sources such as a gift from a collaborator. Purchase from commercial sources include, but are not limited to, purchase from a biotechnology, chemical or specialty company such as via a catalog, phone or internet purchase; and purchase of a specifically designed molecules or biological particles, such as an oligonucleotide or polypeptide with a specific sequence. Synthetic preparation includes, but is not limited to, techniques such as solid-phase peptide syntheses using tBoc (Hackeng et al. Protein Sci. 10(4): 864-870 (2001)) or Fmoc chemistry (Wellings et al. Methods Enzymol. 289: 44-67 (1997)), which can optionally be automated using commercial systems such as the Pioneer™ and ABI 433A systems from Applied Biosystems or the Apex 396 system from Advanced Chemtech; solid-phase oligonucleotide synthesis techniques, such as phosphoramidite techniques (Caruthers et al. Gene Amplif. Anal. 3: 1-26 (1983)), which can be optionally automated using commercial systems such as the Expedite™ 8909 from Applied Biosystems or AKTA® OligoPilot DNA/RNA synthesizer from Amersham Biosciences; and small molecule synthesis using methods well known to those with skill in the art. Isolation from a complex mixture includes, but is not limited to, chromatographic separation techniques, electrophoretic separations, immunological separations, hybridization techniques, growth and expression techniques and spectroscopic techniques.

The methods provided herein rely upon the ability of a capture agent, such as an antibody, to specifically bind to a target molecule, such as a binding partner, which is, in turn, conjugated to a displayed molecule or biological particle, such as a protein. The specificity of each capture agent (or other receptor in the collection) for a particular binding partner is known or can be readily ascertained, such as by arraying the capture agent so that all of the agents at a locus have the same specificity. Capture agents which bind to each locus can be identified.

The collections of capture agents, such as antibodies or enzymes or portions thereof and mixtures thereof, that specifically bind to a binding partner, such as known or knowable defined sequence of amino acids that is typically at least about 3 to 10 amino acids in length, also are provided. These agents include, but are not limited to, immunoglobulins of any subtype (IgG, lgM, IgA, IgE, IgE) or those of any species, such as, for example, IgY of avian species (Romito et al. (2001) Biotechniques 31:670, 672, 674-670, 672, 675.; Lemamy et al. (1999) Int. J. Cancer 80:896-902; Gassmann et al. (1990) FASEB J. 4:2528-2532), or the camelid antibodies lacking a light chain (Sheriff et al. (1996) Nat. Struct. Biol. 3:733-736; Hamers-Casterman et a. (1993) Nature 363:446-448) that can be raised against virtually limitless entities. Polyclonal and monoclonal immunoglobulins can be used as capture agents. Additionally fragments of immunoglobulins derived by enzymatic digestion (Fv, Fab) or produced by recombinant methods (scFv, diabody, Fab, dsFv, single domain Ig) (Arbabi et al. (1997) FEBS Lett. 414:521-526; Martin et al. (1997) Protein Young 10:607-614; Holt et al. (2000) Curr. Opin. Biotechnol. 11:445-449) are suitable capture agents. Further, entirely new synthetic proteins and peptide mimetics and analogs thereof can be designed for use as capture agents (Pessi et al. (1993) Nature 362:367-369).

Many different protein domains have been engineered to introduce variable regions to mimic the diversity seen in antibody molecules. Lipocalin (Skerra (2000) Biochim. Biophys. Acta 1482:337-350), fibronectin type III domains (Koide et al. (1998) J. Mol. Biol. 284:1141-1151), protein A domains (Nord et al. (2001) Eur. J. Biochem. 268:4269-4277; Braisted et al. (1996) Proc. Natl. Acad. Sci. U.S.A. 93:5688-5692), protease inhibitors (Kunitz domains, cysteine knots (Skerra (2000) J. Mol Recognit. 13:167-187; Christmann etal. (1999) Protein Young 12:797-806), thioredoxin (Xu et al. (2001) Biochemistry 40:4512-4520; Westerlund-Wikstrom,B (2000) Int. J. Med. Microbiol. 290:223-230), and GFP (Peelle et al. (2001) Chem. Biol. 8:521-534; Abedi et al. (1998) Nucleic Acids Res. 26:623-630) have been modified to function as binding agents. Many domains in proteins have been implicated in direct protein-protein interactions. With modifications, these interactions can be manipulated and controlled. For example, it is known that src homology-2 (SH2) domains are known to bind proteins containing a phosphorylated tyrosine (Ward et al. (1996) J. Biol Chem. 271:5603-5609). The phosphotyrosine alone does not determine specificity, but amino acids surrounding it contribute to the binding affinity and specificity (Songyang et al. (1993) Cell 72:767-778). The SH2 domain can function as a capture agent. For example, altering amino acids in the binding pocket of SH2 such that new specificities results in the generation of additional capture agents. Similarly, src homology 3 domains, SH3 domains bind a ten-residue consensus sequence, XPXXPPPFXP (where X is any amino acid residue, F is phenylalanine and P is proline; SEQ ID No.35) (Sparks etal. (1998) Methods Mol. Biol. 84:87-103). SH3 domains can function as capture agents. Additional capture agents can be generated by selecting mutant SH3 domains to bind to polypeptide tags with the above consensus sequence. The epidermal growth factor (EGF) domain has a two-stranded beta-sheet followed by a loop to a C-terminal short two-stranded sheet. This domain has been implicated in many protein-protein interactions, it can form the basis for a family of capture agents following manipulation of the loop between the two beta sheets. Long alpha-helical coils are known to interact with other alpha-helical segments to cause proteins to dimerize and trimerize. These coiled-coil interactions can be of very high affinity and specificity (Arndt et al. (2000) J. Mol Biol. 295:627-639), and therefore can be used as capture agents when paired with complementary polypeptide tags. Nearly any protein domain can be modified such that the variability introduced into one or more exposed regions of the molecule can constitute a potential binding site. Mutant enzymes, designated substrate trapping enzymes, that do not exhibit catalytic activity but retain substrate binding activity can also be used as capture agents (see, e.g., International PCT application No. WO 01/02600).

While most of the reagents used for affinity interactions with proteins are proteins, there are many other protein-binding agents. Nucleic acids constitute a family of molecules that have inherent diversity of structure. Although there are only five naturally occurring subunits (ATP, CTP, TTP, GTP and UTP) compared to the twenty naturally occurring amino acids that make up proteins, nucleic acids have the potential to fold into an immense variety of different structures capable of binding to a huge number of protein elements. Selection strategies for single-stranded RNA (Sun (2000) Curr. Opin. Mol Ther. 2:100-105; Hermann et al. (2000) Science 287:820-825; Cox et al. (2001) Bioorg. Med. Chem. 9:2525-2531) and single-stranded DNA (or RNA) aptamers (Ellington et al. (1992) Nature 355:850-852) have been developed. These methods have proven successful for discovery of high affinity binders to small molecules as well as proteins. Using these methods, aptamers that bind with high specificity and affinity to polypeptide tags can be selected and then used as capture agents.

Single-stranded DNA or RNA can fold into diverse structures. Double-stranded nucleic acids, while more restricted in overall structure, can be used as capture agents with the correct polypeptide tags. DNA binding proteins such as proteins containing zinc finger domains (Kim et al. (1998) Proc. Natl. Acad. Sci. U.S.A. 95:2812-2817) and leucine zipper (Alber (1992) Curr. Opin. Genet. Dev. 2:205-210) domains bind with high specificity to double stranded DNA molecules of defined sequence. Zinc finger domains bind to dsDNA in an arrayed format (see, e.g., Bulyk et al. (2001) Proc. Natl. Acad. Sci. U.S.A. 98:7158-7163). Additionally, DNA modifying enzymes can be modified for use as polypeptide tags to bind to DNA used as an affinity capture agent. For example, the DNA restriction endonuclease BamHl has specific target sequence of GGATCC, but with mutation of the active site, a new enzyme is created that recognizes the sequence GCATGC. It also has been demonstrated that base pairs outside the specific target sequence play an important roll in the binding affinity, and that the catalytic event can be eliminated in the absence of the cofactor Mg²+ (Engler et al. (2001) J. Mol Biol. 307:619-636). Mutations in some restriction enzymes abolish the cleavage event and leave the DNA binding domain bound to the dsDNA target (Topal et al. (1993) Nucleic Acids Res. 21:2599-2603; Mucke et al. (2000) J. Biol. Chem. 275:30631-30637). Thus panels of double-stranded nucleic acids can serve as capture agents.

Small chemical entities also can be designed to be capture agents. The highest affinity non-covalent interaction involving a protein is between proteins such as egg-white avidin or the bacterial streptavidin and the small, naturally-occurring chemical entity biotin. Biotin-like molecules can be used as capture agents if the polypeptide tags are avidin-like proteins. Panels of chemically synthesized biotin analogs, and a corresponding panel of avidin mutants each capable of specific, high affinity binding to those biotin analogs can be employed. Other chemical entities have specific affinity for protein sequences. For example, immobilized metal affinity chromatography has been widely used for purification of proteins containing a hexa-histidine tag. Iminodiacetic acid, NTA or other metal chelators are used. The metal used determines the strength of interaction and possibly the specificity. Similarly, proteins that bind to other metals (Patwardhan et al. (1997) J. Chromatogr. A 787:91-100) can be selected.

Similarly, digoxin and a panel of digoxin analogs can be used as capture agents if the polypeptide tags are designed to bind to those analogs. Antibodies and scFvs have been created that bind with high specificity to these analogs (Krykbaev et al. (2001) J. Biol. Chem. 276:8149-8158) and the recombinant scFvs can be used as polypeptide tags. Carbohydrates, lipids, gangliosides can be used as capture agents for polypeptide tags in the form of lectins (Yamamoto et al. (2000) J. Biochem. (Tokyo) 127:137-142; Swimmer et al. (1992)Proc. Natl. Acad. Sci. U.S.A. 89:3756-3760), fatty acid binding proteins (Serrero et al. (2000) Biochim. Biophys. Acta 1488, 245-254.) and peptides (Matsubara et al. (1999) FEBS Lett. 456:253-256). Hence any member of a pair of molecules that specifically bind is contemplated.

For exemplary purposes herein, reference is made to antibody capture agents and polypeptide binding partners to which the antibody specifically binds. The antibodies described herein as capture agents can be identified and generated using experimental methods well known to those with skill in the art such as panning phage displayed peptide libraries and raising antibodies from exposure of a subject to an antigen, such as a polypeptide binding partner. Antibodies can also be purchased commercially from numerous companies, such as SIGMA-Aldrich (www.sigmaaldrich.com), INVITROGEN (www.invitrogen.com), NOVAGEN (www.novagen.com), Covance Research Products (www.crpinc.com), STRATAGENE (www.stratagene.com) and ABCAM (www.abcam.com), or from a depository such as the American Tissue Culture Collection (www.atcc.org) and the European Collection of Cell Cultures (www.ecacc.org.uk). For example, capture agents used in the combinations, kits, methods and systems provided herein can include commercially available antibodies such as those seen in Table 2 below. It is understood that any pair of molecules that specifically bind are contemplated and that the partners are interchangeable as capture agents and binding partners; in many embodiments herein, the molecules such as antibodies, are designated capture agents, and the polypeptides that specifically bind thereto are binding partners. In other embodiments, the polypeptides, such as small peptide sequences can be designated capture agents and the molecules which bind them, such as antibodies, are designated as binding partners.

Capture agents can be positionally addressed. Alternatively, each capture agent can be addressed by associating them with unique identifiers, such as by linkage to optically encoded tags, including colored beads or bar coded beads or supports, or linked to electronic tags, such as by providing microreactors with electronic tags or bar coded supports (see, e.g., U.S. Pat. No. 6,025,129; U.S. Pat. No. 6,017,496; U.S. Pat. No. 5,972,639; U.S. Pat. No. 5,961,923; U.S. Pat. No. 5,925,562; U.S. Pat. No. 5,874,214; U.S. Pat. No. 5,751,629; U.S. Pat. No. 5,741,462), or chemical tags (see, U.S. Pat. No. 5,432,018; U.S. Pat. No. 5,547,839) or colored tags or other such addressing methods that can be used in place of physically addressable arrays. For example, each capture agent type can be bound to a support matrix associated with a color-coded tag (i.e., a colored sortable bead) or with an electronic tag, such as an radio-frequency tag (RF), such as IRORI MICROKANS® and MICROTUBES® microreactors (see, U.S. Pat. No. 6,025,129; U.S. Pat. No. 6,017,496; U.S. Pat. No. 5,972,639; U.S. Pat. No. 5,961,923; U.S. Pat. No. 5,925,562; U.S. Pat. No. 5,874,214; U.S. Pat. No. 5,751,629; U.S. Pat. No. 5,741,462; International PCT application No. WO98/31732; International PCT application No. WO98/15825; and, see, also U.S. Pat. No. 6,087,186). It is understood, however, that other such identifying methods can be readily adapted for use with the methods herein. It is only necessary that the identity (i.e., binding partner specificity) of the capture agent, such as an antibody, is known.

3. Binding partners and Preparation Thereof

As described above, any moiety, such as a polypeptide, that specifically binds to a capture agent is contemplated as a binding partner, also referred to as a polypeptide or epitope tag. The term “epitope” is not to be construed as limited to an antibody-binding polypeptide, but as any specifically binding moiety. A binding partner includes any molecule that specifically binds with sufficient affinity (for further use of the resulting self-assembling arrays) to a particular capture agent. Any of the molecules or biological particles described as possible capture agents also can be used as binding partners and vice versa as long as the capture agents are addressable, such as by arraying, labeling with nanobarcodes or other such codes, encoded with colored beads and other such addressing products.

Molecules that specifically bind to another molecule can be used as a binding partner, including, but not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; sub-cellular structure; sub-cellular compartment or any combination, portion, salt, or derivative thereof; a virus, such as a viral vector or viral capsid with or without packaged nucleic acid; phage, including a phage vector or phage capsid, with or without encapsulated nucleic acid; a cell, including eukaryotic and prokaryotic cells or fragments thereof; a liposome or micellar agent or other packaging particle; and other such biological materials.

A particular binding partner can be selected based on numerous factors including, but not limited to, the ease by which it can be obtained, the ease of its experimental manipulation, and the breadth of experimental

TABLE 2
Antibodies

| | | | | |
|---|---|---|---|---|
| Antibody | Isotype | Specificity | Antigen Sequence | ATCC No. |

| | | | | |
|---|---|---|---|---|
| anti-E-tag antibody | IgG₁ | E-tag peptide | GAPVPYPDPLEPR | NA |
| | | | (SEQ ID No. 1) | |
| anti-FLAG M2 antibody | IgG₁ | FLAG peptide | DYKDDDDK | 1025144 |
| | | | (SEQ ID No. 2) | |
| anti-Glu-Glu antibody | IgG₁ | Polyoma virus medium T antigen | EEEEYMPME | 1021802 |
| | | peptide sequence | (SEQ ID No. 3) | |
| anti-HA.11 antibody | IgG₁ kappa | Influenza hemagglutinin epitope | YPYDVPDYA | 1021801 |
| | | | (SEQ ID No. 4) | |
| anti-HSV-tag antibody | IgG₁ | Herpes Simplex Virus glycoprotein | QPELAPEDPED | NA |
| | | D epitope | (SEQ ID No. 5) | |
| anti-c-myc antibody | IgG₁ kappa | Portion of the human c-myc gene | EQKLISEEDL | 20555 |
| | | product | (SEQ ID No. 6) | |
| anti-T7 tag antibody | IgG_{2b} kappa | Gene 10 leader peptide expressed | MADMTGGQQMG | NA |
| | | by the pET vector | (SEQ ID No. 7) | |
| anti-VSV G antibody | IgG₁ kappa | Vesticular stomatitis virus | YTDIEMNRLGK | NA |
| | | glycoprotein epitope | (SEQ ID No. 8) | |
| anti-V5 antibody | IgG₂ₐ | Epitiope found in P and V proteins | GKPIPNPLLGLDST | NA |
| | | of the paramyxovirus, SV5 | (SEQ ID No. 9) | |
| anti-AB2 antibody | IgG_{2b} | Murine Leukemia virus 1 | LTPPMGPVIDQR | 1019528 |
| | | protein 12 | (SEQ ID No. 10) | |
| anti-AB4 antibody | IgG₁ | Bovine herpesvirus 1 glycoprot, D | QPQSKGFEPPPP | 1006723 |
| | | | (SEQ ID No. 11) | |
| anti-834 antibody | IgG₁ | Generated against purified WT | DLHDERTLQFKL | NA |
| | | Green Fluorescent Protein (GFP) | (SEQ ID No. 12) | |
| anti-P5D4 A antibody | IgG₁ kappa | Vesticular stomatitis virus | HPNLPETRRYAL | NA |
| | | glycoprotein epitope | (SEQ ID No. 13) | |
| anti-P5D4 B antibody | IgG₁ kappa | Vesticular stomatitis virus | SYTGIEFDRLSN | NA |
| | | glycoprotein epitope | (SEQ ID No. 14) | |
| anti-4C10 antibody | IgG_{2b} | Generated against a | MVDPEAQDVPKW | 1025450 |
| | | Glutathione S-transferase (GST) | (SEQ ID No. 15) | |
| | | fusion protein | | |
| anti-AB3 antibody | IgG₁ | WC1 peptide from Bovine | YEYAKGDEPPAL | 1019526 |
| | | lymphocyte | (SEQ ID No. 16) | |
| anti-AB6 antibody | IgG₂ₐ | Bovine herpesvirus | AGTQWCLTRPPC | 1001543 |
| | | glycoprotein D | (SEQ ID No. 17) | |
| anti-KT3 A antibody | IgG₁ | Carboxy terminial sequence of the | KMLPNEFFGLLP | 4361 |
| | | SV40 large T antigen | (SEQ ID No. 18) | |
| anti-KT3 B antibody | IgG₁ | Carboxy terminial sequence of the | KLIPTQLYLLHP | 4361 |
| | | SV40 large T antigen | (SEQ ID No. 19) | |
| anti-KT3 C antibody | IgG₁ | Carboxy terminial sequence of the | SFMPIEFYARKL | 4361 |
| | | SV40 large T antigen | (SEQ ID No. 20) | |
| anti-7.23 antibody | IgG₁ | Cre recombinase | TNMEWMTSHRSA | 1021789 |
| | | | (SEQ ID No. 21) | |
| anti-HOPC1 antibody | IgG₂ₐ lambda | HOPC-1 tumor line | MPQQGDPDWVVP | NA |
| | | | (SEQ ID No. 22) | |
| anti-S1 antibody | IgG₂ₐ kappa | preS1 peptide of | NANNPDWDF | NA |
| | | Hepatitus B virus | (SEQ ID No. 23) | |
| anti-E2 antibody | IgG₁ | Bovine Pappillomavirus type 1 | SSTSSDFRDR | NA |
| | | transvector protein E2 | (SEQ ID No. 24) | |
| anti-His tag antibody | IgG₂ₐ kappa | His tag | HHHHHHGS | NA |
| | | | (SEQ ID No. 25) | |
| anti-AU1 antibody | IgG₃ | Bovine pappillomavirus 1 peptide | DTYRYI | NA |
| | | | (SEQ ID No. 26) | |
| anti-AU5 antibody | IgG₁ | Bovine pappillomavirus 1 peptide | TDFYLK | NA |
| | | | (SEQ ID No. 27) | |
| anti-IRS antibody | IgG₁ | Dodecapeptide NPDSEIARYIRS | RYIRS | NA |
| | | | (SEQ ID No. 28) | |
| anti-NusA antibody | IgG₁ | N utiliztation substance | NusA Protein | NA |
| | | protein A peptide | (SEQ ID No. 29) | |
| anti-MBP antibody | IgG₁ | Maltose Binding Protein | MBP Protein | NA |
| | | | (SEQ ID No. 30) | |
| anti-TBP antibody | IgG₁ | TATA-box Binding Protein | TBP residues 1-20 | NA |
| | | (N-terminal residues 1-20) | (SEQ ID No. 31) | |
| anti-TRX antibody | IgG_{2a kappa} | Thioredoxin Protein | TRX Protein | NA |
| | | | (SEQ ID No. 32) | |

data previously known or unknown in the literature or through personal experience. Binding partners can be obtained by any method known to those of skill in the art. Such methods include, but are not limited to, purchase from commercial sources, synthetic preparation, isolation from complex mixtures and academic sources such as a gift from a collaborator. Purchase from commercial sources include, but are not limited to, purchase from a biotechnology, chemical or specialty company such as via a catalog, phone or internet purchase; and purchase of a specifically designed molecules or biological particles, such as an oligonucleotide or polypeptide with a specific sequence. Synthetic preparation includes, but is not limited to, techniques such as solid-phase peptide syntheses using tBoc (Hackeng et al. Protein Sci. 10(4): 864-870 (2001)) or Fmoc chemistry (Wellings et al. Methods Enzymol. 289: 44-67 (1997)), which can optionally be automated using commercial systems such as the Pioneer™ and ABI 433A systems from Applied Biosystems or the Apex 396 system from Advanced Chemtech; solid-phase oligonucleotide synthesis techniques, such as phosphoramidite techniques (Caruthers et al. Gene Amplif. Anal. 3: 1-26 (1983)), which can be optionally automated using commercial systems such as the Expedite™ 8909 from Applied Biosystems or AKTA® OligoPilot DNA/RNA synthesizer from Amersham Biosciences; and small molecule synthesis using methods well known to those with skill in the art. Isolation from a complex mixture includes, but is not limited to, chromatographic separation techniques, electrophoretic separations, immunological separations, hybridization techniques, growth and expression techniques and spectroscopic techniques.

A binding partner, such as a polypeptide tag, can also refer to a sequence of amino acids that includes the sequence of amino acids to which a capture agent, such as an antibody and any agent described above, specifically binds. For polypeptide (epitope) tags, the specific sequence of amino acids or region of a molecule to which each binds is referred to herein generically as an epitope (but is not an epitope in the immunological sense). Any sequence of amino acids that binds to a capture agent therefor is contemplated for use as a binding partner. The polypeptide binding partners are not necessarily small peptide sequences. For example, in some embodiments the binding partners are antibodies and the capture agents are antigens, such as small peptides.

For purposes herein, a binding partner, such as a polypeptide tag, can be encoded by an oligonucleotide, which is used to recombinantly conjugate the binding partner to another molecule and/or biological particle. When reference is made to a polypeptide or binding partner (i.e., binding pair for a particular receptor (capture agent) or portion thereof) with respect to a nucleic acid, it is the nucleic acid encoding the binding partner to which reference is made. Each binding partner, such as a polypeptide, is referred to as Em (again E is not intended to limit the tags to “epitopes,” but includes any sequence of binding partner, such as a sequence of amino acids, that specifically binds to a capture agent); when nucleic acids are being described, the Em is nucleic acid and refers to the sequence of nucleic acids that encode the binding portion of the polypeptide binding partner; when the translated proteins are described, Em refers to amino acids (the actual binding polypeptide or epitope). The number of E's corresponds to the number of unique capture agents, such as antibodies, in an addressable collection. “m” is typically at least 10, 30 or more, 50 or 100, 250 or more, and can be as high as desired and as is practical. Generally “m” is about 100, 250, 500, 1000 or more.

In some cases, it can be necessary or desirable for the conjugated molecule to have a plurality of tags, in addition to the binding partner, that serve different purposes. Nucleic acid encoding a polypeptide tag (binding partner) also can include sequences of nucleotides that can aid in unique or convenient priming, or can encode amino acids that confer desired properties, such as trafficking signals, detection, solubility alteration, facilitation of purification or conjugation or other functions or provide other functions. For example, tags such as, but not limited to, green fluorescent protein (GFP), red fluorescent protein (RFP), blue fluorescent protein (BFP) or other commercially available tags can be used for the detection of expressed polypeptide tags in culture or as in purified fusion molecule. Tags that result in the secretion of the polypeptide tagged molecule include, but are not limited to, RsaA, CBP, MBP, OmpT, OmpA, PelB or other commercially available tags. Tags that facilitate purification such as, but not limited to, polyhistidine and polylysine tags, FLAG, calmodulin binding peptide (CBP), biotin carboxycarrier protein (BCCP), Strep, maltose-binding protein (MBP) intein/chitin-binding domain, cellulose-binding domain (CBP), myc tags or other commercially available tags are known and can be appended to the polypeptide tagged molecule by any method-known to those skilled in the art. Further, any of the tags listed above can be used as the binding partner. In addition, a capture agent can be used as an affinity ligand for the purification of an polypeptide tagged molecule. A plurality of tags, in number and function, can be used within a single recombinantly conjugated molecule. Selection of the tags, including, but not limited to those listed above, for placement in a particular expression vector for production of recombinantly conjugated molecules or biological particles can be determined by those skilled in the art.

Furthermore, particularly for certain applications, such as profiling, the polypeptide binding partner does not have to be fused to the displayed molecule such that a single protein is synthesized. It is possible to prepare tags that are encoded as a separate polypeptides that are physically or otherwise associated or linked with the displayed molecule. For example, dimerizing domains can be used to couple two separate proteins expressed in the same cell (Chao et al. (1998) J. Chromatogr. B Biomed. Sci. Appl. 715:307-329; Hodges (1996) Biochem. Cell Biol. 74, 133-154; Alber (1992) Curr. Opin. Genet. Dev. 2:205-210). One of the dimerizing-domains is fused to the displayed molecule, and its partner dimerizing-domain is fused to the polypeptide binding partner. The dimerizing domains cause association of the displayed molecule and the binding partner. These binding partners serve the same purpose of subdivision of the library on the addressable array. Also, the DNA encoding such binding partners is still associated with one displayed molecule (since it is in the same plasmid or linear expression construct), and therefore indicates which displayed molecule to recover.

Nucleic acid encoding a polypeptide binding partner can include a tag-specific amplification sequence (recovery or R-tag ) that can be associated with a specific binding partner in a predetermined manner. This R-tag can encode protein, but does not need to be part of the binding portion of the encoded polypeptide tag. An R-tag does not necessarily encode protein, and can be located prior to the translational start site, or following the translational termination site or elsewhere. For example, a different recovery tag is associated with each polypeptide tag. By separating the amplification portion from the binding partner-encoding portion, it is possible to optimize each for the desired function, i.e., the R-tag portion can be an optimal amplification sequence, and the capture-agent-binding portion can be optimized for binding to a selected capture agent. Because the R-tags do not need to encode protein, there is considerable flexibility in designing sequences that allow the specific hybridization (and, thus amplification) of only the correct corresponding sequences. Many available DNA sequence analysis software packages (Lasergene's DNAStar, Informax's VectorNTi, etc.) allow the analysis of oligonucleotides for melting temperature, primer-dimer formation, hairpin formation as well as cross-reactivity and mis-priming.

Furthermore, tags are not necessarily polypeptides. It is possible that the ligand for the capture agent is a protein modification such as a phosphorylated amino acid. Capture agents can distinguish combinations of phosphorylated and non-phosphorylated residues contained in a peptide. For example, mutated SH2 domains are arrayed as capture agents such that one binds the sequence His-PO₄Tyr-Ser-Thr-Leu-Met, a second binds His-Tyr-PO₄Ser-Thr-Leu-Met and a third binds His-Tyr-Ser-PO₄Thr-Leu-Met and a fourth binds PO₄His-Tyr-Ser-Thr-Leu-Met. Each of these peptide sequences is the same, but the position of the phosphate group determines specificity. In each of these cases, the peptide is fused to the library member, but an additional encoded protein (Serine, Histidine, Threonine, or Tyrosine kinases) directs the phosphorylation event separately. In this case the polypeptide tag has two separate determinants, the peptide portion that binds to a capture agent, and the kinase responsible for the phosphorylation event. Recovery entails two sequential amplification steps. As above, these tags serve the same purpose of subdivision of the library in an addressable collection. Also, the nucleic acid encoding this tag (the peptide and the kinase) are associated with one specific subset of a total DNA library, since they are in the same plasmid or linear expression construct, and therefore indicate which subset to recover. Other protein modifying enzymes include but are not limited to those that are involved fatty acid acylation, glycosylation, and methylation.

In addition, enzymatic modifications of the binding partner, such as a polypeptide, before exposure to the capture agent can alter binding specificity. In such embodiments, the enzyme catalyzing the modification is not required to be physically linked to the binding partner. The enzyme-catalyzed modification is used to alter specificity of the binding partner for the capture agent or the specificity of a capture agent for a binding partner.

For exemplary purposes herein, reference is made to polypeptide binding partners of a particular amino acid sequence to which an antibody capture agent specifically binds. Some exemplary polypeptide binding partners provided herein include an E-tag polypeptide (SEQ ID No.1), a FLAG polypeptide (SEQ ID No.2), a Glu-Glu polypeptide (SEQ ID No.3), a HA.11 polypeptide (SEQ ID No.4), a HSV-tag polypeptide (SEQ ID No. 5), a c-myc polypeptide (SEQ ID No.6), a T7 tag polypeptide (SEQ ID No. 7), a VSV-G polypeptide (SEQ ID No.8), a V5 polypeptide (SEQ ID No. 9), an AB2 polypeptide (SEQ ID No.10), an AB4 polypeptide (SEQ ID No. 11), a B34 polypeptide (SEQ ID No.12), a P5D4-A polypeptide (SEQ ID No. 13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a S1 polypeptide (SEQ ID No.23), an E2 polypeptide (SEQ ID No.24), a His tag polypeptide (SEQ ID No.25), an AU1 polypeptide (SEQ ID No. 26), an AU5 polypeptide (SEQ ID No.27), an IRS polypeptide (SEQ ID No. 28), [[a KT3 polypeptide (SEQ ID No.34), a S-tag polypeptide (SEQ ID No.33)]], NusA (SEQ ID No.29), Maltose binding protein (SEQ ID No. 30), TATA-box binding protein (SEQ ID No.31) and thioredoxin (SEQ ID No. 32).

4. Identification of Capture Agents—Binding Partner Pairs

For preparation of the self-assembling arrays herein, pairs of capture agents and binding partners are required. These pairs can be identified and/or designed or otherwise selected. The binding partners are immobilized by the capture agents by any interaction that is specific and of high affinity, generally equal to or greater affinity than that of other moieties, such as molecules, cells and other biological particles, that bind to immobilized tagged molecules in the self-assembled array (capture system). Any interaction, including, but not limited to, covalent, ionic, hydrophobic, van der Waals and other such interactions, that results in the immobilization of a tagged molecule by a capture agent, are contemplated for use herein.

As noted, capture agents and binding partners can be any molecule or compound known in the art. Thus, pairs of capture agents and binding partners can include, but are not limited to, protein:protein, protein:nucleic acid, nucleic acid:nucleic acid, protein:lipid, lipid:lipid, protein:small molecule, receptor:signal, antibody:antigen, peptide nucleic acid:nucleic acid, and small molecule:nucleic acid pairs. Selection of binding pairs can be empirically determined by those with skill in the art, such as with binding assays, or can include pairs with known high specificity and affinity, such as biotin and avidin. Such methods are exemplified herein with respect to antibody capture agents and polypeptide binding partners, but it is understood that any capture agent/binding partner pairs obtained or made by any method known to those of skill in the art are contemplated.

Antibodies or fragments thereof and their cognate antigens can serve as capture agents and/or binding partners, respectively. An antibody binds to a small portion of its cognate antigen, known as its epitope, which contains as few as 3-6 amino acid residues (Pellequer et al. (1991) Methods in Enzymology 208:176). The amino acid residues can be contiguous, or they can be discontinuous within the antigen sequence. When the amino acid residues of the antigen sequence are discontinuous, they are presented in close proximity for recognition by the cognate antibody through three-dimensional folding of the antigen.

Candidate capture agent—polypeptide binding pairs, such as antibody-antigen pairs, can be identified by any method known to the art, including, but are not limited to, one or several of the following methods, such as, for example:

a) phage display of a random peptide library followed by biopanning with the antibody of interest;

b) analysis of complementarity-determining regions (CDRs) of the antibody of interest;

c) theoretical molecular modeling of three-dimensional antibody structure;

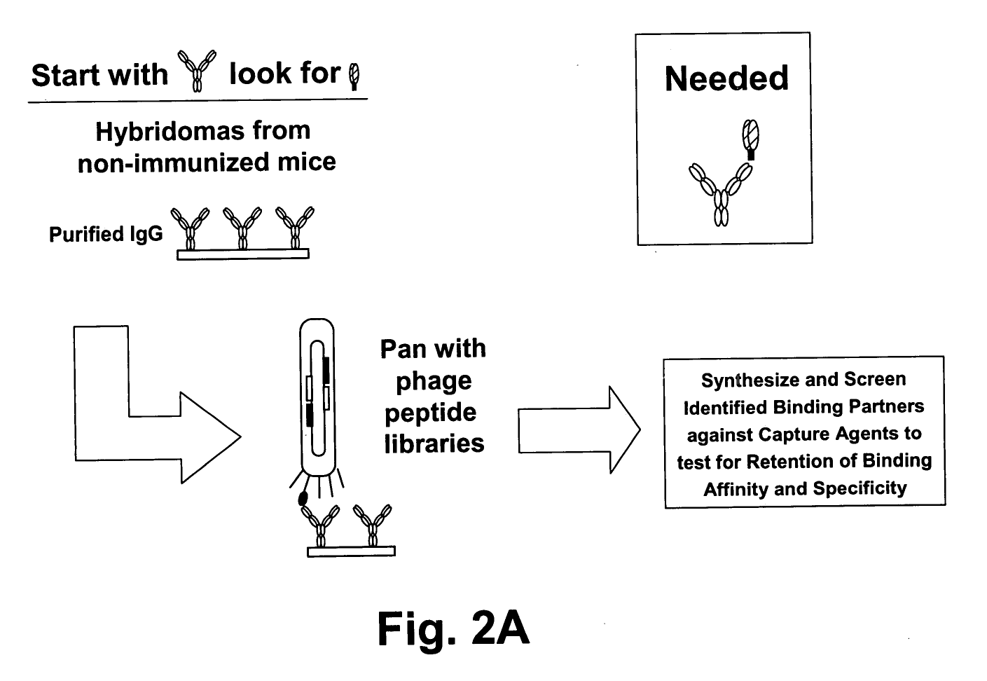

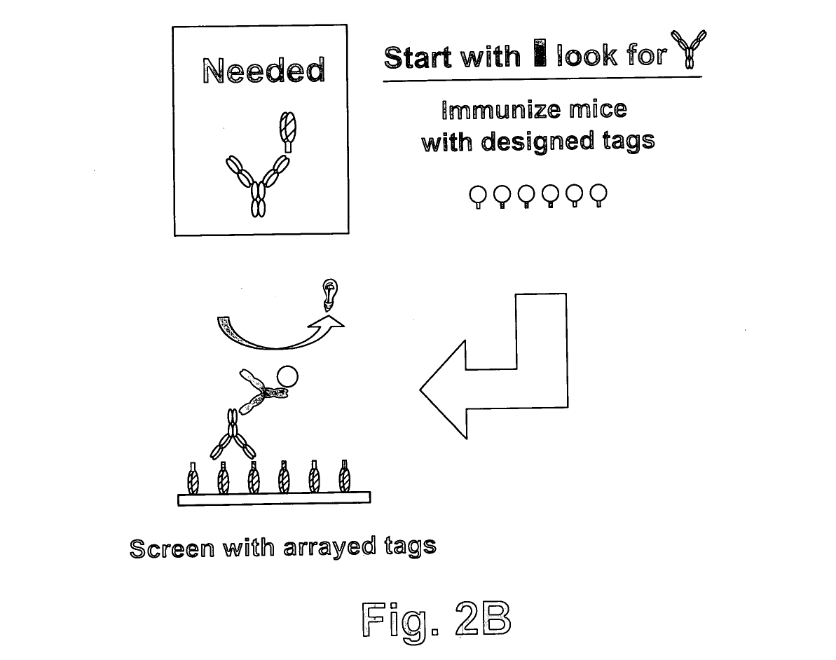

d) raising antibodies from exposure of a subject to an antigen and any method known to those of skill in the art for identifying pairs of molecules that bind with high affinity and specificity. The following discussion provides exemplary methods; others known to those of skill in the art can be ernployed. Exemplary methods are depicted in FIGS. 2A-2B.

a. Panning Phage Displayed Peptide Libraries

One method for identifying capture agent—binding partner pairs employs panning phage displayed peptide libraries, such as random peptide libraries, for molecules, such as peptides, that interact with addressable capture agents. Peptides that interact with a specific capture agent, such as an antibody or a protein, can be identified by displaying random libraries of peptides on the surface of a phage molecule and monitoring their interactions with an addressable collection of capture agents. The bacteriophage that display peptides that interact with the addressable capture agents can be isolated through washing and then enriched through multiple panning steps, resulting in a high population of phage displaying a peptide that can be used as either a binding partner in conjunction with the addressable capture agent.

For example, in order to identify peptide binding partners using panning and phage display, hybridoma cells are first created either from non-immunized mice or mice immunized with a phage expressing a library of random epitopes or other random peptide libraries from which binding partners are to be selected (see, e.g., FIG. 2A). Stable hybridoma cells are initially screened for high immunoglobulin (Ig) production and epitope binding. Ig production can be measured in culture supernatants by ELISA assay using a goat anti-mouse IgG antibody. Epitope binding can also be measured by ELISA assay in which the mixture of haptens (potential binding partner proteins) used for immunization are immobilized to the ELISA plate and bound IgG from the culture supernatants is measured using a goat anti-mouse IgG antibody. Both assays can be performed in 96-well formats or other suitable formats. For example, approximately 10,000 hybridomas can be selected from these screens (see, e.g., Example 1).

Next, the Ig are separately purified using 96-well or higher density purification plates containing filters with immobilized Ig-binding proteins (proteins A, G or L). The quantity of purified Ig is measured using a standard protein assay formatted for 96-well or higher density plates. The purified Ig are spotted separately onto a nitrocellulose filter using, for example, standard pin-style arraying systems as described herein. In addition, a second aliquot of each purified Ig is combined to produce a mixture with equal quantities of each Ig. The mixed Ig are bound to paramagnetic beads which are used as a solid-phase support to pan a library of bacteriophage expressing the random disulfide-constrained heptameric epitopes. The batch panning enriches the phage display library for phage expressing epitopes, such as peptides, that bind to the purified Ig. This enrichment dramatically reduces the diversity in the phage library.

The enriched phage display library is then bound to the array of purified Ig on the nitrocellulose and stringently washed. Ig-binding phage are detected by staining, such as with an anti-phage antibody-HRP conjugate, to produce a detectable signal, such as a chemiluminescent signal, with an imaging system, such as a charge coupled device (CCD)-based imaging system. Loci in the array producing the strongest signals are cut out and the phage eluted and propagated. Epitopes expressed by the recovered phage are identified by DNA sequencing and further evaluated for affinity and specificity. This method generates a collection of high-affinity, high-specificity antibodies that recognize the cognate peptide for use as a capture agent—binding partner pair in the self-assembled arrays provided herein. Continued screening produces larger collections of antibodies of improved quality.

In a similarly manner, panning of phage displayed peptide libraries can be used to map the epitope of an antigen or binding site of a protein, thereby identifying the exact amino acid residues required for interaction with the addressable capture agents, such as an antibody or a protein. For either method, once the peptide or portion of the molecule, such as an antigen, that reacts with the capture agent is identified, the peptide or molecular portion thereof can be synthesized and conjugated to a molecule and/or biological particle, as described below. This conjugate can then be screened against the antibody capture agents identified above to determine whether the peptides or antigenic portion thereof retains the ability to interact with high affinity and specificity with the capture agent, thereby identifying a capture agent—binding partner pair.

b. Analysis of Complementarity-determining Regions (CDRs) of an Antibody

Capture agent-polypeptide pairs can be identified by analyzing complementarity-determining regions (CDR's) in the antibody of interest. Translation of available cDNA sequences of the variable light and variable heavy chains of a particular antibody permit the delineation of the CDRs by comparison to the database of protein sequences compiled in “Sequences of Proteins of Immunological Interest,” Fifth Edition, Volume 1, Editors: Kabat et al. (1991) (see, e.g., table on page xvi). In some cases, CDR peptides can mimic the activity of an antibody molecule (Williams et al. Proc. Natl. Acad. Sci. U.S.A. 86: 5537 (1989)). CDR peptides can bind their cognate antibody, thus effecting displacement of the antibody from the antigen. To increase the efficiency of the above procedures in identifying candidate releasing peptides, biospecific interaction analysis using surface plasmon resonance detection through the use of the Pharmacia BlAcore® system can be used. This technology provides the ability to determine binding constants and dissociation constants of antibody-antigen interactions. Analysis of multiple antibodies and the number of biopanning steps (at set antibody concentrations) required to identify a tight-binding consensus peptide sequence can provide a database on which to compare kinetic binding parameters with the ability to identify tight binding polypeptide tags. The use of the BlAcore® system requires purified antibody and a source of soluble antigen. Phage display-selected clones can be used as a source of peptide antigen and directly analyzed for antibody binding.

C. Theoretical Molecular Modelling of Three-Dimensional Antibody Structure

In silico methods can used to determine capture agent—polypeptide tag pairs. Structural information (NMR and X-ray) is known for numerous immunoglobulins and is accessible, for example, at the Protein Databank (www.rcsb.org/pdb/) and ImMunoGeneTics (www.imgt.cnusc.fr:8104/home.html). Using one of a number of available Molecular Modeling programs such as HyperChem (Hypercube, Inc.), Insightll (Molecular Simulations, Inc.), SpartanPro (Schrodinger, Inc.) Sybyl (Tripos, Inc.) and XtalView (Tripos, Inc.) the structural data can be manipulated in silico to identify potential molecules that can interact with the variable region of the antibody. The energy of interaction between the antibody and potential epitope can be determined using a molecular docking program such as DOCK, which is commercially available; see, also, e.g., (www.cmpharm.ucsf.edu/kuntz/dock.html), AutoDock (www.scripps.edu/pub/olson-web/doc/autodock/), IDock (www.archive.ncsa.uiuc.edu/Vis/Projects/Docker/) or SPIDeR (www.simbiosys.ca/sprout/eccc/spider.html). Once identified and the binding energy is determined in silico, polypeptides that constitute the tags can be synthesized or purchased commercially and tested in vitro for their specificity and affinity for the antibody in question.

d. Raising Antibodies in vivo

Antibodies contemplated herein include polyclonal antibodies, monoclonal antibodies and binding fragments thereof. Polyclonal antibodies are employed where high affinity (avidity) is desired. Polyclonal antibodies are typically obtained by immunizing an animal and isolating the polyclonal antibodies produced by the animal.

For example, antibodies have traditionally been obtained by repeatedly injecting a suitable animal (e.g., rodents, rabbits and goats) with an antigen or antigen with adjuvant (see, e.g., FIG. 2B). If the animal's immune system has responded, specific antibodies are secreted into the serum. The antibody-rich serum (antiserum) that is collected contains a heterogeneous mixture of antibodies, each produced by a different B lymphocyte. The different antibodies recognize different parts of the antigen, and are thus a heterogeneous mixture of antibodies. A homogeneous preparation of antibodies can be prepared by propagating an immortal cell line wherein antibody producing B cells are fused with cells derived from an immortal B-cell tumor. Those hybrids (hybridoma cells) that are producing the desired antibody and have the ability to multiply indefinitely are selected. Such hybridomas are propagated as individual clones, each of which can provide a permanent and stable source of a single antibody (a monoclonal antibody) which is specific for the antigen of interest. The antibodies can be purified from the propagating hybridomas by any method known to those skilled in the art. Fragments of antibodies can be synthesized or produced and modified forms thereof produced.

In one exemplary embodiment, mice are immunized with a collection of peptide binding partners, for example as diptheria toxin-6 mer peptide conjugates. Antibodies are raised against the collection of peptides. A library of hybridoma cells is then generated and clones are screened for their reactivity with individual peptides. Positive clones identify monoclonal antibodies which bind a selected peptide binding partner. The antibodies can be isolated by standard immunopurification techniques or by cloning methods such as by PCR with primers for conserved regions of the anitbody structure. Once the antibody is isolated, the peptide or antigen responsible for the identification of the antibody is conjugated to a molecule and/or biological particle, as described below, and screened against the antibodies isolated above to determine whether the peptides or antigens retain their ability to be captured by the capture agent, thereby identifying a capture agent-binding partner pair.

5. Interactions between Capture Agents and Binding Partners

As noted, the interactions between the capture agents and binding partners are designed or selected to be of relatively high affinity and specificity. Any interaction, including, but are not limited to, hydrophobic, ionic, covalent and van der waals and combinations thereof is contemplated, as long as it meets the criteria of affinity and specificity. Generally the interaction between the capture agent and tag is reversible, such as the interaction between an antibody and an epitope, and has an association or dissociation constant of a value for detection of subsequent binding events between the resulting self-assembled array and other moieties, generally with a K_{d} of at least about 10⁻⁸ M.

Capture agents can be modified following the specific affinity interaction, such as by crosslinking between the binding protein and the capture agent. For example, covalent cross-linking reagents (through chemical, electrical, or photoactivatable methods) can be used to fix or stabilize interactions between proteins (Besemer et al. (1993) Cytokine 5:512-519; Meh et al. (1996) J. Biol Chem. 271:23121-23125; Behar et al. (2000) J. Biol. Chem. 275:9-17; Huber et al. (1993) Eur. J. Biochem. 218, 1031-1039). A cross-link ensures that the interaction between the capture agent and binding partner is long lasting and stable. The initial interaction between the capture agent and the binding partner determines the specificity while the cross-linking agent provides infinite affinity (Chmura et al. (2001) Proc. Natl. Acad. Sci. U.S.A. 98:8480-8484). This affinity can be achieved due to an added synthetic bi-functional cross-linking agent (Besemer et al. (1993) Cytokine 5:512-519; Meh et al. (1996) J. Biol Chem. 271:23121-23125; Behar et al. (2000) J. Biol. Chem. 275:9-17; Huber et al. (1993) Eur. J. Biochem. 218, 1031-1039), or through a reactive group incorporated into the capture agent and the corresponding binding partner, such as a disulfide bond (Chmura et al. (2002) J. Control Release 78:249-258; Kiick et al. (2002) Proc. Natl. Acad. Sci. U.S.A. 99:19-24; Saxon et al. (2000) Org. Lett. 2:2141-2143; Lemieux et al. (1998) Trends Biotechnol. 16:506-513).

The covalent cross-link can also result from the enzymatic function of the binding partner or capture agent. For example, self-splicing proteins known as inteins have been used for the ligation of peptides to a larger protein (Ayers et al. (2000) J. Biol. Chem. 275:9-17), and for the ligation of two subunits of a split-intein protein (Wu et al. (1998) Biochim. Biophys. Acta 1387:422-432; Southworth et al. (1998) EMBO J. 17:918-926). Alternately, several DNA modifying enzymes use a mechanism that involves an intermediate in which the enzyme is covalently bound to its DNA substrate (Chen et al. (1995) Nucleic Acids Res. 23:1177-1183; Topal et al. (1993) Nucleic Acids Res. 21:2599-2603; Thomas et al. (1990) J. Biol. Chem. 265:5519-5530). It is likely that mutation of these enzymes can result in the stabilization of that intermediate, and thus the covalent linkage is retained. These modifying enzymes are highly sequence specific, and presumably can be mutated to create enzymes with distinct specificities. Thus, dsDNA can be used as an effective capture agent with a restriction enzyme or topoisomerase (or binding domain thereof as a binding partner.

6. Molecules and Biological Particles for Displaying

Methods, combinations, kits and systems provided herein initially require the identification of one or more displayed molecules or biological particles for study. As noted, a displayed molecule and/or biological particle is any one or more molecules and/or biological particles whose interaction with other molecules or biological particles is of interest. A selected displayed molecule and/or biological particle can be used to solve a problem, such as, but not limited to, a chemical, biochemical, or biological problem. Problems can be related to diseases, molecular structures, drug discovery, biological activities, biological and/or chemical reactions and mechanisms. For example, the problems can be related to molecular interactions and biological and/or chemical activities, and the displayed molecule and/or biological particle is selected to study these interactions and activities.

a. Exemplary Displayed Molecules and Biological Particles

A displayed molecule and/or biological particle includes any molecule and/or biological particle, such as, but not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; sub-cellular structure; sub-cellular compartment or any combination, portion, salt, or derivative thereof; a virus, such as a viral vector or viral capsid with or without packaged nucleic acid; phage, including a phage vector or phage capsid, with or without encapsulated nucleic acid; a cell, including eukaryotic and prokaryotic cells or fragments thereof; a liposome or micellar agent or other packaging particle, and other such biological materials.

b. Identification of Displayed Molecules and Biological Particles

Any method for identifying molecules and biological particles can be employed to identify a displayed molecule and/or biological particle. Such methods include, but are not limited to, empirical methods, data-mining methods, other methods described herein and by methods apparent to those with skill in the art based upon the description herein.

(1) Empirical

A displayed molecule and/or biological particle can be empirically identified during the course of an experiment. For example, a displayed molecule can be identified during the isolation of molecules and biological particles from complex mixtures. Molecules and biological particles that can be isolated include, but are not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; sub-cellular structure; sub-cellular compartment or any combination, portion, salt, or derivative thereof; a virus, such as a viral vector or viral capsid with or without packaged nucleic acid; phage, including a phage vector or phage capsid, with or without encapsulated nucleic acid; a cell, including eukaryotic and prokaryotic cells or fragments thereof; a liposome or micellar agent or other packaging particle; and other such biological materials. Isolation methods can include, but are not limited to, chromatographic techniques, electrophoretic separations, immunological separations, hybridization techniques, growth and expression techniques and spectroscopic techniques.

Exemplary chromatographic techniques include, but are not limited to ion exchange, size exclusion, affinity, density gradient ultracentrifugation and hydrophobic interaction. Exemplary electrophoretic separation techniques include, but are not limited to, SDS-PAGE, pulsed-field gel electrophoresis, gel mobility or gel shift assays, isoelectric focusing and agarose gel electrophoresis. Exemplary immunological separations include, but are not limited to, Western blotting. Exemplary hybridization techniques include, but are not limited to, Northern blotting and Southern blotting. Exemplary growth and expression techniques include, but are not limited to antibiotic-based selection and isopropylthiogalactoside (IPTG) induction. Exemplary spectroscopic techniques include, but are not limited to, absorbance (ultra violet, visible and infrared), nuclear magnetic resonance, infrared, mass spectrometry, resonance Raman, electron paramagnetic resonance (EPR), electron nuclear double resonance (ENDOreg.), extended x-ray absorption fine structure (EXAFS), circular dichroism (CD), magnetic circular dichroism (MCD), fluorescence, potentiometry and cyclic voltammetry.

(2) Data-Mining

A displayed molecule and/or biological particle can be identified through data-mining techniques based in literature and database analyses. Data-mining techniques include, but are not limited to, search of publicly available databases such as GenBank, Pubmed, SwissProt, EMBL, American Tissue Culture Collection (ATCC), BioMagResBank (BMRB), Protein Data Bank (PDB), Nucleic Acid Database (NDB), and Biological Macromolecule Crystallization Database (BMCD). Data-mining techniques also include sequence homology searches, such as by using the program BLAST, and in silico methods, such as molecular modelling and molecule docking programs. Data-mining techniques also includes search of commercially held databases that include, but are not limited to Comprehensive Medicinal Chemistry (CMC) database (MDL, Inc. San Leandro, Calif.); MACCSII Drug Report (MDDR) database (MDL, Inc. San Leandro, Calif.); Available Chemical Directory (ACD) database (MDL, Inc. San Leandro, Calif.); and the SPECS/BioSPECS database (Specs and BioSPECS, Fijswijk, The Netherlands). Additionally, data-mining techniques includes search of catalogs; journals; newspapers; magazines; the internet; information received via television or radio; information received from a scientific meetings; information obtained through scientific collaboration; and search of any other source well known in the art (see, e.g., Dower et al. (1991) Annu. Rep. Med. Chem. 26:271-280; Fodor et al. (1991) Science 251:767-773; Jung et al. (1992) Angew. Chem. Ind. Ed. Engl. 31:367-383; Zuckerman et al. (1992) Proc. Natl. Acad. Sci. USA 89:4505-4509; Scott et al. (1990) Science 249:386-390; Devlin et al. (1990) Science 249:404-406; Cwirla et al. (1990) Proc. Natl. Acad. Sci. USA 87:6378-6382; and Gallop et al. (1994) J. Medicinal Chemistry 37:1233-1251).

7. Conjugation of a Binding Partner to Displayed Molecul and/or biological particl

The self-assembled arrays include conjugates of one or more displayed molecules or biological particles and a binding partner, which is linked to the displayed molecule and/or biological particle directly or indirectly via a linker. The conjugates typically contain one or more binding partners, where all of the binding partners generally are specific for a single capture agent and one or more molecules or biological particles. Linkage of the components is such that the resulting conjugate when bound to capture agents remains intact. Linkage, for example, can be effected by preparing fusion proteins or by chemically conjugating, such as by covalent bonding, the biological particles or molecules to the binding partner(s).

Thus, the conjugates contain: a) a displayed molecule and/or biological particle; and b) a binding partner, which is linked to the displayed molecule and/or biological particle directly or indirectly via a linker, where the binding partner facilitates attachment of the conjugate to an array containing addressable capture agents.

For convenience and exemplification, the conjugates provided can be represented by the formula:

(BP)ₛ−(L)_{q}−(M)ₚ

wherein q is 0 or an integer of 1 up to n; s and p, which are the same or different, are integers of 1 up to m; and m and n, which are the same or different, are generally 1 or 2, but can be 2, 3, 4, 5, 6 or more as long as the resulting conjugate binds to a capture agent. L is an optional linker, BP is binding partner, M is molecule and/or biological particle and BP is linked either directly or indirectly via one or more linkers to M such that the resulting conjugate remains conjugated when bound to a capture agent. For example, where M is a biological particle such as a cell, each cell can have a plurality of receptors or other surface molecules to which a binding partner binds. In such instances, p can vary from conjugate to conjugate and also can not be readily ascertained. The stoichiometry of each conjugate is not critial to practice of the method. Stoichiometry can be selected and controlled by methods known to those of skill in the art, such as empirically or by selecting appropriate concentrations of the binding partner and moiety to be tagged.

The conjugation can be effected by any method known to those skilled in the art, such as chemically, by recombinant expression of a fusion protein, via a linker molecule and by any combination thereof. For example, the conjugates can be produced by chemical conjugation, such as via thiol linkages, to produce covalent bonds, ionic linkages or linkages via other chemical interactions, such as van der Waals interactions, hydrophobic interactions and other such interaction. The resulting conjugate, however, should be sufficiently stable such that upon binding of a binding partner to a capture agent, the linked molecule and/or biological particle is retained.

Conjugation by recombinant methods results in a fusion protein, where the binding partner or fragment thereof typically is linked to either the N-terminus or C-terminus of molecule, but can be inserted elsewhere. In chemical conjugates, the binding partner or fragment thereof can be linked directly or indirectly via a linker anywhere that conjugation can be effected. As described above, the displayed molecule and/or biological particle can be any molecule and/or biological particle that can be conjugated to a binding partner for use in the self-assembling arrays provided herein.

a. Fusion Proteins

Fusion proteins are exemplary of conjugates provided herein. A fusion protein can contain, for example, a polypeptide displayed molecule and a binding partner. The binding partner can be any molecule that binds to a capture agent, such as an antibody, as described herein, with sufficient affinity, such as, but not limited to, a polypeptide that includes an epitope of known sequence. Exemplary polypeptides for use as binding partners in fusion proteins described herein can, for example, be short polypeptide molecules, such as molecules with at least 5, 6, 8, 10, 15, 20 or more amino acid residues, or can be a full length protein or fragment thereof capable of binding to a capture agent.

A polypeptide binding partner for use in the fusion protein conjugates as described herein can be selected based on known affinity for a specific capture agent, such as the epitope region of an antigen of a known antibody (see, e.g., FIG. 2A). Conversely, a polypeptide binding partner can be selected, and a capture agent can be synthetically designed or prepared to interact with the polypeptide binding partner. For example, an antibody capture agent can be raised from exposure of a host to an antigen, such as a polypeptide (see, e.g., FIG. 2B). Similarly, a full length protein can be selected as the binding partner, such as avidin, and a molecule known to interact with the selected polypeptide, such as biotin, can be used as the capture agent.

The fusion proteins can be produced by recombinant expression of nucleic acids that encode the fusion protein. The formation of a fusion protein involves the placement of two separate coding sequences, such as genes or nucleotides sequences, one encoding the displayed molecule and the second encoding the binding partner, in sequential order in an appropriate cloning vector. Methods for creating an expression vector containing the displayed molecule and the binding partner are well known to those of skill in the art (see, e.g., Sambrook et al. (1989) Molecular Cloning: A Laboratory Manual, Clod Spring Harbor Laboratories, Cold Spring Harbor, New York). Additional methods for the formation of a fusion protein conjugate include, but are not limited to ligation of sequences resulting in linear tagged cDNA molecules; primer extension and PCR for binding partner incorporation; insertion by gene shuffling; recombination strategies; incorporation by transposases; and incorporation by splicing.

Several commercial kits are available for the formation of fusion proteins, which contain the displayed molecule fused to a second protein or nucleotide sequence, including, but not limited to, any of the polypeptides of SEQ ID Nos. 1-34. For example, the GFP Fusion TOPO® cloning vector and the pcDNA-DEST47 Gateway™ vector are available from INVITROGEN (Carlsbad, Calif.) for the expression of a displayed protein fused to GFP; the pET-32a-c(+), the pET-44a-c(+) and the pET-41 a-c(+) vectors are available from NOVAGEN (Madison, Wis.) for the expression of a protein fused to thioredoxin (SEQ ID No.32), NusA (SEQ ID No.29), a HSV tag (SEQ ID No.5), Glutathione S-transferase and a His tag (SEQ ID No.25); and the pShooter vectors from INVITROGEN (Carlsbad, Calif.) for the expression of a protein fused to a c-myc tag (SEQ ID No.6). Further, custom designed and assembled genes and vectors, including those for fusion protein production, can be ordered and prepared by commercial sources, such as by SIGMA GENOSYS (The Woodlands, Tex.).

Some exemplary polypeptides for use as binding partners in the fusion protein conjugates described herein, include, but are not limited to, the peptides with SEQ ID Nos. 1-28, 33 and 34, or full length proteins, including, but not limited to, green fluorescent protein (GFP; SEQ ID No. 35); glutathione S-transferase (GST; SEQ ID No.36); N utilization substance protein A (NusA; SEQ ID No.29), maltose binding protein (MBP; SEQ ID No.30), TATA-box binding protein (TBP; SEQ ID No.31) and thioredoxin (TDX; SEQ ID No.32).

b. Chemical Conjugation or Cross-Linking

To effect chemical conjugation described herein, a displayed molecule and/or biological particle is linked directly or indirectly, such as through a linker, to a binding partner. Chemical conjugation can be used with any molecule, biological particle or binding partner described here, particularly when the displayed molecule and/or biological particle or binding partner is other than a peptide or protein, such as nucleic acid or a non-peptide drug, or production of a fusion protein conjugate is not required. Any methods known to those of skill in the art for chemically conjugating selected moieties can be used.

Any chemical or biological reaction known to those of skill in the art that results in the formation of a linkage between a molecule and/or biological particle and a binding partner can be used to form the conjugates described for use with the self-assembling arrays provided herein. Molecules and biological particles can be coupled to binding partners via direct or indirect linkages, including, but not limited to, covalent, ionic, hydrophobic and van der Waals interactions, as long as the linkage is stable enough to be maintained upon exposure of the conjugate to the addressable capture agents in the self-assembling array. Molecules, such as proteins, and biological particles contain several reactive groups, including, but not limited to, amino, hydroxyl, sulfhydryl, phenolic and carboxyl groups, that can be used as sites of chemical cross-linking to produce novel polymeric structures. Exemplary linkages that are suitable for the formation of chemically linked conjugates include disulfide bonds, thioether bonds, hindered disulfide bonds, and covalent bonds between free reactive groups, such as amine and thiol groups.

Any interaction between molecules and/or biological particles, including, but not limited to, protein:protein, protein:nucleic acid, nucleic acid:nucleic acid, protein:lipid, lipid:lipid, protein:small molecule, receptor:signal, antibody:antigen, peptide nucleic acid:nucleic acid, and small molecule:nucleic acid interactions can be used for the formation of the conjugates used in the self-assembling arrays provided herein. For example, a conjugate can be prepared from the reaction of an enzyme with a mechanism-based inhibitor, which results in the formation of a non-reversible intermediate. Similarly, a conjugate can be formed from the binding interaction of two or more components of a larger molecular complex, such as between an enzyme and a protein co-factor.

Chemical conjugation can also be effected by any method known to those of skill in the art including, but not limited to alteration in environmental conditions, such as alteration in temperature, pH and buffer components, and/or the addition of a compound or molecules known to catalyze the formation of a chemical linkage, such as a cross-linking reagent. For example, cross-linking reagents including, but not limited to, heterobifunctional, homobifunctional and trifunctional reagents, can be used to introduce, produce or utilize reactive groups, such as thiols, amines, hydroxyls and carboxyls, on one or both of the molecules or biological particles or binding partners, which can then be contacted to a target molecule and/or biological particle or binding partner containing a second reactive group, such as a thiol, amine, hydroxyl and carboxyl, to form a chemical linkage between the molecule and/or biological particle or binding partner. These reagents can be used to directly or indirectly, such as through a linker, conjugate a molecule and/or biological particle to a binding partner. Generally, cross-linking reagents have two reactive groups connected by a flexible spacer arm. The reagents differ in their spacer arm length, cleavability, solubility and reactive groups, and can be selected to alter a characteristic of the conjugate complex, such as the solubility, stearic hinderance and permeability. Some cross-linking reagents (i.e., homobifunctional cross-linkers) have the same reactive groups at both ends, others (i.e., hetero-bifunctional cross-linkers) have different reactive groups at the ends and some cross-linkers contain additional functional groups to allow the cross-linker molecule to be labeled. Additionally, some cross-linking reagents (i.e., trifunctional cross-linkers) have three reactive groups to make trimeric complexes.

Cross-linking reactions involving molecules and binding partners, such as proteins, are generally reactive group reactions, such as side chain reactions, and are nucleophilic, resulting in a portion of the end of the cross-linker being displaced in the reaction (the leaving group). Nucleophilic attack is dependent on the pH, temperature and ionic strength of the cross-linking buffer. For example, when the buffer is one to two pH units below the pKa of the reactive group, such as a side chain, the species is highly protonated and is most reactive. One to two pH units above the pKₐ, the species is not protonated and not reactive. The majority of molecules and binding partners, such as proteins, have reactive groups, such as primary amines and free sulfhydrals, available at the surface or terminus of the molecules or binding partner. These are the two most commonly used groups in molecular cross-linking strategies. Cross-linking strategies can also use carbohydrates, carboxyls or other reactive functional groups.

Many factors are considered to obtain optimal cross-linking for a particular application. Factors that affect molecular folding, such as protein folding, (e.g., pH, salt, additives and temperature) can alter conjugation results. Other factors such as molecule or binding partner concentration, cross-linker concentration, number of reactive functional groups available, cross-linker spacer arm length, and conjugation buffer composition should also be considered.

(1) Thiol-Amine and Thiol-Thiol Conjugates The most common schemes for forming a heteroconjugate involve the indirect coupling of an amine group on one molecule and/or biological particle or binding partner to a thiol group on a second molecule and/or biological particle or binding partner, usually by a two- or three-step reaction sequence. The high reactivity of thiols and their relative rarity in most molecules and biological particles make thiol groups ideal targets for controlled chemical cross-linking. If none of the molecule and/or biological particle or binding partner contains a thiol group, then one or more can be introduced using one of several thiolation methods known to those of skill in the art. The thiol-containing molecule and/or biological particle or binding partner is then reacted with an amine-containing molecule and/or biological particle or binding partner using a heterobifunctional crosslinking reagent. Numerous heterobifunctional cross-linking reagents that are used to form covalent bonds between amino groups and thiol groups are known to those of skill in the art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47: 5924-5931; Gordon et al. (1987) Proc. Natl. Acad. Sci. 84:308-312; Walden et al. (1986) J. Mol. Cell Immunol. 2: 191-197; Carlsson et al. (1978) Biochem. J. 173: 723-737; Mahan et al. (1987) Anal. Biochem. 162: 163-170; Wawryznaczak et al. (1992) Br. J. Cancer 66: 361-366; Fattom et al. (1992) Infection & Immun. 60:584-589). These reagents can be used to form covalent bonds between the binding partner and the displayed molecule or biological partner and include, but are not limited to: (4-Succinimidyloxycarbonyl-methyl-a-[2-pyridyldithioltoluene (SMPT); 4-Sulfosuccinimidyl-6-methyl-a-(2-pyridyldithio)toluamido]hexanoate) (Sulfo-LC-SMPT); N-[k-Maleimidoundecanoyloxy]sulfosuccinimide ester (Sulfo-KMUS); Succinimidyl-4-(N-Maleimidomethyl)cyclohexane-1-carboxy-(6-amidocaproate) (LC-SMCC); N-k-Maleimidoundecanoic acid (KMUA); Sulfosuccinimidyl 6-(3-[2-pyridyldithio]-propionamido)hexanoate (Sulfo-LC-SPDP); Succinimidyl 6-(3-[2-pyridyldithio]-propionamido)hexanoate (LC-SPDP); Succinimidyl 4-[p-maleimidophenyl]butyrate (SMPB); Sulfosuccinimidyl-4-(P-Maleimidophenyl)Butyrate (Sulfo-SMPB); Succinimidyl-6-[,β-maleimidopropionamido]hexanoate (SMPH); Sulfosuccinimidyl 4-[N-maleimidomethyl]cyclohexane-1-carboxylate (Sulfo-SMCC); Succinimidyl 4-[N-maleimidomethyl]cyclohexane-1-carboxylate (SMCC); N-Succinimidyl [4-iodoacetyl]aminobenzoate (SIAB); N-Sulfosuccinimidyl [4-iodoacetyl]aminobenzoate (Sulfo-SIAB); N-[g-Maleimidobutyryloxy]sulfosuccinimide ester (Sulfo-GMBS); N-[g-Maleimidobutyryloxy]succinimide ester (GMBS); m-Maleimidobenzoyl-N-hydroxysuccinimide ester (MBS); m-Maleimidobenzoyl-N-hydroxysulfosuccinimide ester (Sulfo-MBS); [N-e-Maleimidocaproyloxy]sulfosuccinimide ester (Sulfo-EMCS); N-e-Maleimidocaproic acid (EMCalif.); [N-e-Maleimidocapropyloxy]succinimide ester (EMCS); N-Succinimidyl-[4-vinylsulfonyl]benzoate (SVSB); N-[β-Maleimidopropyloxy]succinimide ester (BMPS); N-Succinimidyl 3-[2-pyridyldithio]-propionamido (SPDP); Succinimidyl 3-[bromoacetamido]propionate (SBAP); N-[β-Maleimidopropionic acid (BMPA); N-[α-Maleimidoacetoxy]succinimide ester (AMAS); N-Succinimidyl-S-acetylthiopropionate (SATP); and N-Succinimidyl iodoacetate (SIA).

Thiol residues in close proximity can be oxidized to disulfides by either an intra- or intermolecular reaction. In many circumstances, however, this oxidation reaction is reversible and difficult to control. Alternatively, dibromobimane (bBBr) has been used to crosslink thiols in myosin, actin, hemoglobin, Escherichia coli lactose permease and mitochondrial ATPase. It has also been shown to intramolecularly crosslink thiols in a complex of nebulin and calmodulin. In addition, dibromobimane has been used to probe for the proximity of dual-cysteine mutagenesis sites in ArsA ATPase and P-glycoprotein.

The thiol-reactive homobifunctional crosslinker bis-((N-iodoacetyl)piperazinyl)sulfonerhodamine is derived from a relatively rigid rhodamine dye. This crosslinker can also be useful for proximity studies. Other reagents that effect thiol to thiol conjugation include, but are not limited to: 1,4-Di-[3′-(2′-pyridyldithio)-propionamido]butane (DPDPB); 1,11-bis-Maleimidotetraethyleneglycol (BM[PEO]₄); Bis-Maleimidohexane (BMH); 1,8-bis-Maleimidotriethyleneglycol (BM[PEO]₃); 1,6-Hexane-bis-vinylsulfone (HBVS); Dithio-bis-maleimidoethane (DTME); 1,4-bis-Maleimidobutane (BMB); 1,4 bis-Maleimidyl-2,3-dihydroxybutane (BMDB); and Bis-Maleimidoethane (BMOE).

### Introducing Thiol Groups

Several methods are known to those skilled in the art for introducing thiols into molecules, such as polypeptides, or biological particles, including, but not limited to, the reduction of intrinsic disulfides, as well as the conversion of amine, aldehyde or carboxylic acid groups to thiol groups. For example, disulfide crosslinks of cystines in proteins can be reduced to cysteine residues by dithiothreitol (DTT), tris-(2-carboxyethyl)phosphine (TCEP) or tris-(2-cyanoethyl)phosphine.

Reduction can result in loss of protein activity or specificity. Excess DTT should be carefully removed under conditions that prevent reformation of the disulfide, whereas excess TCEP usually does not need to be removed before carrying out the crosslinking reaction. TCEP also is stable at higher pH values than is the air-sensitive DTT reagent.

Amines can be indirectly thiolated by reaction with succinimidyl 3-(2-pyridyidithio)propionate (SPDP; Carlsson et al. Biochem. J. 173, 723-737 (1978)), followed by reduction of the 3-(2-pyridyldithio)propionyl conjugate with DTT or TCEP. Reduction releases the 2-pyridinethione chromophore, which can be used to determine the degree of thiolation. Amines can also be indirectly thiolated by reaction with succinimidyl acetylthioacetate (SATA; Duncan et al. Anal .Biochem. 132: 68-73 (1983)), followed by removal of the acetyl group with 50 mM hydroxylamine or hydrazine at near-neutral pH. This reagent is most useful when disulfides are essential for activity, as is the case for some peptide toxins. SPDP can also be used to thiolate oligonucleotides and to introduce the highly reactive thiol group into peptides, onto cell surfaces or onto affinity matrices for subsequent reaction with fluorescent, enzyme-coupled or other thiol-reactive reagents.

Thiols can be incorporated at carboxylic acid groups by an EDAC-mediated reaction with cystamine, followed by reduction of the disulfide with DTT or TCEP (Lin et al. Biochim Biophys Acta 1038: 382-385 (1990)). Tryptophan residues in thiol-free proteins can be oxidized to mercaptotryptophan residues, which can then be modified by iodoacetamides or maleimides (Wright et al. J. Biol. Chem. 255: 10884-10887 (1980)).

(2) Amine-Amine Conjugates

Conjugation methods for cross-linking two amines also are well known to those skilled in the art. Homobifunctional amine crosslinkers include glutaraldehyde, bis(imido esters), bis(succinimidyl esters), diisocyanates and diacid chlorides (Baumert et al. Methods Enzymol 172: 584-609 (1989)). These reagents tend to yield high molecular weight aggregates, making them unsuitable for preparing conjugates between two different amine-containing biomolecules. Such conjugates are more commonly prepared by thiolating one or more amines on one of the biomolecules and converting one or more amines on the second biomolecule to a thiol-reactive functional group such as a maleimide or iodoacetamide.

Alternatively, direct amine-amine crosslinking routinely occurs during fixation of proteins, cells and tissues with formaldehyde or glutaraldehyde. These common aldehyde-based fixatives also are used to crosslink amine and hydrazine derivatives to proteins and other amine-containing polymers. For example, lucifer yellow CH is nonspecifically conjugated to surrounding biomolecules by aldehyde-based fixatives in order to preserve the dye's staining pattern during subsequent tissue manipulations (Stewart W W. Nature 292: 17-21 (1981)). Also, biotin hydrazides have been directly coupled to nucleic acids with glutaraldehyde, a reaction that is potentially useful for conjugating fluorescent hydrazides to DNA. Cross-linking reagents that conjugate amines to other amines include but are not limited to: Ethylene glycol bis[succinimidylsuccinate](EGS); Ethylene glycol bis[sulfosuccinimidylsuccinate](Sulfo-EGS); Bis[2-(Sulfosuccinimidooxycarbonyloxy)ethyl]sulfone (Sulfo-BSOCOES); Bis[2-(succinimidooxycarbonyloxy)ethyl9 sulfone (BSOCOES); Dithiobis[succinimidylpropionate](DSP); 3,3′-Dithiobis[sulfosuccinimidylpropionate (DTSSP); Dimethyl 3,3′-dithiobispropionimidate.2HCl (DTBP); Disuccinimidyl suberate (DSS); Bis[sulfosuccinimidyl]suberate (BS3); Dimethyl Suberimidate.2HCl (DMS); Dimethyl pimelimidate.2HCl (DMP); Dimethyl adipimidate.2HCl (DMA); Disuccinimidyl glutarate (DSG); Methyl N-succinimidyl adipate (MSA); Disuccinimidyl tartarate (DST); Disulfosuccinimidyl tartarate (Sulfo-DST); and 1,5-Difluoro-2,4-dinitrobenzene (DFDNB)

(3) Conjugates Involving Other Functional Groups

Heterobifunctional conjugates can also be formed from aminecarboxylic acid and thiol-carboxylic acid crosslinking. For example, 1-Ethyl-3-(3-dimethylaminopropyl)carbodiimide (EDAC) can react with biomolecules to form “zero-length” crosslinks, usually within a molecule or between subunits of a protein complex. In this chemistry, the crosslinking reagent is not incorporated into the final product. Rather, the water-soluble carbodiimide EDAC crosslinks a specific amine and carboxylic acid between subunits of a molecule, thereby stabilizing its assembly. Addition of N-hydroxysuccinimide or N-hydroxysulfosuccinimide (NHSS) is reported to enhance the yield of carbodiimide-mediated conjugations, indicating the in situ formation of a succinimidyl ester-activated protein (Staros et al. Anal. Biochem. 156: 220-222 (1986)).

Reaction of carboxylic acids with cystamine (H₂NCH₂CH₂S-SCH₂CH₂NH₂) and EDAC followed by reduction with DTT results in thiolation at carboxylic acids (Lin et al. Biochim. Biophys. Acta 1038: 382-385 (1990)). This indirect route to amine-carboxylic acid coupling is particularly suited to acidic proteins with few amines, carbohydrate polymers, heparin, poly(glutamic acid) and synthetic polymers lacking amines. Other heterobifunctional reagents for the formation of these and other types of conjugates, such as conjugation between a carbohydrate and a thiol, an amine and a non-selective site, include but are not limited to: Sulfosuccinimidyl 2-[7-amino-4-methylcoumarin-3-acetamido]ethyl-1,3′dithiopropionate (SAED); Sulfosuccinimidyl-2-[p-azidosalicylamido]ethyl-1,3′-dithiopropionate (SASD); Sulfosuccinimidyl 2[m-azido-o-nitrobenzamido]-ethyl-1,3′—dithiopropionate (SAND); N-Succinimidyl-6-[4′-azido-2′—nitrophenylaminol hexanoate (SANPAH); N-Sulfosuccinimidyl-6- [4′-azido-2′-nitrophenylaminol hexanoate (Sulfo-SANPAH); Sulfosuccinimidyl [4-azidosalicylamidol-hexanoate (Sulfo-NHS-LC-ASA); Sulfosuccinimidyl-[perfluoroazidobenzamido]ethyl-1,3′-dithiopropionate (SFAD); N-Sulfosuccinimidyl (4-azidophenyl)-1,3′-dithiopropionate (Sulfo-SADP); N-Succinimidyl(4-azidophenyl)-1,3′-dithiopropionate (SADP); N-Hydroxysulfosuccinimidyl-4-azidobenzoate (Sulfo-HSAB); N-Hydroxysuccin-imidyl-4-azidosalicylic acid (NHS-ASA); N-5-Azido-2-nitrobenzoyloxysuccinimide (ANB-NOS); N-[e-Trifluoroacetylcaproyloxy]-succinimide ester (TFCS); Succinimidyl-[4-(psoralen-8-yloxy)lbutyrate (SPB(NHS-Psoralen)); and Sulfosuccinimidyl [2-6-(biotinamido)-2-(p-azidobenzamido)-hexanoamidol-ethyl-1,3′-dithiopropionate (Sulfo-SBED); N-[k-Maleimidoundecanoic acidjhydrazide (KMUH); 4-(4-N-Maleimidophenyl)butyric acid hydrazide hydrochloride (MPBH); 4-(N-Maleimidomethyl)cyclohexane-1-carboxyl hydrazide hydrochloride (M₂C₂H); [N-e-Maleimidocaproic acid]hydrazide (EMCH); 3-(2-Pyridyldithio)propionyl hydrazide (PDPH); 3-Maleimidophenyl boronic acid (MPBA); N-[-Maleimidopropionic acid]hydrazide.TFA (BMPH); N-[4-(p-Azidosalicylamido) butyl]-3′-(2′-pyridyldithio) propionamide (APDP; Thiol to Non-selective); N-[p-Maleimidophenyl]isocyanate (PMPI; Thiol to Hydroxyl); p-Azidobenzoyl hydrazide (ABH; Carbohydrate to Non-selective); p-Azidophenyl glyoxal monohydrate (APG; Non-selective to Non-selective); Bis-[b-(4-Azidosalicylamido)ethyl9 disulfide (BASED; Non-selective to Non-selective); 4-[p-Azidosalicylamido]butylamine (ASBA; Non-selective to Carboxyl); 3-[(2-Aminoethyl) dithiolpropionic acid.HCl (AEDP; Amine to Carboxyl); 1-Ethyl-3-[3-dimethylaminopropyl]carbodiimide Hydrochloride (EDC; Amine to Carboxyl); μ-[Tris(hydroxymethyl)phosphino]proprionic acid (THPP; trifunctional reagent for hydroxyl to amine conjugations); tris-succinimidyl aminotriacetate (TSAT; trifunctional reagent for coupling of primary amines to NHS esters); tris-(2-maleiminoethyl)amine (TMEA; trifunctional reagents for coupling of sulfhydrals with a maleimide group); sulfosuccinimidyl-2-[6-(biotinamido)-2-(p-azidobenzamindo)hexanoamido]ethyl-1,3′-dithiopropionate (Sulfo-SBRD; trifunctional reagent for coupling to primary and secondary amines with a NHS ester group); hydrazide-activated Dextran (trifunctional reagent for coupling aldehyde groups); and aldehyde-activated Dextran (trifunctional reagent for coupling amino groups).

C. Linkers

Any linker known to those of skill in the art for preparation of conjugates can be used herein. These linkers are typically used in the preparation of chemical conjugates. Peptide linkers can be incorporated into fusion proteins. Linkers can be any moiety suitable to associate a molecule and/or biological particle and a binding partner. Such linkers and linkages include, but are not limited to, peptidic linkages, amino acid and peptide linkages, typically containing between one and about 60 amino acids, more generally between about 10 and 30 amino acids, and chemical linkers, such as the heterobifunctional, homobifunctional and trifunctional cross-linkers described above. Other linkers include, but are not limited to, acid cleavable linkers, such as bismaleimideothoxy propane, acid labile-transferrin conjugates and adipic acid dihydrazide, that would be cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30:379-386).

Chemical linkers and peptide linkers can be inserted by covalently coupling the linker to the binding partner and displayed molecule. The heterobifunctional agents, described above, can be used to effect such covalent coupling. Peptide linkers can also be linked by expressing DNA encoding the linker and displayed molecule as a fusion protein as described above. Flexible linkers and linkers that alter the characteristics, including, but not limited to the solubility, stearic hinderance, overall charge, pH stability and cleavability, of the conjugated molecules are contemplated for use, either alone or with other linkers are contemplated herein. In some embodiments, several linkers can be included in order to take advantage of desired properties of each linker.

(1) Acid Cleavable, Photocleavable and Heat Sensitive Linkers

Acid cleavable linkers, photocleavable and heat sensitive linkers can also be used, particularly where it can be necessary to cleave the displayed agent to permit it to be more readily accessible to reaction or retrievable following analysis. Acid cleavable linkers include, but are not limited to, bismaleimideothoxy propane; and adipic acid dihydrazide linkers (see, e.g., Fattom et al. (1992) Infection & Immun. 60: 584-589) and acid labile transferrin conjugates that contain a sufficient portion of transferrin to permit entry into the intracellular transferrin cycling pathway (see, e.g., Welhöner et al. (1991) J. Biol. Chem. 266: 4309-4314).

Photocleavable linkers are linkers that are cleaved upon exposure to light (see, e.g., Goldmacher et al. (1992) Bioconj. Chem. 3: 104-107, which linkers are herein incorporated by reference), thereby releasing the displayed agent upon exposure to light. Photocleavable linkers that are cleaved upon exposure to light are known (see, e.g., Hazum et al. (1981) in Pept., Proc. Eur. Pept Symp., 16th, Brunfeldt, K (Ed), pp. 105-110, which describes the use of a nitrobenzyl group as a photocleavable protective group for cysteine; Yen et al. (1989) Makromol. Chem 190: 69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3: 104-107, which describes a cross-linker and reagent that undergoes photolytic degradation upon exposure to near UV light (350 nm); and Senter et al. (1985) Photochem. Photobiol 42: 231-237, which describes nitrobenzyloxycarbonyl chloride cross linking reagents that produce photocleavable linkages). These reagents are available commercially from sources such as SIGMA-Aldrich, Solulink, PIERCE and Molecular Probes, or can be prepared synthetically using basic techniques known to those skilled in the art.

(2) Peptide Linkers

The linker moieties can also be peptides. Peptide linkers can be employed in fusion proteins and also in chemically linked conjugates. The peptide typically has from about 2 to about 60 amino acid residues, for example from about 5 to about 40, or from about 10 to about 30 amino acid residues. The length selected can depend upon factors, such as the use for which the linker is included.

Peptide linkers are advantageous when the displayed molecule and the binding partners are proteinaceous. For example, the linker moiety can be a flexible spacer amino acid sequence, such as those known in single-chain antibody research. Examples of such known linker moieties include, but are not limited to, peptides, such as (GlyₘSer)ₙ and (SerₘGly)ₙ, in which n is 1 to 6, 1 to 4, typically 2 to 4, and m is 1 to 6, typically 1 to 4, more typically 2 to 4, enzyme cleavable linkers and others. The linker can be incorporated into the conjugate by any method known to those of skill in the art, including, but not limited to, as part of a fusion protein as described above.

(3) Other Linkers

Other linkers include, but are not limited to, those heterobifunctional, homobifunctional and trifunctional cross-linkers described above. These cross-linkers are available from numerous commercial sources, such as PIERCE (Rockford Ill.; www.piercenet.com), Molecular Probes (www.probes.com; Eugene Oreg.) and SIGMA-Aldrich (St. Louis, Mo.; www.sigmaaldrich.com) with a variety of reactive groups for conjugation, lengths of space arm between the reactive groups, cleavability, solubility and permeability characteristics. Other linkers include trityl linkers, particularly, derivatized trityl groups to generate a genus of conjugates that provide for release of therapeutic agents at various degrees of acidity or alkalinity. The flexibility thus afforded by the ability to preselect the pH range at which the therapeutic agent can be released allows selection of a linker based on the known physiological differences between tissues in need of delivery of a therapeutic agent (see, e.g., U.S. Pat. No. 5,612,474). For example, the acidity of tumor tissues appears to be lower than that of normal tissues.

Additional linking moieties are described, for example, in Huston et al., Proc. Natl. Acad. Sci. U.S.A. 85:5879-5883, 1988; Whitlow, M., et al., Protein Engineering 6:989-995, 1993; Newton et al., Biochemistry 35:545-553, 1996; A. J. Cumber et al., Bioconj. Chem. 3:397-401, 1992; Ladurner et al., J. Mol Biol. 273:330-337, 1997; and U.S. Pat. No. 4,894,443.

d. Indirect Linkages

Linkages between a biological molecule and/or particle and a binding partner can also be accomplished through an intermediate molecule as a linker. Intermediate molecules can include any solid or semisolid or insoluble support to which a binding partner and a molecule and/or biological particle can be attached. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials to which binding partners and molecules and/or biological particles can be attached. A intermediate molecule can be of any geometry, such as particulate. When particulate, typically the particles have at least one dimension in the 5-10 mm range or smaller. Such particles, referred collectively herein as “beads,” are often, but not necessarily, spherical. Such reference, however, does not constrain the geometry of the matrix, which can be any shape, including random shapes, needles, fibers, and elongated. Roughly spherical “beads,” particularly microspheres that can be used in the liquid phase, are contemplated.

The intermediate molecules can include additional components, such as magnetic or paramagnetic particles (see, e.g., Dyna beads® (Dynal, Oslo, Norway)) for separation using magnets, as long as the additional components do not interfere with the methods and analyses herein. Such intermediate molecules can also contain identifiers such as electronic, chemical, optical or color-coded labels.

Binding partners can be bound or conjugated to beads by any method known in the art. For example, binding partners can be bound by adhesion to the intermediate molecule or by association of charged groups between them. Binding partners can also be covalently attached to the intermediate molecules by a cross-linker, chemical conjugation or by a chemical linkage such as described herein. Biological molecules and/or particles are attached to the intermediate molecules using non-covalent interactions including electrostatic and hydrogen bonds, covalent interactions or a combination thereof. Such attachments can include adhesion and charge association, as well as covalent binding, such as cross-linking, chemical conjugation or chemical linkage.

Single molecules of a binding partner or multiple molecules of a binding partner can be bound or conjugated to the intermediate molecule. Similarly, single biological molecules and/or particles or multiple biological molecules and/or particles can be bound or conjugated to the intermediate molecule.

In one embodiment, the intermediate molecule is a bead. A binding partner is bound or conjugated to the bead. On the same bead, a biological molecule and/or particle also is conjugated or bound. The bead acts as a linker or intermediate molecule to associate the binding partner and the biological molecule and/or particle so that when the binding partner is bound by the capture agent, the biological molecule and/or particle on the bead becomes associated with the capture agent locus.

8. Imaging and Analytical Software

Provided are software, computer-readable media, computer systems and systems for analyzing the results of experiments and studies that employ self-assembled addressable arrays provided herein.

Software, computer-readable media, computer systems and systems also can be used to analyze results obtained using other addressable arrays. The software, computer-readable media, computer systems and systems provide methods and products for analyzing and imaging the data produced when addressable arrays, such as self-assembled arrays, are employed and certain loci are labeled or stained with luminous or other detectable labels. Because signals from neighboring loci interfere and also because the configurations of the arrays depart from the predicted loci, software, computer-readable media, computer systems and systems to compensate for such differences is provided. Described below is exemplary software, computer-readable media, computer systems and systems. It is understood that those of skill in the art can modify such software, computer-readable media, computer systems and systems based upon this disclosure and that such modifications are included herein.

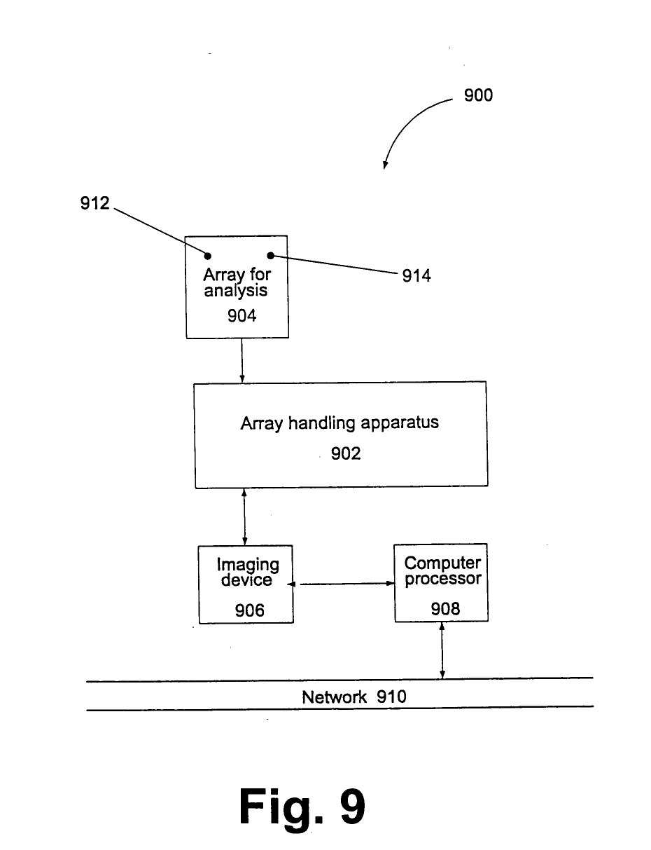

The software, computer-readable media, computer systems and systems can include image analysis software that permits automatic processing of digital image capture data received from a device that images an array, such as a self-assembled array. FIG. 9 is a block diagram of a system 900 that can perform such processing. The system 900 includes an array handling apparatus 902 that can include, for example, an array-receiving table that receives an array 904 to be imaged, wherein the array, such as a self-assembled array, is constructed as described above. If desired, a robotic system can retrieve one or a collection of arrays, such as self-assembled array, from a sample processing station and can deliver them to the array handling apparatus 902. After the processing described herein, the arrays 904 can be removed from the apparatus 902, either manually or by a robotic system.

The array handling apparatus 902 interfaces with an imaging device 906, such as a digital camera or optical scanner, that can produce digital data comprising graphical images of the array or collection of arrays. The array handling apparatus 902 can include a table or other mounting that receives each array 904 such that the array is in the field of view of the imaging device 906. The graphical images can include, for example, video frames that provide pictures of the collection of arrays. The array images are produced in accordance with the imaging device configuration.

An array or collection of arrays 904 can include, for example, an array or collection of arrays, such as self-assembled arrays, that are printed with collections of capture agents, which are bound to binding partners conjugated to molecules or biological particles, that are arranged in a grid arrangement of locus points, thereby providing an array or collection of arrays with loci that are subjected to reagent materials or other processing for observation of chemical or biological reactions. A variety of array sizes and arrangements can be selected as desired. For example, a collection of arrays can contain an arrangement of arrays, such as a 2×5 collection of arrays, each of which contains an array, such as a 8×11 matrix of loci of interest, providing a collection of arrays having 880 loci of interest. Other sizes and arrangements of arrays and collections of arrays can be selected and used, as desired.

The imaging device 906 is typically a conventional CCD camera or optical scanner device that generates digital image data in 8-bit, 1 2-bit, or 16-bit monochrome format, or in 24-bit or 36-bit RGB (red-green-blue) color images. The data can be useful for depicting an optical appearance or characteristic of each locus on the array or collection of arrays 904, such as indicating luminosity of the loci, or reflectivity of the loci under particular types of illuminating light. The image data is useful for detecting a change in the optical appearance or characteristic of each locus after biological processing, such a chemical or biological reaction.

The imaging device 906 typically includes software (or interfaces with a computer device that executes software) that can produce digital graphics files that include image data that is not compressed, such that image intensity data is provided for each locus of interest on the array or collection of arrays, such as the self-assembled arrays provided herein. The uncompressed data accurately provides information relating to luminosity of the loci within the collections of arrays. Such image data can be in bitmap format or in the TIFF (tagged image file format) specification.

Software that permits high-throughput automatic processing of digital image capture data can be executed by a computer processor 908 that retrieves the graphics files comprising the array(s) image data from the imaging device 906. The files can be retrieved from the imaging device or from a network data storage location. The software implements automatic processing of image data for each locus within an array, such as a self-assembled array, of a collection of arrays 904. The computer processor 908 can be connected to a network 910, if network communications are desired, or can be connected to other devices through a communications interface, such as a USB connector or “FireWire” (IEEE 1394) connector.

As described more fully below, the computer processor 908 can include a conventional Personal Computer (PC) desktop system, including a computer processor, keyboard, mouse, and computer display. The computer processor 908 receives the image data from the imaging device 906. The computer processor 908 can, if desired, communicate over a network 910 with other computers. The connection between the computer processor 908 and the network 910 can include a wired connection or a wireless connection. Alternatively, the computer can communicate with other devices using communication interfaces such as a USB connection or “FireWire” connection or the like.

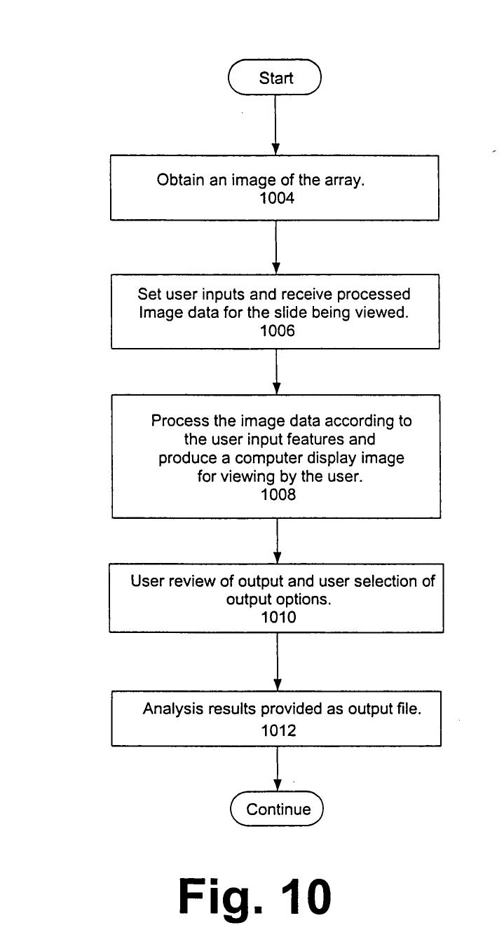

FIG. 10 is a flow diagram that illustrates the processing that is controlled by the computer 908. Each array or collection of arrays is printed and prepared for processing, which involves exposing the array or collection of arrays to reagents, such as chemical or biological reagents, or other biological components for observing reactions or interactions of interest. Each array or collection of arrays is then inserted into the array handling apparatus chamber one at a time, or the array or collection of arrays can be provided into the chamber by an automatic robotic array handling subsystem that positions each array in its respective desired location for imaging. The processing to which the array or collection of arrays are subjected prior to insertion inside the chamber can typically involve application of the reagents and other processing referred to above, as known to those skilled in the art.

The flow diagram box 1004 indicates that the first operation of the computer system is to obtain an image of the array or collection of arrays, such as self-assembled arrays. The image is typically produced by a charge coupled device (CCD) camera or an optical scanner apparatus that produces digital video image data output as TIFF files. Suitable cameras can be obtained, for example, from the EASTMAN KODAK Company (Rochester N.Y., USA). The TIFF image data can be received directly from the imaging device or can be received as a data file over a computer network or over a device communication interface. In the next operation, indicated by the box 1006, the user provides array processing input selections for the image data that is to be processed and analyzed.

The array processing input selections of box 1006 includes selections by the system user according to menu options presented on the computer system display, and can include specification of input features of the array or collection of arrays under examination. For example, the user might choose an array image geometry configuration from among a menu presenting choices of a 96-locus configuration or an 880-locus configuration. Alternatively, a user might explicitly set the geometry configuration of the collection of arrays, such as by specifying that the loci within an array in the collection are laid out in four rows and twenty-four columns (to provide a 96-locus configuration), or the user might specify loci arranged in a 16×16 locus array, thereby providing a 256-locus configuration, or the user might specify an 2×5 collection of arrays, each of which includes an 8×11 array of loci. Other configurations can be provided, according to the likely collections of arrays with which the system 900 can be used.

Another configuration input parameter that can be provided by a user for a particular array or collection of arrays, such as self-assembled arrays, is the designation of array or collection of arrays calibration position registration marks. Each array or collection of arrays includes two or more control loci 912, 914 (see FIG. 9) that are used to calibrate the position of the array image in the device field of view. When the array(s) image data is received by the computer, a corresponding real-time image is provided to the display device of the computer, where it is viewed by the user. The system user moves a display cursor over the display image by using a computer mouse and clicks a mouse button when the cursor is centered over each control locus, in turn. For an array or collection of arrays, the control loci 912, 914 are typically arranged to be in a top left location of a top left array in the collection and in a top left location of a top right array in the collection. The user can move the display cursor and designate the location of each control locus in the display image, to designate the location of the control loci relative to the imaging device field of view and thereby determine the expected location of all the loci of the array or collection of arrays in the image.

Finally, another input parameter that the user can specify in box 1006 is to select image correction processing. The image correction processing can involve background luminosity removal and compensation for locus neighbor effects in the locus image data. The background removal processing determines background luminosity values received by the imaging device over the top surface of the array or collection of arrays, such as self-assembled arrays, and subtracts the background value from the luminosity value otherwise determined for each locus. The neighbor effects compensation involves examining luminosity data for all the array loci adjacent to each locus of interest. Each of these techniques removes luminosity effects from sources other than the locus of interest and therefore can contribute to more accurate luminosity data for the array loci over the surface of the array or collection of arrays.

At box 1008, the input parameters specified by the user are implemented as the image data from the array or collection of arrays is processed. As described further below, the image data from the array(s) is processed on a pixel by pixel basis. The background subtraction, neighbor effects compensation, and any other processing parameters specified by the user in box 1006 are carried out in the next operation.

The background subtraction substantially removes any luminosity that is contributed by reflection from the surface of the array or collection of arrays, such as self-assembled array(s), or luminosity (that is, self-illumination) from the array(s) upper surface or the like. Those skilled in the art understand that reflection from the surface of the array(s) can be received in the field of view of the imaging device and can lead to increased luminosity values for a locus, incorrectly increasing the luminosity value for the locus. When the user selects background subtraction as an input processing option, the system automatically subtracts a background luminosity value from the illumination detected by each locus. Thus, the system assumes a predetermined amount of background luminosity over the upper surface of the array(s), and compensates accordingly.

In contrast to the background illumination removal, the input processing option of compensation for locus neighbor effects is a method of reducing luminosity from particular loci that are adjacent to a locus of interest. Those skilled in the art can understand that, depending on how closely spaced the array loci are, the luminosity (illumination contribution) from neighboring loci can enter the imaging device field of view of a particular locus and can increase the luminosity value of that particular locus. For example, a locus located centrally in an array would be surrounded by eight adjacent loci. A locus located along the outer edge of an array has five surrounding loci. These loci can have sufficient luminosity to enter the imaging device field of view for the central locus and can incorrectly increase the luminosity value that otherwise would be observed for the central locus. Similarly, a corner location has only three neighbor loci. The magnitude of the inter-locus luminosity effects, which can be referred to as cross-talk, can be dependent on the spacing of the loci and also on the particular imaging device construction, configuration, and sensitivity.

Therefore, using what is referred to as a decay model for luminosity of an array locus and information for the imaging device in question, the computer processing can determine the effect on the luminosity data for the locus by determining the luminosity from the neighbor loci. The system determines the luminosity value from each locus of the array. Then, for a particular locus or array locus, the system applies a “decay model” (such as an exponential decay function) to determine the amount of light contribution likely to be received in the imaging device from each of the neighbor loci in addition to the image intensity value from the center of the particular locus of interest, and subtracts the sum of the neighbor luminosity values from the particular locus luminosity data. The decay model typically incorporates neighbor effects data determined for the particular imaging device specified by the user in requesting the neighbor effects processing. Alternatively, the neighbor effects data might be based on the class of imaging device being used, such as CCD still camera, or video camera, or the like. The distance from the particular locus to each of the particular locus neighbors is determined on a locus-by-locus basis, as described further below.

One of the user input parameters is to specify the array geometry within a collection of arrays in terms of the grid configuration in number of rows and number of columns within an individual array, which determines a resultant spacing between loci. That is, the expected spacing from locus to locus can be known once the geometric configuration and number of loci within each array is specified by the user. It is known, however, that the printing of the displayed loci and actual laying down of any reagent materials might result in deviations in the expected spacing. Therefore, one locus in a row or column might not be aligned with the other loci in that row or column. If the array or collection of arrays is moved relative to the imaging device field of view so as to center over an expected locus location, and if that locus is not correctly centered, then the luminosity value for that locus can be artificially reduced from its actual value. The computer processing of the software can determine the actual spacing of the locus relative to the expected locus location within the array or collection of arrays.

Therefore, the actual locations of all the loci within the array or collection of arrays can be determined, thereby determining the distance from locus to locus. These distances are used in the compensation for neighbor locus illumination effects processing.

To determine the actual location of a locus and to determine the locus deviation from an expected array location, the computer processing detects pixel-by-pixel illumination values in the digital image data received from the imaging device as an expected locus location is approached. Because of the control locus designation by the user, and the array geometry within the collection of arrays indicated by the user, the expected location of a locus center is known for each locus in the array(s). It is expected that a locus within an array(s) can have greatest luminosity in the center of the locus and can have decreased luminosity toward the edges of the locus. The software processing therefore can detect increasing pixel illumination values as the digital image pixel data being processed comes first from the edge of a locus and then comes from locations toward the center of the locus. Once the center of the locus is passed, the pixel image data being processed can start to decrease. The software processing notes the local maximum of illumination data for a particular locus and records that corresponding canvas grid coordinate as the center of that locus. The corresponding grid location can be stored in the computer memory. The software processing repeats the operations for each locus and identifies the center of illuminosity data for each locus, thereby also determining the offset or misregistration of each locus from the expected grid array coordinates. Those skilled in the art can understand that knowing the expected location of each locus can be used to prevent excessive hunting or location divergence by the computer processing that could otherwise jeopardize the efficient determination of actual locus location.

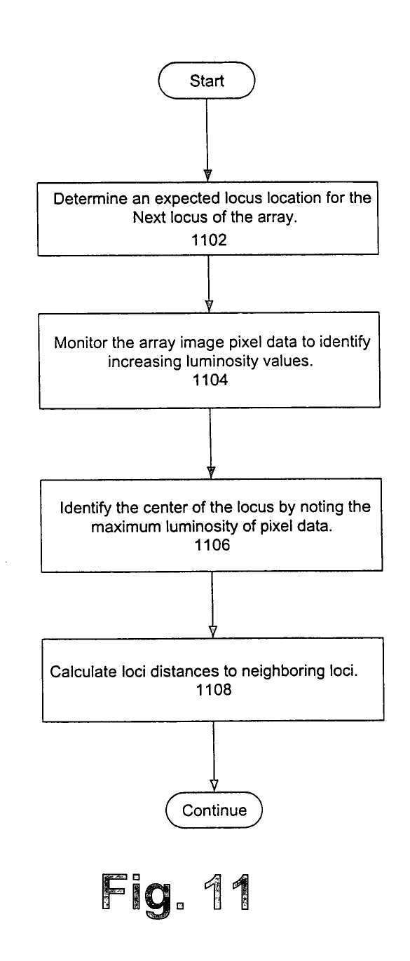

FIG. 11 is a flow diagram that represents the operations executed by the computer system for each locus to determine actual locus location in an array(s), in accordance with the description above. In the first operation, represented by the flow diagram box numbered 1102, an expected locus location is determined. As noted above, this can be known for all loci within the array(s) after the user input of grid geometry and spacing for the array(s). The next operation, box 1104, is to monitor image pixel data from the array(s) once a prior locus center (or edge of the array(s) grid) to identify the next locus center. The system processes the digital image data on a pixel by pixel basis. The operation of box 1104 involves detecting the increasing pixel luminosity values that are expected as the center of the next locus is approached.

When pixel luminosity value decreases, the identification of the locus center is established. This identification is represented by box 1106. In accordance with the size of loci being used and the nature of the detected biological reactions at each locus, the software processing can use an averaging approach to identify a local locus maximum. Those skilled in the art understand appropriate averaging techniques for the reactions involved. For example, it might be appropriate to average values over a running average of four pixel values, or over some other range of pixel image data. When the locus center is identified, its grid coordinates for the array(s) are determined in relation to the two control loci that were previously aligned by the user. Therefore, the exact distance from a locus relative to each of its neighbor loci also can be calculated. These calculations are represented by box 1108 of FIG. 11. These distance values are used in the processing of the neighbor effects compensation indicated by the user in the input parameters, as implemented by the processing of box 1008 in FIG. 10.

The user can designate a set of output parameters that can be applied after the user input processing (box 1006) is applied to the image data received from the imaging device. This processing is part of the box 1010 processing. The output processing is applied after the viewer has an opportunity to view the image data. That is, after the user input parameters have been used to process the received image data, the user can view the processed data. The user can then indicate which output options, if any, can be used for modified processing followed by delivery to the user in a format that can be more easily accessed. That is, a user can select output options that differ from a default set of options. The user can designate, for example, that output be provided in a spreadsheet representation that arranges the data for each of the array loci into a table. Such data arrangements are commonly available through software applications such as provided, for example, by the “EXCEL” program application from MICROSOFT Corporation (Redmond Wash., USA). If desired, the output tables can be arranged in a text table that provides array coordinates and corresponding intensity values for each array locus of interest. Alternatively, a more graphic representation of the intensity data can be provided, such as a chart or histogram. In addition, coordinates of array loci can be provided with microscope settings to permit rapid settings of an accompanying system microscope for viewing of the loci of interest with a microscope.

Once the user has provided the input options, the received image data is processed at the computer system, as indicated in box 1008 of FIG. 10. After the user has selected the desired output options, processing of the computer continues with processing in accordance with the user selections. The output processing is represented by the flow diagram box numbered 1012 in FIG. 10.

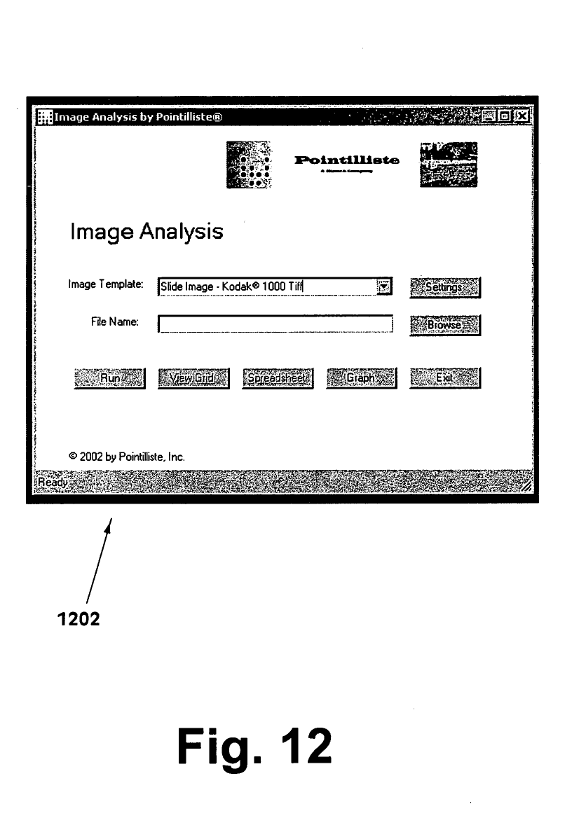

FIG. 12 is a representation of a display window 1202 from which a user can designate the input features that can be invoked. The display window is produced on a display image of the system. This is part of the processing for box 1006 of FIG. 10. The window 1202 represents the main user interface display from which the user selects processing options and specifies input. The Image Template drop down menu box is the means for selecting a particular input (imaging) device and configuration. The illustration shows “Slide Image—KODAK 1000 Tiff” to indicate that the imaging device is a “KODAK” brand Model 1000 camera with TIFF output files. Other selections can be chosen from the drop-down list, in accordance with the family of devices, configurations, and output formats that are supported by the image analysis software. The File Name box is the means by which the user specifies the TIFF input file to be processed. The “Browse” button lets the user select from among all available files on the computer 908 or otherwise available from a computer network.

The “Run” display button of FIG. 12 initiates processing of the selected input file for image analysis and for output. The user does not select this operation until all the input specifications and parameters have been entered. The “View Grid” button lets the user view a display that indicates the actual location of loci (that is, canvas loci where a reaction is indicated) after the software has performed image analysis. The “Spreadsheet” button calls up the spreadsheet output for viewing. The “Graph” button shows a graphical output (described further below) of the software. “Exit” ends the software execution. The “Settings” button causes display of an input window through which the user specifies input options.

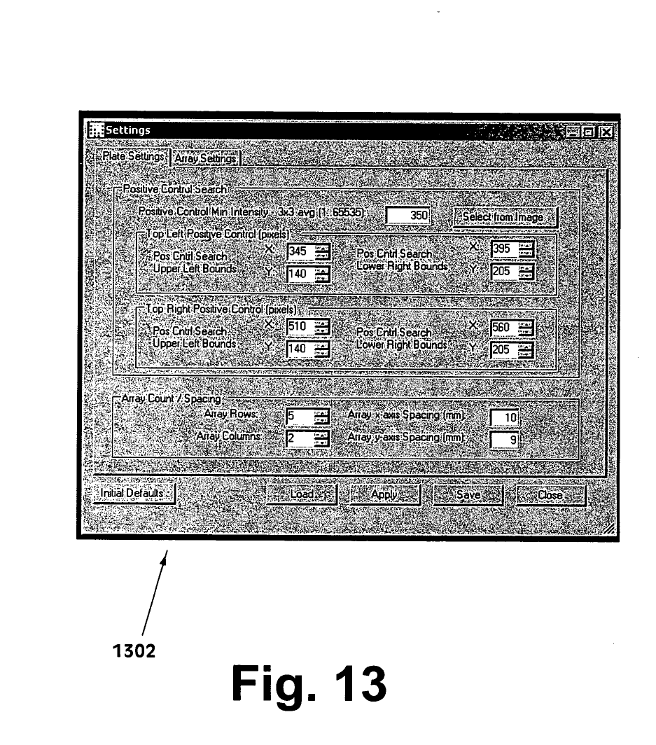

FIG. 13 shows the window 1302 that is produced when the user selects the “Settings” display button from the FIG. 12 window and then selects the “Plate Settings” tab from the resulting “Settings” window. The “Positive Control Min Intensity” permits a user to specify a luminosity value (over a pixel averaging number of data points) to serve as a minimum intensity for indicating a reaction. The “Select from Image” button calls up a representation of the array or collection of arrays image and permits the user to select a particular point or area of an array or collection of arrays for image analysis. The other display boxes permit direct entry of values for the position control marks described above and for entering the coordinates of maximum array or collection of arrays dimensions. The “Array Count/Spacing” area of the display includes display boxes for entry of array dimensions and point spacing. The values shown in the window 1302 includes default values; they appear in the “Plate Settings” display without any intervention or action by the user. A user is free to alter any of the input parameter specifications, as needed. The default values are provided as a convenience to the user, to reduce the amount of data entry needed, because the default values are automatically entered in the boxes by the software. It should be understood, however, that a user is expected to enter the appropriate numbers where it is known that the actual parameters for an array or collection of arrays are different from the default values. The “Select from Image” button of FIG. 13 permits selection of an image file from the computer and permits direct setting of the control points.

The “Load” button at the bottom of FIG. 13 loads the input parameter settings from a saved file and populates all the boxes of the input parameter settings with those values. The “Apply” button causes the software to be run with those settings. The “Save” button causes the parameter settings in the boxes to be stored into a memory file location for recall later.

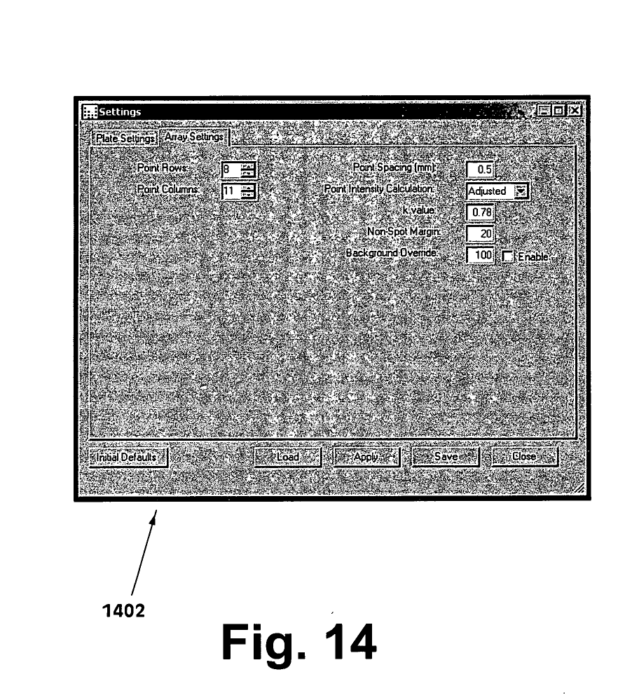

FIG. 14 shows the window 1402 that is produced when the “Array Settings” tab of the “Settings” display is selected. Array settings for an array or each of the arrays within a collection of arrays can be provided (for example, where the collection of arrays is a 2×5 collection of arrays, each of which is an 8×11 array). FIG. 14 shows an example of an Array Settings display in which the array(s) are specified as having eight rows and eleven columns. Other array or collections of arrays input settings can be provided, as illustrated.

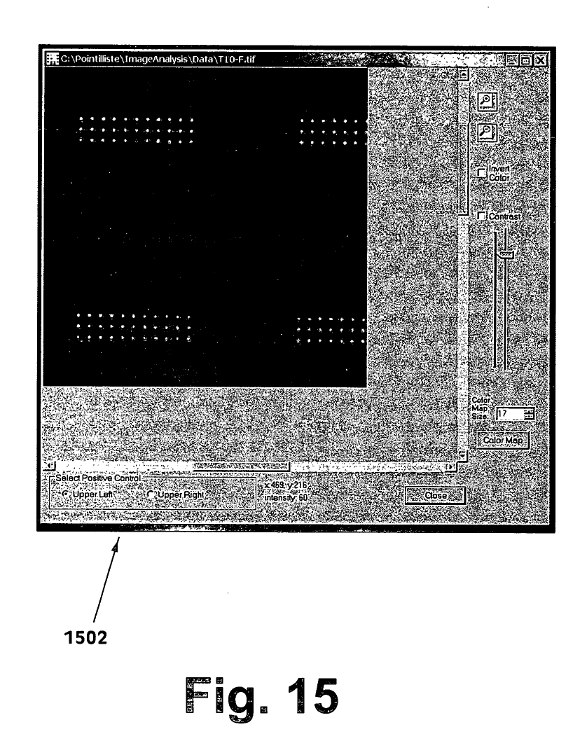

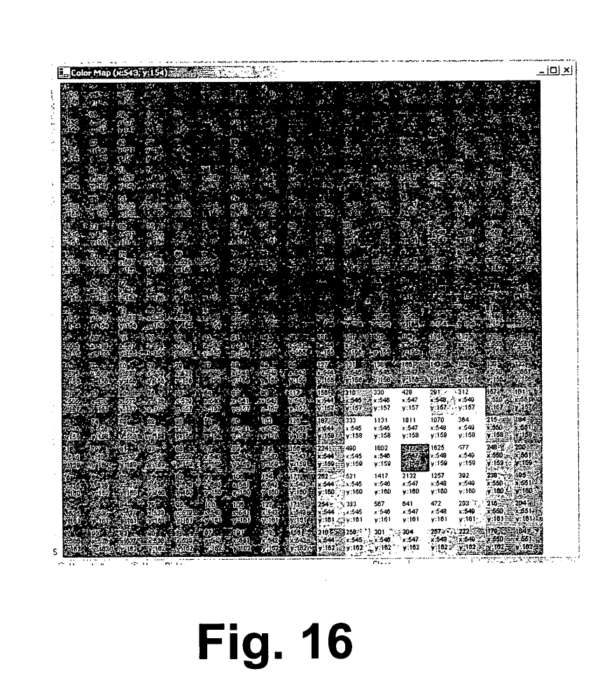

FIG. 15 shows the display window 1502 that results from choosing the “Select from Image” button of FIG. 13. Choosing “Select from Image” provides the user with a visual image of the array or collection of arrays, which occupies the darkened area of FIG. 15. The user can select the “Color Map” button of FIG. 15 (shown in the lower right corner) to view a color map of a particular area of the array or collection of arrays, based on the TIFF data that was read by the software. An example of a color map is depicted in FIG. 16. The color map provides a color-coded representation of the array or collection of arrays image. The color map includes a value for image intensity (luminosity) for each locus or point in the image, and a value for an x-coordinate and a y-coordinate of the array or collection of arrays. The color of each x-y pair or data cell is keyed to image intensity value. For example, a red square can represent the cells of greatest intensity (luminosity) values, and a blue square can represent the cells of least intensity values.

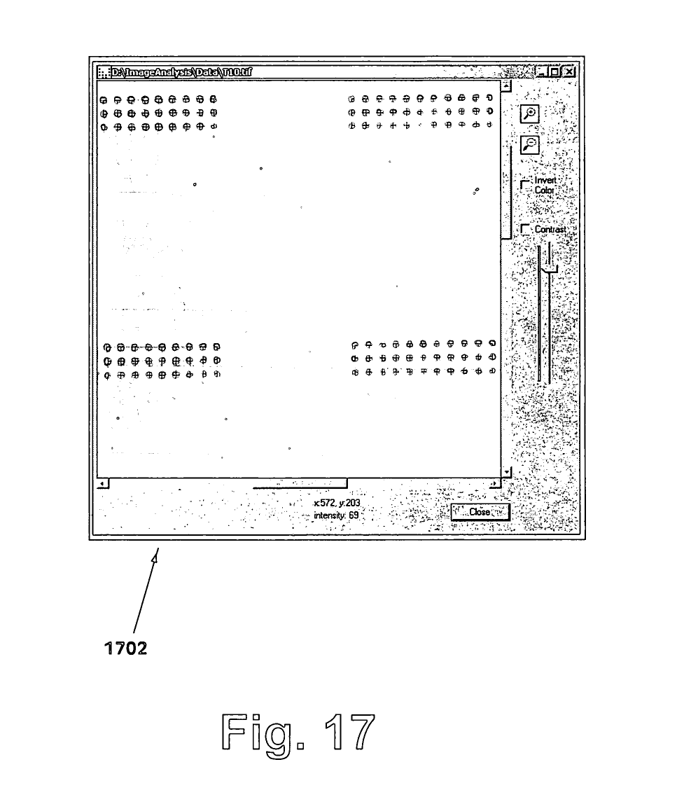

FIG. 17 shows a graph output window 1702 that is produced after image analysis by the software. FIG. 17 depicts a set of 8×9 grids that are overlaid on a collection of data points. In FIG. 17, each grid is associated with a 3×9 collection of light-colored points. These points are the locus of a detected reaction on the array(s). The dark, irregular grid below each distinct collection of light-colored points indicates locations where the image analysis software did not detect a reaction or otherwise find data that indicated an actual locus of interest. A slider can be used to adjust image contrast and location of the canvas area being viewed can be changed with horizontal and vertical image bars.

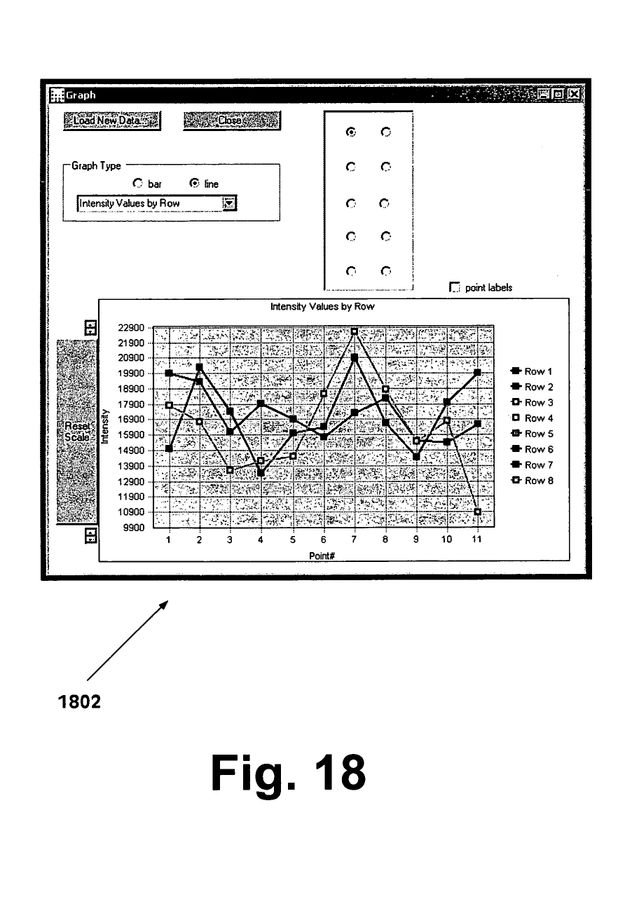

FIG. 18 shows a graph output window 1802 that results from selecting the “Graph” button on the image analysis main window (see, e.g., FIG. 12). The graph is provided by a chart plug-in module or the like that operates within the image analysis software. A visual representation of the array or collection of arrays is provided in the upper right area of the display window and the intensity values are plotted below. In FIG. 18, the graph is selected as intensity values row-by-row in the “Graph Type” box. Other configurations, such as a bar graph, can be. selected from the drop-down menu. It should be noted that FIG. 18 shows only three rows of graph data, rather than the eight rows indicated as available in the graph legend at the right side of the window 1802, because only three of the eight rows contained actual data (see FIG. 17).

### C. Combinations and Kits

Provided herein are combinations of chemical and/or biological reagent(s), including, but not limited to addressed capture agents, such as capture agents printed on a solid support, binding partners and conjugation reagents. Kits containing such reagents in packaged form, optionally including instructions for use thereof, also are provided. The instructional information typically can be in printed form, but also can be in an electronic or computer readable format on a computer readable medium or on the internet, such as, but not limited to, CD-ROM disks (CD-R, CD-RW), DVD-RAM disks, DVD-RW disks, floppy disks and magnetic tape.

In addition to the reagents, the kits optionally include software or means for viewing, modifying, processing, analyzing or manipulating the image data, e.g., array images, such as, but not limited to, highlighting a specific locus of interest; moving and zooming in on the loci; removing background an neighboring loci luminosity; and permitting analysis of the image pattern. Further, the kits optionally contain in a paper and/or computer-readable format instructions and/or information, such as, but not limited to, information on array assembly, on conjugation protocols, on tutorials, on experimental procedures, on reagents, on related products, on available experimental data, on using kits, on additional pattern recognition information, on literature, and on other information. The kits optionally also contain in a paper and/or computer-readable format information on minimum hardware requirements and instructions for running and/or installing the software. The kits optionally also include, in a paper and/or computer readable format, information on the manufacturers, warranty information, availability of additional reagents, technical services information, and purchasing information. The kits and combinations optionally include a video or other viewable medium or a link to a viewable format on the internet or a network that depicts the use of the reagents, assembly of the arrays, use of the software, and/or use of the kits. The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

In other embodiments, the kits contain reagents, such as an addressed capture agent and binding partner pair and a conjugation reagent; a computer readable medium, such as CD ROM disk(s), containing computer-readable instructions for viewing, manipulating and analyzing image data; and, optionally, additional information on paper and/or on the CD-ROM disk as described herein. The computer-readable information (data) provided in the kits described herein, optionally, includes program instructions that, when executed by the computer, provide a viewer that produces one or more graphical images on a display corresponding to the self-assembled array image(s). The viewer provides the user display controls that support manipulation, modification, and analysis of the images. The data for the viewer can be recorded on the same computer-readable medium as the image data, can be provided on a separate medium or can be downloaded or accessed via a network connection or internet.

In other embodiments, the kits contain reagents, such as an addressed capture agent and a conjugation reagent; a list of sequence information, such as amino acid sequences of the binding partner or the sequence of nucleic acid molecules that encodes the binding partner, for binding partner molecules that specifically interact with the provided capture agents; a computer readable medium, such as CD ROM disk(s), containing computer-readable instructions for viewing, manipulating and analyzing images; and, optionally, additional information on paper and/or on the computer readable medium, such as a CD-ROM disk, as described herein. The computer-readable information (data) provided in the kits described herein, optionally, includes program instructions that, when executed by the computer, provide a viewer that produces one or more graphical images on a display corresponding to the self-assembled array image(s). The viewer provides the user display controls that support manipulation, modification, processing and analysis of the images. The data for the viewer can be recorded on the same computer-readable medium as the image data, can be provided on a separate medium or can be downloaded or accessed via a network connection or internet.

1. Reagents

Reagents provided herein include, but are not limited to, addressed capture agents, such as printed arrays thereof, binding partners and conjugation reagents. Such reagents can be provided as kits optionally including instructions for preparing self-assembled arrays.

The addressed capture agents and binding partners include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; sub-cellular structure; sub-cellular compartment or any combination, portion, salt, or derivative thereof; a virus, such as a viral vector or viral capsid with or without packaged nucleic acid; phage, including a phage vector or phage capsid, with or without encapsulated nucleic acid; a cell, including eukaryotic and prokaryotic cells or fragments thereof; a liposome or micellar agent or other packaging particle, and other such biological materials.

Reagents for kits can also include linkers and intermediate molecules, such as beads, for linking with binding partners and biological molecules and/or particles. In one embodiment, binding partners are provided conjugated to beads. In another embodiment, suitable beads are provided along with a plurality of binding partners. Conjugation agents can be provided for linking binding partners to beads and for linking biological molecules and/or particles to the beads.

The conjugation reagents include any compound known to effect cross-linking between or among two or more molecules, such as between capture agents and solid supports, between intermediate molecules and binding partners and/or molecules and biological particles, and between binding partners and biological molecules and/or particles. The conjugation reagents provided herein include, but are not limited to, ethylene glycol bis[succinimidylsuccinate](EGS); Ethylene glycol bis[sulfosuccinimidylsuccinatel (Sulfo-EGS); Bis[2-(Sulfosuccinimidooxycarbonyloxy)ethyl]sulfone (Sulfo-BSOCOES); Bis[2-(succinimidooxycarbonyloxy)ethyl]sulfone (BSOCOES); Dithiobis[succinimidylpropionate](DSP); 3,3′-Dithiobis[sulfosuccinimidylpropionate (DTSSP); Dimethyl 3,3′-dithiobispropionimidate.2HCl (DTBP); Disuccinimidyl suberate (DSS); Bis[sulfosuccinimidyl]suberate (BS3); Dimethyl Suberimidatee2HCl (DMS); Dimethyl pimelimidate.2HCl (DMP); Dimethyl adipimidate*2HCl (DMA); Disuccinimidyl glutarate (DSG); Methyl N-succinimidyl adipate (MSA); Disuccinimidyl tartarate (DST); Disulfosuccinimidyl tartarate (Sulfo-DST); 1,5-Difluoro-2,4-dinitrobenzene (DFDNB); (4-Succinimidyloxycarbonyl-methyl-a-[2-pyridyldithio]toluene (SMPT); 4-Sulfosuccinimidyl-6-methyl-a-(2-pyridyldithio)toluamido]hexanoate) (Sulfo-LC-SMPT); N-[k-Maleimidoundecanoyloxy]sulfosuccinimide ester (Sulfo-KMUS); Succinimidyl-4-(N-Maleimidomethyl)cyclohexane-1-carboxy-(6-amidocaproate) (LC-SMCC); N-k-Maleimidoundecanoic acid (KMUA); Sulfosuccinimidyl 6-(3-[2-pyridyidithio]-propionamido)hexanoate (Sulfo-LC-SPDP); Succinimidyl 6-(3-[2-pyridyidithio]-propionamido)hexanoate (LC-SPDP); Succinimidyl 4-[p-maleimidophenyl]butyrate (SMPB); Sulfosuccinimidyl-4-(P-Maleimidophenyl)Butyrate (Sulfo-SMPB); Succinimidyl-6-[β-maleimidopropionamido]hexanoate (SMPH); Sulfosuccinimidyl 4-[N-maleimidomethyl]cyclohexane-1-carboxylate (Sulfo-SMCC); Succinimidyl 4-[N-maleimidomethyl]cyclohexane-1-carboxylate (SMCC); N-Succinimidyl [4-iodoacetyl]aminobenzoate (SIAB); N-Sulfosuccinimidyl [4-iodoacetyl]aminobenzoate (Sulfo-SIAB); N-[g-Maleimidobutyryloxy]sulfosuccinimide ester (Sulfo-GMBS); N-[g-Maleimidobutyryloxy]succinimide ester (GMBS); m-Maleimidobenzoyl-N-hydroxysuccinimide ester (MBS); m-Maleimidobenzoyl-N-hydroxysulfosuccinimide ester (Sulfo-MBS); [N-e-Maleimidocaproyloxy]sulfosuccinimide ester (Sulfo-EMCS); N-e-Maleimidocaproic acid (EMCalif.); [N-e-Maleimidocaproyloxy]succinimide ester (EMCS); N-Succinimidyl-[4-vinylsulfonyl]benzoate (SVSB); N-[β-Maleimidopropyloxylsuccinimide ester (BMPS); N-Succinimidyl 3-[2-pyridyldithiol-propionamido (SPDP); Succinimidyl 3-[bromoacetamidolpropionate (SBAP); N-[β-Maleimidopropionic acid (BMPA); N-[α-Maleimidoacetoxy]succinimide ester (AMAS); N-Succinimidyl-S-acetylthiopropionate (SATP); N-Succinimidyl iodoacetate (SIA); Sulfosuccinimidyl 2-[7-amino-4-methylcoumarin-3-acetamido]ethyl-1,3′dithiopropionate (SAED); Sulfosuccinimidyl-2-[p-azidosalicylamidolethyl-1,3′-dithiopropionate (SASD); Sulfosuccinimidyl 2[m-azido-o-nitrobenzamido]-ethyl-1,3′-dithiopropionate (SAND); N-Succinimidyl-6-[4′-azido-2′-nitrophenylaminol hexanoate (SANPAH); N-Sulfosuccinimidyl-6-[4′-azido-2′-nitrophenylaminol hexanoate (Sulfo-SANPAH); Sulfosuccinimidyl [4-azidosalicylamido]-hexanoate (Sulfo-NHS-LC-ASA); Sulfosuccinimidyl-[perfluoroazidobenzamido]ethyl-1,3′-dithiopropionate (SFAD); N-Sulfosuccinimidyl (4-azidophenyl)-1,3′-dithiopropionate (Sulfo-SADP); N-Succinimidyl(4-azidophenyl)-1,3′-dithiopropionate (SADP); N-Hydroxysulfosuccinimidyl-4-azidobenzoate (Sulfo-HSAB); N-Hydroxysuccinimidyl-4-azidosalicylic acid (NHS-ASA); N-5-Azido-2-nitrobenzoyloxysuccinimide (ANB-NOS); N-[e-Trifluoroacetylcaproyloxy]-succinimide ester (TFCS); Succinimidyl-[4-(psoralen-8-yloxy)]butyrate (SPB(NHS-Psoralen)); Sulfosuccinimidyl [2-6-(biotinamido)-2-(p-azidobenzamido)-hexanoamido]-ethyl-1,3′-dithiopropionate (Sulfo-SBED); 1,4-Di-[3′-(2′-pyridyldithio)-propionamido]butane (DPDPB); 1,11-bis-Maleimidotetraethyleneglycol (BM[PEO]₄); Bis-Maleimidohexane (BMH); 1,8-bis-Maleimidotriethyleneglycol (BM[PEO]₃); 1,6-Hexane-bis-vinylsulfone (HBVS); Dithio-bis-maleimidoethane (DTME); 1,4-bis-Maleimidobutane (BMB); 1,4 bis-Maleimidyl-2,3-dihydroxybutane (BMDB); Bis-Maleimidoethane (BMOE); N-[k-Maleimidoundecanoic acidlhydrazide (KMUH); 4-(4-N-Maleimidophenyl)butyric acid hydrazide hydrochloride (MPBH); 4-(N-Maleimidomethyl)cyclohexane-1-carboxyl hydrazide hydrochloride (M₂C₂H); [N-e-Maleimidocaproic acidlhydrazide (EMCH); 3-(2-Pyridyidithio)propionyl hydrazide (PDPH); 3-Maleimidophenyl boronic acid (MPBA); N-[β-Maleimidopropionic acid]hydrazide.TFA (BMPH); N-[4-(p-Azidosalicylamido) butyl]-3′-(2′-pyridyldithio) propionamide (APDP); N-[p-Maleimidophenyl]isocyanate (PMPI); p-Azidobenzoyl hydrazide (ABH); p-Azidophenyl glyoxal monohydrate (APG); Bis-[b-(4-Azidosalicylamido)ethyl9 disulfide (BASED); 4-[p-Azidosalicylamidol butylamine (ASBA); 3-[(2-Aminoethyl) dithiolpropionic acid*HCl (AEDP); and 1-Ethyl-3-[3-dimethylaminopropyl]carbodiimide Hydrochloride (EDC).

2. Types of Kits

The kits, containing reagents, including, but not limited to, addressed capture agents, such as capture agents printed on a solid support, binding partners and conjugation reagents, and, optionally, additional information in a paper and/or computer readable format, additional reagents and instructions for conjugation of the binding partner to a displayed molecule and/or biological particle, and imaging and processing software in a computer readable format or available via the internet, provided herein, can be used as kits for, but not limited to, the following experiments, assays, and/or protocols: cloning kits, yeast expression systems, insect expression systems, bacterial expression systems, prokaryotic expression systems, cell culture systems, genomic analysis kits, protein analysis kits, recombination kits, protein detection kits, nucleic acid sequencing kits, protein sequencing kits, electrophoresis kits, transfection kits, genomic detection kits, labeling kits, PCR kits, gene expression kits, hybridization kits, mutagenesis kits, transcription kits, translation kits, DNA purification kits, RNA purification kits, protein purification kits, genomic library kits, DNA synthesis kits, RNA synthesis kits, protein and peptide synthesis kits, antibody kits, enzyme kits and other kits. The combinations, systems and kits provided herein can also be used with commercially available kits to perform such assays, experiments and treatments. In one embodiment, combinations, systems, and kits provided herein can be used to identify compounds that interact with polypeptides, such as antibodies, that are conjugated to the self-assembled array.

In another embodiment, imaging and processing software and information contained in a computer readable format, and, optionally, additional information, is provided in a kit with reagents for protein analysis. In another embodiment, image information contained in a computer readable format, and, optionally, additional information, is provided in a kit with reagents for drug screening.

In another embodiment, imaging and processing software and information contained in a computer readable format, and, optionally, additional information, is provided in a kit with reagents for the analysis of various biochemical and chemical processes, products and collections, including, but not limited to, gene expression; a genomic library; PCR products; DNA transcription; RNA translation; DNA and RNA synthesis products and intermediates; nucleic acid sequencing; protein sequencing; transfection; protein and peptide synthesis products and intermediates; enzyme activity analysis; antibody-antigen interactions; antibody specificity; protein or nucleic acid mutagenesis; DNA and RNA purification; nucleic acid hybridization; recombination processes; binding affinity assays; drug screening; protein interaction; cell morphology; signal transduction; complexation; membrane translocation; electron transfer; conversion of a reactant to a product via a catalytic mechanism; chaperoning of compounds inter- and intracellularly; fusion of liposomes to membranes; infection of a foreign pathogen into a host cell or organism, such as a virus (HIV, influenza virus, polio virus, adenovirus, etc.) or bacteria (Escherichia coli, Pseudomonas aeruginosa, Salmonella enteritidis, etc.); initiation of a regulatory cascade; detoxification of cells and organisms; and cell replication and division. In another embodiment, image information contained in a computer readable format, and, optionally, additional information, is provided in a kit with reagents for protein labeling, detection, synthesis, and/or purification.

### D. Computer Systems

The kits and combinations provided herein contain chemical and/or biological reagents, such as capture agents, binding partners and conjugation reagents, and image data processing information, such as image pattern databases, and, optionally, additional information as described herein in an electronic and/or computer readable format. The computer-readable information (data) optionally includes program instructions that, when executed by the computer, provide a viewer that produces one or more graphical images on a display corresponding to the image data, such as an image pattern. The viewer provides user display controls that support manipulation, modification, processing and analysis of the images. The data for the viewer can be recorded on the same computer-readable medium as the image data, such as image pattern data, can be provided on a separate medium or can be downloaded or accessed via a network connection. In one embodiment, the image data are provided on CD-ROM disks, but a variety of data storage techniques are available to a skilled artisan for creating a computer readable medium having recorded thereon image data as described herein. The choice of the data storage structure is generally based on the method chosen to access the stored information.

A variety of data processor programs and formats can be used to store the image data information on computer readable medium. The image pattern information can be represented, for example, in a word processing text file, formatted in commercially-available software such as MICROSOFT Word®, graphics files or represented in the form of an ASCII file, stored in a database application, such as, but are not limited to, DB2®, Sybase® and Oracle®. A skilled artisan can adapt any number of data processor structuring formats (e.g., text file or database) in order to obtain computer readable medium having recorded thereon the information as described herein.

Computer systems are readily available. The processing that provides the displaying and analysis of image data for example, can be performed on multiple computers or can be performed by a single, integrated computer or any variation thereof. For example, each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM). Each computer typically includes a program product reader that accepts a program product storage device from which the program product reader can read data (and to which it can optionally write data). The program product reader can include, for example, a disk drive, and the program product storage device can include a removable storage medium such as, for example, a magnetic floppy disk, an optical CD-ROM disc, a CD-R disc, a CD-RW disc and a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods understood by those skilled in the art. The computer that implements the client side processing, and the computer that implements the server side processing or any other computer device of the system, can include any conventional computer suitable for implementing the functionality described herein.

To implement the functionality described herein, a suitable computer for viewing and analyzing image data, such as array image patterns, includes a “Pentium III” level CPU having at least 128 MB of memory, 500 MB of disk storage, at least 32MB video card, at least 24× CDROM drive, and optionally peripherals such as a 3-button mouse. A configuration for performing user tasks includes, for example, a “Pentium III” processor at 500 MHz or faster, memory of 128 MB or greater, disk storage space of 500 MB or more, and a CD-ROM 24× or faster. Other configurations, depending on the applications being used and the computer performance desired, can be selected by the skilled artisan.

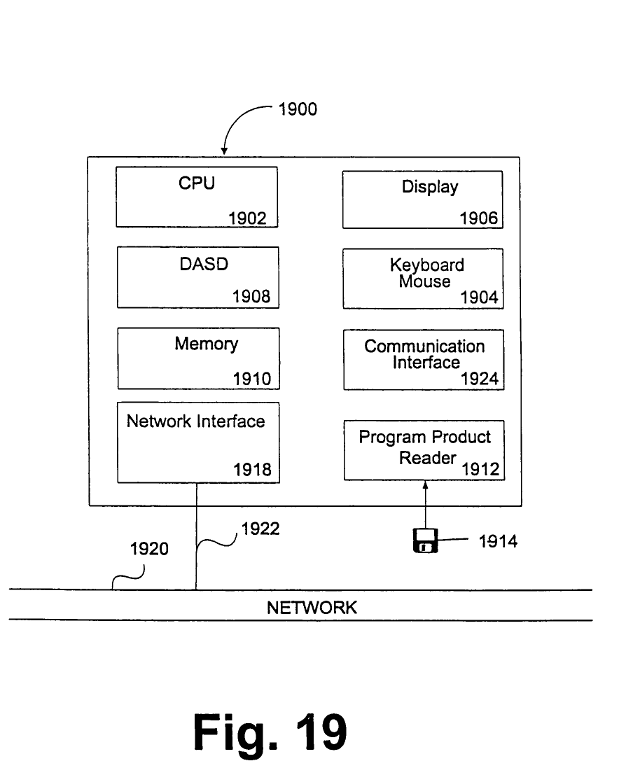

FIG. 19 is an example of a suitable computer system 1900 that can implement the functionality described herein. FIG. 19 depicts an exemplary computer 1900 that can include the computer processor 908 (FIG. 9). Each computer 1900 operates under control of a central processor unit (CPU) 1902, such as a “Pentium 4” microprocessor and associated integrated circuit chips, available from Intel Corporation (Santa Clara, Calif., USA). A computer user can input commands and data from a keyboard and computer mouse 1904, and can view inputs and computer output at a display 1906. The display is typically a video monitor or flat panel display. The computer 1900 also includes a direct access storage device (DASD) 1908, such as a hard disk drive. The memory 1910 typically includes volatile semiconductor random access memory (RAM). Each computer typically includes a program product reader 1912 that accepts a program product storage device 1914, from which the program product reader can read data (and to which it can optionally write data). The program product reader can include, for example, a disk drive, and the program product storage device can include removable storage media such as a magnetic floppy disk, a CD-R disc, a CD-RW disc, or DVD disc.

Each computer 1900 can communicate with the others over a computer network 1920 (such as the Internet or an intranet) through a network interface 1918 that enables communication over a connection 1922 between the network 1920 and the computer. The network interface 1918 typically includes, for example, a Network Interface Card (NIC) and a modem that permits communications over a variety of networks. The computer 1900 also can communicate with other devices or computers through a communication interface 1924. The communication interface can include, for example, a USB connector or a “FireWire” (IEEE 1394) connector.

The CPU 1902 operates under control of programming steps that are temporarily stored in the memory 1910 of the computer 1900. When the programming steps are executed, the computer performs its functions. Thus, the programming steps implement the functionality of the respective client or server. The programming steps can be received from the DASD 1908, through the program product storage device 1914, or through the network connection 1922. The program product reader 1912 can receive a program product 1914, read programming steps recorded thereon, and transfer the programming steps into the memory 1910 for execution by the CPU 1902. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation in accordance with the methods can be embodied on a program product.

Alternatively, the program steps can be received into the operating memory 1910 over the network 1920. In the network method, the computer receives data including program steps into the memory 1910 through the network interface 1918 after network communication has been established over the network connection 1922 by well-known methods understood by those skilled in the art without further explanation. The program steps are then executed by the CPU 1902 thereby comprising a computer process. E. Uses of Combinations, Kits and Systems The methods, combinations, kits and systems provided herein can be used to monitor and analyze interactions of target molecules and/or biological particles, including, but not limited to, a ligand, receptor, cell or drug, with a self-assembled array. The displayed molecules and/or biological particles, such as antibodies, enzymes and cells, are conjugated with a specific binding partner then contacted with addressed capture agents, such as capture agents printed on an a solid support as a positionally addressable array. Target molecules and/or biological particles can then be exposed to the self-assembled array and interactions among or variations in activity of the target molecules and/or biological particles and the self-assembled array can be assessed. Optionally, a perturbation, such as a candidate compound or condition, can be added prior to, simultaneously with or after exposure of the target molecule and/or biological particle to the self-assembled array and the interaction or activity reassessed, thereby determining the effect of the perturbation on the interaction or activity being monitored. Thus, the self-assembled arrays (capture systems) provided herein enable one to sort displayed molecules or biological particles into discrete loci and then monitor and analyze interactions between the self-assembled array and target molecules and/or biological particles in the presence or absence of a perturbation.

The target molecules and/or biological particles that can be exposed to the self-assembling arrays described herein, include, but not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; sub-cellular structure; sub-cellular compartment or any combination, portion, salt, or derivative thereof; a virus, such as a viral vector or viral capsid with or without packaged nucleic acid; phage, including a phage vector or phage capsid, with or without encapsulated nucleic acid; a cell, including eukaryotic and prokaryotic cells or fragments thereof; a liposome or micellar agent or other packaging particle, and other such biological materials.

The following sections and subsections describe the use of self-assembled arrays to identify, monitor and assess interactions between displayed molecules and/or biological particles and target molecules and/or biological particles in the presence or absence of a perturbation. It is understood that these are exemplary only and other applications are intended to be included.

1. Identifying Perturbations that Modulate an Interaction or Secondary Effect of an Interaction between a Self-Assembled Array and a Target Molecule and/or Biological Particle

Methods using self-assembling arrays to identify perturbations that modulate an interaction or secondary effect of an interaction between displayed molecules and/or biological particles and target molecules and/or biological particles are provided. In some embodiments, the displayed molecule, such as antibodies, or biological particle are captured, exposed to a target molecule and/or biological particle and a readout, i.e., stimulation of a particular pathway, expression of a reporter or other detectable event, is assessed. Alternatively, perturbations, such as candidate compounds or conditions, can be added to the self-assembled array prior to, simultaneously with or after exposure to a target molecule and/or biological particle and their effect on the interaction of the target molecule and/or biological particle and self-assembled array can be determined (FIGS. 4A and 4B). In another embodiment, a perturbation, such as a candidate compound or condition, can be added to or mixed with a target molecule and/or biological particle prior to exposure to the self-assembled array.

Perturbations include conditions and compounds that modulate interactions of molecules and/or biological particles. The perturbations can be conditions and/or candidate compounds that are known to modulate interactions; such perturbations are employed in methods in which the interaction is studied. Perturbations also can be conditions and candidate compounds whose effect is unknown; such perturbations are screened against a particular interaction, such as in the screening of a drug compound against a particular interaction. Such perturbations are identified using known interactions and effects of such interactions.

Conditions include environmental parameters which can be varied to determine the alteration of an interaction or the secondary effect resulting from an interaction, and include, but are not limited to, pH, ionic strength, aerobic versus anaerobic environment, temperature, pressure, time, concentration of components, agitation, and organic versus aqueous interaction medium. The alteration of environmental conditions can include varying one experimental parameter or multiple parameters simultaneously or sequentially.

Candidate compounds used in the methods provided herein include, but are not limited to, an organic compound, inorganic compound, metal complex, receptor, enzyme, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, biopolymer, polymer, sub-cellular structure, sub-cellular compartment or any combination, portion, salt, or derivative thereof. Libraries of any of these molecules and biological particles, such as but not limited to, cyclic peptide and small molecule libraries, can also be used in the methods provided herein.

Candidate compounds can be obtained from any source, including commercial sources (e.g., MAYBRIDGE Chemical Co. (Trevillet, Cornwall, UK), COMGENEX (Princeton, N.J.), Brandon Associates (Merrimack, N.H.), and Microsource (New Milford, Conn., Aldrich (Milwaukee, Wis.), Pan Laboratories (Bothell, Wash.) or MycoSearch (N.C.)) synthetic production, collaborative exchange, compound libraries, expression, isolation, or purification techniques, or any other source known to those skilled in the art. Additionally, candidate compounds can be obtained from natural and synthetically produced libraries that are readily modified through conventional chemical, physical, and biochemical methods and products. Candidate compounds can be optionally labelled, such as with a luminescent molecule, to facilitate detection of the interaction or the effect of the interaction using any methods known to those skilled in the art.

Candidate compounds and/or conditions identified or utilized by the methods described herein are molecules and/or biological particles that are screened against an interaction or used to modulate molecular interactions or chemical and/or biological activity. Candidate compounds and/or conditions can affect an interaction between molecules and/or biological particles of an interaction in a negative or positive fashion. As a non-limiting example, a candidate compound and/or condition can enhance an interaction between molecules and/or biological particles by facilitating the interaction of molecules and/or biological particles of the interaction with one another. In contrast, a candidate compound and/or condition can reduce or inhibit an interaction by preventing molecules and/or biological particles of an interaction from interacting with one another. Thus, candidate compounds and/or conditions can serve as, for example, activators, inhibitors, competitive inhibitors, agonists, partial antagonists, partial agonists, inverse agonists, antagonists, cytotoxic agents, and drugs for target interactions and chemical and/or biological activity that are studied.

If a particular interaction is implicated in diseases and/or disorders, the self-assembled arrays provided herein can be used to identify perturbations, such as candidate compounds and/or conditions that modulate the interaction. Candidate compounds and/or conditions that are identified by these methods can have remedial, therapeutic, palliative, rehabilitative, preventative, prophylactic or disease-impeditive effects on patients suffering from, or potentially predisposed to developing, such diseases and disorders. Alternatively, the self-assembled arrays provided herein can be used to screen candidate compounds or conditions against a target interaction to aid in the diagnosis and prognosis of patients suffering from such diseases and disorders. If a particular interaction is part of a biological mechanism or reaction, the self-assembled arrays provided herein can be used to identify candidate compounds and/or conditions that can serve as a modulator of that mechanism or activity. As a non-limiting example, screening candidate compounds or conditions with an interaction using the self-assembled arrays provided herein can aid in understanding a biological and/or chemical mechanism and/or activity.

a. Perturbations and Screening Methods

Provided herein are methods for use of the self-assembled arrays for screening perturbations, such as candidate compounds and/or conditions, for modulatory effects on an interaction (FIG. 4A) or the secondary effect of an interaction (FIG. 4B). Perturbations, such as candidate compounds and/or conditions, are identified by contacting the perturbation with a self-assembled array (capture system) either prior to, simultaneously with or after exposure of a sample containing a target molecule and/or biological particle and detecting a modulation of an interaction between the target molecule and/or biological particle and the self-assembled array or a secondary effect of the interaction. A variation in the interaction or the secondary effect of the interaction in the presence of a perturbation, such as a candidate compound and/or condition, in comparison to the interaction in the absence of the perturbation is indicative of the effect of the candidate compound and/or condition on the interaction. Perturbations, such as candidate compounds and/or conditions, shown to modulate interactions or alter the effect of an interaction between target molecules and/or biological particles and the self-assembled array can be selected for further analyses or for use in the modulation of the interaction or the effect of the interaction, including, but not limited to, as activators, inhibitors, competitive inhibitors, agonists, partial antagonists, partial agonists, inverse agonists, antagonists, cytotoxic agents, and drugs.

b. Use of Perturbations to Identify Interactions

Also provided herein are methods of use of the self-assembled arrays for identifying interactions between molecules and/or biological particles. Interactions between molecules and/or biological particles can be identified by contacting a perturbation, such as a candidate compound and/or condition, that has a known effect on a particular interaction (FIG. 4A and 4B) prior to, simultaneously with or after exposing a self-assembled array to a sample containing target molecules and/or biological particles and assessing interaction or the effect of the interaction. The presence of the known effect of the perturbation, such as a candidate compound and/or condition, indicates that a particular interaction is present among the self-assembled array and the target molecules and/or biological particles. In this type of screening, many target molecules and/or biological particles can be screened against known perturbations, such as candidate compounds and/or conditions, in order to pinpoint specific interactions. Optionally, once a particular interaction or effect of an interaction is identified, the interaction or effect of the interaction can then be further screened as stated in the section above for other perturbations, such as candidate compounds and/or conditions, that further modulate the interaction or effect of the interaction.

2. Cell Surface Profiling

The cell membrane in eukaryotic and prokaryotic cells is a fluid phospholipid bilayer embedded with proteins and glycoproteins. The phospholipid bilayer is arranged so that the polar ends of the molecules form the outermost and innermost surface of the membrane while the non-polar ends form the center of the membrane. In addition, it contains glycolipids as well as complex lipids called sterols, such as the cholesterol molecules found in animal cell membranes, that are not found in prokaryotic membranes. The sterols make the membrane less permeable to most biological molecules, help to stabilize the membrane, and probably add rigidity to the membranes aiding in the ability of eukaryotic cells lacking a cell wall to resist osmotic lysis. The proteins and glycoproteins in the cytoplasmic membrane are quite diverse and include, but are not limited to, channel proteins to form pores for the free transport of small molecules and ions across the membrane; carrier proteins for facilitated diffusion and active transport of molecules and ions across the membrane; cell recognition proteins that identify a particular cell; receptor proteins that bind specific molecules such as hormones, cytokines, and antibodies; and enzymatic proteins that catalyze specific chemical reactions.

Various cell types differ in the types and number of biomolecules present on the surface of the cell. This variation can be correlated to their function within the larger organism. For example, B cells function as a source of antibodies for the immune system. T cells help to eliminate pathogens that reside inside host cells. For this function, T cells display a surface molecules, such epitope receptors called T-cell receptors (TCRs).

3. Receptor Agonist/Antagonist Discovery

All hydrophilic molecules and the hydrophobic prostaglandins effect cellular responses via specific cell membrane receptors on the target cell. These protein receptors bind the signalling molecule with great affinity and transduce the signal into intracellular signals that affect cellular behavior. Cell surface receptors do not regulate gene expression directly, rather they relay a signal across the cell membrane and the response of the target cell depends on intracellular second messenger molecules such as cAMP, inositol phosphate, or calcium.

There are several families of cell surface receptors based on signal transduction mechanism. Channel-linked receptors are transmitter gated ion channels involved in rapid synaptic signalling as in nervous tissue or the neuromuscular junction. A specific transmitter can rapidly open or close ion channels upon binding to its receptor thus changing the ion permeability of the cell membrane. All of these receptors belong to a family of similar multipass transmembrane proteins. Catalytic receptors behave as enzymes when activated by a specific ligand. Most of these have a cytoplasmic catalytic region that behaves as a tyrosine kinase. Target proteins are phosphorylated at specific tyrosine residues thus changing their activation state. When bound to a specific ligand, G-protein linked receptors indirectly activate or inactivate a separate plasma membrane bound enzyme or ion channel. The interaction between the receptor and the affected enzyme or ion channel is mediated by a GTP binding protein. G-protein linked receptors initiate a cascade of chemical events within the target cell that usually alter the concentration of small intracellular messengers such as cAMP or inositol triphosphate. These intracellular messengers in turn alter the behavior of other intracellular proteins. The effects of all these second messengers are rapidly reversible when the extracellular signal is removed. The response of cells to external signals initiates signalling cascades that can greatly amplify and regulate various inputs.

The combinations, kits, methods and systems provided herein can be used to identify molecules that interact with a cell surface receptor. The interaction between the molecule and the receptor can be monitored either directly or indirectly by observing a secondary response. For example, a sample containing cells with G protein-linked receptors can be exposed to a library of displayed molecules on a self-assembled array and allowed to interact. The interaction between the displayed molecules and the G cell surface receptor within the sample can be monitored directly through any method known to those skilled in the art. Optionally, a secondary response to the interaction, such as, but not limited to, transcription of a gene, immunostaining of secondary messenger such as cAMP and detection of the stimulation of a secondary enzyme, such as a protein kinase can be monitored. In addition, exogenous perturbations, such as candidate compounds and/or conditions, can be added to the self-assembled array prior to, simultaneously with or after exposure to the target sample. Alteration in the interaction between the displayed molecule and the target sample and/or secondary effect of the interaction can be detected. This detection can result in the identification of candidate compounds and/or conditions that modulate the interaction between the biological particle and the self-assembled array (capture system) or the secondary effect of the interaction. 4. Protein-Protein Interactions Including Association-Dissociation Assays and Changes in Protein Conformation

Interactions among proteins are responsible for many of the enzymatic reactions found in nature. Interactions include, but are not limited to, electron transport from an electron source by a shuttle protein to an enzymatic protein for the conversion of reactants to products at the active site; chemical cleavage reactions, such as the formation of a mature protein from its zymogen; hetero- or homomultimer formation for catalytic activity or complex stability; protective shuttling of toxic compounds from the source within the cell to the enzyme responsible for detoxification; chaperoning of metal or other cofactors within the cell for incorporation into an apoprotein; the post-translational modification, such as glycosylation or the hydroxylation of specific residues, of nascent polypeptides; and the more efficient folding of proteins following translation.

The methods provided herein can be used to identify molecules, biological particles and other moieties that bind to other target molecules and/or biological particles, such as cell-surface receptors or enzymes. For example, a target cell can be any cell type which contains a naturally-occurring or engineered protein or proteins, which includes a conformation-specific readout (e.g., myosins) or an interaction-specific readout (e.g., BRET (bioluminescence resonance energy transfer)-based NF-kB/IF-kB interactions). A library of molecules and/or biological particles can be displayed by the self-assembled array, then exposed to a sample containing the target cells. By using a detection method, such as resonance energy transfer techniques or spectroscopic techniques, receptor-induced changes in protein conformation or protein-protein interactions can be monitored and assessed.

5. Biopolymer Degradation Assays

Biopolymers and small molecules often undergo chemical cleavage reactions as part of their respective synthesis and/or reaction mechanism. Most proteins undergo some manner of proteolytic cleavage during post-translational modification. For example, many proteins, such as proteolytic enzymes, are biosynthesized as larger, inactive precursors known as zymogens or proenzymes. An exemplary group, the serine proteases, are synthesized and stored in the pancreas as inactive precursors. Storage of these enzymes in their zymogenic form prevents damage to proteins in the pancreatic cells. After secretion from the pancreas into the small intestine, the zymogens are activated by selective proteolysis of one or a few select peptide bonds, resulting the formation of the active form of the proteolytic enzymes. Similarly, many transmembrane proteins or proteins that are destined to be secreted are synthesized with an N-terminal signal peptide. A signal recognition particle (SRP) binds a ribosome synthesizing a signal peptide to a receptor on the membrane and conducts the signal peptide and the following nascent polypeptide through it. Once the signal peptide has passed through the membrane, it is specifically cleaved from the nascent polypeptide by a signal peptidase.

For oligonucleotides, an example of chemical cleavage can be found in the processing of messenger RNA (mRNA). In eukaryotic systems, the formation of mRNA begins with the transcription of an entire structural gene, including its introns, to form pre-mRNA. Following capping and polyadenylation, the introns are excised and their flanking exons spliced together to yield the mature mRNA. A spliceosome, a large assembly of RNA and protein molecules, performs the pre-mRNA splicing. The spliceosome is a dynamic machine, which is assembled on the pre-mRNA from separate components and parts enter and leave it as the splicing reaction proceeds.

The methods provided herein can be used for monitoring chemical cleavage reactions of biopolymers. For example, resonance energy transfer-based systems can be used by tagging a single protein with two fluorescent probes. When the protein is intact, the two fluorophores are in close proximity and a signal can be detected. When the protein is degraded, there is no signal. A sample containing the labelled protein or a sample containing cells that have been transfected with this construct can be exposed to a displayed library of molecules on a self-assembled array and a signal from the labelled protein is detected. Using this system, molecules can be identified which lead to the degradation of a specific protein within a sample or a cellular system.

6. Protein Trafficking Assays

The interior of the cell is organized into an array of membrane-bound compartments, each of which is composed of a specific set of resident proteins. The localization of integral membrane proteins to these compartments is, in many cases, mediated by short linear sequences of amino acids that function as specific sorting signals. The signals are recognized by receptor-like molecules that connect the signals to the sorting machinery. The methods provided herein can be used to define the molecular basis for protein biogenesis at specific sub-cellular locations, to elucidate the mechanisms responsible for intracellular protein transport and membrane fusion and to monitor the movement of proteins within a biological particle.

For example, to monitor movement (trafficking) of polypeptides within a biological particle, fusion proteins can be made with fluorescent tags such as GFP. Once cells are transfected, they can be exposed to a displayed library of molecules, such as signalling peptides and other extracellular signals, and molecules can be identified that lead to alternate localization of the protein of interest. In addition, proteins of unknown function can be tagged and tracked in a similar manner to determine their sub-cellular localization to gather some information leading towards a function determination.

7. Analysis of Modulation of Subcellular Conditions and Processes

Cells includes a variety of subcellular compartments including, for example, organelles. An organelle is a structural component of a cell that is physically separated, typically by one or more membranes, from other cellular components, and which carries out specialized cellular functions. Organelles and other subcellular compartments vary in terms of their composition and number in cells derived from different tissues, among normal and abnormal cells, and in cells derived from different species. Accordingly, organelles and other subcellular compartments, and macromolecules specifically associated therewith, represent targets for the development of agents that specifically impact, respectively, a particular tissue within an animal, abnormal (diseased) but not normal (healthy) cells, or cells from an undesired species but not cells from a desirable species.

For example, members of the Bcl-2 family of proteins associate with the outer membranes of mitochondria and with other cellular membranes. Translocation of Bcl-2 proteins from one intracellular position to another occurs during apoptosis, a process by which some abnormal (e.g., pre-cancerous) cells are directed to undergo programmed cell death (PCD), thus eliminating their threat to their host organism. Methods for monitoring modulations in the accumulation of Bcl-2 proteins in various subcellular compartments, or their translocation from one intracellular location to another, can allow identification of agents designed to impact apoptosis, and to assay the effects of such agents in cells.

Provided herein are methods that can be used to monitor the modulation of the intracellular movement of a target as well as any simultaneous structural or chemical transformations that occur within the target as a result of or resulting in its translocation. For example, by selecting an appropriate set of labels, such as luminescent labels, a subcellular compartment such as the mitochondria or a biomolecule such as a Bcl-2 protein can labeled. The cells containing the labelled displaying molecules and/or other moieties to assess their effects on the cells. Modulations in the location of interaction on the membrane as well as the conformational adjustment on the protein or the membrane surface due to interaction between the displayed molecules and/or biological particles and cellular sample can be assessed by detecting and monitoring interactions, such as by detecting FRET. Similarly, a protein, such as Bcl-2, which is transported intracellularly, can be labelled, such as with luminescent labels. The sample containing the labelled protein can then be exposed to a self-assembled array displaying molecules and/or biological particles. The suspected source of the protein and the suspected final destination of the protein can be monitored, such as by monitoring the location of the luminescent labels or resonance energy transfer among the labels. Alterations in the location of the binding interactions and any conformational changes that occur as a result of exposure of the sample to the self-assembled array, as determined by monitoring the labels as well as a timeline for the movement of the protein from its source to its destination can be visualized.

8. Assays for Assessing Cell Growth and Proliferation

Cells reproduce by duplicating their contents and dividing into two separate entities. Coordinating cell proliferation, growth and differentiation is crucial for the development and survival of an organism. Cells divide only when they receive the proper signals from growth factors that circulate in the bloodstream or from a cell they directly contact. When a cell receives the message to divide, it goes through the cell cycle, which includes several phases for the division to be completed. To be affected by a growth factor, the target cell should have a receptor molecule for the growth factor, such as a membrane bound protein. When the growth factor binds to its receptor, a series of enzymes inside the cell are activated, which in turn activates proteins called transcription factors inside the cell's nucleus. The activated transcription factors turn on genes required for cell growth and proliferation.

In some instances, a cell, such as a cancer cell, grows out of control. Unlike normal cells, cancer cells ignore signals to stop dividing, to specialize, or to die and be shed. Growing in an uncontrollable manner and unable to recognize its own natural boundary, the cancer cells can spread to other areas of the body. In a cancerous cell, several genes mutate causing the cell becomes defective. Abnormal cell division can occur either when active oncogenes, mutated normal genes, are turned on, or tumor suppressor genes are lost.

The combinations, kits, systems and methods provided herein can be used to identify molecules that modulate cell growth and proliferation. For example, a library of growth factors can be displayed on the self-assembled array. A sample of cells can then be exposed to the displayed growth factors and the proliferation of the cells monitored, allowing identification of molecules that are involved in the regulation of cell growth. In addition, perturbations, such as candidate compounds and/or conditions, can be added to the self-assembled array prior to, simultaneously with or after the sample is exposed to displayed molecules and alteration in cell proliferation can be monitored. Candidate compounds or conditions that increase or decrease cell proliferation can be identified.

9. Assays for Assessing Apoptosis

Apoptosis, or programmed cell death, is a normal component of the development and health of multicellular organisms. Cells die in response to a variety of stimuli and during apoptosis, they do so in a controlled, regulated fashion. This makes apoptosis distinct from another form of cell death called necrosis in which uncontrolled cell death leads to lysis of cells, inflammatory responses and, potentially, to serious health problems. Apoptosis, by contrast, is a process in which cells play an active role in their own death (which is why apoptosis is often referred to as cell suicide).

There are a number of mechanisms through which apoptosis can be induced in cells. The sensitivity of cells to any of these stimuli can vary depending on a number of factors such as the expression of pro- and anti-apoptotic proteins (e.g. the Bcl-2 proteins or the Inhibitor of Apoptosis Proteins), the severity of the stimulus and the stage of the cell cycle. In some cases the apoptotic stimuli include extrinsic signals such as the binding of death inducing ligands, such as CD95 (or Fas), TNFR1 (TNF receptor-1) and the TRAIL. (TNF-related apoptosis inducing ligand) receptors DR4 and DR5, to cell surface receptors or the induction of apoptosis by cytotoxic T-lymphocytes by granzyme. The latter occurs when T-cells recognize damaged or virus infected cells and initiate apoptosis in order to prevent damaged cells from becoming neoplastic (cancerous) or virus-infected cells from spreading the infection.

In other cases, apoptosis is initiated following intrinsic signals that are produced following cellular stress. Cellular stress can occur from exposure to radiation or chemicals or to viral infection. lt can also be a consequence of growth factor deprivation or oxidative stress. In general, intrinsic signals initiate apoptosis via the involvement of the mitochondria. The relative ratios of the various bcl-2 proteins can often determine how much cellular stress is necessary to induce apoptosis.

Upon receiving specific signals instructing the cells to undergo apoptosis, a number of distinctive biochemical and morphological changes occur in the cell. A family of proteins known as caspases are typically activated in the early stages of apoptosis. These proteins breakdown or cleave key cellular substrates that are required for normal cellular function, including structural proteins in the cytoskeleton and nuclear proteins such as DNA repair enzymes. The caspases can also activate other degradative enzymes such as DNases, which begin to cleave the DNA in the nucleus. The result of these biochemical changes is appearance of morphological changes in the cell.

The combinations, kits, systems and methods provided herein allow for detection of the modulation of cellular apoptosis resulting from the interaction of a biological particle with displayed target molecules in a self-assembling array. Stains specific for cell viability, such as trypan blue or propidium iodide, can be used to determine cell viability after exposure to a displayed molecule library on the self-assembled array (capture system). Necrotic cells are detected by intense propidium iodide staining of the cytoplasm, due to the complete disruption of the plasma membrane. ApopNexin™ Kits (Serological Corp.) also are used to discriminate apoptotic from necrotic cells, and to label the progression of a cell through the various stages of apoptosis. As apoptosis progresses into the late-stage, the plasma membrane becomes permeable to DNA dyes such as propidium iodide, which enter the cell and stain yellow/orange.

In addition, other biomolecules involved in apoptosis, such as caspases, can be detected by using biomolecule specific substrates. Caspases are a family of proteins that are one of the main effectors of apoptosis. The caspases are a group of cysteine proteases that exist within the cell as inactive pro-forms or zymogens. These zymogens can be cleaved to form active enzymes following the induction of apoptosis. The production of these proteins from their zymogenic form is indicative of the advent of apoptosis and is therefore a target for detection.

For example, cell permeant caspase substrates such as PhiPhiLu® (ONCOIMMUNIN, Inc.); cell permeant caspase 3 and caspase 7 fluorogenic substrates from Molecular Probes; CaspSCREEN Apoptosis Detection Substrate (CHEMICON); and CaspaTag™ Fluorescein Caspase Activity Kits (Serologicals Inc.) can all be used to monitor production and activity of the caspases. In addition, immunostains, such as anti-active caspase 3 monoclonal antibodies (BD PHARMINGEN), also are available for detection of apoptosis via the caspases.

In normal cells, most of the phosphatidylserine (PS) contained in the plasma membrane is oriented towards the cytoplasmic side of the cell membrane. In early stage apoptosis, the cell undergoes surface membrane blebbing, cytoplasmic shrinkage, nuclear DNA fragmentation, chromatin condensation and PS translocation across the plasma membrane to the exposed outer surface of the cell. It is thought that the PS on the membrane surface identifies the cell as a target for destruction by the immune system. ApopNexin™ Apoptosis Detection Kits (Serological Corp.) exploit this biochemical event using the annexin V protein labeled with either FITC or biotin. Annexin V is a calcium-dependent phospholipid binding protein with a high affinity for PS. In the presence of calcium, annexin V binds rapidly and specifically to PS and is visualized by flow cytometry or microscopy.

Mitochondria have the ability to promote apoptosis through release of cytochrome C, which together with Apaf-1 and ATP forms a complex with pro-caspase 9, leading to activation of caspase 9 and the caspase cascade. Bax, and other Bcl-2 proteins, show structural similarities with pore-forming proteins. It has therefore been suggested that Bax can form a transmembrane pore across the outer mitochondrial membrane, leading to loss of membrane potential and efflux of cytochrome C and AIF (apoptosis inducing factor). Fluorescent probes of mitochondrial membrane potential, which drops in apoptotic cells, are available and include, MITOTRACKER Red, Rhodamine 123, and JC-1 (Molecular Probes); MITOLIGHT (CHEMICON); and the MitoTag™ JC-1 Assay Kit (Serologicals Corp.). Anti-cytochrome C monoclonal antibodies with a conjugated enzyme or fluorophore also can be used to detect apoptosis. Additional assays for apoptosis stages such as chromatin condensation and fragmentation, are readily available for microscopic detection of DNA fragmentation.

10. Assays to Ass ss Changes in Cell Morphology

The combinations, kits, systems and methods provided herein can be used to identify molecules of that lead to alteration of the morphology of biological particles, such as cells. A sample containing target biological particles can be contacted with molecules displayed on a self-assembling array. The target biological particles, such as cells, can then be observed, such as by light microscopy, to identify changes in their physical characteristics, such as morphology. Alternatively, the target biological particles, such as cells, can be labeled, such as with a luminescent label, and changes detected or identified by monitoring changes in luminescence.

To serve as an effective tracer of cell morphology, a fluorescent probe or other detectable molecule can have the capacity for localized introduction into a biological particle, as well as long-term retention within that structure. If used with live cells and tissues, the tracer can be biologically inert and nontoxic. When these conditions are satisfied, the fluorescence or other detectable properties of the tracer can be used to track the position of the tracer over time. A diverse selection of fluorescent tracers, as well as biotinylated, spin-labeled and other tracers are available commercially from Molecular Probes, and include, but are not limited to, cell-permeant cytoplasmic labels (CellTracker Blue CMAC, CellTracker Green CMFDA or CellTracker Orange CMTMR); microinjectable cytoplasmic labels (lucifer yellow CH, CASCADE BLUE hydrazide, the ALEXA FLUOR hydrazides, sulforhodamine 101 and biocytin); membrane tracers (DiI, DiO, DiD, DiR, DiA, R18, FM 1-43, FM 4-64 and their analogs); fluorescent and biotinylated dextran conjugates, fluorescent microspheres (FluoSpheres and TransFluoSpheres fluorescent microspheres); and proteins and protein conjugates (Albumin Conjugates, Casein Conjugates, Peroxidase Conjugates, Phycobiliproteins, Fluorescent Histones, and ALEXA FLUOR 488 Soybean Trypsin Inhibitor). These tracers can be introduced into the target biological particle using any method known to those skilled in the art including, but not limited to, microinjection, hypoosomotic shock, scrape loading, sonication, high-velocity microprojectiles, glass beads, and electroporation (McNeil, PL Methods Cell Biol 29: 153-173 (1989)).

11. Receptor Internalization Assays

The combinations, kits, systems and methods provided herein can be utilized to monitor the internalization of cell-surface receptors of biological particles resulting from exposure of the biological particle to molecules and/or biological particles displayed on self-assembled arrays. For example, a receptor can be tagged with a marker that is either chemically conjugated (e.g., fluorochrome conjugated to the extracellular region) or genetically fused (e.g., GFP-receptor) and the cells expressing the receptor incubated with the self-assembled arrays of displayed molecules and/or biological particles. After incubation, cells are fixed and the tag is visualized with a detection device to localize the receptor in intracellular compartments (Ghosh et al. (2000) Biotechniques 29(1): 170-175).

Many fluorescent ligands available first bind to cell surface receptors, then are internalized and, in some cases, recycled to the cell's surface. Consequently, it can be difficult to assess whether the fluorescent signal is emanating from the cell surface, the cell interior or, as is more typical, a combination of the two sites. Furthermore, the fluorophore's sensitivity to environmental factors, principally intracellular pH, can affect the signal of the fluorescent ligand. Molecular Probes has commercially available products by which these signals can be separated and, in some cases, quantitated, for example, antibodies directed to the ALEXA FLUOR 488, BODIPY FL, fluorescein/Oregon Green, tetramethylrhodamine, TEXAS RED and CASCADE BLUE dyes to quench most of the fluorescence of surface-bound or exocytosed probes.

12. Receptor-mediated Cell Activation Assays

The combinations, kits, systems and methods provided herein can be used to monitor receptor-mediated cell activation resulting from the interaction of target biological particles with self-assembled arrays of displayed molecules and/or biological particles. For example, cells expressing a receptor of interest are incubated with a molecular library displayed on a self-assembled array and activation of cells is assayed by staining cells for activation markers including, but not limited to, cytokines, receptors, cell adhesion molecules and transcription factors. Staining can be done using specific antibodies using standard methods.

13. Receptor Activated Cell Signaling

The combinations, kits, systems and methods provided herein can be utilized to monitor or identify receptor activated cell signalling. For example, cells expressing a target receptor can be transfected with reporter constructs that read out activation of transcription factors following a signal transduction cascade transmitting signal via intracellular proteins upon activation of receptor at cell surface. Exposure of the cells to molecules displayed on a self-assembled array followed by monitoring of the transcription of the reporter gene identify molecules that cause activation of surface receptors upon incubation of cells with a displayed molecular library. Optionally, perturbations, such as candidate compounds and/or conditions, can be added to the self-assembled array prior to, simultaneously with or after exposure of the cells to the displayed molecules, and alterations in the activation of the cell signalling cascade can be assessed.

14. Epitope Mapping

The methods provided herein can be used to map epitopes for receptors displayed on the surface of cells or within a sample. For example, a library of tagged T cell receptors (TCRs) can be displayed on a self-assembled array. The self-assembled array (capture system) can then be exposed to a sample containing T cells and the interaction among the cells and the self-assembled array (capture system) determined. The resulting interactions can be used to map T cell epitope specificity of naturally occurring peptides, or libraries of synthetic peptides.

In another embodiment, TCR libraries are conjugated with the binding partners and displayed as such on the self-assembling array. Biological particles, such as antigen presenting cells (APCs) or recombinant cells that are modified to express peptides in the context of the major histocompatibility complex (MHC, class I or class II) on their surfaces, are “pulsed” or otherwise induced to express peptide epitopes in the context of major histocompatability complex (MHC), then exposed to the self-assembled array. Specific TCR-peptide MHC (pMHC) interactions bring APCs into contact with cognate, displayed TCRs. The interactions between the APCs and the self-assembled array (capture system) allows for visualization of components within the system including, but not limited to, specifically bound APCs, various fluorescently labeled secondary stains, and various fluorescently labeled, engineered cell-specific proteins.

15. Expression of Secreted Polypeptides by Tumor Cells

The combinations, kits, systems and methods provided herein can be utilized to discover or identify tumor or other cell-surface receptors which trigger expression of secreted proteins, e.g., B7-H1, which in turn induce apoptosis or other forms of cell death in secondary target cells (Nat Med 8(8): 793-800 (2002)). Primary target cells are tumor cells of any relevant type, specifically bound to the self-assembled array (capture system) through interactions between cell-surface receptors and a displayed molecular library. Secondary target cells are HLA-matched T cells (cytotoxic CD8 +T cells, CTLs) with TCR specificity for tumor cell-surface pMHC. Specific pMHC-TCR interactions brings CTL into contact with array-bound tumor cells. CTLs lyse and kill bound tumor cells unless tumor cells have been activated to express molecules, e.g., B7-H1, which interact with one or more CTL-surface receptors, in turn inducing apoptosis. The methods provided herein can be used to initially monitor specific interaction of displayed CTLs with tumor cells. The methods also can be used to detect apoptotic death of CTLs as measured by, for example, biochemical dye staining for mitochondrial membrane changes and DNA fragmentation.

16. Differentiation/Dedifferentiation Assays

The combinations, kits, systems and methods provided herein can be used to discover or identify cell-surface receptors which, when bound to a specific ligand displayed on the self-assembling arrays, induce differentiation or de-differentiation. Target cell sources are relevant cell types of choice, such as those that possess a specific, differentiation-stage-specific morphology and/or cell-surface marker which is either up-regulated or down-regulated in a stage-specific manner. Target cells specifically interact with libraries of displayed molecules and/or biological particles on a self-assembled array through cell-surface receptors. Once exposed to the displayed molecules and/or biological particles, changes, such as in differentiation state-specific morphology, an increase/decrease or loss/gain of cell-surface-expressed, differentiation stage-specific marker (revealed via binding of fluorescently labeled secondary Ab or other ligand) can be monitored.

17. Discovery of Molecules that Block Binding, Cleavage and/or Post-translational Modifications

The combinations, kits, systems and methods provided herein can be used to identify molecules and/or biological particles that block binding, cleavage and/or post-translations modifications of other molecules within a sample. The interaction of an exogenous molecule with a molecule in a sample or on the surface of a biological particle can result numerous functions including, but not limited to, the blockage of binding either on the surface or intracellularly, the generation of a signal for the cleavage of a second surface molecule, the generation of a signal for the post-translational modification of a second molecule, binding to a known molecule, such as, but not limited to, a protein, polypeptide, DNA, lipid, carbohydrate, and organic molecule, and enzymatic activity such as proteolysis, phosphorylation, methylation, acylation and prenylation. Detection methods, such as immunostaining, detection of the transcription of reporter genes and resonance energy transfer, can be used to monitor these functions.

For example, cleavage of surface proteins, termed protein shedding, is the proteolytic release of a cell surface protein. This shedding can serve a regulatory role by liberating soluble molecules into circulation while decreasing their concentration on the cell surface (Hooper et al. Biochem. J. 321:265-279 (1997)). Proteins that are shed from the cell surface include, but are not limited to, growth factors, cytokine receptors, cell adhesion molecules and leukocyte receptors. Shedding of cell surface molecules is initiated by interaction between a ligand and cell-surface receptor, which results in the recruitment of a soluble proteinase that cleaves the surface protein. For example, L-selectin, a member of a family of adhesion molecules, is constitutively expressed on the surface of circulating leukocytes. The soluble, active form is released from the surface by proteolytic cleavage following cell activation.

Post-translational modification of molecules can, for example, result in the activation of a proenzyme or the formation of the final molecular product, such as conversion of a molecule from its precursor form to its mature form or a secondary form. For example, the amyloid beta (Aβ) peptide, a 40 or 42 amino acid residue peptide, has been implicated the pathology of Alzheimer's disease. This peptide is generated from the post-translational processing of the amyloid-β precursor protein (APP) through initial cleavage by α-secretase followed by cleavage by α-secretase. Alternatively, APP can be processed by α-secretase, which cleaves at a varied site from the α-secretase, yielding a final 23 amino acid residue peptide fragment following cleavage by the α-secretase. This smaller peptide is not believed to contribute to the Alzheimer's disease pathology (Selkoe D.J. in The Molecular and Genetic Basis of Neurological Disease (Rosenberg et al., Eds.) pp. 601-612, Butterworth-Heinemann, Boston). The regulation of these two post-translational processing pathways can provide potential drug candidates for the regulation of amyloidβ production and Alzheimer's disease.

The combinations, kits, systems and methods provided herein can be used to identify molecules and/or perturbations, such as candidate compounds and/or conditions, that modulate the blockage of binding either on the surface or intracellularly, the generation of a signal for the cleavage of a second surface molecule or the generation of a signal for the post-translational modification of a second molecule. For example, a library of molecules and/or biological particles can be displayed on a self-assembled array. A sample containing target biological particles containing the amyloid-β precursor protein can be exposed to the displayed molecules and/or biological particles on the self-assembled array. Target biological particles showing the formation of the 23 amino acid post-translational product can be identified and the displayed molecule and/or biological particle interacting with the target biological particle can be selected for further study in its effect on the regulation of the formation of the 23 amino acid post-translational product of the amyloid-β precursor protein.

In another embodiment, a sample containing target biological particles can be exposed to a self-assembled array displaying a library of molecules and allowed to bind in the presence of a specific proteinase, such as a metalloproteinase. The self-assembled array can then be specifically stained for a soluble surface protein thought to be cleaved by the proteinase in the presence of a transduced signal. The loci that show a positive reaction with the stain indicate the target biological particles where a signal due to the interaction of the target biological particle with the self-assembled array (capture system) has been transduced, thereby allowing identification of displayed molecules that modulate the cleavage of molecules on the surface of the biological particles.

18. Discovery of Antibodies to Apically-localized Cell-surface Proteins, Carbohydrates and Lipids

The combinations, kits, systems and methods provided herein can be used to identify antibodies to apically-localized cell-surface proteins, carbohydrates and lipids. For example, epithelial mono-layers can be grown in culture. The molecules can be displayed on a self-assembled array that is, for example, immobilized on the surface of beads. These coated beads can then be applied to the apical cell surface. After washing, those beads that still adhere to the cell surface indicate which displayed molecules should be further investigated. This procedure, optionally, can be carried out in a 96 well format, with only one species of beads (containing only one specific tag) used per well. This option eliminates a need for bead encoding.

19. Detection of Phosphorylation and Dephosphorylation Activities

The combinations, kits, systems and methods provided herein can be used to detect phosphorylations and/or dephosphorylation activities within a sample as well as perturbations, such as candidate compounds and/or conditions, that modulate such activities. Eukaryotes employ phosphorylation and dephosphorylation of specific proteins to regulate many cellular processes (Hunter Cell 80:225-236 (1995); Karin Curr. Opin. Cell Biol. 3: 467-473 (1991)). These processes include signal transduction, cell division, and initiation of gene transcription. Thus, significant events in an organism's maintenance, adaptation, and susceptibility to disease are controlled by protein phosphorylation and dephosphorylation. These phenomena are so extensive that it has been estimated that humans have around 2,000 protein kinase genes and 1,000 protein phosphatase genes (Hunter Cell 80: 225-236 (1995)), some of these likely coding for disease susceptibility. For these reasons, protein kinases and phosphatases are prospective targets for the development of drug therapies.

The combinations, kits, systems and methods provided herein can be used to detect and monitor alterations in the dephosphorylation and phosphorylation reactions within a sample or a biological particle. For example, an appropriate set of labels, such as luminescent labels, can be attached to a suspected molecule being phosphorylated (or dephosphorylated) and/or an enzyme thought to be responsible for the activity. These molecules can then be transfected into target biological particles. The target biological particles can then be exposed to a self-assembled array displaying molecules and/or biological particles. Monitoring of the labels or interactions among the labels, such as resonance transfer energy, can yield information about the effect of the interaction between the target biological particle and the self-assembled array on the interaction between the enzyme and its substrate, and the rate of the phosphorylation (or dephosphorylation) reaction. Additionally, the effect that any added perturbation, such as candidate compounds and/or conditions, has on the native reaction can be monitored.

20. Determination and Monitoring of Chemical or Enzymatic Kinetics

The combinations, kits, systems and methods provided herein can be used to determine and monitor chemical or enzymatic reactions to gain information about the kinetics of the reactions. Chemical reactions proceed at a certain rate dependent on the components of the reaction and the environment in which the reaction occurs. Measurement of these rates often yields valuable information regarding the mechanism of the reaction and the resulting formation of products. Kinetic rates can be determined for catalytic reactions between an enzyme and its substrate including, but not limited to, conversion of a protein from one conformational state to another, formation of multimers from individual components and the translocation of an electron.

For example, the target reaction can include an enzyme, whose activity is regulated by cell-surface signalling. Attachment of the appropriate set of labels, such as luminescent labels, to the enzyme as well as its substrate in optimal positions permits study of the interaction between the molecules while simultaneously determining the rate of product formation by monitoring resonance energy transfer among the labels. The transfection of these molecules into a target biological particle, such as a cell, followed by exposure of the target biological particle to a self-assembled array displaying molecules and/or biological particles can yield information about the effect of the interaction between the target biological particle and the self-assembled array (capture system) on the chemical or enzymatic reaction.

Additionally, the combinations, kits, systems and methods provided herein can be used to monitor changes in the rate of the formation and decomposition of reactive intermediates, either chemical or conformational, which are difficult to isolate using standard spectroscopic or isolation techniques. Further, the combinations, kits, systems and methods provided herein can be used to monitor alterations in the binding of a protein, such as an electron transfer protein, to its enzymatic binding partner and the resulting enzymatic reaction that converts substrate to products. The rate at which an electron is transferred from an electron transport protein to the active site of its enzymatic binding partner can be measured by placing labels, such as luminescent labels, at the distant sites and monitoring changes in the labels, such as resonance energy transfer, as a result of conformational or chemical changes as electron transfer and catalysis occurs.

21. Screening and Identification of Cyclic Peptides with Antibiotic Activity

The combinations, kits, systems and methods provided herein can be used to identify and screen cyclic peptides in order to assess their potential as an antibiotic agent. The appearance and proliferation of microbial antibiotic resistance demonstrates the need for the development of new classes of antibiotics with novel modes of action. One such area being developed is peptide-based antibiotics. Many vertebrates, including humans, produce antibiotic peptides as part of their innate immune response. These antibiotic peptides exhibit a fast and lethal mode of action that is quite different from the mode of action of other synthetic antibiotics, making peptide antibiotics attractive therapeutic agents (Borman Annu. Rev. Immunol. 13: 61-92 (1995)).

Over 400 natural antimicrobial peptides have been isolated and characterized. Based on chemical structure, these peptides can be classified into two main groups: linear and cyclic. The mode of action of a majority of these peptides is believed to involve membrane disruption, leading to cell leakage (Mor Drug Develop. Reds. 50: 440-447 (2000)). Nearly all known natural cyclic peptides display high antibacterial activity. Many also are highly hemolytic and thus lack the selectivity required for a human antibiotic (Kondejewski et al. J. Biol. Chem. 274:13181-13192 (1999)). Efforts to develop cyclic peptides as antibiotics in vivo have been directed toward the development of analogs that possess greater selectivity of bacterial cells over erythrocytes. The combinations, kits, systems and methods provided herein can be used identify and monitor cyclic peptide and libraries of cyclic peptide analogs capable of selective permeation of only bacterial membranes.

### F. Identification of Binding Partner Polypeptides

Any method for identifying or selecting binding partner polypeptides specific for particular capture agents can be employed. A variety are described herein and are known to those of skill in the art. Also provided herein is a method for designing polypeptide binding partners that are highly antigenic and that induce, upon administration to a host, antibodies that are specific for the polypeptides or other for screening antibody and single chain antibody or other libraries. Monoclonal antibodies and fragments thereof can be produced from the antibodies or the selected single chains or other binding agents identified from libraries are used as capture agents that are paired with the designed or generated polypeptide.

1. Overview of the Methods

The methods provided herein start with a set of amino acids, which typically includes some or all of the naturally-occurring amino acids and also can include selected non-naturally occurring amino acids. For exemplification, the naturally occurring 20 amino acids are included. In addition, the polypeptide that is to be designed can be any length, typically is short, at least two amino acids up to 50, but generally is 4, 5, 6, 7, 8, 9, 10, 12, 16, 20 or more. For exemplification, the polypeptides are 6 amino acids in length and contain 4 critical residues. The exemplary initial analysis is performed for 4-mers that contain any of the 20 naturally-occurring amino acids. The host for which antigenicity is targeted is mice. Accordingly, there are 204 combinations possible. The methods herein provide a way to select highly antigenic specific binding polypeptides from among these combinations of amino acids. The members of the set of possible polypeptides are selected by imposing criteria based upon empirical data regarding antigenicity in a particular host and also upon properties of particular amino acids. The method for selecting polypeptides can be performed manually or by using or developing a program to impose the criteria. An exemplary process is described herein. A polypeptide of 6 amino acids in length and 4 critical residues is selected for exemplification herein.

Step 1: Select length of polypeptide and critical residue number. For exemplification a length of 6 is selected with 4 critical residues.

Step 2: Generate all combinations of 4 residues using 10 amino acids such that there are no duplications of amino acids in any polypeptide. The ten amino acids were selected based upon antigenicity ranking (see table herein and cited references for the amino acids that occur most often in antigenic polypeptides) that had been empirically determined. The resulting set contained 5040 members.

Step 3: Using the similarity table (described herein), arbitrarily select one polypeptide. Using the selected polypeptide, pick a set of predetermined number of members. These polypeptides are selected to contain a sequence of amino acids that is as dissimilar as possible from the other members in the final selected set. This is done using the similarity table to create an indexing number, a similarity score, representative of the dissimilarity. This is done by combining the numbers from the table for each amino acid in a particular polypeptide compared to the reference polypeptide to create a score for each of the 30,240 polypeptides and the selecting a predetermined number by setting a threshold similarity index.

Step 4: Since 4 residues are selected from the total selected length of 6 (step 3), the remaining 2 residues, designated “non-critical” are assigned. For exemplary purposes, the 2 non-critical residues are assigned adjacent positions and only critical residues occupy the N-terminal and C-terminal positions, thereby generating the possible 6-mers into which non-critical residues are placed. For naturally occurring amino acids, non-critical residues are those that can be replaced with more than 10 amino acids and retain the specific binding properties of resulting polypeptide. These non-critical residues are known (see, description here and publications cited) and can be empirically determined. For exemplification two possible combinations of non-critical residues were selected. These were Tyr-Gly, and Ser-Gly. These were chosen herein since they confer solubility and permit hairpin folding which is advantageous for generating capture agents/binding partners for the methods and products herein.

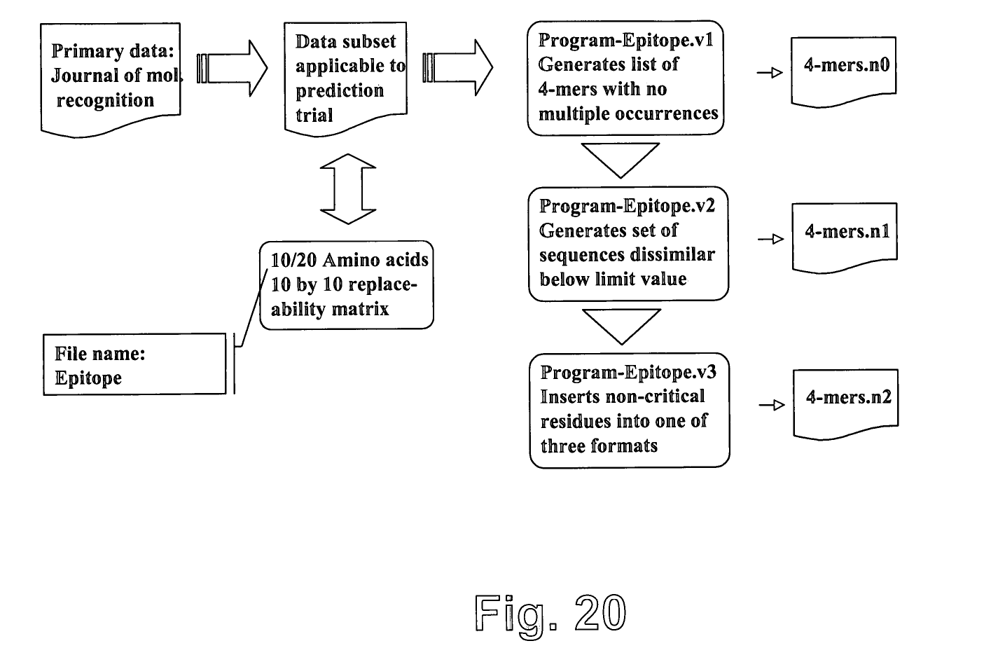

An exemplary process to carry out the steps as described is shown in FIG. 20. The final exemplary set chosen is provided herein (see discussion and Sequence Listing). As shown in the Examples, all tested polypeptides resulted in antibodies useful as capture agents specific for the 6-mer polypeptides. Thus, this method permits design of polypeptides that predictably induce production of specific antibodies upon administration, thereby providing highly specific capture agent/tag (binding polypeptides) pairs for use in the methods and products provided herein.

Provided are exemplary polypeptides produced by these methods. Included among these polypeptides are those of SEQ ID Nos. 38-948 or those that include SEQ ID Nos. 38-948. Such polypeptides are at least 6, 7, 8, 9, 10, 11, 12, 15, 20, 25, 30, 35, 40, 45, 50, 60 up to about 100 (or 100) amino acids in length.

Also provided are collections of binding partner polypeptides. Exemplary collections include at least, 2, 3, 4, 5, 6, 7, 8, 9, 10, 15, 20, 50, 100, 150, 200, 250, 300, 350, 400, 500 or more of the polypeptides of any of SEQ ID Nos. 38-948. Also provided are collections of capture agents and binding partner polypeptide pairs, where the binding partner polypeptides include at least, 2, 3, 4, 5, 6, 7, 8, 9, 10, 15, 20, 50, 100, 150, 200, 250, 300, 350, 400, 500 or more the polypeptides of any of SEQ ID Nos. 38-948. Kits containing the collections optionally including instructions preparing capture agents that specifically bind to members of the collection also are provided.

2. Description of the Methods

Provided herein are methods for obtaining highly specific, highly antigenic (HAHS) polypeptides for use as partners with capture agents (binding proteins) such as antibodies. The polypeptides contain any number of amino acids against which a specific capture agent (binding protein) can be generated or synthesized to bind. Typically such polypeptides are at least 2, 3, 4, 5, 6 to about 100 amino acids in length, usually between 2-50, 2-40, 2-30, 2-20, 4-20, 5-20, 2-50, 4-50, 5-50, and 6-20 amino acids in length. Also provided are methods for generating the binding proteins (capture agents), such as antibodies, which bind to HAHS polypeptides. Thus, methods generate pairs of HAHS polypeptides and capture agents. There is no detectable cross-reactivity, such as by ELISA assay, between or among different pairs of HAHS polypeptides and capture agents.

The method of designing highly antigenic, highly specific polypeptides constructs or designs polypeptides that contain sequences of amino acids that are antigenic (i.e., they are more likely to be antigenic than a randomly selected or generated polypeptide of the same or similar size). These polypeptides are more likely to raise an immune response in a subject and/or bind antibodies or a portion thereof with a high affinity and specificity than a randomly selected polypeptide.

The methods provided herein, which are described in detail below, use statistical probabilities that a particular amino acid appears in an antigenic polypeptide. These statistical probabilities can be generated empirically or calculated. Statistical probabilities for naturally occurring amino acids are exemplified herein. The same or similar methods can be applied to any sets of amino acids including non-naturally occurring amino acids and analogs thereof.

For example, sequences of antigenic polypeptides can be obtained by empirical methods, such as by injecting mice with polypeptides representing all the possibilities of a set length of polypeptides. The polypeptides are injected into mice and antisera is collected. The antisera then is tested on collections of polypeptides and the antigenic polypeptides are identified based on their reactivity with the antisera. Non-antigenic polypeptides are identified by their lack of reactivity with the antisera. The frequency of an amino acid appearing in a polypeptide that is antigenic is used to determine which amino acids are more likely to be found in an antigenic polypeptide.

The number of polypeptides possible for all sequence combinations is high. For example, a 4 mer has 20×20×20×20 possibilities (160,000 total). It is time consuming, costly and undesirable to test each and every polypeptide to determine its antigenicity. The methods described herein obviate the need for such tedious testings. The methods use a statistical prediction based on the frequency of an amino acid appearing in a polypeptide that is antigenic. The likelihood that an amino acid appears in a polypeptide that is antigenic can be determined based on a representative set of data, for example, based on immunizing animals with a representative subset of all the possibilities of that polypeptide length. Based on the subset of polypeptides injected which are antigenic and non-antigenic, amino acids are identified that either are more likely to be present in antigenic polypeptides or are more likely to be present on non-antigenic polypeptides. The likelihood of a amino acid's presence in an antigenic polypeptide gives an observed antigenic ranking. Using polypeptides of the 20 naturally occurring amino acids, a ranking of antigenicity for each amino acid can be obtained. Similarly, an antigenic ranking of amino acids also can be obtained by mapping epitopes in known proteins. Antibodies to known proteins are used to determine the sequence of amino acids to which they bind, for example by deletion or replacement mutagenesis or by synthesizing subsets of amino acid sequence found within the protein sequence. The antibodies are tested for reactivity with the mutants or with subsets of peptide sequences from the protein. The shortest sequence of amino acids from the protein which retains binding to the antibody defines the epitope. Epitope mapping can be performed with a representative number of proteins and antibodies and the statistical occurrence of each of the 20 amino acids found in the epitopes is determined to generate the antigenic ranking of the amino acids (see, e.g., Geysen et al., (1988). J. Molecular Recognition 1:32-41; Getzoff et al., (1988). The Chemistry and Mechanism of Antibody Binding to Protein Antigens. Academic Press. Advances in Immunology. Vol 43:1-98). Epitope mapping and antigenic ranking such as with known proteins or by injecting collections of random polypeptides can be done in any species of interest that raises an immune response, for example mice, rabbit, rat, human, monkey, dog, chicken, and goat. For example, using data obtained from epitope mapping (Geysen et al., (1988). J. Molecular Recognition 1:32-41), the amino acids were assigned the following antigenic rankings, with 1 being the highest and 20 the lowest probability (Table 3).

TABLE 3

| Ranking | amino acid |
|---|---|
| 1 | E |
| 2 | P |
| 3 | Q |
| 4 | N |
| 5 | F |
| 6 | H |
| 7 | T |
| 8 | K |
| 9 | L |
| 10 | D |
| 11 | V |
| 12 | I |
| 13 | G |
| 14 | Y |
| 15 | S |
| 16 | C |
| 17 | A |
| 18 | M |
| 19 | R |
| 20 | W |

Epitope mapping and antigenic ranking can also be performed using recombinant means, by screening libraries of antibodies or antibody fragments with polypeptides containing sequences of epitopes, such as collections of sequences of critical amino acids. The polypeptides which are bound by the antibodies can be sequenced and the frequency of the amino acids appearing in polypeptides bound by the antibodies can be determined. Experimental conditions such as washing conditions in a phage library panning assay can be used to control the affinity of the interaction between the antibodies and the peptides.

For a given length of polypeptides, amino acids are selected from the antigenic ranking list. Polypeptides can be any length sufficient for an antibody epitope, generally less than 20 amino acids. For example, the polypeptides length is between 2 and 20 amino acids, such as 2, 3, 4, 5, 6, 7, 8, 9, 10, 11, 12, 1 3, 14, 15, 16, 17, 18, 19 and 20 amino acids in length. In one exemplary embodiment, 4mers are selected using the antigenic ranking list of amino acids.

A threshold ranking of antigenicity can be chosen to limit the possible number of polypeptides in the subset (subset A) and to bias the subset to more antigenic sequences. For example, if the polypeptide length is 20 amino acids, each of the 20 positions can be selected from the top 19 antigenic ranking amino acids, limiting the subset from the total possibilities of all 20 amino acids at each position. The threshold can be set according to the number of polypeptides desired in the subset and the level of dissimilarity chosen for the subset. In one embodiment, the amino acids are chosen from the top n-1 antigenic ranking amino acids, where n is the total amino acids in the polypeptide length. In one aspect of the embodiment, the top 19, 18, 17, 16, 15, 14, 13, 12, 11, 10, 9, 8, 7, 6, or 5 antigenic ranking amino acids are used to design and construct the polypeptide sequences. In one exemplary embodiment, the top 10 antigenic ranking amino acids are used to design and construct polypeptide sequences. In another exemplary embodiment, the amino acids E, P, Q, N, F, H, T, K, L, and D are used to design and construct polypeptide sequences.

In a given length of polypeptides, to further bias the specificity of the polypeptides and reduce potential cross reactivity between binding proteins and polypeptides outside the partner pairs, each amino acid in the length can be unique. This further reduces the number of polypeptides in the subset (subset B). For example, if the polypeptide is a 4 mer and 10 amino acids are chosen from the antigenic ranking list, the number of possibilities in 10×9×8×7, where each amino acid is unique within a 4-mer (i.e., there is no duplication or any multiples of a chosen amino acid within the polypeptide length). Thus, for a 4 mer there are 5040 possibilities in this subset B.

Subset B represents the list of antigenic polypeptide possibilities for the chosen length of polypeptide. Optionally, these polypeptides can be incorporated in larger polypeptides, such that the polypeptides derived from subset B are designated the critical residues in the polypeptide, composed of antigenic amino acids and the remaining positions in the polypeptide length are noncritical positions (subset C). The length of such polypeptides can be generally less than 50 amino acids, typically less than 20 amino acids. For example, the polypeptides length can be between 2 and 20 amino acids, such as 2, 3, 4, 5, 6, 7, 8, 9, 10, 11, 12, 13, 14, 15, 16, 17, 18, 19 and 20 amino acids in length. The number of critical residues is larger than the number of non-critical residues. Generally, for peptides of 9 or less amino acids, the number of critical residues is approximately 55%, 60%, 70%, 80%, 85%, 90% or 95% of the total number of amino acids in the polypeptide.

The non-critical positions can be any amino acid. The non-critical positions can also be utilized to introduce added functionalities into the polypeptide, such an solubility and folding. In one exemplary embodiment, amino acids which increase solubility and permit flexibility and folding are used at the non-critical positions. For example, the amino acids S, G and Y are utilized at the non-critical positions.

The non-critical positions can be designated at specific sites within the polypeptide length to construct subset D. For example, it can be designated that the N and C terminal residues of the polypeptide are critical residues. In another example, it can be designated that the non-critical residues are found in pairs. In one exemplary embodiment 6 mer polypeptides are designed whereby the first and last (N and C terminal) positions are critical residues and 2 additional positions of the remaining 4 residues of the 6-mer also are critical residues chosen from a set of antigenic amino acids. The remaining 2 positions are non-critical residues and are designated to be in adjacent positions in the 6 mer.

In the above example, with 6 mers, 5040×3 (15120) possible polypeptides are generated for subset D as follows:

X N N X X X
X X N N X X
X X X N N X

where X's are critical residues and N's are non-critical residues and the 3 polypeptides show the possible arrangement to generate adjacent non-critical residues and polypeptides with critical residues at the ends.

Subset D can then be further restricted to generate a set of polypeptides that are dissimilar from each other, subset E. To extract a subset E, a single polypeptide is chosen at random from subset D as the first, reference polypeptide. A similarity ranking is calculated for all of the polypeptides in subset D using a replaceability matrix which compares the similarity of the amino acids at the critical positions to each other. An example of a similarity matrix is given in Table 4:

TABLE 4
Similarity Matrix

| | E | P | Q | N | F | H | T | K | L | D | G | S | Y |
|---|---|---|---|---|---|---|---|---|---|---|---|---|---|
| E | 100 | 13 | 33 | 13 | 2 | 8 | 10 | 6 | 8 | 42 | 13 | 15 | 6 |
| P | 5 | 100 | 16 | 11 | 8 | 11 | 11 | 16 | 3 | 3 | 14 | 14 | 0 |
| Q | 15 | 10 | 100 | 25 | 5 | 10 | 10 | 5 | 5 | 5 | 20 | 15 | 10 |
| N | 4 | 0 | 13 | 100 | 4 | 9 | 4 | 9 | 4 | 4 | 4 | 9 | 0 |
| F | 11 | 11 | 11 | 11 | 100 | 5 | 26 | 5 | 37 | 16 | 0 | 32 | 21 |
| H | 8 | 23 | 23 | 15 | 0 | 100 | 15 | 15 | 0 | 0 | 23 | 8 | 8 |
| T | 15 | 6 | 12 | 12 | 6 | 9 | 100 | 12 | 9 | 6 | 3 | 44 | 6 |
| K | 0 | 3 | 26 | 23 | 10 | 26 | 23 | 100 | 10 | 10 | 10 | 29 | 0 |
| L | 2 | 4 | 12 | 6 | 22 | 8 | 4 | 18 | 100 | 8 | 2 | 4 | 10 |
| D | 50 | 4 | 12 | 42 | 4 | 23 | 15 | 0 | 4 | 100 | 0 | 27 | 0 |
| G | 3 | 0 | 9 | 3 | 6 | 12 | 3 | 12 | 6 | 6 | 100 | 24 | 3 |
| S | 17 | 6 | 0 | 0 | 11 | 39 | 22 | 11 | 6 | 0 | 6 | 100 | 6 |
| Y | 0 | 0 | 0 | 0 | 29 | 0 | 0 | 14 | 14 | 0 | 0 | 0 | 100 |

A similarity score is determined for each polypeptide in subset D as compared with the first reference polypeptide chosen for subset E. The similarity score can be determined for example, by combining the similarity probabilities (represented in Table 4 above as 0-100%) to determine an overall score for the polypeptide. For example, if subset D is a collection of 6-mer polypeptides and the first polypeptide chosen is EPNGYF, each polypeptide in subset D is compared with the reference first polypeptide, EPNGYF, using the similarity matrix to calculate a similarity score by combining the similarity value at each of the 4 critical positions to the corresponding positions in the reference polypeptide. The maximum score is 100% (identical polypeptide) and the minimum score is zero.

A size for subset E is set at the desired number of polypeptides, for example 10, 20, 30, 40, 50, 100, 200 or 1000 polypeptides. A threshold value is determined which will generate the desired number of polypeptides for subset E. For example, if the threshold is set very low, and therefore the degree of similarity is very low and a smaller subset E of polypeptides will be generated. Conversely, if the threshold of similarity is set high, the subset E will be a larger number of polypeptides. The number of polypeptides can be determined by one skilled in the art based on the intended subsequent use of the polypeptides. For example, if a library of polypeptides of several thousand polypeptides is desired, the threshold can be set higher. If only 10 polypeptides are desired which are dissimilar from each other, the threshold can be set lower.

a. Use of Non-naturally Occurring Amino Acids for Polypeptide Design and Generation

The use of non-naturally occurring amino acids increases the diversity and thus uniqueness of the polypeptides that can be generated. For example, there are several hundred non-naturally occurring amino acids that are commercially available and a even larger number that can be synthesized by standard chemistry methods known in the art. The ability to incorporate non-naturally occurring amino acids also permits linear, cyclic and branched polypeptide structures to be designed and constructed.

Non-natural amino acids include, but are not limited to, non-natural β-amino acids; amino acids having alkyl, cycloalkyl, heterocyclyl, aromatic, heteroaromatic, electroactive, conjugated, azido, carbonyl and unsaturated side chain functionalities; isomeric N-substituted glycine, wherein the side chain of an α-amino acid is attached to the amino nitrogen instead of to the α-carbon of that molecule. The following are representative examples of non-natural amino acids:

Non-natural amino acids that are modifications of natural amino acids such that the amino group is attached to β-carbon atom of the natural amino acid (e.g.β-tyrosine). Non-natural amino acids that are modifications of natural amino acids in the side chain functionality, such that the imino groups or divalent non-carbon atoms such as oxygen or sulfur of the side chain of the natural amino acids have been substituted by methylene groups, or, alternatively, amino groups, hydroxyl groups or thiol groups have been substituted by methyl groups, olefin, or azido groups, so as to eliminate their ability to form hydrogen bonds, or to enhance their hydrophobic properties (e.g. methionine to norleucine).

Non-natural amino acids that are modifications of natural amino acids in the side chain functionality, such that the methylene groups of the side chain of the natural amino acids have been substituted by imino groups or divalent non-carbon atoms or, alternatively, methyl groups have been substituted by amino groups, hydroxyl groups or thiol groups, so as to add ability to form hydrogen bonds or to reduce their hydrophobic properties (e.g. leucine to 2-aminoethylcysteine, or isolecine to o-methylthreonine).

Non-natural amino acids that are modifications of natural amino acids in the side chain functionality, such that a methylene group or methyl groups have been added to the side chain of the natural amino acids to enhance their hydrophobic properties (e.g. Leucine to gamma-Methylleucine, Valine to beta-Methylvaline (t-Leucine)).

Non-natural amino acids that are modifications of natural amino acids in the side chain functionality, such that a methylene groups or methyl groups of the side chain of the natural amino acids have been removed to reduce their hydrophobic properties (e.g. Isoleucine to Norvaline).

Non-natural amino acids that are modifications of natural amino acids in the side chain functionality, such that the amino groups, hydroxyl groups or thiol groups of the side chain of the natural amino acids have been removed or methylated to eliminate their ability to form hydrogen bonds (e.g. Threonine to o-methylthreonine or Lysine to Norleucine). Non-natural amino acids that are optical isomers of the side chains of natural amino acids (e.g. Isoleucine to Alloisoleucine).

Non-natural amino acids that are modifications of natural amino acids in the side chain functionality, such that the substituent groups have been introduced as side chains to the natural amino acids (e.g. Asparagine to beta-fluoroasparagine). Non-natural amino acids that are modifications of natural amino acids where the atoms of aromatic side chains of the natural amino acids have been replaced to change the hydrophobic properties, electrical charge, fluorescent spectrum or reactivity (e.g. Phenylalanine to Pyridylalanine, Tyrosine to p-Aminophenylalanine).

Non-natural amino acids that are modifications of natural amino acids where the rings of aromatic side chains of the natural amino acids have been expanded or opened so as to change hydrophobic properties, electrical charge, fluorescent spectrum or reactivity (e.g. Phenylalanine to Naphthylalanine, Phenylalanine to Pyrenylalanine). Non-natural amino acids that are modifications of the natural amino acids in which the side chains of the natural amino acids have been oxidized or reduced so as to add or remove double bonds (e.g. Alanine to Dehydroalanine, Isoleucine to Beta-methylenenorvaline).

Non-natural amino acids that are modifications of proline in which the five-membered ring of proline has been opened or, additionally, substituent groups have been introduced (e.g. Proline to N-methylalanine). Non-natural amino acids that are modifications of natural amino acids in the side chain functionality, in which the second substituent group has been introduced at the alpha-position (e.g. Lysine to alpha-difluoromethyllysine).

Non-natural amino acids that are combinations of one or more alterations, as described supra (e.g. Tyrosine to p-Methoxy-m-hydroxyphenylalanine). Non-natural amino acids that are isomeric N-substituted glycines, wherein the side chain of an α-amino acid is attached to the amino nitrogen instead of to the α-carbon of that molecule (e.g. N-methyl glycine, N-isopropyl glycine). Non-natural amino acids which differ in chemical structures from natural amino acids but are compatible, in protected or unprotected form, with a hybrid synthesis of peptide chemistry. Non-natural amino acids are readily available and widely known. Exemplary non-natural amino acids (with their abbreviations) include, but are not limited to, for example: Aib for 2-amino-2-methylpropionic acid, β-Ala for β-alanine, α-Aba for L-α-aminobutanoic acid; D-α-Aba for D-α-aminobutanoic acid; Ac₃c for 1-aminocyclopropane-carboxylic acid; Ac₄c for 1-amino-cyclobutanecarboxylic acid; Ac₅c for 1-aminocyclopentanecarboxylic acid; Ac₆c for 1-aminocyclohexanecar-boxylic acid; Ac₇c for 1-amino-cycloheptanecarboxylic acid; D-Asp(ONa) for sodium D-aspartate; D-Bta for D-3-(3-benzo[b]thienyl)alanine; C₃al for L-3-cyclopropylalanine; C₄al for L-3-cyclobutylalanine; C₅al for L-3-cyclopentylalanine; C₆al for L-3-cyclohexylalanine; D-Chg for D-2-cyclohexylglycine; CmGly for N-(carboxymethyl)glycine; D-Cpg for D-2-cyclopentylglycine; CpGly for N-cyclopentylglycine; Cys(O₃Na) for sodium L-cysteate; D-Cys(0₃H) for D-cysteic acid; D-Cys(O₃Na) for sodium D-cysteate; D-Cys(O₃Bu₄N) for tetrabutylammonium D-cysteate; D-Dpg for D-2-(1,4-cyclohexadienyl)-glycine; D-Etg for (2S)-2-ethyl-2-(2-thienyl)glycine; D-Fug for D-2-(2-furyl)glycine; Hyp for 4-hydroxy-L-proline; IeGly for -[2-(4-imidazolyl)ethyl]glycine; alle for L-L-alloisoleucine; D-alle for D-alloisoleucine; D-Itg for D-2-(isothiazolyl)glycine; D-tertLeu for D-2-amino-3,3-dimethylbutanoic acid; Lys(CHO) for N⁶-formyl-L-lysine; MeAla for N-methyl-L-alanine; MeLeu for N-methyl-L-leucine; MeMet for N-methyl-L-methionine; Met(O) for L-methionine sulfoxide; Met(0₂) for L-methionine sulfone; D-Nal for D-3-(1-naphthyl)alanine; Nie for L-norleucine; D-Nle for D-norleucine; Nva for L-norvaline; D-Nva for D-norvaline; Orn for L-ornithine; Orn(CHO) for N⁵-formyl-L-ornithine; D-Pen for D-penicillamine; D-Phg for D-phenylglycine; Pip for L-pipecolinic acid; iPrGly for N-isopropylglycine; Sar for sarcosine; Tha for L-3-(2-thienyl)alanine; D-Tha for D-3(2-thienyl)alanine; D-Thg for D-2-(2-thienyl)glycine; Thz for L-thiazolidine-4-carboxylic acid; D-Trp(CHO) for Nin-formyl-D-tryptophan; D-trp(O) for D-3-(2,3-dihydro-2-oxoindol-3-yl)alanine; D-trp((CH₂)mCOR¹) for D-tryptophan substituted by a -(CH₂)mCOR′ group at the 1-position of the indole ring; Tza for L-3-(2-thiazolyl)alanine; D-Tza for D-3-(2-thiazolyl)alanine; D-Tzg for D-2-(thiazolyl)glycine.

Non-naturally occurring amino acids can be ranked for antigenicity using methods applied to the naturally occurring amino acids, for example by testing sequences against antisera or libraries of antibodies (described herein) and can be ranked along-side naturally occurring amino acids. For example, a representive set of polypeptides composed of non-naturally occurring amino acids and/or a combination of non-naturally occurring and naturally occurring amino acids of a chosen polypeptide length can be used to immunize animals. Based on the subset of polypeptides injected which are antigenic and non-antigenic, amino acids are identified which either are more likely to be present in antigenic polypeptides or are more likely to be present on non-antigenic polypeptides. The likelihood of a amino acid's presence in antigenic polypeptide gives an observed antigenic ranking. Some non-ntural amino acids are very structurally similar to naturally occurring amino acids and to other non-naturally occurring amino acids. This similarity can be factored in to provide antigenicity rankings based on these similarities. Non-naturally occurring amino acids can also be assigned a similarity ranking for use with the methods as described, based on their structural and functional similarity to each other and to naturally occurring amino acids.

b. Generation of Polypeptides

Once the polypeptides are designed, any of the subsets of polypeptides desrcibed herein can be generated by standard methods known in the art. The petides can be chemically synthesized by standard and/or combinatorial chemistry. polypeptides can also be synthesized using recombinant means such as by expression of nucleic acids encoding the polypeptide sequences. For recombinant expression, the polypeptides are limited to the 20 naturally occurring amino acids and additionally non-naturally occurring amino acids where the expression organism of choice has been genetically engineered to generate such modifications.

### I. Identification of Binding Proteins for Polypeptide Binding Partner Pairs

Binding proteins are generated and/or selected that specifically bind the binding partners. The pairs of binding proteins and binding partners can then be used in applications such as addressable collections and capture systems. As noted, the polypeptide binding partners provided herein and the methods for generating such polypeptide binding partners provide polypeptides that are designed to be antigenic and thus antibodies or antibody fragments can be isolated which specifically bind to the polypeptides.

Candidate binding protein—polypeptide binding partner pairs can be identified by any method known to the art, including, but are not limited to, one or several of the following methods, such as, for example raising antibodies from exposure of a subject to the binding partner polypeptides and phage display of an antibody library followed by biopanning with the polypeptide binding partner of interest and any method known to those of skill in the art for identifying pairs of molecules that bind with high affinity and specificity. The following discussion provides exemplary methods; others can be employed.

1. Raising Antibodies

Antibodies contemplated herein include polyclonal antibodies, monoclonal antibodies and binding fragments thereof. Polyclonal antibodies are employed where high affinity (avidity) is desired. Polyclonal antibodies are typically obtained by immunizing an animal and isolating the polyclonal antibodies produced by the animal.

For example, antibodies have traditionally been obtained by repeatedly injecting a suitable animal (e.g., rodents, rabbits and goats) with an antigen or antigen with adjuvant. If the animal's immune system has responded, specific antibodies are secreted into the serum. The antibody-rich serum (antiserum) that is collected contains a heterogeneous mixture of antibodies, each produced by a different B lymphocyte. The different antibodies recognize different parts of the antigen, and are thus a heterogeneous mixture of antibodies. A homogeneous preparation of antibodies can be prepared by propagating an immortal cell line wherein antibody producing B cells are fused with cells derived from an immortal B-cell tumor. Those hybrids (hybridoma cells) that are producing the desired antibody and have the ability to multiply indefinitely are selected. Such hybridomas are propagated as individual clones, each of which can provide a permanent and stable source of a single antibody (a monoclonal antibody) which is specific for the antigen of interest. The antibodies can be purified from the propagating hybridomas by any method known to those skilled in the art. Fragments of antibodies can be synthesized or produced and modified forms thereof produced.

In one exemplary embodiment, mice are immunized with a collection of polypeptide binding partners generated by the methods provided herein, for example as diphtheria toxin-6 mer polypeptide conjugates. The 6-mer has 2 non critical positions and 4 critical positions. The 2 non-critical positions of the 6-mer are adjacent to each other. The non-critical positions are not found at the ends of the polypeptide and thus are represented at two positions of positions 2, 3, 4 and 5. The 2 non-critical positions are chosen from S, G and Y. The remaining 4 critical residues are selected from the top 10 antigenic amino acids in table X: E, P, Q, N, F, H, T, K, L, and D.

Antibodies are raised against the collection of polypeptides. A library of hybridoma cells is then generated and clones are screened for their reactivity with individual polypeptides. Positive clones identify monoclonal antibodies which bind a selected polypeptide binding partner. The antibodies can be isolated by standard immunopurification techniques or by cloning methods such as by PCR with primers for conserved regions of the antibody structure.

Once the antibody is isolated, the polypeptide responsible for the identification of the antibody can be conjugated to a molecule and/or biological particle, as described below, and screened against the antibodies isolated above to determine whether the antibodies retain the ability to specifically bind the polypeptide, thereby identifying a binding protein—binding partner pair.

2. Phage Display

Antibodies can also be selected, for example by screening an antibody library, for example a single chain antibody library for antibodies which bind to each polypeptide. Phage display, protein expression library screening and antibody arrays as well as other screening methods well known in the art can be used to screen antibodies and antibody libraries for binding the polypeptides.

Polypeptides that interact with a specific binding protein, such as an antibody or antibody fragment, can be identified by displaying random libraries of binding proteins on the surface of a phage molecule and monitoring their interactions with the polypeptides. The bacteriophage that display binding proteins that interact with the polypeptides can be isolated through washing and then enriched through multiple panning steps, resulting in a high population of phage displaying a binding partner that can be used as a binding protein—binding partner pair.

For example, in order to identify binding proteins using panning and phage display, hybridoma cells are first created either from non-immunized mice or mice immunized with a library of random epitopes or immunized with groups or libraries of binding partners polypeptides. The mice (or other immunized animals) are initially screened for high immunoglobulin (Ig) production and epitope/peptide binding. Ig production can be measured in culture supernatants by ELISA assay using a goat anti-mouse IgG antibody. Epitope/peptide binding can also be measured by ELISA assay in which the mixture of haptens used for immunization are immobilized to the ELISA plate and bound IgG from the culture supernatants is measured using a goat anti-mouse IgG antibody. Both assays can be performed in 96-well formats or other suitable formats.

To produce an antibody library, recombinant antibody genes from mRNA isolated from spleenocytes or peripheral blood lymphocytes (PBLs). Functional antibody fragments can be created by genetic cloning and recombination of the variable heavy (V_{H}) chain and variable light (V_{L}) chain genes. The V_{H} and V_{L} chain genes are cloned by first reverse transcribing mRNA isolated from spleen cells or PBLs into cDNA. Specific amplification of the V_{H} and V_{L} chain genes is accomplished with sets of PCR primers that correspond to consensus sequences flanking these genes. The V_{H} and V_{L} chain genes are joined with a linker DNA sequence. A typical linker sequence for a single-chain antibody fragment (scFv) encodes the amino acid sequence (Gly₄Ser)₃. After the V_{H}-linker-V_{L} genes have been assembled and amplified by PCR, the products can be transcribed and translated directly or cloned into an expression plasmid such as for phage display and then expressed to produce functional recombinant antibody fragments displayed on the phage.

The phage library of binding proteins such as antibodies, is panned against the polypeptide binding partners and those which specifically bind are isolated.

3. Generation of Binding Protein-binding Partner Pairs

As described herein, binding proteins can be used as capture agents in the collections of capture agents and binding partners, addressable collections and capture systems described herein. Once antibodies and/or antibody fragments are identified which bind to the HAHS polypeptides, they can be used as capture agents. The antibodies can optionally be purified such as by hybridoma selection and affinity purification. The antibodies or fragments thereof can be cloned, such as described herein and known in the art and expressed by recombinant means for use as capture agents.

The HAHS polypeptides can be used as binding partners in capture agent-binding partner pairs in the collections of capture agents and binding partners, addressable collections and capture systems described herein. The HAHS peptides are conjugated to molecules and/or biological particles as tags that specifically bind capture agents. The HAHS polypeptides can be conjugated to molecules and/or biological particles by any means known in the art such as those described herein, including, but not limited to, recombinant means and chemical linkages. The conjugation can be direct or indirectly via a linker. The HAHS polypeptides can be encoded by nucleic acid molecules which can be joined with nucleic acid molecules encoding another polypeptide to create tagged-polypeptides such as described herein. For example, a collection of nucleic acid molecules encoding HAHS polypeptides can be used to create a tagged library of molecules.

### G. EXAMPLES

The following examples are included for illustrative purposes only and are not intended to limit the scope of the invention.

### Example 1

### Preparation of Capture Agent Antibody Collections

### A. Generating a collection of Capture Agent—Binding Partner Pairs

A collection of capture agents, such as antibodies, that bind to binding partners, such as peptide tags, is used to sort molecules linked to the tags. The collection of antibodies that specifically bind to the polypeptide tags can be generated by a variety of methods. One example is described below.

1. Hybridoma Screening

High affinity and high specificity antibodies for the array were identified by screening a randomly selected collection of individual hybridoma cells against a phage display library expressing a random collection of peptide epitopes. The hybridoma cells were created by fusion of spleenocytes isolated from a naive (non-immunized) mouse with myeloma cells. After a stable culture was generated, approximately 10-30,000 individual cell clones (monoclonals) were isolated and grown separately in 96-well plates. The culture supernatants from this collection were screened by ELISA with an anti-lgG antibody to identify cultures secreting significant amounts of antibody. Cultures with low antibody production were discontinued. Antibodies from this monoclonal collection were separated from culture supernatants using HiTrap Protein G-columns using the AKTA® Prime chromatography system following the manufacturer's protocol (AP Biotech).

Purified antibodies were used to screen for high affinity epitopes on phage-displayed peptide libraries (PhD7, PhD12 or C7C from New England Biolabs) as described below.

a. Biopanning

The antibodies were diluted in 0.1 M NaHCO₃ to give a final concentration of 5 μg/ml. Wells of a 8 well strip were coated with 50 μl of antibody and left at 4° C. overnight. Four 8 well strips were coated per antibody for use in all 4 rounds of biopanning. The following day, a loopful of ER2738 E. coli cells was inoculated in 20 ml 2×YT and grown on the shaker at 37° C. until the OD was between 0.5-0.8. Meanwhile, the coating antibodies were aspirated off and 200 μl of 3% non-fat milk (NFM) in 1×TBS-T was added and incubated at 37° C. for 1 hour. The wells were washed with 100 μl 1×TBS-T two times. The phage library was added at 1×10¹¹ particles per well (dilution was made in 3% NFM in 1×TBS-T to a final volume of 100 μl). This solution was the INPUT.

The wells were incubated at 37° C. for 1 hour followed by 5 washes with 1×TBS-T (1 minute per wash) for round 1. The bound phage were eluted by addition of 100 μl of 0.1 M glycine, pH 2.2. This eluate was transferred into an Eppendorf tube, followed by addition of 10 μl Tris, pH 8.0 to the same Eppendorf tube. The glycine and Tris steps were repeated once more and this solution was now the OUTPUT. The OUTPUT from the first round was now to be used as INPUT for the second round.

The grown ER2738 cells were centrifuged at 3500 rpm for 15 min and the cells resuspended in 1/20 of the original volume (1 ml) using Min A salts. One hundred microliters of the cells suspension was aliquoted into 15 ml Falcon tubes to which the OUTPUT (220 μl) was added and incubated at 37° C. for 30 min. The volume was increased to 1.0 ml with 2×YT (add 680 μl 2×YT) and incubated at 30° C. for 4 hours. The cells were spun at 8000 rpm for 15 min and the supernatants were transferred to Eppendorfs for use the next day as INPUT. These solutions were stored at 4° C.

Round 2 panning was a repeat of Round 1, however the wells were washed 10 times with 1×-BS-T (1 min per wash).

Round 3 panning was a repeat of Round 1, however the wells were washed 20 times with 1×-BS-T (1 min per wash).

Round 4 panning was a repeat of Round 1, however the wells were washed 20 times with 1×-BS-T (1 min per wash).

b. Titering of the INPUT and the OUTPUT

Appropriate dilutions were taken from the phage in culture tubes (e.g. 10⁸, 10¹⁰ and 100 μl for each dilution) and 300 μl of ER2738 E. coli cells were added to each aliquot. This suspension was kept at room temperature for 10 minutes. Three ml of Top Agar was added to each tube and poured on top of an LB Agar plate. The plate was incubated at 37° C. overnight and the number of plaques counted.

C. Making Hybridomas

Hybridoma cells were prepared by methods well known to those of skill in the art (see, e.g., Harlow et al. (1988) Antibodies: A Laboratory Manual, Cold Spring Harbor Laboratory, Cold Spring Harbor). Hybridoma cells were created by the fusion of mouse spleenocytes and mouse myeloma cells. For the fusion, antibody-producing cells were isolated from the spleen of a non-immunized mouse, mixed with the myeloma cells and fused. Alternatively, the hybridoma cells were created from spleenocytes isolated from a mouse previously immunized chicken IgY.

A healthy, rapidly dividing culture of mouse myeloma cells was diluted into 20 ml of medium containing 20% fetal bovine serum (FBS) and 2×OPI. Growth medium is typically Dulbecco's modified Eagle's (DME) or RPMI 1640 medium. Ingredients of mediums are well known (see, e.g., Harlow et al. (1988) Antibodies: A Laboratory Manual, Cold Spring Harbor Laboratory, Cold Spring Harbor).

Antibody producing cells were prepared by aseptic removal of a spleen from a mouse, disruption of the spleen into cells and removal of the larger tissue by washing with 2×OPI medium. A typical mouse spleen contains approximately 5×10⁷ to 2×10⁸ lymphocytes. Equal numbers of spleen cells and myeloma cells were pelleted by centrifugation (400×g for 5 min) and the pellets were separately resuspended 5 ml of medium without serum and then combined. Polyethylene glycol (PEG) is added to 0.84% from a 43% solution. The cells were gently resuspended in the PEG-containing medium and then repelleted by centrifugation at 400×g for 5 minutes, washed by resuspension in 5 ml of medium containing 20% FBS, repelleted and washed a second time in medium supplemented with 20% FBS, 1×OPI, and 1×AH (AH is a selection medium; 1×AH contains 5.8 μM azaserine and 0.1 mM hypoxanthine). Cells were incubated at 37° C. in a CO₂ incubator. Clones generally are visible by microscopy after 4 days.

d. Isolating Hybridoma-cells

Stable hybridomas were selected by growth for several days in poor medium. The medium was replaced with fresh medium and single hybridomas were isolated by limited dilution cloning. Because hybridoma cells have a very low plating efficiency, single cell cloning was performed in the presence of feeder cells or conditioned medium. Freshly isolated spleen cells can be used as feeder cells as they do not grow in normal tissue culture conditions and are lost during expansion of the hybridoma cells. In this procedure, a spleen was aseptically removed from a mouse and disrupted. Released cells were washed repeatedly in medium containing 10% FBS. A spleen typically produces 100 ml of 10⁶ cells per ml. The feeder cells were plated in 96-well plates, 50 μl per well, and grown for 24 hours. Healthy hybridoma cells were diluted in medium containing 20% FBS, 2×OPI to a concentration of 20 cells per milliliter. Cells should be as free of clumps as possible. Fifty μl of the diluted hybridoma cells were added to the feeder cells to a final volume is 100 μl. Clones began to appear in 4 days.

Alternatively single cells can be isolated by single-cell picking by individually pipetting single cells and then depositing in wells containing feeder cells. Single cells also can be obtained by growth in soft agar. Once healthy, stable cultures were achieved, the cells are maintained by growth in DME (or RPMI 1640) medium supplemented with 10% FBS. Stable cells were stored in liquid nitrogen by slow freezing in medium containing a cryoprotectant such as dimethylsulfoxide (DMSO). The amount of antibody being produced by the cells was determined by measuring the amount of antibody in the culture supernatants by the ELISA method.

2. Recovery of Phage after Panning and Sequencing the Epitopes

a. Identification of Positive Phage Clones by ELISA

In a 96-deep well plate, 100 μl of E. coli 2738 cells grown previously to an OD of 0.5 were added. To each well, 96 individual plaques from the titer plates were added and the plates then were kept at 37° C. for 30 minutes. To each well was added 400 μl of 2×YT with tetracycline. The plates then were kept at 30° C. overnight with shaking. In the meantime, 96-well polystyrene plates (Maxisorp, NUNC) were coated with the appropriate antibody for detection and kept overnight at 4° C.

The following day, the antibody was aspirated off, 1 μl of 3% non-fat milk in 1×TBST was added to each well and the plate incubated at 37° C. for 1 hour. The plate was washed with 2× with TBS-T. Ten μl of 10% milk in 5×TBS-T was added to each well followed by addition of 40 μl of sample from deep well plate to the corresponding well in the ELISA plate. The ELISA plate was incubated at 37° C. for 1 hour. The plate was washed 4 times with TBS-T.

Then, 50 pi of the anti-M13 antibody-HRP conjugate was added to each well at 1 in 5000 dilution prepared in 3% non-fat milk in 1×TBS-T and incubated at 37° C. for 1 hour. The plate was washed 4 times with TBS-T, followed by addition of 50 μl OPD in each well. After yellow color developed, the reaction was stopped by the addition of 13 μl 3 N HCl. The absorbance was read at 492 nm.

b. Sample Preparation for Sequencing

Eight positive phage clones were picked and added to a 96-deep well plate that contained 100 μl of E. coli 2738 cells. The plate was incubated at 37° C. for 30 min followed by addition of 900 μl of 2×YT media and an additional incubation at 37° C. for 4 hour. This plate was sent to MJ Research (Waltham, Calif.) for sequencing.

### B. Selective Infection

Selective infection technologies, such as phage display, are used to identify interacting protein-peptide pairs. These systems take advantage of the requirement for protein-protein interactions to mediate the infection process between a bacteria and an infecting virus (phage). The filamentous M13 phage normally infects E. coli by first binding to the F pilus of the bacteria. The virus binds to the pilus at a distinct region of the F pilin protein encoded by the traA gene. This binding is mediated by the minor coat protein (protein 3) on the tip of the phage. The phage binding site on the F pilin protein (a 13 amino acid sequence on the traA gene) can be engineered to create a large population of bacteria expressing a random mixture of phage binding sites.

The phage coat protein (protein 3) also can be engineered to display a library of diverse single chain antibody structures. Infection of the bacteria and internalization of the virus is therefore mediated by an appropriate antibody-peptide epitope interaction. By placing appropriate antibiotic resistance markers on the bacteria and virus DNA, individual colonies can be selected that contain both genes for the antibody and its corresponding peptide epitope. The recombinant antibody phage display library prepared from non-immunized mice and the bacterial strains containing a random peptide sequence in the phage binding site in the traA gene are commercially available (Biolnvent, Lund, Sweden). Creation of a recombinant antibody library is described below.

C. Expression and Purification of Antibodies

Purification of antibodies from hybridoma supernatants was achieved by affinity binding. A number of affinity binding substrates are commercially available. The procedure described below is based on commercially available substrates (Protein A-SEPHAROSE) and follows the procedure described above.

Recombinant antibodies were expressed and purified as described (McCafferty et al. (1996) Antibody engineering: A practical Approach, Oxford University Press, Oxford). Briefly, the gene encoding the recombinant antibody was cloned into an expression plasmid containing an inducible promoter. The production of an active recombinant antibody was dependent on the formation of a number of intramolecular disulfide bonds. The environment of the bacterial cytoplasm is reducing, thus preventing disulfide bond formation. One solution to this problem was to genetically fuse a secretion signal peptide onto the antibody which directs its transport to the non-reducing environment of the periplasm (Hanes et al. (1997) Proc. Natl. Acad. Sci. U.S.A. 94:4937-4942).

Alternatively, the antibodies can be expressed as insoluble inclusion bodies and then refolded in vitro under conditions that promote the formation of the disulfide bonds.

### D. Exemplary Array and use Thereof for Capture of Proteins with Polypeptide Tags and Detection Thereof

To demonstrate the functioning of the methods herein, capture antibodies, specific, for example, for various peptide epitopes, such as the human influenza virus hemagglutinin (HA) protein epitope, which has the amino acid sequence YPYDVPDYA (SEQ ID No.4), were used to tag, for example, scFvs. For example, an scFv with antigen specificity for human fibronectin (HFN) was tagged with an HA epitope, thus generating a molecule (HA-HFN), which was recognized by an antibody specific for the HA peptide and which has antigen specificity of HFN. After depositing various concentrations of the capture antibodies (from 800 μg/ml to 200 μg/ml), including anti-HA tag capture antibodies, onto a glass slide coated with a surface for capturing proteins, such as a nitrocellulose-coated slide (FAST™, Schleicher and Schuell), they were allowed to bind at ambient temperature and humidity of 50 to 60%. After binding, slides with deposited anti-HA capture antibodies were blocked with a protein-containing solution such as Blocker™ BSA (PIERCE) diluted to 1× in phosphate-buffered saline (PBS) with TWEEN-20 (poly-oxyethylenesorbitan monolaurate; SIGMA) added to a final concentration of 0.05% (vol:vol) or with a 3% non-fat milk in the same buffer to eliminate background signal generated by non-specific protein binding to the membrane. For subsequent description contained herein PBS with 0.05% (vol:vol) TWEEN-20 is referred to as PBS-T. Blocking times can be varied from 60 min at ambient temperature to longer hours at ambient temperature or at 4° C., for example. Incubation temperatures for all subsequent steps can be varied from ambient temperature to about 37° C. In all instances, the precise conditions are determined empirically.

After blocking the membranes containing the deposited anti-HA capture antibodies, an incubation with peptide epitope-tagged scFvs can be performed. Purified scFvs (or bacterial culture supernatants, or various crude subcellular fractions obtained during purification of such scFvs from E. coli cultures harboring plasmid constructs that direct the expression of such scFvs upon induction, for example HA-HFN scFv, containing the HA peptide tag), can be diluted to various concentrations (for example, between 0.1 and 100 μg/ml) in BBSA-T. Membranes with deposited anti-peptide tag capture antibodies then were incubated with this HA-HFN scFv antigen solution. Membranes with deposited anti-HA capture antibodies and bound HA-HFN scFv antigen then were washed three times with PBST for suitable periods of time (e.g., 3-5 min per wash).

Membranes with deposited anti-HA capture antibodies and bound HA-HFN scFv then were incubated with, for purposes of demonstration, biotinylated human fibronectin (Bio-HFN), which is an antigen that can be recognized by the capture HA-HFN scFv. Bio-HFN was serially diluted (e.g., from 1 to 10 μg/ml) in BBSA-T. The resulting membranes were washed as before and then were incubated with Neutravidin.HRPO (PIERCE) diluted 1 in 10000 in BBSA-T. The resulting slides were washed as before, rinsed with PBS and developed with a 1:1 mixture of freshly prepared Supersignal ELISA Femto Stable Peroxide Solution and Supersignal™ ELISA Femto Lumino Enhancer Solution (PIERCE), and then imaged using an imaging system, such as, for example, a KODAK Image Station 440CF or IS1000 or other such imaging system. A small volume of the Supersignal solution was plated on the platen of the image station.

Slides then were placed array-side down into the center of the platen, thus placing the surface area of the antibody-containing portion of the membrane into the center of the imaging field of the camera lens. In this way, the small volume of developer, present on the platen, can then contact the entire surface area of the antibody-containing portion of the slide. The Image Station cover was closed for antibody array image capture. Camera focus (zoom) varies depending on the size of the membrane being imaged. Exposure times can vary depending on the signal strength (brightness) emanating from the developed membrane. Camera f-stop settings are infinitely adjustable between 1.2 and 16.

Archiving and analysis of array images can be performed, for example, using the KODAK ID 3.5.2 software package. Intensity values for loci were measured using software. These data then were transformed, for example into MICROSOFT EXCEL, for statistical analyses.

### Example 2

### Self-assembling Array Printing

### A. Exemplary Capture Agent Printing Methods

1. Capture Agent Printing Using a Modified Inkjet Printer

Capture agents (CytoSets™ capture antibodies) were printed with an inkjet printer (CANON model BJC 8200 color inkjet) modified for this application. The six color ink cartridges were first removed from the print head. One-milliliter pipette tips then were cut to fit, in a sealed fashion, over the inkpad reservoir wells in the print head. Various concentrations of capture antibodies, in glycerol, then were pipetted into the pipette tips which were seated on the inkpad reservoirs (typically the pad for the black ink reservoir was used).

For generation of printed images using the modified printer, MICROSOFT POWERPOINT was used to create various on-screen images in black-and-white. The images then were printed onto nitrocellulose paper (Schleicher and Schuell (S&S) PROTRAN BA85, pore size 0.45 μm, VWR catalog # 10402588, lot # C.F0628-1) which was cut to fit and taped over the center of an 8.5×11 inch piece of printer paper. This two-paper set was hand fed into the printer immediately prior to printing. After printing of the image, the antibodies were dried at ambient temperature for 30 min. The nitrocellulose was removed from the printer paper, and processed as described below (see Basic protocol for antibody and antigen incubations: FAST™ slides and nitrocellulose filters printed with CytoSets™ capture antibodies).

2. Capture Agent Printing Using a Pin-style Array Printer

Capture agent antibody dilutions were printed onto nitrocellulose slides (Schleicher and Schuell FAST™ slides; VWR catalog # 10484182, lot # EMDZO18) using a pin-printer-style arrayer (MicroSys 5100; Cartesian Technologies; TeleChem Arraylt™ Chipmaker 2 microspotting pins, catalog # C.MP2). Printing was performed using the manufacturer's printing software program (Cartesian Technologies′ AxSys version 1, 7, 0, 79) and a single pin (for some experiments), or four pins (for some experiments). Typical print program parameters were as follows: source well dwell time 3 sec; touch-off 16 times; microspots printed at 0.5 mm pitch; pins down speed to slide (start at 10 mm/sec, top at 20 mm/sec, acceleration at 1000 mm/sec²); slide dwell time 5 millisec; wash cycle (2 moves +5 mm in rinse tank; vacuum dry 5 sec); vacuum dry 5 sec at end. Array patterns were pre-programmed (in-house) to suit a particular array configuration. In many cases, replicate arrays were printed onto a single slide, allowing subsequent analyses of multiple analyte parameters (as one example) to be performed on a single printed slide. This in turn maximized the amount of experimental data generated from such slides. Microtiter plates (96-well for most experiments, 384-well for some experiments) containing capture antibody dilutions were loaded into the array printer for printing onto the slides. Based on the reported print volume (post-touch-off, see above) of 1 μl/microspot for the Chipmaker 2 pins, the capture antibody concentrations contained in the printed microspots typically ranged from 800 to 6 μg/mlcrospot.

Printing was performed at 50-55% relative humidity (RH) as recommended by the array printer manufacturer. RH was maintained at 50-55% via a portable humidifier built into the array printer. Average printing times ranged from 5-15 min; print times were dependent on the particular array that was printed. When printing was completed, slides were removed from the printer and dried at ambient temperature and RH for 30 minutes.

### B. Peptide Arrays

Peptides to be immobilized by printing on to a solid support, such as those shown in Table 5 below, were chemically synthesized and designed to contain either an N-terminal biotin molecule or a cysteine residue.

TABLE 5
Peptide Epitopes

| | | |
|---|---|---|
| Epitope name | Sequence | SEQ ID No. |

| | | |
|---|---|---|
| myc | EQKLISEEDL | 6 |
| HA | YPYDVPDYA | 4 |
| FLAG | DYKDDDDK | 2 |
| GluGlu | EEEEYMPME | 3 |
| V5 | GKPIPNPLLGLDST | 9 |
| T7 | MASMTGGQQMG | 7 |
| HSV | QPELAPEDPED | 5 |
| S-tag | KETAAAKFERQHMDS | 33 |
| KT3 | KPPTPPPEPET | 34 |
| E-tag | GAPVPYPDPLEPR | 1 |
| VSV-g | YTDIEMNRLGK | 8 |
| B34 | DLHDERTLQFKL | 12 |
| VSV-1 | HPNLPETRRYAL | 13 |
| VSV-2 | SYTGIEFDRLSN | 14 |
| 4C10 | MVDPEAQDVPKW | 15 |
| AB2 | LTPPMGPVIDQR | 10 |
| AB4 | QPQSKGFEPPPP | 11 |
| AB3 | YEYAKGSEPPAL | 16 |
| AB6 | AGTQWCLTRPPC | 17 |
| KT3-A | KLMPNEFFGLLP | 18 |
| KT3-B | KLIPTQLYLLHP | 19 |
| KT3-C | SFMPIEFYARKL | 20 |
| 7.23 | TNMEWMTSHRSA | 21 |
| S1 | NANNPDWDF | 23 |
| E2 | SSTSSDFRDR | 24 |
| His tag | HHHHHHGS | 25 |
| AU1 | DTYRYI | 26 |
| AU5 | TDFYLK | 27 |
| IRS | RYIRS | 28 |
| NusA | NusA Protein | 29 |
| MBP | Maltose Binding Protein | 30 |
| TBP | TATA-box Binding Protein | 31 |
| TRX | Thioredoxin | 32 |
| GFP | Green Fluorescent Protein | 35 |
| GST | Glutathione S transferase | 36 |
| HOPC1 | MPQQGDPDWVVP | 22 |

A small quantity (1 to 2 mg) of each peptide was dissolved in DMSO. Peptides insoluble in DMSO can be dissolved in any suitable buffer. Freshly dissolved peptides were used directly. The N-terminal cysteine of the peptides in the stored solutions was kept reduced with the addition of 1 mM DTT. Each of the peptide solutions was prepared at a concentration of 2 mg/ml with PBS. A 40 μl aliquot of each peptide solution then was added to a tube along with 40 p1 of 2× Print Buffer (2×PBS, 40% glycerol and 0.002% TWEEN-20) to give a final peptide concentration of 1 mg/ml. The solution then was mixed by vortexing for 10 sec at low speed and then spun briefly in a Micro Centrifuge. Two-fold serial dilutions were made for each of the peptides such that all peptides were at three different concentrations (1, 0.5 and 0.25 mg/ml). The peptide solutions were added into a 96-well PCR plate.

Each of the peptide solutions prepared above were printed on polystyrene 96-well plates (Maxisorp, NUNC), as shown in Table 6 below, coated either with Streptavidin (for biotinylated peptides) or with maleimide (for cysteine-containing peptides) using Telechem pins (CM-2) on a Cartesian printer (MicroSys 5100).

TABLE 6
Peptide Array Map

| | 1 | 2 | 3 | 4 | 5 | 6 | 7 |
|---|---|---|---|---|---|---|---|
| 1 | T7 | T7 | T7 | HSV | HSV | HSV | Control |
| | 1 mg/ml | 0.5 mg/ml | 0.25 mg/ml | 1 mg/ml | 0.5 mg/ml | 0.25 mg/ml | |
| 2 | VSV | VSV | VSV | V5 | V5 | V5 | Control |
| | 1 mg/ml | 0.5 mg/ml | 0.25 mg/ml | 1 mg/ml | 0.5 mg/ml | 0.25 mg/ml | |
| 3 | Glu—Glu | Glu—Glu | Glu—Glu | HA | HA | HA | Control |
| | 1 mg/ml | 0.5 mg/ml | 0.25 mg/ml | 1 mg/ml | 0.5 mg/ml | 0.25 mg/ml | |
| 4 | myc | myc | myc | E-tag | E-tag | E-tag | Control |
| | 1 mg/ml | 0.5 mg/ml | 0.25 mg/ml | 1 mg/ml | 0.5 mg/ml | 0.25 mg/ml | |
| 5 | Flag | Flag | Flag | YEEI | YEEI | YEEI | Control |
| | 1 mg/ml | 0.5 mg/ml | 0.25 mg/ml | 1 mg/ml | 0.5 mg/ml | 0.25 mg/ml | |

Printing was performed under 55 to 60% humidity and the plates air-dried for 1 hour followed by storage at 4° C. overnight.

In addition, pre-selected wells contained either mouse-lgG conjugated to horse radish peroxidase (mulgG-HRP) at a concentration of 20 μg/ml in Print Buffer or a biotinylated antibody at 100 μg/ml. These spots served as alignment markers and reagent controls for each sub-array within the array. The Flag and YEEI peptides were printed to serve as negative controls.

C. Anti-Peptide Antibody Arrays

Each of the anti-peptide antibody solutions (T7, VSV, Glu-Glu, myc, Flag, HSV, V5, HA, E-tag, UPC10 and anti-mouse IgG HRP antibodies) were prepared at a concentration of 1 mg/ml in PBS. A 40 μl aliquot of each antibody solution was added to a tube along with 40 μl of 2× Print Buffer (2×PBS, 40% glycerol and 0.002% TWEEN-20) to give a final antibody concentration of 0.5 mg/ml based upon the number and final concentration of protein required per tube. The solution was mixed by vortexing for 10 sec at low speed and then spun briefly in a Micro Centrifuge. Two-fold serial dilutions were made for each of the antibodies such that all antibodies were at three different concentrations (0.5, 0.25 and 0.125 mg/ml). The antibody solutions were added into a 96-well PCR plate as shown in Table 7 below.

TABLE 7
Antibody Array Map

| | 1 | 2 | 3 | 4 | 5 | 6 | 7 |
|---|---|---|---|---|---|---|---|
| 1 | T7 | HSV | VSV | V5 | Glu-Glu | HA | myc |
| | 0.125 mg/ml | 0.125 mg/ml | 0.5 mg/ml | 0.5 mg/ml | 0.5 mg/ml | 0.25 mg/ml | 0.125 mg/ml |
| 2 | E-tag | Flag | UPC10 | IgG-HRP | T7 | HSV | VSV |
| | 0.125 mg/ml | 0.25 mg/ml | 0.25 mg/ml | 0.2 mg/ml | 0.5 mg/ml | 0.5 mg/ml | 0.125 mg/ml |
| 3 | V5 | Glu—Glu | HA | myc | E-tag | Flag | UPC10 |
| | 0.125 mg/ml | 0.25 mg/ml | 0.5 mg/ml | 1 mg/ml | 0.5 mg/ml | 0.5 mg/ml | 0.125 mg/ml |
| 4 | IgG-HRP | T7 | HSV | VSV | V5 | Glu—Glu | HA |
| | 0.2 mg/ml | 0.5 mg/ml | 0.25 mg/ml | 0.25 mg/ml | 0.25 mg/ml | 0.125 mg/ml | 0.125 mg/ml |
| 5 | myc | E-tag | Flag | UPC10 | IgG-HRP | T7 | HSV |
| | 1 mg/ml | 0.25 mg/ml | 0.125 mg/ml | 0.5 mg/ml | 0.2 mg/ml | 0.25 mg/ml | 0.25 mg/ml |

Each of the antibodies prepared above were printed on polystyrene 96-well plates (Maxisorp. NUNC), as shown in Table 7 above, using Telechem pins (CM-2) on a Cartesian printer (MicroSys 5100). Printing was performed under 55 to 60% humidity and the plates air-dried for 1 hour followed by storage at 4° C. overnight.

In addition, pre-selected wells contained mouse-lgG conjugated to horse radish peroxidase (mulgG-HRP) at a concentration of 20 μg/ml in Print Buffer. These spots served as alignment markers for orientation of the array. Antibodies obtained from non-immunized mouse hybridomas were printed to serve as negative controls.

### D. Conjugation of Antibodies to Peptides

1. Activation of the Antibodies

A 10 mg/ml stock solution of m-Maleimidobenzoyl-N-hydroxysuccinamide ester (Sulfo-MBS) was prepared in 20 mM sodium phosphate buffer (0.15 M NaCl, pH 7.0 (PBS)) just prior to use. Each of the antibody solutions (50 to 100 μl) was equilibrated in PBS using MicroBiospin P6 gel filtration columns.

An aliquot of each antibody (<100 μl) was mixed with a 20-fold molar excess of Sulfo-MBS to a final volume of 100 μl in PBS. Reactions were performed at room temperature for 1 hour in the dark. Each sample then was desalted in two rounds with MicroBiospin P6 gel filtration columns. The activated antibodies were stored on ice if conjugation to the peptide was to be performed immediately or stored at -20° C. for later use.

2. Coupling of Peptides

Peptides to be conjugated were chemically synthesized and designed as described above to contain an N-terminal cysteine residue. A small quantity (1 to 2 mg) of each peptide was dissolved in DMSO. Peptides insoluble in DMSO can be dissolved in another suitable buffer. Freshly dissolved peptides were used directly. The N-terminal cysteine of the stored peptides in solution was kept reduced with the addition of 1 mM DTT.

A 20-fold molar excess of peptide was added to the activated antibody and the reaction performed at room temperature for 2 hours in the dark. Each sample then was desalted using in two rounds with MicroBiospin P6 gel filtration columns. The peptide-conjugated antibodies were stored at -20° C.

### E. ELISA Assay

1. For Peptide Arrays

All wells of the 96-well plate were incubated with Blocking Buffer I (3% non-fat milk in PBS containing 0.1% TWEEN-20 (PBS-T)) for 1 hour. The Blocking Buffer was aspirated off and each well was incubated with the appropriate dilution of anti-peptide antibody prepared in Blocking Buffer 11 (1% BSA in PBS-T). Incubation was performed at 37° C. for 1 hour. After aspiration, the wells were rinsed three times for 1 min each with PBS-T. The wells then were incubated with the appropriate dilution of goat anti-mouse antibody conjugated with HRP for 1 hour. After aspiration, wells were rinsed three times for 1 min each with PBS-T, followed by two 5 min rinses with PBS. HRP substrate was added to each well and the plate imaged using a CCD-based imaging device (KODAK Image Station 1000).

2. For Anti-Peptide Antibody Arrays

All wells of the 96-well plate were incubated with Blocking Buffer I (3% non-fat milk in PBS containing 0.1% TWEEN-20 (PBS-T)) for 1 hour. The Blocking Buffer was aspirated off and each well was incubated with the mixture of antibody-peptide conjugate at appropriate concentrations in Blocking Buffer 11 (1% BSA in PBS-T). Incubation was performed at 37° C. for 2 hours. After aspiration, the wells were rinsed three times for 1 min each with PBS-T. The wells then were incubated with antigens (E-tag, Flaf, Glu-Glu, HA, VSV-G and V5 peptides; Table 5) at various dilutions for an additional hour. The wells were rinsed three times for 1 min each with PBS-T. The wells then were incubated with either the detection antibody conjugated with HRP or with NeutrAvidin-HRP working solution for 1 hour. After aspiration, wells were rinsed three times for 1 min each with PBS-T, followed by two 5 min rinses with PBS. HRP substrate was added to each well and the plate imaged using a CCD-based imaging device (KODAK Image Station 1000).

### F. Results

1. Array ELISA Using Anti-Peptide Antibodies on Peptide Arrays

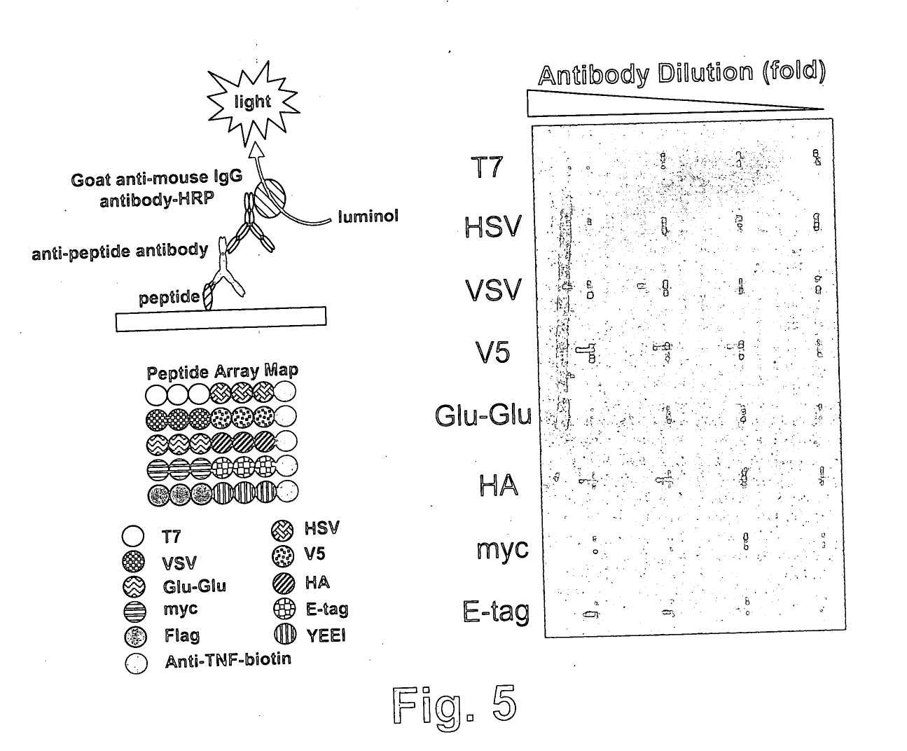

Anti-peptide antibodies (Stock concentration of 1 mg/ml) were diluted 1600-fold and additional 2-fold serial dilutions were made in subsequent columns of wells to finally obtain a 25,600-fold dilution for each antibody. Specific anti-peptide antibodies (T7, HSV, VSV, V5, Glu-Glu, HA, myc and E-tag antibodies; Table 2) were added to individual wells of each row. Specific peptide spots were detected for the appropriate antibody at dilutions of 1 in 1600 and detectable signal was obtained at antibody dilutions up to 12,800-fold (Table 6 and FIG. 5).

2. Array ELISA Using Peptides on Anti-Peptide Antibody Arrays

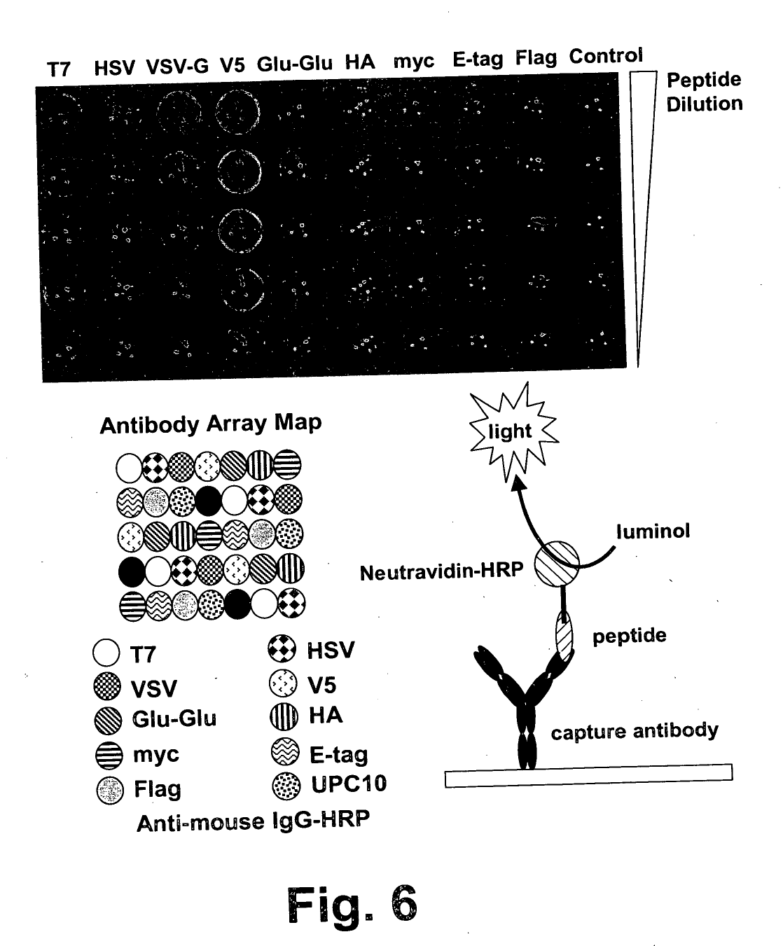

Two-fold serial dilutions of biotinylated peptides (T7, HSV, VSV-G, V5, Glu-Glu, HA, myc, E-tag and Flag peptides; Table 5) were prepared starting from 100 μg/ml and added to individual wells of each column of a 96-well plate containing anti-peptide antibody arrays. Specific antibody spots were detected for the appropriate peptide at the starting concentration and detectable signal was obtained for some peptides at a concentration of 0.78 μg/ml (Table 7 and FIG. 6).

3. Array ELISA Using Peptide-Antibody Conjugates Anti-Peptide Antibody Arrays

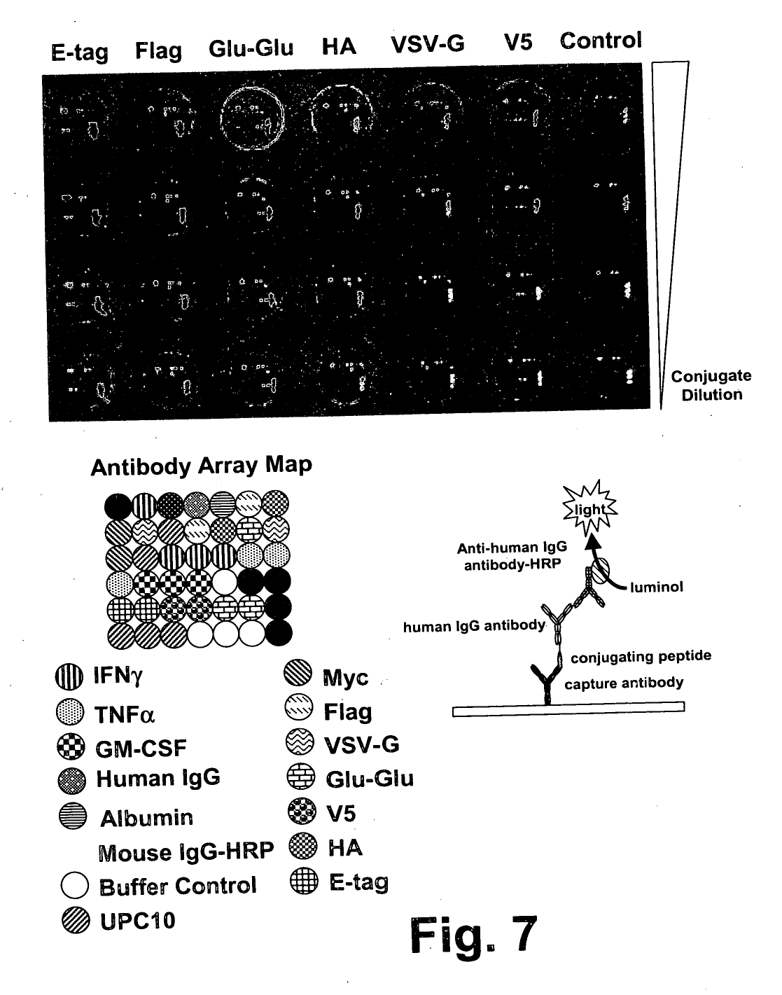

Human IgG (2 mg) was activated with a 20-fold molar excess of sulfo-MBS, followed by incubation with a 20-fold molar excess of various peptides (E-tag, Flag, Glu-Glu, HA, VSV-G and V5 peptides; Table 5) prepared as described above. A control reaction was set up replacing the peptide with buffer. The antibody-peptide conjugate was incubated at a concentration of 50, g/ml in Row 1 of a 96-well place followed by a 2-fold serial dilution down each column to reach a final concentration of 6.25 μg/ml. After incubation of the conjugate, arrays (Table 8 below) were developed using anti-human IgG-HRP and the various anti-peptide antibody spots were detected (FIG. 7).

TABLE 8
Antibody Array Map

| | 1 | 2 | 3 | 4 | 5 | 6 | 7 |
|---|---|---|---|---|---|---|---|
| 1 | M.IgG-HRP | IFNγ | TNFα | Human IgG | Albumin | Flag | HA |
| | 0.5 mg/ml | 0.5 mg/ml | 0.5 mg/ml | 0.5 mg/ml | 0.5 mg/ml | 0.25 mg/ml | 0.5 mg/ml |
| 2 | myc | VSV-G | UPC10 | Flag | HA | Glu—Glu | VSV-G |
| | 0.5 mg/ml | 0.5 mg/ml | 0.5 mg/ml | 0.5 mg/ml | 0.5 mg/ml | 0.5 mg/ml | 0.25 mg/ml |
| 3 | myc | UPC10 | IFNγ | IFNγ | IFNγ | TNFα | TNFα |
| | 0.5 mg/ml | 0.5 mg/ml | 0.5 mg/ml | 0.5 mg/ml | 0.5 mg/ml | 0.5 mg/ml | 0.5 mg/ml |
| 4 | TNFα | GM-CSF | GM-CSF | GM-CSF | Control | M.IgG HRP | M.IgG-HRP |
| | 0.5 mg/ml | 0.5 mg/ml | 0.5 mg/ml | 0.5 mg/ml | | 0.5 mg/ml | 0.5 mg/ml |
| 5 | E-tag | E-tag | V5 | V5 | Glu—Glu | Glu—Glu | M.IgG-HRP |
| | 0.5 mg/ml | 0.25 mg/ml | 0.5 mg/ml | 0.5 mg/ml | 0.5 mg/ml | 0.5 mg/ml | 0.5 mg/ml |
| 6 | UPC10 | UPC10 | UPC10 | Control | Control | Control | M.IgG-HRP |
| | 0.5 mg/ml | 0.5 mg/ml | 0.5 mg/ml | | | | 0.5 mg/ml |

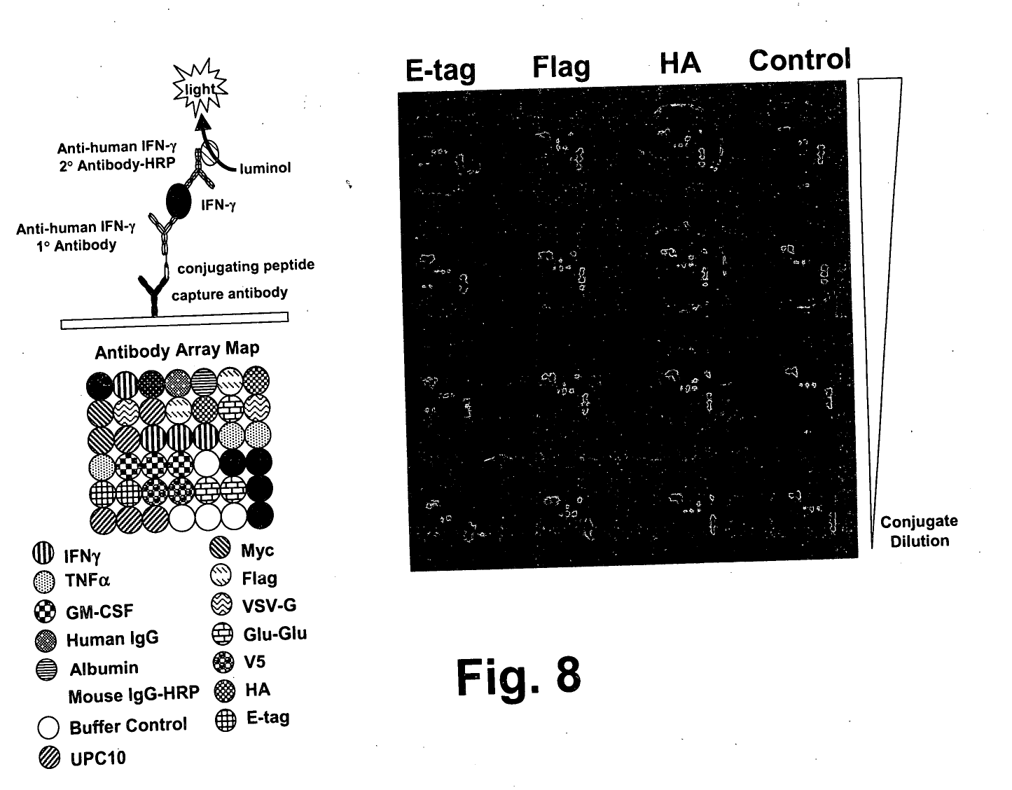

Anti-human interferon-gamma (IFNy) antibody (0.8 mg) was conjugated with various peptides (E-tag, Flag, and HA peptides; Table 5) prepared as described above. The antibody-peptide conjugate was incubated at concentration of 100 μg/ml in Row 1 followed by a 2-fold serial dilution down each column to reach a final concentration of 12.5 μg/ml. After incubation of the conjugate, each array (see Table 8 above) was incubated with 200 μg/ml human IFNy, followed by development using anti-human IFNy-HRP and the various anti-peptide antibody spots were detected (FIG. 8).

### Example 3

### Beads for Self-assembled Arrays

### A. Protocol for Binding Antibodies to Beads

1. Binding by Adhesion

Carboxylate-modified polystyrene beads (SIGMA, Catalog No. CLB-4) were washed twice with Washing buffer (25mM MES, pH 6.1 containing 0.01% TWEEN-20) and resuspended in 10 volumes of Washing buffer. A 1mg/ml anti-HA11 antibody solution was added to the beads (1/10th the volume of bead suspension) and the antibody allowed to adsorb to the beads at ambient temperature for 2 hours with slow mixing. The beads were washed twice with Washing buffer and then resuspended in 10 volumes of Storage buffer (Phosphate-buffered saline containing 1% BSA and 0.1% TWEEN-20).

2. Covalent Binding

Carboxylate-modified polystyrene beads (SIGMA, Catalog No. CLB-4) were washed twice with Washing buffer (25mM MES, pH 6.1 containing 0.01% TWEEN-20) and resuspended in 10 volumes of Washing buffer. EDC (1-Ethyl-3-[3-dimethylaminopropyl]carbodiimide Hydrocloride) was added to activate the beads at a final concentration of 1 mM. The beads were incubated at ambient temperature for 15 min with slow mixing. The beads were washed and following resuspension in 10 volumes of Washing buffer, a 1mg/ml anti-HA11 antibody solution was added to the beads (1/10th the volume of bead suspension) and the antibody allowed to bind to the beads at ambient temperature for 2 hours with slow mixing. The reaction was stopped by addition of Glycine at 50 mM final concentration and incubation at ambient temperature for 30 min. The beads were washed twice with Washing buffer and then resuspended in 10 volumes of Storage buffer.(Phosphate-buffered saline containing 1% BSA and 0.1% TWEEN-20).

### B. Capture of Antibody-coated Beads on Peptide Arrays

1. Printing Peptide Arrays

A polystyrene plate (NUNC Maxisorp) was coated with 5 μg/ml Neutravidin and then washed thrice with distilled water. After air-drying the plate at 37° C. for 60 min, an array of biotinylated peptides was printed in 1× Print buffer at concentration of 100 μg/ml, along with alignment and control spots according to the Plate Map shown below. After printing the plate was kept overnight at 4° C. prior to use. Peptides included E-tag, Flag, Glu, HA-11, HSV, myc, T7, V5 and VSV-G peptides.

| | | | | |
|---|---|---|---|---|
| Mouse IgG-HRP | E-tag | E-tag | myc | myc |
| anti-human IgG | Flag | Flag | T7 | T7 |
| Peptide 8 | Glu-Glu | Glu-Glu | V5 | V5 |
| Peptide 8 | HA-11 | HA-11 | VSV-G | VSV-G |
| Mouse IgG | HSV | HSV | Peptide 6 | Peptide 6 |

2. Assay to Determine Binding of Antibody-coated Beads on Peptide Arrays

The wells in the plate were blocked by addition of Blocking buffer (1% BSA in PBS containing 0.1% TWEEN-20) and incubated at 37° C. for 60 min. Various dilutions of the antibody conjugated beads in Blocking buffer were added to the wells and the plate incubated at 37° C. for 60 min. The plate was washed thrice with PBS-T, followed by incubation with the goat anti-mouse IgG-HRP conjugate at 37° C. for 60 min. The plate was washed as before and developed with Luminol and imaged on a KODAK IS1000.

3. Results

The anti-HA-11 antibody beads bound to the peptide spots corresponding to the HA-11 peptide. The antibody bound to the other peptide spots with much lower or insignificant binding (as measured bu the luminosity). The positive control spots (mouse IgG and Mouse IgG-HRP) gave detectable signal, similar to the level of signal with the HA-11 peptide spot.

### Example 11

### Generation of Binding Partner-capture Agent Pairs

### A. Generation of 6-mer Polypeptide Epitope Tags

A collection of 6 amino acid polypeptides (6-mers) were designed using the method described in Example A. The polypeptides were designed for screening suitability and use as binding partners paired with capture agents.

Peptides (6-mers) were synthesized with a C-terminal cysteine residue as: cysteine-(amino acid)₆-NH2. Diphtheria toxoid was activated using MCS to add maleimido groups to lysine side chains (Lee ACJ, Powell JE, Tregear GW, Niall HD and Stevens VC (1985) Mol. Immunol. 17:749-756). A 1.5 molar excess of the activated carrier protein was incubated with the polypeptides. The ratio ensures the lack of free unconjugated polypeptides such that unconjugated polypeptides or carrier proteins are not separated from the conjugated sample.

The 6mer polypeptides also are synthesized with biotin at the C-terminal end with a 4-mer linker polypeptide for use in screening assays: Biotin-SGSG-(amino acid)6-NH2.

### B. Immunization of Mice with DT-peptide Conjugates

The DT-peptide conjugates were dissolved in PBS. To formulate the mixture of conjugates, 0.5 mg of each of 4 peptides is added into one tube and the volume made to 2 ml with sterile PBS. The conjugates are mixed well before dispensing so that any particulate is well suspended. Each group of 4 polypeptide conjugates is designated by a group name, for example, as Grpl, Grp2, Grp3, and so on.

Three mice were immunized with each group of polypeptide conjugates. Mice were immunized with 200 μg protein/mouse for initial immunization (day 0) and boosts of 100 μg protein/mouse at days 21, 35, 49 and 63. Tail bleeds were taken at day 42 and day 70 and analyzed by ELISA assays. Samples of serum were taken from tail bleeds of the mice before day 0 immunizations to serve as pre-immune control serum.

Mice were analyzed by ELISA as follows. Biotinylated polypeptides were dissolved in DMSO at final concentrations of 5 mg/ml. NUNC Maxisorp plates are coated with 5 μg/ml Neutravidin in PBS and incubated at 4° C. until use (up to 30 days). The NeutrAvidin is aspirated off and the plates incubated with biotinylated polypeptides at 5 μg/ml in PBS for 60 min at 37° C. as indicated in the table below.

| | Plate 1 | Plate 2 | Plate 3 | Plate 4 | Plate 5 | Plate 6 |
|---|---|---|---|---|---|---|
| A | Peptide | Peptide 9 | Peptide 17 | Peptide 25 | Peptide 33 | Peptide 41 |
| | 1 | | | | | |
| B | Peptide | Peptide 10 | Peptide 18 | Peptide 26 | Peptide 34 | Peptide 42 |
| | 2 | | | | | |
| C | Peptide | Peptide 11 | Peptide 19 | Peptide 27 | Peptide 35 | Peptide 43 |
| | 3 | | | | | |
| D | Peptide | Peptide 12 | Peptide 20 | Peptide 28 | Peptide 36 | Peptide 44 |
| | 4 | | | | | |
| E | Peptide | Peptide 13 | Peptide 21 | Peptide 29 | Peptide 37 | Peptide 45 |
| | 5 | | | | | |
| F | Peptide | Peptide 14 | Peptide 22 | Peptide 30 | Peptide 38 | Peptide 46 |
| | 6 | | | | | |
| G | Peptide | Peptide 15 | Peptide 23 | Peptide 31 | Peptide 39 | Peptide 47 |
| | 7 | | | | | |
| H | Peptide | Peptide 16 | Peptide 24 | Peptide 32 | Peptide 40 | Peptide 48 |
| | 8 | | | | | |

The plates were blocked with 1× Blocker BSA in PBS-T for 60min at 37° C. One hundred microliters of each tail-bleed sample is added to Row A at a 1:100 dilution (2.5 μl of a 1:10 diluted tail-bleed and 22.5 μl Blocker BSA). To each plate, tail bleeds were added as follows (group refers to the groups of polypeptide-conjugates used for immunization, Mu1-Mu9 refer to the individual mice that were immunized with each group of peptides, described above).

| 1 | 2 | 3 | 4 | 5 | 6 | 7 | 8 | 9 |
|---|---|---|---|---|---|---|---|---|
| Tail | Tail | Tail | Tail | Tail | Tail | Tail | Tail | Tail |
| bleed | bleed | bleed | bleed | bleed | bleed | bleed | bleed | bleed |
| Grp1 | Grp1 | Grp1 | Grp2 | Grp2 | Grp2 | Grp3 | Grp3 | Grp3 |
| Mu1 | Mu2 | Mu3 | Mu4 | Mu5 | Mu6 | Mu7 | Mu8 | Mu9 |

The plates were incubated for 60 min at 37° C. and then washed 3× with 1×TBS-T. They then were incubated with 100 μl of a 1:2000 dilution of goat anti-mouse IgG-HRP conjugate for 60 min at 37° C., washed again 3 times with TBS-T and developed with OPD. The absorbance was measured at 492 nm.

### C. Generation of a Library of Hybridoma Cells

An additional 1.2 mg of conjugate-peptide mixtures (0.3 mg of each) was prepared for injection into mice prior to fusion. The mice were boosted with injections of polypeptides for three days prior to fusion. Fusion of spleen cells with mouse myeloma cells was performed on Day 84 and the hybridoma cells were grown in selection medium for 4 weeks. The medium was removed 3 weeks after fusion and fresh medium was added. The medium was harvested on Week 4 after fusion and tested for presence of anti-peptide antibodies by ELISA as described above. The assay was performed only for determination of antibodies to the immunized polypeptides and not for cross-reactivity. The cells were harvested, aliquoted and stored (Fusion library) until the results from analysis of supernatants were obtained.

### D. Cloning of Hybridomas to Generate Monoclonal Antibodies

A vial of the fusion library was thawed and the cells grown in medium for 2 weeks. Cells then were sorted using a FACS into ten 96-well plates such that each well received a single cell. The cells were grown for 2 weeks and the supernatant from each clone analyzed for presence of anti-peptide antibody as for the fusion library supernatant.

Positive clones were identified and ranked in order of ELISA signal intensities. Twelve clones with the highest signal intensities were scaled-up and assayed for polypeptide-specific antibody after 2 weeks. The supernatants then were assayed for antibody titre determination and two clones showing the highest anti-peptide antibody titre were selected for scale-up and storage. The clones were grown to obtain 100 ml of medium and the cells then were frozen at -80° C.

### E. Purification and Isotyping of IgG from Hybridoma Lines

The selected clones were grown for 2 weeks and the medium was used for analysis of antibody class and for specificity of binding to polypeptides by performing the assay described above. IgG was isotyped using Isotype mouse isotyping kits (Roche). The antibody from the supernatant was purified using Protein G affinity chromatography and stored in liquid nitrogen.

### F. Results

Peptides used for the immunizations were as follows:

!SEQ ID NO:? Peptide? SEQ ID NO:? Peptide

| | | | |
|---|---|---|---|
| 38 | EPNGYF | 324 | QGKEYF |
| 42 | EGYPNF | 381 | NSFEGP |
| 174 | PEQGYN | 383 | NFKSGH |
| 178 | PGYEQN | 387 | NSGFKH |
| 273 | QESGPD | 388 | NGFKYH |
| 288 | QPGYEH | 409 | NTSGHK |
| 366 | NQHGYD | 416 | NKGYHL |
| 378 | NGYFEP | 465 | FPSGNE |
| 45 | ESPNGF | 487 | FNPSGE |
| 47 | EPHSGK | 491 | FSGNPE |
| 51 | ESGPHK | 492 | FGNPYE |
| 52 | EGPHYK | 518 | FTLGYQ |
| 56 | EQGYPN | 522 | FGYTLQ |
| 65 | EQSGFH | 525 | FSTLGQ |
| 181 | PSEQGN | 603 | HSGQEL |
| 183 | PEFSGQ | 607 | HQTSGN |
| 187 | PSGEFQ | 622 | HNDGYT |
| 188 | PGEFYQ | 632 | HFGYTK |
| 192 | PEGYKD | 673 | HDSGTL |
| 209 | PNSGEF | 728 | TLGYNF |
| 298 | QGYNHE | 772 | KGQNYT |
| 301 | QSNHGE | 784 | KNGYDQ |
| 302 | QFEGYK | 810 | KGYHPD |
| 319 | QKESGF | 813 | KSHPGD |

Peptides were injected singly or in groups of 2-4 polypeptides/animal as described above. Antisera were analyzed as described. All of the injected polypeptides raised antisera that was high specificity and affinity.

Since modifications will be apparent to those of skill in this art, it is intended that this invention be limited only by the scope of the appended claims.

1. A combination, comprising:
(a) an addressable collection of capture agents;
(b) a plurality of sets of binding partners, wherein each set of binding partners specifically binds to a unique capture agent; and
(c) either or both of:
one or more conjugation reagents for effecting covalent linkage of a binding partner to a displayed molecule and/or displayed biological particle; and/or
instructions for use of the addressable collection of capture agents and binding partners to prepare self-assembled arrays.

2. The combination of claim 1, where the number of different sets of binding partners is equal to the number of unique capture agents.

3. The combination of claim 1, wherein the capture agents comprise antibodies.

4. The combination of claim 3, wherein the antibodies are monoclonal antibodies or fragments thereof that retain the ability to specifically bind to a binding partner.

5. The combination of claim 1, wherein the binding partners comprise polypeptides.

6. The combination of claim 1, comprising one or more conjugation regents for effecting covalent linkage of a binding partner to a molecule or biological particle.

7. The combination of claim 6, wherein the covalent linkage further comprises a linker between the binding partner and the molecule and/or biological particle.

8. The combination of claim 7, wherein the linker is a peptide linker, a chemical linker, or a cleavable linker.

9. The combination of claim 8, wherein the cleavable linker is selected from the group consisting of acid-cleavable, heat labile and photocleavable linkers.

10. The combination of claim 7, wherein the linkage comprises an intermediate molecule.

11. The combination of claim 10, wherein the intermediate molecule is a bead.

12. The combination of claim 11, wherein the bead is linked to an electronic, chemical, optical, or color-coded label.

13. The combination of claim 1, wherein: the capture agents comprise polyclonal antibodies; the collection is addressed as loci on a solid support; and each locus on the solid support comprises polyclonal antibodies specific for one binding partner.

14. The combination of claim 13, wherein the avidity of the polyclonal antibodies for the binding partner at each locus is about 108-1012.

15. The combination of claim 1, wherein the capture agents and/or binding partners comprise scFvs.

16. The combination of claim 15, wherein the capture agents comprise scFvs.

17. The combination of claim 1, wherein the addressable collection is positionally addressable; and each address comprises a spot on a solid support.

18. The combination of claim 17 that is a array.

19. The combination of claim 17, wherein the solid support is selected from the group consisting of silicon, cellulose, metal, polymeric surfaces and radiation grafted supports.

20. The combination of claim 17, wherein the solid support is selected from the group consisting of gold, nitrocellulose, polyvinylidene difluoride (PVDF), radiation grafted polytetrafluoroethylene, polystyrene, glass and activated glass.

21. The combination of claim 17, wherein the solid support comprises a well or pit or plurality thereof in a surface of the solid support.

22. The combination of claim 17, wherein the solid support is selected from the group consisting of plates, beads, microbeads, whiskers, combs, hybridization chips, membranes, single crystals, ceramics and self-assembling monolayers.

23. The combination of claim 17, wherein the collections of capture agents are conjugated with biotin or a biotin derivative and the solid support is conjugated with avidin, streptavidin or a derivative thereof, whereby the capture agents are linked to the support.

24. The combination of claim 17, wherein the capture agents are attached to the solid support directly or via a linker by a covalent bond, an electrostatic bond, a hydrogen bond or a combination thereof.

25. The combination of claim 17, wherein one or more capture agents are linked to the solid supports via a linker or linkers.

26. The combination of claim 25, wherein the linker is selected from the group consisting of oligopeptides, oligonucleotides, oligopolyamides, oligoethyleneglycerol, oligoacrylamides, alkyl chains of between about 6 to about 20 carbon atoms, and combinations thereof.

27. The combination of claim 24, wherein the attachment is a cleavable attachment.

28. The combination of claim 27, wherein the cleavable attachment is cleavable by an enzyme, a chemical agent or electromagnetic radiation.

29. The combination of claim 28, wherein the chemical agent is selected from the group consisting of reducing agents, oxidizing agents, hydrolyzing agents and combinations thereof.

30. The combination of claim 28, wherein the electromagnetic radiation is selected from the group consisting of visible, ultraviolet and infrared radiation.

31. The combination of claim 1, wherein the collection of capture agents are addressably tagged by linking them to electronic, chemical, optical or color-coded labels.

32. The combination of claim 6, wherein the covalent linkage effected by the conjugation reagent is selected from the group consisting of thiol-thiol, thiol-amine, amine-amine, amine-carboxylic acid, thiol-carboxylic acid, thiol-carbohydrate and amine-non selective linkage.

33. The combination of claim 1, further comprising a computer readable medium containing pattern recognition software.

34. A kit comprising the combination of claim 1 and one or more of the following:
(a) packaging material; and
(b) instructions for using the kit for preparation, use and/or analysis of self-assembled arrays.

35. A combination, comprising:
an addressable collection of capture agents; and
a list setting forth the amino acid sequences that comprise the binding portion of polypeptide binding partners for each member of the collection of capture agents or setting forth sequences of nucleotides that encode the sequences of amino acids of binding partners that specifically bind to each member of the collection of capture agents.

36. The combination of claim 35, further comprising:
one or more conjugation regents for effecting covalent linkage of a binding partner to a displayed molecule or displayed biological particle.

37. The combination of claim 35, wherein the capture agents comprise antibodies.

38. The combination of claim 37, wherein the antibodies are monoclonal antibodies or fragments thereof that retain the ability to specifically bind to a binding partner.

39. The combination of claim 35, wherein:
the capture agents comprise polyclonal antibodies;
the collection is addressed as loci on a solid support; and
each locus on the solid support comprises polyclonal antibodies specific for one binding partner.

40. The combination of claim 39, wherein the avidity of the polyclonal antibodies for the binding partner at each locus is about 108-1012.

41. The combination of claim 35, wherein the capture agents and/or binding partners comprise scFvs.

42. The combination of claim 37, wherein the antibodies comprise an antibody fragment that comprises a scFv.

43. The combination of claim 35, wherein polypeptide binding partners are selected from the group consisting of an E-tag polypeptide (SEQ ID No.), a FLAG polypeptide (SEQ ID No.2), a Glu-Glu polypeptide (SEQ ID No.3), a HA.11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ ID No.12), a P5D4-A polypeptide (SEQ ID No.13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a sl polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

44. The combination of claim 35, wherein a sequence of nucleotides encodes a polypeptide selected from the group consisting of an E-tag polypeptide (SEQ ID No.1), a FLAG polypeptide (SEQ ID No.2), a Glu-Glu polypeptide (SEQ ID No.3), a HA11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ ID No.12), a P5D4-A polypeptide (SEQ ID No.13), a P5D4-B polypeptide (SEQ ID No.14), a 4C10 polypeptide (SEQ ID No. 15), an AB3 polypeptide (SEQ ID No.16), an AB6 polypeptide (SEQ ID No.1 7), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a S1 polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

45. The combination of claim 35, wherein the addressable collection is positionally addressable; and
each address comprises a spot on a solid support.

46. The combination of claim 45 that is an array.

47. The combination of claim 45, wherein the solid support is selected from the group consisting of silicon, celluloses, metal, polymeric surfaces and radiation grafted supports.

48. The combination of claim 45, wherein the solid support is selected from the group consisting of gold, nitrocellulose, polyvinyldiene fluoride polyvinylidene difluoride (PVDF), radiation grafted polytetrafluoroethylene (PFTE), polystyrene, glass and activated glass.

49. The combination of claim 45, wherein the solid support comprises a well or pit or plurality thereof in a surface of the solid support.

50. The combination of claim 45, wherein the solid support is selected from the group consisting of plates, beads, microbeads, whiskers, combs, hybridization chips, membranes, single crystals, ceramics and self-assembling monolayers.

51. The combination of claim 45, wherein the collections of capture agents are conjugated to biotin or a biotin derivative and the solid support is conjugated to avidin, streptavidin or a derivative thereof, whereby the capture agents are linked to the solid support.

52. The combination of claim 45, wherein the capture agents are attached to the solid support by a covalent bond, an electrostatic bond, a hydrogen bond or a combination thereof.

53. The combination of claim 45, further comprising a linker between the collections of capture agents and the solid support.

54. The combination of claim 53, wherein the linker is selected from the group consisting of oligopeptides, oligonucleotides, oligopolyamides, oligoethyleneglycerol, oligoacrylamides, alkyl chains of between about 6 to about 20 carbon atoms, and combinations thereof.

55. The combination of claim 52, wherein the attachment is a cleavable attachment.

56. The combination of claim 55, wherein the cleavable attachment is cleavable by an enzyme, a chemical agent or electromagnetic radiation.

57. The combination of claim 56, wherein the chemical agent is selected from the group consisting of reducing agents, oxidizing agents, hydrolyzing agents and combinations thereof.

58. The combination of claim 56, wherein the electromagnetic radiation is selected from the group consisting of visible, ultraviolet and infrared radiation.

59. The combination of claim 35, wherein the collection of capture agents are addressably tagged by linking them to electronic, chemical, optical or color-coded labels.

60. The combination of claim 35, further comprising one or more conjugation regents for effecting covalent linkage of a binding partner to a molecule or biological particle.

61. The combination of claim 35, further comprising a computer readable medium containing pattern recognition software.

62. A kit comprising a combination of claim 35 and one or more of the following:
(a) packaging material; and
(b) instructions for using the kit for preparation, use and/or analysis of self-assembled arrays.

63. A combination, comprising:
an addressable collection of capture agents;
a collection of sets of nucleic acid molecules, wherein:
the nucleic acid molecules of each set encode all or a portion of a polypeptide binding partner;
the encoded polypeptide binding partner or portion binds to a capture agent; and
one or more conjugation reagent(s) for linking the encoded polypeptides to molecules and/or biological particles.

64. The combination of claim 63, wherein the capture agents comprise antibodies.

65. The combination of claim 64, wherein the antibodies are monoclonal antibodies or fragments thereof that retain the ability to specifically bind to a binding partner.

66. The combination of claim 63, wherein:
the capture agents comprise polyclonal antibodies;
the collection is addressed as loci on a solid support; and
each locus on the solid support comprises polyclonal antibodies specific for one binding partner.

67. The combination of claim 66, wherein the avidity of the polyclonal antibodies for the binding partner at each locus is about 108-1012.

68. The combination of claim 63, wherein a capture agent and/or binding partner comprise(s) a scFv.

69. The combination of claim 64, wherein an antibody or fragment thereof is an anti-peptide antibody selected from the group consisting of an anti-E-tag antibody, an anti-FLAG M2 antibody, an anti-Glu-Glu antibody, an anti-HA.11 antibody, an anti-HSV-tag antibody, an anti-c-myc antibody, an anti-T7 tag antibody, an anti-VSV G antibody, an anti-V5 antibody, an anti-AB2 antibody, an anti-AB4 antibody, an anti-B34 antibody, an anti-P5D4 A antibody, an anti-P5D4 B antibody, an anti-4C10 antibody, an anti-AB3 antibody, an anti-AB6 antibody, an anti-KT3 A antibody, an anti-KT3 B antibody, an anti-KT3 C antibody, an anti-7.23 antibody, an anti-HOPCl antibody, an anti-S1 antibody, an anti-E2 antibody, an anti-His tag antibody, an anti-AU1 antibody, an anti-AU5 antibody, an anti-IRS antibody, an anti-NusA antibody, an anti-MBP antibody, an anti-TBP antibody and an anti-TRX antibody.

70. The combination of claim 64, wherein one or more antibodies comprise a fragment that comprises a scFv.

71. The combination of claim 63, wherein a polypeptide binding partner is selected from the group consisting of an E-tag polypeptide (SEQ ID No.1), a FLAG polypeptide (SEQ ID No.2), a Glu-Glu polypeptide (SEQ ID No.3), a HA.11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ ID No.12), a P5D4-A polypeptide (SEQ ID No.13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a sl polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

72. The combination of claim 63, wherein one or more of the sets of nucleotides encode a polypeptide selected from the group consisting of an E-tag polypeptide (SEQ ID No. 1), a FLAG polypeptide (SEQ ID No.2), a Glu-Glu polypeptide (SEQ ID No.3), a HA.11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ iD No.12), a P5D4-A polypeptide (SEQ ID No.13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a S1 polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

73. The combination of claim 63, wherein the addressable collection is positionally addressable; and
each address comprises a spot on an a solid support.

74. The combination of claim 73 that is a array.

75. The combination of claim 73, wherein the solid support is selected from the group consisting of silicon, celluloses, metal, polymeric surfaces and radiation grafted supports.

76. The combination of claim 73, wherein the solid support is selected from the group consisting of gold, nitrocellulose, polyvinylidene difluoride (PVDF), radiation grafted polytetrafluoroethylene (PFTE), polystyrene, glass and activated glass.

77. The combination of claim 73, wherein the solid support comprises a well or pit or plurality thereof in a surface of the solid support.

78. The combination of claim 73, wherein the solid support is selected from the group consisting of plates, beads, microbeads, whiskers, combs, hybridization chips, membranes, single crystals, ceramics and self-assembling monolayers.

79. The combination of claim 73, wherein the collections of capture agents are conjugated with biotin or a biotin derivative and the solid support is conjugated with avidin, streptavidin or a derivative thereof.

80. The combination of claim 73, wherein the capture agents are attached to the solid support by a covalent bond, an electrostatic bond, a hydrogen bond or a combination thereof.

81. The combination of claim 73, further comprising a linker between the collections of capture agents and the solid support.

82. The combination of claim 81, wherein the linker is selected from the group consisting of oligopeptides, oligonucleotides, oligopolyamides, oligoethyleneglycerol, oligoacrylamides, alkyl chains of between about 6 to about 20 carbon atoms, and combinations thereof.

83. The combination of claim 80, wherein the attachment is a cleavable attachment.

84. The combination of claim 83, wherein the cleavable attachment is cleavable by an enzyme, a chemical agent or electromagnetic radiation.

85. The combination of claim 84, wherein the chemical agent is selected from the group consisting of reducing agents, oxidizing agents, hydrolyzing agents and combinations thereof.

86. The combination of claim 84, wherein the electromagnetic radiation is selected from the group consisting of visible, ultraviolet and infrared radiation.

87. The combination of claim 63, wherein the collection of capture agents are addressably tagged by linking them to electronic, chemical, optical or color-coded labels.

88. The combination of claim 63, wherein the linking effected by the conjugation reagent(s) is selected from the group consisting of thiol-thiol, thiol-amine, amine-amine, amine-carboxylic acid, thiol-carboxylic acid, thiol-carbohydrate and amine-non selective linkage.

89. The combination of claim 63, further comprising a computer readable medium containing pattern recognition software.

90. A kit comprising the combination of claim 63 and one or more of the following:
(a) packaging material; and
(b) instructions for using the kit for preparation, use and/or analysis of self-assembled arrays.

91. A method for preparing a self-assembled array, comprising:
a) providing an addressable array of capture agents that have predetermined binding partners;
b) preparing a plurality of sets of conjugates, wherein:
each set of conjugates comprises a biological particle and/or molecule linked to a binding partner or plurality thereof, wherein the binding partner binds to one of the capture agents in the array; and
c) contacting the addressable array of capture agents with the sets of conjugates to produce the self-assembled array.

92. A method for preparing a self-assembled array, comprising:
a) providing an addressable array of capture agents that have predetermined binding partners;
b) preparing sets of binding partners, wherein a binding partner is a polypeptide encoded by a nucleic acid;
c) preparing a plurality of sets of conjugates, wherein:
each set of conjugates comprises a biological particle and/or molecule linked to a binding partner or plurality thereof from one set of binding partners, wherein the binding partner binds to one of the capture agents in the array; and
d) contacting the addressable array of capture agents with the sets of conjugates to produce the self-assembled array.

93. The method of claim 91, wherein the biological particle and/or molecule is linked to the binding partner by a linker or intermediate molecule.

94. The combination of claim 93, wherein the linker is selected from the group consisting of peptide linker, a chemical linker, and a cleavable linker.

95. The combination of claim 94, wherein the cleavable linker is selected from the group consisting of acid-cleavable, heat labile and photocleavable linkers.

96. The combination of claim 93, wherein the intermediate molecule is a bead.

97. The combination of claim 96, wherein the bead is linked to an electronic, chemical, optical, or color-coded label.

98. The combination of claim 93, wherein the linking of the binding partner to the linker or intermediate molecule comprises a covalent bond, an electrostatic bond, a hydrogen bond, or a combination thereof.

99. The combination of claim 93, wherein a displayed molecule and/or a displayed particle is attached to the linker or intermediate molecule.

100. The combination of claim 99, wherein the attachment of the displayed molecule and/or a displayed particle to the linker or intermediate molecule comprises a covalent bond, an electrostatic bond, a hydrogen bond, or a combination thereof.

101. The method of claim 92, wherein the nucleic acid is synthetically prepared.

102. A method for preparing a self-assembled array, comprising:
a) providing an addressable array of capture agents that have predetermined binding partners;
b) providing a plurality of sets binding partners, wherein each set of binding partners specifically binds to a unique capture agent, and each set of binding partners is separate from each other set; and
c) crosslinking each set of binding partners to molecules and/or biological particles to produce sets of conjugates.

103. A method for preparing a self-assembled array, comprising:
a) providing an addressable array of capture agents that have predetermined binding partners;
b) providing a plurality of sets binding partners, wherein each set of binding partners specifically binds to a unique capture agent, and each set of binding partners is separate from each other set; and
c) linking each set of binding partners to beads to produce sets of binding partner beads.

104. The method of claim 103, further comprising: contacting the sets of binding partner beads with biological particles and/or molecules; wherein each set of binding partner beads is associated with a specific biological particle and/or molecule.

105. The method of claim 104, further comprising: contacting the binding partner beads linked to biological particles and/or molecules with the addressable array to form a self-assembled array.

106. The method of claim 102, further comprising: contacting the crosslinked biological particles and/or molecules with the array to form the self-assembled array.

107. The method of claim 91, wherein the number of sets of conjugates is equal to the number of unique capture agents.

108. The method of claim 91, wherein the conjugates are chemical conjugates.

109. The method of claim 108, wherein the chemical conjugate is linked by an interaction selected from the group consisting of covalent, ionic, hydrophobic and van der Waals interactions; and
the interaction is sufficiently stable to maintain the conjugation of the conjugate upon binding to the capture agent.

110. The method of claim 91, wherein the conjugates are recombinant conjugates.

111. The method of claim 110, wherein the recombinant conjugate is a fusion protein.

112. The method of claim 91, wherein the capture agents comprise antibodies.

113. The method of claim 112, wherein the antibodies are monoclonal antibodies or fragments thereof that retain the ability to specifically bind to a binding partner.

114. The method of claim 91, wherein the binding partners comprise polypeptides.

115. The method of claim 108, wherein the chemical conjugates are prepared using one or more conjugation regents covalent linkage of a binding partner to a molecule or biological particle.

116. The method of claim 115, wherein the linkage effected by the conjugation reagent(s) is selected from the group consisting of thiol-thiol, thiol-amine, amine-amine, amine-carboxylic acid, thiol-carboxylic acid, thiol-carbohydrate and amine-non selective linkage.

117. The method of claim 112, wherein:
the capture agents comprise polyclonal antibodies;
the collection is addressed as loci on a solid support; and
each locus on the solid support comprises polyclonal antibodies specific for one binding partner.

118. The method of claim 112, wherein the avidity of the polyclonal antibodies for the binding partner at each locus is about 108-1012.

119. The method of claim 91, wherein the capture agents and/or binding partners comprise scFvs.

120. The method of claim 113, wherein antibodies or fragments thereof are anti-peptide antibodies selected from the group consisting of an anti-E-tag antibody, an anti-FLAG M2 antibody, an anti-Glu-Glu antibody, an anti-HA.11 antibody, an anti-HSV-tag antibody, an anti-c-myc antibody, an anti-T7 tag antibody, an anti-VSV G antibody, an anti-V5 antibody, an anti-AB2 antibody, an anti-AB4 antibody, an anti-B34 antibody, an anti-P5D4 A antibody, an anti-P5D4 B antibody, an anti-4C10 antibody, an anti-AB3 antibody, an anti-AB6 antibody, an anti-KT3 A antibody, an anti-KT3 B antibody, an anti-KT3 C antibody, an anti-7.23 antibody, an anti-HOPC1 antibody, an anti-S1 antibody, an anti-E2 antibody, an anti-His tag antibody, an anti-AU1 antibody, an anti-AU5 antibody, an anti-IRS antibody, an anti-NusA antibody, an anti-MBP antibody, an anti-TBP antibody and an anti-TRX antibody.

121. The method of claim 112, wherein antibodies a comprise an antibody fragment that comprises a scFv.

122. The method of claim 114, wherein polypeptides are selected from the group consisting of an E-tag polypeptide (SEQ ID No.1), a FLAG polypeptide (SEQ ID No.2), a Glu-Glu polypeptide (SEQ ID No.3), a HA.11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ ID No.12), a P5D4-A polypeptide (SEQ ID No.13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.18), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a s1 polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

123. The method of claim 91, wherein the addressable collection is positionally addressable; and
each address comprises a spot on an a solid support.

124. The method of claim 123 that is a array.

125. The method of claim 123, wherein the solid support is selected from the group consisting of silicon, celluloses, metal, polymeric surfaces and radiation grafted supports.

126. The method of claim 124, wherein the solid support is selected from the group consisting of gold, nitrocellulose, polyvinylidene difluoride (PVDF), radiation grafted polytetrafluoroethylene (PFTE), polystyrene, glass and activated glass.

127. The method of claim 123, wherein the solid support comprises a well or pit or plurality thereof in a surface of the solid support.

128. The method of claim 123, wherein the solid support is selected from the group consisting of plates, beads, microbeads, whiskers, combs, hybridization chips, membranes, single crystals, ceramics and self-assembling monolayers.

129. The method of claim 123, wherein the capture agents are attached to the solid support by a covalent bond, an electrostatic bond, a hydrogen bond or a combination thereof.

130. The method of claim 123, wherein the collections of capture agents are conjugated with biotin or a biotin derivative and the solid support is conjugated with avidin, streptavidin or a derivative thereof.

131. The method of claim 123, further comprising a linker between the collections of capture agents and the solid support.

132. The method of claim 131, wherein the linker is selected from the group consisting of oligopeptides, oligonucleotides, oligopolyamides, oligoethyleneglycerol, oligoacrylamides, alkyl chains of between about 6 to about 20 carbon atoms, and combinations thereof.

133. The method of claim 129, wherein the attachment is a cleavable attachment.

134. The method of claim 133, wherein the cleavable attachment is cleavable by an enzyme, a chemical agent or electromagnetic radiation.

135. The method of claim 134, wherein the chemical agent is selected from the group consisting of reducing agents, oxidizing agents, hydrolyzing agents and methods thereof.

136. The method of claim 134, wherein the electromagnetic radiation is selected from the group consisting of visible, ultraviolet and infrared radiation.

137. The method of claim 91, wherein the collection of capture agents are addressably tagged by linking them to electronic, chemical, optical or color-coded labels.

138. The method of claim 115, wherein the conjugation reagent is m-maleimidobenzoyl-N-hydroxysuccinamide ester (Sulfo-MBS).

139. The method of claim 91, wherein the molecule is a cyclic peptide.

140. A method for monitoring an interaction of an exogenous molecule and/or biological particle with a self-assembled array, comprising:
a) performing the method of claim 91;
b) contacting the exogenous molecule and/or biological particle to the self-assembled array; and
c) monitoring the interaction between the exogenous molecule and/or biological particle and the self-assembled array.

141. The method of claim 140, further comprising:
(d) adding a candidate compound or exposing the self-assembled array to a condition before, simultaneously with or after contacting the exogenous molecule and/or biological particle with the self-assembled array; and
(e) assessing an effect of the candidate compound or condition on the interaction between the exogenous molecules and/or biological particles and the self-assembled array to monitor or identify the effect of the candidate compound, condition or both on the occurrence of the events.

142. The method of claim 141, wherein the contacting and assessing are performed simultaneously or sequentially.

143. The method of claim 140, wherein the effect is selected from the group consisting of a change in structure, function, a physical change, a chemical or a morphological change, signal transduction, protein trafficking, gene expression, translation, the pattern (profile) of captured molecules, degradation of a biopolymer in or on the biological particle, proliferation, cell death, apoptosis, morphological changes, gene expression, transcription, translation, receptor internalization, receptor shedding, receptor-mediated activation of the biological particle or a receptor thereon or therein, differentiation, dedifferentiation, interactions among biological particles, endocytosis, phagocytosis, exocytosis, phosphorylation, dephosphorylation and change in kinetics of an intra-particle reaction.

144. The method of claim 141, wherein the candidate compound is selected from the group consisting of an organic compound, an inorganic compound, a metal complex, a receptor, a ligand, an enzyme, an antibody, a protein, a nucleic acid, a peptide nucleic acid, DNA, RNA, a polynucleotide, an oligonucleotide, an oligosaccharide, a lipid, a lipoprotein, an amino acid, a peptide, a cyclic peptide, a polypeptide, a peptidomimetic, a carbohydrate, a cofactor, a drug, a prodrug, a lectin, a sugar, a glycoprotein, a biomolecule, a macromolecule, a biopolymer, a polymer, a sub-cellular structure, a sub-cellular compartment, a virus, a phage, a cell, a liposome, and a micellar agent.

145. The method of claim 141, wherein the condition is selected from the group consisting of a variation in buffer or solution components, pH, temperature, exposure to light, aerobic or anaerobic conditions, concentration of components, duration of experimental detection, ionic strength, pressure, agitation, and organic or aqueous interaction medium.

146. The method of claim 140, wherein the interaction is known.

147. A method of identifying a molecule that modulates trafficking in biological particles, comprising:
a) performing the method of claim 91;
b) contacting an exogenous biological particle to the self-assembled array; and
c) monitoring trafficking in the exogenous biological particle, to thereby identifying the conjugated molecule(s) from among the self-assembled array that modulate the trafficking in the exogenous biological particle.

148. A method of identifying an exogenous molecule that modulates trafficking in biological particles, comprising:
a) performing the method of claim 91;
b) adding a candidate compound or exposing the self-assembled array to a condition before, during or after contacting the self-assembled array with an exogenous biological particle; and
c) monitoring trafficking in the exogenous biological particle, to thereby identify the candidate compound(s) and/or condition(s) that modulate trafficking in the exogenous biological particle.

149. The method of claim 147, wherein the conjugated molecules or candidate compounds that modulate trafficking are selected from the group consisting of oligonucleotides, oligonucleosides, polypeptides, amino acids, nucleotides, nucleosides, peptide nucleic acids, oligosaccharides, monosaccharides, organic compounds, inorganic compounds, metal complexes, metal ions, lipids, lipoproteins, peptidomimetics, carbohydrates, cofactors, drugs, prodrugs, lectins, sugars, glycoproteins, biomolecule, macromolecule, biopolymer, polymer, sub-cellular structure, sub-cellular compartment or any combination, portion, salt, or derivative thereof.

150. The method of claim 149, wherein the polypeptides are selected from the group consisting of: enzymes, proteins, receptors, cellular adhesion molecules, antibodies and fragments thereof.

151. A method for identifying a molecule that modulates activity or functional or structural property in or of molecules and/or biological particles, comprising:
a) performing the method of claim 91;
b) contacting the self-assembled array with exogenous molecules; and
c) monitoring the activity, function or structural property in or of the conjugated molecules and/or biological particles in the self-assembled array, to thereby identify the exogenous molecule(s) that modulate the activity, function or structural property in or of the conjugated molecules and/or biological particles in the self-assembled array.

152. A method for identifying a molecule that modulates an activity or functional or structural property in or of a conjugated molecule and/or biological particle in a self-assembled array, comprising:
a) performing the method of claim 91;
b) adding a candidate compound or exposing the self-assembled array to a condition before, during or after contacting the self-assembled array with exogenous molecules; and
c) monitoring the activity, function or structural property in or of the conjugated molecules and/or biological particles, to thereby identify the candidate compound(s) and/or condition(s) that modulate the activity, function or structural property in or of the conjugated molecules and/or biological particles in the self-assembled array.

153. The method of claim 151, wherein the activity, function or structural property are selected from the group consisting of proliferation, apoptosis, morphology, transcription, translation, receptor internalization, receptor shedding, signal transduction, receptor-mediated activation of a biological particle, receptor-activated signaling in a biological particle, differentiation, dedifferentiation, interactions among constituent proteins and/or protein complexes and components thereof, interactions among biological particles, endocytosis, phagocytosis, exocytosis, phosphorylation, dephosphorylation and change in kinetics of an intra-particle reaction.

154. A self-assembled array produced by a method of claim 91.

155. A self-assembled array, comprising:
a) an addressable array of capture agents that have predetermined binding partners; and
b) a plurality of sets of conjugates, wherein:
each set of conjugates comprises a biological particle and/or molecule linked to a binding partner or plurality thereof, wherein the binding partners are specifically bound to their capture agents.

156. The self-assembled array of claim 155, wherein the capture agents comprise antibodies.

157. The self-assembled array of claim 156, wherein the antibodies are monoclonal antibodies or fragments thereof that retain the ability to specifically bind to a binding partner.

158. The self-assembled array of claim 157, wherein the binding partners comprise polypeptides.

159. The self-assembled array of claim 155, wherein:
the capture agents comprise polyclonal antibodies;
the collection is addressed as loci on a solid support; and
each locus on the solid support comprises polyclonal antibodies specific for one binding partner.

160. The self-assembled array of claim 159, wherein the avidity of the polyclonal antibodies for the binding partner at each locus is about 108-1012.

161. The self-assembled array of claim 155, wherein the capture agents and/or binding partners comprise scFvs.

162. The self-assembled array of claim 157, wherein the antibody or fragment thereof is an anti-peptide antibody selected from the group consisting of an anti-E-tag antibody, an anti-FLAG M2 antibody, an anti-Glu-Glu antibody, an anti-HA.11 antibody, an anti-HSV-tag antibody, an anti-c-myc antibody, an anti-T7 tag antibody, an anti-VSV G antibody, an anti-V5 antibody, an anti-AB2 antibody, an anti-AB4 antibody, an anti-B34 antibody, an anti-P5D4 A antibody, an anti-P5D4 B antibody, an anti-4C10 antibody, an anti-AB3 antibody, an anti-AB6 antibody, an anti-KT3 A antibody, an anti-KT3 B antibody, an anti-KT3 C antibody, an anti-7.23 antibody, an anti-HOPC1 antibody, an anti-S1 antibody, an anti-E2 antibody, an anti-His tag antibody, an anti-AU1 antibody, an anti-AU5 antibody, an anti-IRS antibody, an anti-NusA antibody, an anti-MBP antibody, an anti-TBP antibody and an anti-TRX antibody.

163. The self-assembled array of claim 159, wherein the polyclonal antibodies comprise an antibody fragment that comprises a scFv.

164. The self-assembled array of claim 158, wherein the polypeptide is selected from the group consisting of an E-tag polypeptide (SEQ ID No.1), a FLAG polypeptide (SEQ ID No.2), a Glu-Glu polypeptide (SEQ ID No.3), a HA.11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ ID No.12), a P5D4-A polypeptide (SEQ ID No.13), a P5D4-B polypeptide (SEQ ID No.14), a 4C10 polypeptide (SEQ ID No.15), an AB3 polypeptide (SEQ ID No.16), an AB6 polypeptide (SEQ ID No.17), a KT3-A polypeptide (SEQ ID No.1 8), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a S1 polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

165. The self-assembled array of claim 155, wherein the addressable collection is positionally addressable; and
each address comprises a spot on a solid support.

166. The self-assembled array of claim 165, wherein the solid support is selected from the group consisting of silicon, celluloses, metal, polymeric surfaces and radiation grafted supports.

167. The self-assembled array of claim 165, wherein the solid support is selected from the group consisting of gold, nitrocellulose, polyvinylidene difluoride (PVDF), radiation grafted polytetrafluoroethylene, polystyrene, glass and activated glass.

168. The self-assembled array of claim 165, wherein the solid support comprises a well or pit or plurality thereof in a surface of the solid support.

169. The self-assembled array of claim 165, wherein the solid support is selected from the group consisting of plates, beads, microbeads, whiskers, combs, hybridization chips, membranes, single crystals, ceramics and self-assembling monolayers.

170. The self-assembled array of claim 165, wherein the collections of capture agents are conjugated with biotin or a biotin derivative and the solid support is conjugated with avidin, streptavidin or a derivative thereof, whereby the capture agents are linked to the support.

171. The self-assembled array of claim 165, wherein the capture agents are attached to the solid support by a covalent bond, an electrostatic bond, a hydrogen bond or a combination thereof.

172. The self-assembled array of claim 165, further comprising a spacer between the collections of capture agents and the solid support.

173. The self-assembled array of claim 172, wherein the spacer is selected from the group consisting of oligopeptides, oligonucleotides, oligopolyamides, oligoethyleneglycerol, oligoacrylamides, alkyl chains of between about 6 to about 20 carbon atoms, and combinations thereof.

174. The self-assembled array of claim 171, wherein the attachment is a cleavable attachment.

175. The self-assembled array of claim 174, wherein the cleavable attachment is cleavable by an enzyme, a chemical agent or electromagnetic radiation.

176. The self-assembled array of claim 175, wherein the chemical agent is selected from the group consisting of reducing agents, oxidizing agents, hydrolyzing agents and combinations thereof.

177. The self-assembled array of claim 175, wherein the electromagnetic radiation is selected from the group consisting of visible, ultraviolet and infrared radiation.

178. The self-assembled array of claim 155, wherein the collection of capture agents are addressably tagged by linking them to electronic, chemical, optical or color-coded labels.

179. A combination, comprising:
an addressable collection of capture agents;
a plurality of sets of binding partners, wherein each set of binding partners specifically binds to a unique capture agent; and
wherein the binding partner is linked to a linker or intermediate molecule; and wherein a displayed molecule and/or a displayed particle is attached to the linker or intermediate molecule.

180. The combination of claim 179, wherein the linker is selected from the group consisting of peptide linker, a chemical linker, and a cleavable linker.

181. The combination of claim 180, wherein the cleavable linker is selected from the group consisting of acid-cleavable, heat labile and photocleavable linkers.

182. The combination of claim 179, wherein the intermediate molecule is a bead.

183. The combination of claim 182, wherein the bead is linked to an electronic, chemical, optical, or color-coded label.

184. The combination of claim 179, wherein the linking of the binding partner to the linker or intermediate molecule comprises a covalent bond, an electrostatic bond, a hydrogen bond, or a combination thereof.

185. The combination of claim 179, wherein the attachment of the displayed molecule and/or a displayed particle to the linker or intermediate molecule comprises a covalent bond, an electrostatic bond, a hydrogen bond, or a combination thereof.

186. A kit comprising the combination of claim 179 and one or more of the following:
(a) packaging material; and
(b) instructions for using the kit for preparation, use and/or analysis of self-assembled arrays.

187. A method of analyzing and/or processing a signal or plurality thereof at loci on an exposed positionally addressable array, comprising:
a) receiving image data corresponding to pixel luminosity information at the exposed locus within the positionally addressable array, wherein the locus comprises capture agents;
b) specifying pre-determined input parameters for processing the received image data that include parameters that specify a predetermined array locus or plurality thereof on a surface of the array;
c) determining an actual location of a locus or plurality thereof on the array; and
d) processing the image data for each exposed locus in accord with the determined actual locus and the input parameters.

188. The method of claim 187, wherein processing comprises determining or detecting luminosity at the actual locus on the array.

189. The method of claim 187, wherein a plurality of arrays are processed.

190. The method of claim 187, wherein determining the actual locus comprises:
processing image data that corresponds to pixels located a distance from a pre-selected locus such that luminosity information for the pixels indicates increasing values; and
recording an array location that corresponds to a local maximum of intensity values as the actual location of the locus.

191. The method of claim 187, wherein the pre-determined input parameters for processing the received image data that include parameters that specify all loci on the surface of the array.

192. A method for processing a signal or plurality thereof at loci that display a detectable signal in or on an array, comprising:
a) receiving image data corresponding to pixel luminosity information for locations for a plurality of the loci of the array;
b) for a particular locus of the array, determining neighbor luminosity effects on the image data for the particular locus that are produced from adjacent neighbor loci; and
c) compensating for the neighbor luminosity effects of the adjacent neighbor loci from the image data for the particular locus.

193. The method of claim 192, wherein a plurality of arrays are processed.

194. The method of claim 187, wherein the array is a self-assembled array.

195. A method of processing a signal or plurality thereof at loci on a positionally addressable array, comprising:
a) receiving image data corresponding to pixel luminosity information of the locations for a plurality of the loci of the array;
b) for a particular locus of the array, determining neighbor luminosity effects on the image data for the particular locus that are produced from adjacent neighbor loci; and
c) compensating for the neighbor luminosity effects of the adjacent neighbor loci from the image data for the particular locus.

196. The method of claim 195, wherein a plurality of arrays are processed.

197. The method of claim 195, wherein determining neighbor luminosity effects comprises:
determining a luminosity value corresponding to an illumination value for a location at each neighbor locus of the particular locus;
determining an actual location on the top surface of the canvas for each locus;
determining an array distance from the particular locus to each of the associated neighbor loci; and
compensating for the luminosity effects from each of the neighbor loci in accord with their respective distance to the particular locus.

198. The method of claim 197, wherein determining an actual location for each particular loci comprises:
processing image data that corresponds to pixels located a distance from each particular locus of interest such that luminosity information for the pixels indicates increasing values; and
recording an array location that corresponds to a local maximum of intensity values as the actual location of the particular locus of interest.

199. The method of claim 198, wherein determining the array distance comprises subtracting the determined actual locations of the particular locus to determine distances between the respective locations of the particular locus and the asssociated associated neighbor loci.

200. The method of claim 195, wherein the array is a self-assembled array.

201. A program product for use in a computer device that executes program instructions recorded in a computer-readable medium to perform a method for analyzing arrays that have loci that display a detectable signal in or on the array, the program product comprising:
a recordable medium; and
a plurality of computer-readable instructions executable by the computer device to perform a method comprising:
receiving image data corresponding to pixel luminosity information of the loci;
specifying input parameters for processing the received image data that include parameters that specify a predicted array location for each locus of the array,
determining an actual location for each locus; and
processing the image data for each location in accord with the determined actual location and the input parameters.

202. The program product of claim 201, wherein determining the actual location comprises:
processing image data that corresponds to pixels located a distance from a locus such that luminosity information for the pixels indicates increasing values; and
recording an array location that corresponds to a local maximum of intensity values as the actual locus.

203. A program product for use in a computer device that executes program instructions recorded in a computer-readable medium to perform a method for processing an array with loci that display a detectable signal in or on the array, comprising:
a recordable medium; and
a plurality of computer-readable instructions executable by the computer device to perform the method comprising
receiving image data corresponding to pixel luminosity information of the loci for a plurality of the locations of the array,
for a particular locus in the array, determining neighbor luminosity effects on the image data for the particular location that are produced from adjacent neighbor locations, and
compensating for the neighbor luminosity effects of the adjacent neighbor locations from the image data for the particular location.

204. The program product of claim 203, wherein determining neighbor luminosity effects comprises:
determining a luminosity value corresponding to an illumination value for a biological material location at each neighbor location of the particular location;
determining an actual location on the top surface of the canvas for each biological material location;
determining an array distance from the location of the particular biological material to each of the associated neighbor locations; and
compensating for the luminosity effects from each of the neighbor locations in accord with their respective distance to the location of the particular biological material.

205. The program product of claim 204, wherein determining an actual locus, comprises:
processing image data that corresponds to pixels located a distance from a locus such that luminosity information for the pixels indicates increasing values; and
recording the array location that corresponds to a local maximum of intensity values as the actual location of the biological material of interest.

206. The program product of claim 205, wherein determining the array distance comprises subtracting the determined actual locations of the biological locations to determine distances between the respective locations of the biological material and the neighbor locations.

207. An apparatus that processes data produced from imaging detectable loci on an array, comprising:
a computer processor that executes program instructions;
an input processor that receives input parameters for processing received image data,
including input parameters that specify a predicted array location for each locus on the array, wherein the image data corresponds to pixel luminosity information for loci in the array; and
an image analysis processor that determines an actual location on the array for each locus, and processes the image data for each location in accord with the determined actual location and the input parameters.

208. The apparatus of claim 207, wherein the image analysis processor determines the actual location by processing image data that corresponds to pixels located a distance from a particular locus such that luminosity information for the pixels indicates increasing values, and recording an array location that corresponds to a local maximum of intensity values as the actual location of the biological material of interest.

209. An apparatus that processes data produced from imaging detectable loci in an array, the apparatus comprising:
a computer processor that executes program instructions;
an input processor that receives image data comprising pixel luminosity information for loci in the array; and
an image analysis processor that determines neighbor luminosity effects on image data for a particular locus produced from adjacent neighbor locations, and that compensates for the neighbor luminosity effects of the adjacent neighbor locations from the image data for the particular location.

210. The apparatus of claim 209, wherein the image analysis processor determines neighbor luminosity effects by determining a luminosity value corresponding to an illumination value for a biological material location at each neighbor location of the particular locus, determining an actual location on a surface of the array for each detectable locus, determining an array distance from the location of the particular biological material to each of the associated neighbor locations, and compensating for the luminosity effects from each of the neighbor locations in accord with their respective distance to the location of the detectable locus.

211. The apparatus of claim 170, wherein the image analysis processor determines the actual location for each detectable locus by processing image data that corresponds to pixels located a distance from each detectable locus such that luminosity information for the pixels indicates increasing values, and recording an array location that corresponds to a local maximum of intensity values as the actual location of the biological material of interest.

212. The apparatus of claim 211, wherein the image analysis processor determines the array distance by subtracting the determined actual locations of the biological locations to determine distances between the respective locations.

213. A system for analysis of a collection of self-assembled arrays, comprising:
an addressable collection of capture agents and binding partners, comprising sets of capture agents and binding partners, wherein a set of capture agents is selected to specifically bind to a set of binding partners with sufficiently high affinity to produce collections of self-assembled arrays;
a conjugation reagent, comprising a compound or molecule sufficient for the conjugation of the sets of binding partners to sets of molecules or biological particles;
a computer programmed with instructions for controlling and directing production of an image of the conjugated sets of molecules or biological particles displayed on the collections of self-assembled arrays; and
software for processing of image data produced by the collections of self-assembled arrays.

214. The system of claim 213 that is an automated system.

215. The system of claim 213, further comprising a microplate reader.

216. The system of claim 213, further comprising a charge coupled device (CCD) camera.

217. A combination, comprising:
a program product of claim 201;
an addressable collection of capture agents; and
a plurality of sets of binding partners, wherein each set of binding partners specifically binds to a unique capture agent

218. The combination of claim 217, further comprising;
either or both of:
one or more conjugation regents for effecting covalent linkage of a binding partner to a displayed molecule and/or displayed biological particle; and/or
instructions for use of the addressable collection of capture agent and binding partners to prepare self-assembled arrays.

219. A combination, comprising:
the program product of claim 201;
an addressable collection of capture agents; and
a list setting forth the amino acid sequences that comprise the binding portion of polypeptide binding partners for each member of the collection of capture agents or setting forth sequences of nucleotides that encode the sequences of amino acids of binding partners that specifically bind to each member of the collection of capture agents.

220. A combination, comprising:
the program product of claim 201;
an addressable collection of capture agents;
a collection of sets of nucleic acid molecules, wherein:
the members nucleic acid molecules of each set encode all or a portion of a polypeptide binding partner;
the encoded polypeptide binding partner or portion binds to a capture agent; and
a conjugation reagent for linking the encoded polypeptides to molecules and/or biological particles.

221. The combination of claim 217, wherein the capture agents comprise antibodies.

222. The combination of claim 221, wherein the antibodies are monoclonal antibodies or fragments thereof that retain the ability to specifically bind to a binding partner.

223. The combination of claim 217, wherein the binding partners comprise polypeptides.

224. The combination of claim 217, further comprising one or more conjugation regents for effecting covalent linkage of a binding partner to a molecule or biological particle.

225. The combination of claim 217, wherein:
the capture agents comprise polyclonal antibodies;
the collection is addressed as loci on a solid support; and each locus on the solid support comprises polyclonal antibodies specific for one binding partner.

226. The combination of claim 225, wherein the avidity of the polyclonal antibodies for the binding partner at each locus is about 108-1012.

227. The combination of claim 217, wherein the capture agents and/or binding partners comprise scFvs.

228. The combination of claim 222, wherein the antibodies or fragments thereof are anti-peptide antibodies selected from the group consisting of an anti-E-tag antibody, an anti-FLAG M2 antibody, an anti-Glu-Glu antibody, an anti-HA.11 antibody, an anti-HSV-tag antibody, an anti-c-myc antibody, an anti-T7 tag antibody, an anti-VSV G antibody, an anti-V5 antibody, an anti-AB2 antibody, an anti-AB4 antibody, an anti-B34 antibody, an anti-P5D4 A antibody, an anti-P5D4 B antibody, an anti-4C10 antibody, an anti-AB3 antibody, an anti-AB6 antibody, an anti-KT3 A antibody, an anti-KT3 B antibody, an anti-KT3 C antibody, an anti-7.23 antibody, an anti-HOPC1 antibody, an anti-S1 antibody, an anti-E2 antibody, an anti-His tag antibody, an anti-AU1 antibody, an anti-AU5 antibody, an anti-IRS antibody, an anti-NusA antibody, an anti-MBP antibody, an anti-TBP antibody and an anti-TRX antibody.

229. The combination of claim 225, wherein the antibody is a fragment that comprises a scFv.

230. The combination of claim 219, wherein the polypeptides are selected from the group consisting of an E-tag polypeptide (SEQ ID No.1), a FLAG polypeptide (SEQ ID No.2), a Glu-Glu polypeptide (SEQ ID No.3), a HA.11 polypeptide (SEQ ID No.4), a HSV-tag polypeptide (SEQ ID No.5), a c-myc polypeptide (SEQ ID No.6), a T7 tag polypeptide (SEQ ID No.7), a VSV-G polypeptide (SEQ ID No.8), a V5 polypeptide (SEQ ID No.9), an AB2 polypeptide (SEQ ID No.10), an AB4 polypeptide (SEQ ID No.11), a B34 polypeptide (SEQ ID No.12), a P5D4-A polypeptide (SEQ ID No.13), a P5D4-B polypeptide (SEQ ID No.14), a 4C10 polypeptide (SEQ ID No.15), an AB3 polypeptide (SEQ ID No.16), an AB6 polypeptide (SEQ ID No.1 7), a KT3-A polypeptide (SEQ ID No.1 8), a KT3-B polypeptide (SEQ ID No.19), a KT3-C polypeptide (SEQ ID No.20), a 7.23 polypeptide (SEQ ID No.21), a HOPC1 polypeptide (SEQ ID No.22), a S1 polypeptide (SEQ ID No.23), an E2 polypeptide (SEQ ID No.24), a His tag polypeptide (SEQ ID No.25), an AU1 polypeptide (SEQ ID No.26), an AU5 polypeptide (SEQ ID No.27), an IRS polypeptide (SEQ ID No.28), a KT3 polypeptide (SEQ ID No.34), NusA (SEQ ID No.29), Maltose binding protein (SEQ ID No.30), TATA-box binding protein (SEQ ID No.31) and thioredoxin (SEQ ID No.32).

231. The combination of claim 217, wherein the addressable collection is positionally addressable; and
each address comprises a spot on a solid support.

232. The combination of claim 231 that is a array.

233. The combination of claim 231, wherein the solid support is selected from the group consisting of silicon, celluloses, metal, polymeric surfaces and radiation grafted supports.

234. The combination of claim 231, wherein the solid support is selected from the group consisting of gold, nitrocellulose, polyvinylidene difluoride (PVDF), radiation grafted polytetrafluoroethylene, polystyrene, glass and activated glass.

235. The combination of claim 231, wherein the solid support comprises a well or pit or plurality thereof in a surface of the solid support.

236. The combination of claim 231, wherein the solid support is selected from the group consisting of plates, beads, microbeads, whiskers, combs, hybridization chips, membranes, single crystals, ceramics and self-assembling monolayers.

237. The combination of claim 231, wherein the collections of capture agents are conjugated with biotin or a biotin derivative and the solid support is conjugated with avidin, streptavidin or a derivative thereof, whereby the capture agents are linked to the support.

238. The combination of claim 231, wherein the capture agents are attached to the solid support by a covalent bond, an electrostatic bond, a hydrogen bond or a combination thereof.

239. The combination of claim 231, further comprising a linker between the collections of capture agents and the solid support.

240. The combination of claim 239, wherein the linker is selected from the group consisting of oligopeptides, oligonucleotides, oligopolyamides, oligoethyleneglycerol, oligoacrylamides, alkyl chains of between about 6 to about 20 carbon atoms, and combinations thereof.

241. The combination of claim 238, wherein the attachment is a cleavable attachment.

242. The combination of claim 241, wherein the cleavable attachment is cleavable by an enzyme, a chemical agent or electromagnetic radiation.

243. The combination of claim 242, wherein the chemical agent is selected from the group consisting of reducing agents, oxidizing agents, hydrolyzing agents and combinations thereof.

244. The combination of claim 242, wherein the electromagnetic radiation is selected from the group consisting of visible, ultraviolet and infrared radiation.

245. The combination of claim 217, wherein the collection of capture agents are addressably tagged by linking them to electronic, chemical, optical or color-coded labels.

246. The combination of claim 220, wherein the linking effected by a conjugation reagent(s) selected from the group consisting of thiol-thiol, thiol-amine, amine-amine, amine-carboxylic acid, thiol-carboxylic acid, thiol-carbohydrate and amine-non selective linkage.

247. A kit comprising the combination of claim 217 and one or more of the following:
(a) packaging material; and
(b) instructions for using the kit for preparation, use and/or analysis of self-assembled arrays.


SEQUENCE LISTING (948 sequences)

SEQ ID NO: 1 - protein, 13 aa, Artificial Sequence
  E-tag Peptide
GAPVPYPDPLEPR

SEQ ID NO: 2 - protein, 8 aa, Artificial Sequence
  Flag Peptide
DYKDDDDK

SEQ ID NO: 3 - protein, 9 aa, Artificial Sequence
  Glu-Glu Peptide
EEEEYMPME

SEQ ID NO: 4 - protein, 9 aa, Artificial Sequence
  HA.11 Peptide
YPYDVPDYA

SEQ ID NO: 5 - protein, 11 aa, Artificial Sequence
  HSV Tag Peptide
QPELAPEDPED

SEQ ID NO: 6 - protein, 10 aa, Artificial Sequence
  c-myc Peptide
EQKLISEEDL

SEQ ID NO: 7 - protein, 11 aa, Artificial Sequence
  T7 Tag Peptide
MASMTGGQQMG

SEQ ID NO: 8 - protein, 11 aa, Artificial Sequence
  VSV-G Peptide
YTDIEMNRLGK

SEQ ID NO: 9 - protein, 14 aa, Artificial Sequence
  V5 Peptide
GKPIPNPLLGLDST

SEQ ID NO: 10 - protein, 12 aa, Artificial Sequence
  AB2 Peptide
LTPPMGPVIDQR

SEQ ID NO: 11 - protein, 12 aa, Artificial Sequence
  AB4 Peptide
QPQSKGFEPPPP

SEQ ID NO: 12 - protein, 12 aa, Artificial Sequence
  B34 Peptide
DLHDERTLQFKL

SEQ ID NO: 13 - protein, 12 aa, Artificial Sequence
  P5D4-A Peptide
HPNLPETRRYAL

SEQ ID NO: 14 - protein, 12 aa, Artificial Sequence
  P5D4-B Peptide
SYTGIEFDRLSN

SEQ ID NO: 15 - protein, 12 aa, Artificial Sequence
  4C10 Peptide
MVDPEAQDVPKW

SEQ ID NO: 16 - protein, 12 aa, Artificial Sequence
  AB3 Peptide
YEYAKGSEPPAL

SEQ ID NO: 17 - protein, 12 aa, Artificial Sequence
  AB6 Peptide
AGTQWCLTRPPC

SEQ ID NO: 18 - protein, 12 aa, Artificial Sequence
  KT3-A Peptide
KLMPNEFFGLLP

SEQ ID NO: 19 - protein, 12 aa, Artificial Sequence
  KT3-B Peptide
KLIPTQLYLLHP

SEQ ID NO: 20 - protein, 12 aa, Artificial Sequence
  KT3-C Peptide
SFMPIEFYARKL

SEQ ID NO: 21 - protein, 12 aa, Artificial Sequence
  723 Peptide
TNMEWMTSHRSA

SEQ ID NO: 22 - protein, 12 aa, Artificial Sequence
  HOPC1 Peptide
MPQQGDPDWVVP

SEQ ID NO: 23 - protein, 9 aa, Artificial Sequence
  S1 Peptide
NANNPDWDF

SEQ ID NO: 24 - protein, 10 aa, Artificial Sequence
  E2 Peptide
SSTSSDFRDR

SEQ ID NO: 25 - protein, 8 aa, Artificial Sequence
  His-tag Peptide
HHHHHHGS

SEQ ID NO: 26 - protein, 6 aa, Artificial Sequence
  AU1 Peptide
DTYRYI

SEQ ID NO: 27 - protein, 6 aa, Artificial Sequence
  AU5 Peptide
TDFYLK

SEQ ID NO: 28 - protein, 5 aa, Artificial Sequence
  IRS Peptide
RYIRS

SEQ ID NO: 29 - protein, 495 aa, Escherichia coli CFT073 [sequence omitted]

SEQ ID NO: 30 - protein, 396 aa, Escherichia coli
  ref <308>: DATABASE ACCESSION NUMBER: Genbank AAC43128
  ref <309>: DATABASE ENTRY DATE: 1993-09-03
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 31 - protein, 318 aa, Mus musculus
  ref <308>: DATABASE ACCESSION NUMBER: Genbank NP_037812
  ref <309>: DATABASE ENTRY DATE: 2002-01-07
MDQNNSLPPYAQGLASPQGAMTPGIPIFSPMMPYGTGLTPQPIQNTNSLSILEEQQRQQQQQQQQQQQQQQQAVATAAASVQQSTSQQPTQGASGQTPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 32 - protein, 105 aa, Mus musculus
  ref <308>: DATABASE ACCESSION NUMBER: Genbank BAA04881
  ref <309>: DATABASE ENTRY DATE: 2002-12-25
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEASITEYA

SEQ ID NO: 33 - protein, 15 aa, Artificial Sequence
  S-tag Peptide
KETAAAKFERQHMDS

SEQ ID NO: 34 - protein, 11 aa, Artificial Sequence
  KT3 Peptide
KPPTPPPEPET

SEQ ID NO: 35 - protein, 238 aa, Artificial Sequence
  Cloning vector p35S-GFP
  ref <308>: DATABASE ACCESSION NUMBER: Genbank AAA69675
  ref <309>: DATABASE ENTRY DATE: 1995-07-08
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 36 - protein, 201 aa, Escherichia coli CFT073
  ref <308>: DATABASE ACCESSION NUMBER: Genbank NP_753922
  ref <309>: DATABASE ENTRY DATE: 2003-01-27
MKLFYKPGACSLASHITLRESGKDFTLVSVDLMKKRLENGDDYFAVNPKGQVPALLLDDGTLLTEGVAIMQYLADSVPDRQLLAPVNSISRYKTIEWLNYIATELHKGFTPLFRPDTPEEYKSTVRAQLEKKLQYVNEALKDEHWICGQRFTIADAYLFTVLRWAYAVKLNLEGLEHIAAFMQRMAERPEVQDALSAEGLK

SEQ ID NO: 37 - protein, 10 aa, Artificial Sequence
  SH3 Consensus Sequence
  VARIANT 1, 2, 3, 9: Xaa is any amino acid
XPXXPPPFXP

SEQ ID NO: 38 - protein, 6 aa, Artificial Sequence
  synthetic peptide
EPNGYF

SEQ ID NO: 39 - protein, 6 aa, Artificial Sequence
  synthetic peptide
EPNSGF

SEQ ID NO: 40 - protein, 6 aa, Artificial Sequence
  synthetic peptide
EPGYNF

SEQ ID NO: 41 - protein, 6 aa, Artificial Sequence
  synthetic peptide
EPSGNF

SEQ ID NO: 42 - protein, 6 aa, Artificial Sequence
  synthetic peptide
EGYPNF

SEQ ID NO: 43 - protein, 6 aa, Artificial Sequence
  synthetic peptide
ESGPNF

SEQ ID NO: 44 - protein, 6 aa, Artificial Sequence
  synthetic peptide
EGPNYF

SEQ ID NO: 45 - protein, 6 aa, Artificial Sequence
  synthetic peptide
ESPNGF

SEQ ID NO: 46 - protein, 6 aa, Artificial Sequence
  synthetic peptide
EPHGYK

SEQ ID NO: 47 - protein, 6 aa, Artificial Sequence
  synthetic peptide
EPHSGK

SEQ ID NO: 48 - protein, 6 aa, Artificial Sequence
  synthetic peptide
EPGYHK

SEQ ID NO: 49 - protein, 6 aa, Artificial Sequence
  synthetic peptide
EPSGHK

SEQ ID NO: 50 - protein, 6 aa, Artificial Sequence
  synthetic peptide
EGYPHK

SEQ ID NO: 51 - protein, 6 aa, Artificial Sequence
  synthetic peptide
ESGPHK

SEQ ID NO: 52 - protein, 6 aa, Artificial Sequence
  synthetic peptide
EGPHYK

SEQ ID NO: 53 - protein, 6 aa, Artificial Sequence
  synthetic peptide
ESPHGK

SEQ ID NO: 54 - protein, 6 aa, Artificial Sequence
  synthetic peptide
EQPGYN

SEQ ID NO: 55 - protein, 6 aa, Artificial Sequence
  synthetic peptide
EQPSGN

SEQ ID NO: 56 - protein, 6 aa, Artificial Sequence
  synthetic peptide
EQGYPN

SEQ ID NO: 57 - protein, 6 aa, Artificial Sequence
  synthetic peptide
EQSGPN

SEQ ID NO: 58 - protein, 6 aa, Artificial Sequence
  synthetic peptide
EGYQPN

SEQ ID NO: 59 - protein, 6 aa, Artificial Sequence
  synthetic peptide
ESGQPN

SEQ ID NO: 60 - protein, 6 aa, Artificial Sequence
  synthetic peptide
EGQPYN

SEQ ID NO: 61 - protein, 6 aa, Artificial Sequence
  synthetic peptide
ESQPGN

SEQ ID NO: 62 - protein, 6 aa, Artificial Sequence
  synthetic peptide
EQFGYH

SEQ ID NO: 63 - protein, 6 aa, Artificial Sequence
  synthetic peptide
EQFSGH

SEQ ID NO: 64 - protein, 6 aa, Artificial Sequence
  synthetic peptide
EQGYFH

SEQ ID NO: 65 - protein, 6 aa, Artificial Sequence
  synthetic peptide
EQSGFH

SEQ ID NO: 66 - protein, 6 aa, Artificial Sequence
  synthetic peptide
EGYQFH

SEQ ID NO: 67 - protein, 6 aa, Artificial Sequence
  synthetic peptide
ESGQFH

SEQ ID NO: 68 - protein, 6 aa, Artificial Sequence
  synthetic peptide
EGQFYH

SEQ ID NO: 69 - protein, 6 aa, Artificial Sequence
  synthetic peptide
ESQFGH

SEQ ID NO: 70 - protein, 6 aa, Artificial Sequence
  synthetic peptide
ENPGYT

SEQ ID NO: 71 - protein, 6 aa, Artificial Sequence
  synthetic peptide
ENPSGT

SEQ ID NO: 72 - protein, 6 aa, Artificial Sequence
  synthetic peptide
ENGYPT

SEQ ID NO: 73 - protein, 6 aa, Artificial Sequence
  synthetic peptide
ENSGPT

SEQ ID NO: 74 - protein, 6 aa, Artificial Sequence
  synthetic peptide
EGYNPT

SEQ ID NO: 75 - protein, 6 aa, Artificial Sequence
  synthetic peptide
ESGNPT

SEQ ID NO: 76 - protein, 6 aa, Artificial Sequence
  synthetic peptide
EGNPYT

SEQ ID NO: 77 - protein, 6 aa, Artificial Sequence
  synthetic peptide
ESNPGT

SEQ ID NO: 78 - protein, 6 aa, Artificial Sequence
  synthetic peptide
ENFGYD

SEQ ID NO: 79 - protein, 6 aa, Artificial Sequence
  synthetic peptide
ENFSGD

SEQ ID NO: 80 - protein, 6 aa, Artificial Sequence
  synthetic peptide
ENGYFD

SEQ ID NO: 81 - protein, 6 aa, Artificial Sequence
  synthetic peptide
ENSGFD

SEQ ID NO: 82 - protein, 6 aa, Artificial Sequence
  synthetic peptide
EGYNFD

SEQ ID NO: 83 - protein, 6 aa, Artificial Sequence
  synthetic peptide
ESGNFD

SEQ ID NO: 84 - protein, 6 aa, Artificial Sequence
  synthetic peptide
EGNFYD

SEQ ID NO: 85 - protein, 6 aa, Artificial Sequence
  synthetic peptide
ESNFGD

SEQ ID NO: 86 - protein, 6 aa, Artificial Sequence
  synthetic peptide
ENDGYP

SEQ ID NO: 87 - protein, 6 aa, Artificial Sequence
  synthetic peptide
ENDSGP

SEQ ID NO: 88 - protein, 6 aa, Artificial Sequence
  synthetic peptide
ENGYDP

SEQ ID NO: 89 - protein, 6 aa, Artificial Sequence
  synthetic peptide
ENSGDP

SEQ ID NO: 90 - protein, 6 aa, Artificial Sequence
  synthetic peptide
EGYNDP

SEQ ID NO: 91 - protein, 6 aa, Artificial Sequence
  synthetic peptide
ESGNDP

SEQ ID NO: 92 - protein, 6 aa, Artificial Sequence
  synthetic peptide
EGNDYP

SEQ ID NO: 93 - protein, 6 aa, Artificial Sequence
  synthetic peptide
ESNDGP

SEQ ID NO: 94 - protein, 6 aa, Artificial Sequence
  synthetic peptide
EFQGYP

SEQ ID NO: 95 - protein, 6 aa, Artificial Sequence
  synthetic peptide
EFQSGP

SEQ ID NO: 96 - protein, 6 aa, Artificial Sequence
  synthetic peptide
EFGYQP

SEQ ID NO: 97 - protein, 6 aa, Artificial Sequence
  synthetic peptide
EFSGQP

SEQ ID NO: 98 - protein, 6 aa, Artificial Sequence
  synthetic peptide
EGYFQP

SEQ ID NO: 99 - protein, 6 aa, Artificial Sequence
  synthetic peptide
ESGFQP

SEQ ID NO: 100 - protein, 6 aa, Artificial Sequence
  synthetic peptide
EGFQYP

SEQ ID NO: 101 - protein, 6 aa, Artificial Sequence
  synthetic peptide
ESFQGP

SEQ ID NO: 102 - protein, 6 aa, Artificial Sequence
  synthetic peptide
EFKGYT

SEQ ID NO: 103 - protein, 6 aa, Artificial Sequence
  synthetic peptide
EFKSGT

SEQ ID NO: 104 - protein, 6 aa, Artificial Sequence
  synthetic peptide
EFGYKT

SEQ ID NO: 105 - protein, 6 aa, Artificial Sequence
  synthetic peptide
EFSGKT

SEQ ID NO: 106 - protein, 6 aa, Artificial Sequence
  synthetic peptide
EGYFKT

SEQ ID NO: 107 - protein, 6 aa, Artificial Sequence
  synthetic peptide
ESGFKT

SEQ ID NO: 108 - protein, 6 aa, Artificial Sequence
  synthetic peptide
EGFKYT

SEQ ID NO: 109 - protein, 6 aa, Artificial Sequence
  synthetic peptide
ESFKGT

SEQ ID NO: 110 - protein, 6 aa, Artificial Sequence
  synthetic peptide
EFDGYH

SEQ ID NO: 111 - protein, 6 aa, Artificial Sequence
  synthetic peptide
EFDSGH

SEQ ID NO: 112 - protein, 6 aa, Artificial Sequence
  synthetic peptide
EFGYDH

SEQ ID NO: 113 - protein, 6 aa, Artificial Sequence
  synthetic peptide
EFSGDH

SEQ ID NO: 114 - protein, 6 aa, Artificial Sequence
  synthetic peptide
EGYFDH

SEQ ID NO: 115 - protein, 6 aa, Artificial Sequence
  synthetic peptide
ESGFDH

SEQ ID NO: 116 - protein, 6 aa, Artificial Sequence
  synthetic peptide
EGFDYH

SEQ ID NO: 117 - protein, 6 aa, Artificial Sequence
  synthetic peptide
ESFDGH

SEQ ID NO: 118 - protein, 6 aa, Artificial Sequence
  synthetic peptide
EHNGYQ

SEQ ID NO: 119 - protein, 6 aa, Artificial Sequence
  synthetic peptide
EHNSGQ

SEQ ID NO: 120 - protein, 6 aa, Artificial Sequence
  synthetic peptide
EHGYNQ

SEQ ID NO: 121 - protein, 6 aa, Artificial Sequence
  synthetic peptide
EHSGNQ

SEQ ID NO: 122 - protein, 6 aa, Artificial Sequence
  synthetic peptide
EGYHNQ

SEQ ID NO: 123 - protein, 6 aa, Artificial Sequence
  synthetic peptide
ESGHNQ

SEQ ID NO: 124 - protein, 6 aa, Artificial Sequence
  synthetic peptide
EGHNYQ

SEQ ID NO: 125 - protein, 6 aa, Artificial Sequence
  synthetic peptide
ESHNGQ

SEQ ID NO: 126 - protein, 6 aa, Artificial Sequence
  synthetic peptide
EHKGYP

SEQ ID NO: 127 - protein, 6 aa, Artificial Sequence
  synthetic peptide
EHKSGP

SEQ ID NO: 128 - protein, 6 aa, Artificial Sequence
  synthetic peptide
EHGYKP

SEQ ID NO: 129 - protein, 6 aa, Artificial Sequence
  synthetic peptide
EHSGKP

SEQ ID NO: 130 - protein, 6 aa, Artificial Sequence
  synthetic peptide
EGYHKP

SEQ ID NO: 131 - protein, 6 aa, Artificial Sequence
  synthetic peptide
ESGHKP

SEQ ID NO: 132 - protein, 6 aa, Artificial Sequence
  synthetic peptide
EGHKYP

SEQ ID NO: 133 - protein, 6 aa, Artificial Sequence
  synthetic peptide
ESHKGP

SEQ ID NO: 134 - protein, 6 aa, Artificial Sequence
  synthetic peptide
ETNGYK

SEQ ID NO: 135 - protein, 6 aa, Artificial Sequence
  synthetic peptide
ETNSGK

SEQ ID NO: 136 - protein, 6 aa, Artificial Sequence
  synthetic peptide
ETGYNK

SEQ ID NO: 137 - protein, 6 aa, Artificial Sequence
  synthetic peptide
ETSGNK

SEQ ID NO: 138 - protein, 6 aa, Artificial Sequence
  synthetic peptide
EGYTNK

SEQ ID NO: 139 - protein, 6 aa, Artificial Sequence
  synthetic peptide
ESGTNK

SEQ ID NO: 140 - protein, 6 aa, Artificial Sequence
  synthetic peptide
EGTNYK

SEQ ID NO: 141 - protein, 6 aa, Artificial Sequence
  synthetic peptide
ESTNGK

SEQ ID NO: 142 - protein, 6 aa, Artificial Sequence
  synthetic peptide
EKPGYH

SEQ ID NO: 143 - protein, 6 aa, Artificial Sequence
  synthetic peptide
EKPSGH

SEQ ID NO: 144 - protein, 6 aa, Artificial Sequence
  synthetic peptide
EKGYPH

SEQ ID NO: 145 - protein, 6 aa, Artificial Sequence
  synthetic peptide
EKSGPH

SEQ ID NO: 146 - protein, 6 aa, Artificial Sequence
  synthetic peptide
EGYKPH

SEQ ID NO: 147 - protein, 6 aa, Artificial Sequence
  synthetic peptide
ESGKPH

SEQ ID NO: 148 - protein, 6 aa, Artificial Sequence
  synthetic peptide
EGKPYH

SEQ ID NO: 149 - protein, 6 aa, Artificial Sequence
  synthetic peptide
ESKPGH

SEQ ID NO: 150 - protein, 6 aa, Artificial Sequence
  synthetic peptide
ELNGYD

SEQ ID NO: 151 - protein, 6 aa, Artificial Sequence
  synthetic peptide
ELNSGD

SEQ ID NO: 152 - protein, 6 aa, Artificial Sequence
  synthetic peptide
ELGYND

SEQ ID NO: 153 - protein, 6 aa, Artificial Sequence
  synthetic peptide
ELSGND

SEQ ID NO: 154 - protein, 6 aa, Artificial Sequence
  synthetic peptide
EGYLND

SEQ ID NO: 155 - protein, 6 aa, Artificial Sequence
  synthetic peptide
ESGLND

SEQ ID NO: 156 - protein, 6 aa, Artificial Sequence
  synthetic peptide
EGLNYD

SEQ ID NO: 157 - protein, 6 aa, Artificial Sequence
  synthetic peptide
ESLNGD

SEQ ID NO: 158 - protein, 6 aa, Artificial Sequence
  synthetic peptide
EDPGYF

SEQ ID NO: 159 - protein, 6 aa, Artificial Sequence
  synthetic peptide
EDPSGF

SEQ ID NO: 160 - protein, 6 aa, Artificial Sequence
  synthetic peptide
EDGYPF

SEQ ID NO: 161 - protein, 6 aa, Artificial Sequence
  synthetic peptide
EDSGPF

SEQ ID NO: 162 - protein, 6 aa, Artificial Sequence
  synthetic peptide
EGYDPF

SEQ ID NO: 163 - protein, 6 aa, Artificial Sequence
  synthetic peptide
ESGDPF

SEQ ID NO: 164 - protein, 6 aa, Artificial Sequence
  synthetic peptide
EGDPYF

SEQ ID NO: 165 - protein, 6 aa, Artificial Sequence
  synthetic peptide
ESDPGF

SEQ ID NO: 166 - protein, 6 aa, Artificial Sequence
  synthetic peptide
EDFGYP

SEQ ID NO: 167 - protein, 6 aa, Artificial Sequence
  synthetic peptide
EDFSGP

SEQ ID NO: 168 - protein, 6 aa, Artificial Sequence
  synthetic peptide
EDGYFP

SEQ ID NO: 169 - protein, 6 aa, Artificial Sequence
  synthetic peptide
EDSGFP

SEQ ID NO: 170 - protein, 6 aa, Artificial Sequence
  synthetic peptide
EGYDFP

SEQ ID NO: 171 - protein, 6 aa, Artificial Sequence
  synthetic peptide
ESGDFP

SEQ ID NO: 172 - protein, 6 aa, Artificial Sequence
  synthetic peptide
EGDFYP

SEQ ID NO: 173 - protein, 6 aa, Artificial Sequence
  synthetic peptide
ESDFGP

SEQ ID NO: 174 - protein, 6 aa, Artificial Sequence
  synthetic peptide
PEQGYN

SEQ ID NO: 175 - protein, 6 aa, Artificial Sequence
  synthetic peptide
PEQSGN

SEQ ID NO: 176 - protein, 6 aa, Artificial Sequence
  synthetic peptide
PEGYQN

SEQ ID NO: 177 - protein, 6 aa, Artificial Sequence
  synthetic peptide
PESGQN

SEQ ID NO: 178 - protein, 6 aa, Artificial Sequence
  synthetic peptide
PGYEQN

SEQ ID NO: 179 - protein, 6 aa, Artificial Sequence
  synthetic peptide
PSGEQN

SEQ ID NO: 180 - protein, 6 aa, Artificial Sequence
  synthetic peptide
PGEQYN

SEQ ID NO: 181 - protein, 6 aa, Artificial Sequence
  synthetic peptide
PSEQGN

SEQ ID NO: 182 - protein, 6 aa, Artificial Sequence
  synthetic peptide
PEFGYQ

SEQ ID NO: 183 - protein, 6 aa, Artificial Sequence
  synthetic peptide
PEFSGQ

SEQ ID NO: 184 - protein, 6 aa, Artificial Sequence
  synthetic peptide
PEGYFQ

SEQ ID NO: 185 - protein, 6 aa, Artificial Sequence
  synthetic peptide
PESGFQ

SEQ ID NO: 186 - protein, 6 aa, Artificial Sequence
  synthetic peptide
PGYEFQ

SEQ ID NO: 187 - protein, 6 aa, Artificial Sequence
  synthetic peptide
PSGEFQ

SEQ ID NO: 188 - protein, 6 aa, Artificial Sequence
  synthetic peptide
PGEFYQ

SEQ ID NO: 189 - protein, 6 aa, Artificial Sequence
  synthetic peptide
PSEFGQ

SEQ ID NO: 190 - protein, 6 aa, Artificial Sequence
  synthetic peptide
PEKGYD

SEQ ID NO: 191 - protein, 6 aa, Artificial Sequence
  synthetic peptide
PEKSGD

SEQ ID NO: 192 - protein, 6 aa, Artificial Sequence
  synthetic peptide
PEGYKD

SEQ ID NO: 193 - protein, 6 aa, Artificial Sequence
  synthetic peptide
PESGKD

SEQ ID NO: 194 - protein, 6 aa, Artificial Sequence
  synthetic peptide
PGYEKD

SEQ ID NO: 195 - protein, 6 aa, Artificial Sequence
  synthetic peptide
PSGEKD

SEQ ID NO: 196 - protein, 6 aa, Artificial Sequence
  synthetic peptide
PGEKYD

SEQ ID NO: 197 - protein, 6 aa, Artificial Sequence
  synthetic peptide
PSEKGD

SEQ ID NO: 198 - protein, 6 aa, Artificial Sequence
  synthetic peptide
PQTGYE

SEQ ID NO: 199 - protein, 6 aa, Artificial Sequence
  synthetic peptide
PQTSGE

SEQ ID NO: 200 - protein, 6 aa, Artificial Sequence
  synthetic peptide
PQGYTE

SEQ ID NO: 201 - protein, 6 aa, Artificial Sequence
  synthetic peptide
PQSGTE

SEQ ID NO: 202 - protein, 6 aa, Artificial Sequence
  synthetic peptide
PGYQTE

SEQ ID NO: 203 - protein, 6 aa, Artificial Sequence
  synthetic peptide
PSGQTE

SEQ ID NO: 204 - protein, 6 aa, Artificial Sequence
  synthetic peptide
PGQTYE

SEQ ID NO: 205 - protein, 6 aa, Artificial Sequence
  synthetic peptide
PSQTGE

SEQ ID NO: 206 - protein, 6 aa, Artificial Sequence
  synthetic peptide
PNEGYF

SEQ ID NO: 207 - protein, 6 aa, Artificial Sequence
  synthetic peptide
PNESGF

SEQ ID NO: 208 - protein, 6 aa, Artificial Sequence
  synthetic peptide
PNGYEF

SEQ ID NO: 209 - protein, 6 aa, Artificial Sequence
  synthetic peptide
PNSGEF

SEQ ID NO: 210 - protein, 6 aa, Artificial Sequence
  synthetic peptide
PGYNEF

SEQ ID NO: 211 - protein, 6 aa, Artificial Sequence
  synthetic peptide
PSGNEF

SEQ ID NO: 212 - protein, 6 aa, Artificial Sequence
  synthetic peptide
PGNEYF

SEQ ID NO: 213 - protein, 6 aa, Artificial Sequence
  synthetic peptide
PSNEGF

SEQ ID NO: 214 - protein, 6 aa, Artificial Sequence
  synthetic peptide
PFEGYQ

SEQ ID NO: 215 - protein, 6 aa, Artificial Sequence
  synthetic peptide
PFESGQ

SEQ ID NO: 216 - protein, 6 aa, Artificial Sequence
  synthetic peptide
PFGYEQ

SEQ ID NO: 217 - protein, 6 aa, Artificial Sequence
  synthetic peptide
PFSGEQ

SEQ ID NO: 218 - protein, 6 aa, Artificial Sequence
  synthetic peptide
PGYFEQ

SEQ ID NO: 219 - protein, 6 aa, Artificial Sequence
  synthetic peptide
PSGFEQ

SEQ ID NO: 220 - protein, 6 aa, Artificial Sequence
  synthetic peptide
PGFEYQ

SEQ ID NO: 221 - protein, 6 aa, Artificial Sequence
  synthetic peptide
PSFEGQ

SEQ ID NO: 222 - protein, 6 aa, Artificial Sequence
  synthetic peptide
PFHGYL

SEQ ID NO: 223 - protein, 6 aa, Artificial Sequence
  synthetic peptide
PFHSGL

SEQ ID NO: 224 - protein, 6 aa, Artificial Sequence
  synthetic peptide
PFGYHL

SEQ ID NO: 225 - protein, 6 aa, Artificial Sequence
  synthetic peptide
PFSGHL

SEQ ID NO: 226 - protein, 6 aa, Artificial Sequence
  synthetic peptide
PGYFHL

SEQ ID NO: 227 - protein, 6 aa, Artificial Sequence
  synthetic peptide
PSGFHL

SEQ ID NO: 228 - protein, 6 aa, Artificial Sequence
  synthetic peptide
PGFHYL

SEQ ID NO: 229 - protein, 6 aa, Artificial Sequence
  synthetic peptide
PSFHGL

SEQ ID NO: 230 - protein, 6 aa, Artificial Sequence
  synthetic peptide
PHEGYK

SEQ ID NO: 231 - protein, 6 aa, Artificial Sequence
  synthetic peptide
PHESGK

SEQ ID NO: 232 - protein, 6 aa, Artificial Sequence
  synthetic peptide
PHGYEK

SEQ ID NO: 233 - protein, 6 aa, Artificial Sequence
  synthetic peptide
PHSGEK

SEQ ID NO: 234 - protein, 6 aa, Artificial Sequence
  synthetic peptide
PGYHEK

SEQ ID NO: 235 - protein, 6 aa, Artificial Sequence
  synthetic peptide
PSGHEK

SEQ ID NO: 236 - protein, 6 aa, Artificial Sequence
  synthetic peptide
PGHEYK

SEQ ID NO: 237 - protein, 6 aa, Artificial Sequence
  synthetic peptide
PSHEGK

SEQ ID NO: 238 - protein, 6 aa, Artificial Sequence
  synthetic peptide
PHTGYF

SEQ ID NO: 239 - protein, 6 aa, Artificial Sequence
  synthetic peptide
PHTSGF

SEQ ID NO: 240 - protein, 6 aa, Artificial Sequence
  synthetic peptide
PHGYTF

SEQ ID NO: 241 - protein, 6 aa, Artificial Sequence
  synthetic peptide
PHSGTF

SEQ ID NO: 242 - protein, 6 aa, Artificial Sequence
  synthetic peptide
PGYHTF

SEQ ID NO: 243 - protein, 6 aa, Artificial Sequence
  synthetic peptide
PSGHTF

SEQ ID NO: 244 - protein, 6 aa, Artificial Sequence
  synthetic peptide
PGHTYF

SEQ ID NO: 245 - protein, 6 aa, Artificial Sequence
  synthetic peptide
PSHTGF

SEQ ID NO: 246 - protein, 6 aa, Artificial Sequence
  synthetic peptide
PTLGYD

SEQ ID NO: 247 - protein, 6 aa, Artificial Sequence
  synthetic peptide
PTLSGD

SEQ ID NO: 248 - protein, 6 aa, Artificial Sequence
  synthetic peptide
PTGYLD

SEQ ID NO: 249 - protein, 6 aa, Artificial Sequence
  synthetic peptide
PTSGLD

SEQ ID NO: 250 - protein, 6 aa, Artificial Sequence
  synthetic peptide
PGYTLD

SEQ ID NO: 251 - protein, 6 aa, Artificial Sequence
  synthetic peptide
PSGTLD

SEQ ID NO: 252 - protein, 6 aa, Artificial Sequence
  synthetic peptide
PGTLYD

SEQ ID NO: 253 - protein, 6 aa, Artificial Sequence
  synthetic peptide
PSTLGD

SEQ ID NO: 254 - protein, 6 aa, Artificial Sequence
  synthetic peptide
PKHGYT

SEQ ID NO: 255 - protein, 6 aa, Artificial Sequence
  synthetic peptide
PKHSGT

SEQ ID NO: 256 - protein, 6 aa, Artificial Sequence
  synthetic peptide
PKGYHT

SEQ ID NO: 257 - protein, 6 aa, Artificial Sequence
  synthetic peptide
PKSGHT

SEQ ID NO: 258 - protein, 6 aa, Artificial Sequence
  synthetic peptide
PGYKHT

SEQ ID NO: 259 - protein, 6 aa, Artificial Sequence
  synthetic peptide
PSGKHT

SEQ ID NO: 260 - protein, 6 aa, Artificial Sequence
  synthetic peptide
PGKHYT

SEQ ID NO: 261 - protein, 6 aa, Artificial Sequence
  synthetic peptide
PSKHGT

SEQ ID NO: 262 - protein, 6 aa, Artificial Sequence
  synthetic peptide
PLDGYN

SEQ ID NO: 263 - protein, 6 aa, Artificial Sequence
  synthetic peptide
PLDSGN

SEQ ID NO: 264 - protein, 6 aa, Artificial Sequence
  synthetic peptide
PLGYDN

SEQ ID NO: 265 - protein, 6 aa, Artificial Sequence
  synthetic peptide
PLSGDN

SEQ ID NO: 266 - protein, 6 aa, Artificial Sequence
  synthetic peptide
PGYLDN

SEQ ID NO: 267 - protein, 6 aa, Artificial Sequence
  synthetic peptide
PSGLDN

SEQ ID NO: 268 - protein, 6 aa, Artificial Sequence
  synthetic peptide
PGLDYN

SEQ ID NO: 269 - protein, 6 aa, Artificial Sequence
  synthetic peptide
PSLDGN

SEQ ID NO: 270 - protein, 6 aa, Artificial Sequence
  synthetic peptide
QEPGYD

SEQ ID NO: 271 - protein, 6 aa, Artificial Sequence
  synthetic peptide
QEPSGD

SEQ ID NO: 272 - protein, 6 aa, Artificial Sequence
  synthetic peptide
QEGYPD

SEQ ID NO: 273 - protein, 6 aa, Artificial Sequence
  synthetic peptide
QESGPD

SEQ ID NO: 274 - protein, 6 aa, Artificial Sequence
  synthetic peptide
QGYEPD

SEQ ID NO: 275 - protein, 6 aa, Artificial Sequence
  synthetic peptide
QSGEPD

SEQ ID NO: 276 - protein, 6 aa, Artificial Sequence
  synthetic peptide
QGEPYD

SEQ ID NO: 277 - protein, 6 aa, Artificial Sequence
  synthetic peptide
QSEPGD

SEQ ID NO: 278 - protein, 6 aa, Artificial Sequence
  synthetic peptide
QETGYF

SEQ ID NO: 279 - protein, 6 aa, Artificial Sequence
  synthetic peptide
QETSGF

SEQ ID NO: 280 - protein, 6 aa, Artificial Sequence
  synthetic peptide
QEGYTF

SEQ ID NO: 281 - protein, 6 aa, Artificial Sequence
  synthetic peptide
QESGTF

SEQ ID NO: 282 - protein, 6 aa, Artificial Sequence
  synthetic peptide
QGYETF

SEQ ID NO: 283 - protein, 6 aa, Artificial Sequence
  synthetic peptide
QSGETF

SEQ ID NO: 284 - protein, 6 aa, Artificial Sequence
  synthetic peptide
QGETYF

SEQ ID NO: 285 - protein, 6 aa, Artificial Sequence
  synthetic peptide
QSETGF

SEQ ID NO: 286 - protein, 6 aa, Artificial Sequence
  synthetic peptide
QPEGYH

SEQ ID NO: 287 - protein, 6 aa, Artificial Sequence
  synthetic peptide
QPESGH

SEQ ID NO: 288 - protein, 6 aa, Artificial Sequence
  synthetic peptide
QPGYEH

SEQ ID NO: 289 - protein, 6 aa, Artificial Sequence
  synthetic peptide
QPSGEH

SEQ ID NO: 290 - protein, 6 aa, Artificial Sequence
  synthetic peptide
QGYPEH

SEQ ID NO: 291 - protein, 6 aa, Artificial Sequence
  synthetic peptide
QSGPEH

SEQ ID NO: 292 - protein, 6 aa, Artificial Sequence
  synthetic peptide
QGPEYH

SEQ ID NO: 293 - protein, 6 aa, Artificial Sequence
  synthetic peptide
QSPEGH

SEQ ID NO: 294 - protein, 6 aa, Artificial Sequence
  synthetic peptide
QNHGYE

SEQ ID NO: 295 - protein, 6 aa, Artificial Sequence
  synthetic peptide
QNHSGE

SEQ ID NO: 296 - protein, 6 aa, Artificial Sequence
  synthetic peptide
QNGYHE

SEQ ID NO: 297 - protein, 6 aa, Artificial Sequence
  synthetic peptide
QNSGHE

SEQ ID NO: 298 - protein, 6 aa, Artificial Sequence
  synthetic peptide
QGYNHE

SEQ ID NO: 299 - protein, 6 aa, Artificial Sequence
  synthetic peptide
QGYNHE

SEQ ID NO: 300 - protein, 6 aa, Artificial Sequence
  synthetic peptide
QGNHYE

SEQ ID NO: 301 - protein, 6 aa, Artificial Sequence
  synthetic peptide
QSNHGE

SEQ ID NO: 302 - protein, 6 aa, Artificial Sequence
  synthetic peptide
QFEGYK

SEQ ID NO: 303 - protein, 6 aa, Artificial Sequence
  synthetic peptide
QFESGK

SEQ ID NO: 304 - protein, 6 aa, Artificial Sequence
  synthetic peptide
QFGYEK

SEQ ID NO: 305 - protein, 6 aa, Artificial Sequence
  synthetic peptide
QFSGEK

SEQ ID NO: 306 - protein, 6 aa, Artificial Sequence
  synthetic peptide
QGYFEK

SEQ ID NO: 307 - protein, 6 aa, Artificial Sequence
  synthetic peptide
QSGFEK

SEQ ID NO: 308 - protein, 6 aa, Artificial Sequence
  synthetic peptide
QGFEYK

SEQ ID NO: 309 - protein, 6 aa, Artificial Sequence
  synthetic peptide
QSFEGK

SEQ ID NO: 310 - protein, 6 aa, Artificial Sequence
  synthetic peptide
QTFGYN

SEQ ID NO: 311 - protein, 6 aa, Artificial Sequence
  synthetic peptide
QTFSGN

SEQ ID NO: 312 - protein, 6 aa, Artificial Sequence
  synthetic peptide
QTGYFN

SEQ ID NO: 313 - protein, 6 aa, Artificial Sequence
  synthetic peptide
QTSGFN

SEQ ID NO: 314 - protein, 6 aa, Artificial Sequence
  synthetic peptide
QGYTFN

SEQ ID NO: 315 - protein, 6 aa, Artificial Sequence
  synthetic peptide
QSGTFN

SEQ ID NO: 316 - protein, 6 aa, Artificial Sequence
  synthetic peptide
QGTFYN

SEQ ID NO: 317 - protein, 6 aa, Artificial Sequence
  synthetic peptide
QSTFGN

SEQ ID NO: 318 - protein, 6 aa, Artificial Sequence
  synthetic peptide
QKEGYF

SEQ ID NO: 319 - protein, 6 aa, Artificial Sequence
  synthetic peptide
QKESGF

SEQ ID NO: 320 - protein, 6 aa, Artificial Sequence
  synthetic peptide
QKGYEF

SEQ ID NO: 321 - protein, 6 aa, Artificial Sequence
  synthetic peptide
QKSGEF

SEQ ID NO: 322 - protein, 6 aa, Artificial Sequence
  synthetic peptide
QGYKEF

SEQ ID NO: 323 - protein, 6 aa, Artificial Sequence
  synthetic peptide
QSGKEF

SEQ ID NO: 324 - protein, 6 aa, Artificial Sequence
  synthetic peptide
QGKEYF

SEQ ID NO: 325 - protein, 6 aa, Artificial Sequence
  synthetic peptide
QSKEGF

SEQ ID NO: 326 - protein, 6 aa, Artificial Sequence
  synthetic peptide
QLHGYT

SEQ ID NO: 327 - protein, 6 aa, Artificial Sequence
  synthetic peptide
QLHSGT

SEQ ID NO: 328 - protein, 6 aa, Artificial Sequence
  synthetic peptide
QLGYHT

SEQ ID NO: 329 - protein, 6 aa, Artificial Sequence
  synthetic peptide
QLSGHT

SEQ ID NO: 330 - protein, 6 aa, Artificial Sequence
  synthetic peptide
QGYLHT

SEQ ID NO: 331 - protein, 6 aa, Artificial Sequence
  synthetic peptide
QSGLHT

SEQ ID NO: 332 - protein, 6 aa, Artificial Sequence
  synthetic peptide
QGLHYT

SEQ ID NO: 333 - protein, 6 aa, Artificial Sequence
  synthetic peptide
QSLHGT

SEQ ID NO: 334 - protein, 6 aa, Artificial Sequence
  synthetic peptide
QLDGYE

SEQ ID NO: 335 - protein, 6 aa, Artificial Sequence
  synthetic peptide
QLDSGE

SEQ ID NO: 336 - protein, 6 aa, Artificial Sequence
  synthetic peptide
QLGYDE

SEQ ID NO: 337 - protein, 6 aa, Artificial Sequence
  synthetic peptide
QLSGDE

SEQ ID NO: 338 - protein, 6 aa, Artificial Sequence
  synthetic peptide
QGYLDE

SEQ ID NO: 339 - protein, 6 aa, Artificial Sequence
  synthetic peptide
QSGLDE

SEQ ID NO: 340 - protein, 6 aa, Artificial Sequence
  synthetic peptide
QGLDYE

SEQ ID NO: 341 - protein, 6 aa, Artificial Sequence
  synthetic peptide
QSLDGE

SEQ ID NO: 342 - protein, 6 aa, Artificial Sequence
  synthetic peptide
NEPGYL

SEQ ID NO: 343 - protein, 6 aa, Artificial Sequence
  synthetic peptide
NEPSGL

SEQ ID NO: 344 - protein, 6 aa, Artificial Sequence
  synthetic peptide
NEGYPL

SEQ ID NO: 345 - protein, 6 aa, Artificial Sequence
  synthetic peptide
NESGPL

SEQ ID NO: 346 - protein, 6 aa, Artificial Sequence
  synthetic peptide
NGYEPL

SEQ ID NO: 347 - protein, 6 aa, Artificial Sequence
  synthetic peptide
NSGEPL

SEQ ID NO: 348 - protein, 6 aa, Artificial Sequence
  synthetic peptide
NGEPYL

SEQ ID NO: 349 - protein, 6 aa, Artificial Sequence
  synthetic peptide
NSEPGL

SEQ ID NO: 350 - protein, 6 aa, Artificial Sequence
  synthetic peptide
NEFGYH

SEQ ID NO: 351 - protein, 6 aa, Artificial Sequence
  synthetic peptide
NEFSGH

SEQ ID NO: 352 - protein, 6 aa, Artificial Sequence
  synthetic peptide
NEGYFH

SEQ ID NO: 353 - protein, 6 aa, Artificial Sequence
  synthetic peptide
NESGFH

SEQ ID NO: 354 - protein, 6 aa, Artificial Sequence
  synthetic peptide
NGYEFH

SEQ ID NO: 355 - protein, 6 aa, Artificial Sequence
  synthetic peptide
NSGEFH

SEQ ID NO: 356 - protein, 6 aa, Artificial Sequence
  synthetic peptide
NGEFYH

SEQ ID NO: 357 - protein, 6 aa, Artificial Sequence
  synthetic peptide
NSEFGH

SEQ ID NO: 358 - protein, 6 aa, Artificial Sequence
  synthetic peptide
NPEGYF

SEQ ID NO: 359 - protein, 6 aa, Artificial Sequence
  synthetic peptide
NPESGF

SEQ ID NO: 360 - protein, 6 aa, Artificial Sequence
  synthetic peptide
NPGYEF

SEQ ID NO: 361 - protein, 6 aa, Artificial Sequence
  synthetic peptide
NPSGEF

SEQ ID NO: 362 - protein, 6 aa, Artificial Sequence
  synthetic peptide
NGYPEF

SEQ ID NO: 363 - protein, 6 aa, Artificial Sequence
  synthetic peptide
NSGPEF

SEQ ID NO: 364 - protein, 6 aa, Artificial Sequence
  synthetic peptide
NGPEYF

SEQ ID NO: 365 - protein, 6 aa, Artificial Sequence
  synthetic peptide
NSPEGF

SEQ ID NO: 366 - protein, 6 aa, Artificial Sequence
  synthetic peptide
NQHGYD

SEQ ID NO: 367 - protein, 6 aa, Artificial Sequence
  synthetic peptide
NQHSGD

SEQ ID NO: 368 - protein, 6 aa, Artificial Sequence
  synthetic peptide
NQGYHD

SEQ ID NO: 369 - protein, 6 aa, Artificial Sequence
  synthetic peptide
NQSGHD

SEQ ID NO: 370 - protein, 6 aa, Artificial Sequence
  synthetic peptide
NGYQHD

SEQ ID NO: 371 - protein, 6 aa, Artificial Sequence
  synthetic peptide
NSGQHD

SEQ ID NO: 372 - protein, 6 aa, Artificial Sequence
  synthetic peptide
NGQHYD

SEQ ID NO: 373 - protein, 6 aa, Artificial Sequence
  synthetic peptide
NSQHGD

SEQ ID NO: 374 - protein, 6 aa, Artificial Sequence
  synthetic peptide
NFEGYP

SEQ ID NO: 375 - protein, 6 aa, Artificial Sequence
  synthetic peptide
NFESGP

SEQ ID NO: 376 - protein, 6 aa, Artificial Sequence
  synthetic peptide
NFGYEP

SEQ ID NO: 377 - protein, 6 aa, Artificial Sequence
  synthetic peptide
NFSGEP

SEQ ID NO: 378 - protein, 6 aa, Artificial Sequence
  synthetic peptide
NGYFEP

SEQ ID NO: 379 - protein, 6 aa, Artificial Sequence
  synthetic peptide
NSGFEP

SEQ ID NO: 380 - protein, 6 aa, Artificial Sequence
  synthetic peptide
NGFEYP

SEQ ID NO: 381 - protein, 6 aa, Artificial Sequence
  synthetic peptide
NSFEGP

SEQ ID NO: 382 - protein, 6 aa, Artificial Sequence
  synthetic peptide
NFKGYH

SEQ ID NO: 383 - protein, 6 aa, Artificial Sequence
  synthetic peptide
NFKSGH

SEQ ID NO: 384 - protein, 6 aa, Artificial Sequence
  synthetic peptide
NFGYKH

SEQ ID NO: 385 - protein, 6 aa, Artificial Sequence
  synthetic peptide
NFSGKH

SEQ ID NO: 386 - protein, 6 aa, Artificial Sequence
  synthetic peptide
NGYFKH

SEQ ID NO: 387 - protein, 6 aa, Artificial Sequence
  synthetic peptide
NSGFKH

SEQ ID NO: 388 - protein, 6 aa, Artificial Sequence
  synthetic peptide
NGFKYH

SEQ ID NO: 389 - protein, 6 aa, Artificial Sequence
  synthetic peptide
NSFKGH

SEQ ID NO: 390 - protein, 6 aa, Artificial Sequence
  synthetic peptide
NHPGYT

SEQ ID NO: 391 - protein, 6 aa, Artificial Sequence
  synthetic peptide
NHPSGT

SEQ ID NO: 392 - protein, 6 aa, Artificial Sequence
  synthetic peptide
NHGYPT

SEQ ID NO: 393 - protein, 6 aa, Artificial Sequence
  synthetic peptide
NHSGPT

SEQ ID NO: 394 - protein, 6 aa, Artificial Sequence
  synthetic peptide
NGYHPT

SEQ ID NO: 395 - protein, 6 aa, Artificial Sequence
  synthetic peptide
NSGHPT

SEQ ID NO: 396 - protein, 6 aa, Artificial Sequence
  synthetic peptide
NGHPYT

SEQ ID NO: 397 - protein, 6 aa, Artificial Sequence
  synthetic peptide
NSHPGT

SEQ ID NO: 398 - protein, 6 aa, Artificial Sequence
  synthetic peptide
NHTGYD

SEQ ID NO: 399 - protein, 6 aa, Artificial Sequence
  synthetic peptide
NHTSGD

SEQ ID NO: 400 - protein, 6 aa, Artificial Sequence
  synthetic peptide
NHGYTD

SEQ ID NO: 401 - protein, 6 aa, Artificial Sequence
  synthetic peptide
NHSGTD

SEQ ID NO: 402 - protein, 6 aa, Artificial Sequence
  synthetic peptide
NGYHTD

SEQ ID NO: 403 - protein, 6 aa, Artificial Sequence
  synthetic peptide
NSGHTD

SEQ ID NO: 404 - protein, 6 aa, Artificial Sequence
  synthetic peptide
NGHTYD

SEQ ID NO: 405 - protein, 6 aa, Artificial Sequence
  synthetic peptide
NSHTGD

SEQ ID NO: 406 - protein, 6 aa, Artificial Sequence
  synthetic peptide
NTHGYK

SEQ ID NO: 407 - protein, 6 aa, Artificial Sequence
  synthetic peptide
NTHSGK

SEQ ID NO: 408 - protein, 6 aa, Artificial Sequence
  synthetic peptide
NTGYHK

SEQ ID NO: 409 - protein, 6 aa, Artificial Sequence
  synthetic peptide
NTSGHK

SEQ ID NO: 410 - protein, 6 aa, Artificial Sequence
  synthetic peptide
NGYTHK

SEQ ID NO: 411 - protein, 6 aa, Artificial Sequence
  synthetic peptide
NSGTHK

SEQ ID NO: 412 - protein, 6 aa, Artificial Sequence
  synthetic peptide
NGTHYK

SEQ ID NO: 413 - protein, 6 aa, Artificial Sequence
  synthetic peptide
NSTHGK

SEQ ID NO: 414 - protein, 6 aa, Artificial Sequence
  synthetic peptide
NKHGYL

SEQ ID NO: 415 - protein, 6 aa, Artificial Sequence
  synthetic peptide
NKHSGL

SEQ ID NO: 416 - protein, 6 aa, Artificial Sequence
  synthetic peptide
NKGYHL

SEQ ID NO: 417 - protein, 6 aa, Artificial Sequence
  synthetic peptide
NKSGHL

SEQ ID NO: 418 - protein, 6 aa, Artificial Sequence
  synthetic peptide
NGYKHL

SEQ ID NO: 419 - protein, 6 aa, Artificial Sequence
  synthetic peptide
NSGKHL

SEQ ID NO: 420 - protein, 6 aa, Artificial Sequence
  synthetic peptide
NGKHYL

SEQ ID NO: 421 - protein, 6 aa, Artificial Sequence
  synthetic peptide
NSKHGL

SEQ ID NO: 422 - protein, 6 aa, Artificial Sequence
  synthetic peptide
NLFGYD

SEQ ID NO: 423 - protein, 6 aa, Artificial Sequence
  synthetic peptide
NLFSGD

SEQ ID NO: 424 - protein, 6 aa, Artificial Sequence
  synthetic peptide
NLGYFD

SEQ ID NO: 425 - protein, 6 aa, Artificial Sequence
  synthetic peptide
NLSGFD

SEQ ID NO: 426 - protein, 6 aa, Artificial Sequence
  synthetic peptide
NGYLFD

SEQ ID NO: 427 - protein, 6 aa, Artificial Sequence
  synthetic peptide
NSGLFD

SEQ ID NO: 428 - protein, 6 aa, Artificial Sequence
  synthetic peptide
NGLFYD

SEQ ID NO: 429 - protein, 6 aa, Artificial Sequence
  synthetic peptide
NSLFGD

SEQ ID NO: 430 - protein, 6 aa, Artificial Sequence
  synthetic peptide
NDLGYF

SEQ ID NO: 431 - protein, 6 aa, Artificial Sequence
  synthetic peptide
NDLSGF

SEQ ID NO: 432 - protein, 6 aa, Artificial Sequence
  synthetic peptide
NDGYLF

SEQ ID NO: 433 - protein, 6 aa, Artificial Sequence
  synthetic peptide
NDSGLF

SEQ ID NO: 434 - protein, 6 aa, Artificial Sequence
  synthetic peptide
NGYDLF

SEQ ID NO: 435 - protein, 6 aa, Artificial Sequence
  synthetic peptide
NSGDLF

SEQ ID NO: 436 - protein, 6 aa, Artificial Sequence
  synthetic peptide
NGDLYF

SEQ ID NO: 437 - protein, 6 aa, Artificial Sequence
  synthetic peptide
NSDLGF

SEQ ID NO: 438 - protein, 6 aa, Artificial Sequence
  synthetic peptide
FEQGYP

SEQ ID NO: 439 - protein, 6 aa, Artificial Sequence
  synthetic peptide
FEQSGP

SEQ ID NO: 440 - protein, 6 aa, Artificial Sequence
  synthetic peptide
FEGYQP

SEQ ID NO: 441 - protein, 6 aa, Artificial Sequence
  synthetic peptide
FESGQP

SEQ ID NO: 442 - protein, 6 aa, Artificial Sequence
  synthetic peptide
FGYEQP

SEQ ID NO: 443 - protein, 6 aa, Artificial Sequence
  synthetic peptide
FSGEQP

SEQ ID NO: 444 - protein, 6 aa, Artificial Sequence
  synthetic peptide
FGEQYP

SEQ ID NO: 445 - protein, 6 aa, Artificial Sequence
  synthetic peptide
FSEQGP

SEQ ID NO: 446 - protein, 6 aa, Artificial Sequence
  synthetic peptide
FEKGYT

SEQ ID NO: 447 - protein, 6 aa, Artificial Sequence
  synthetic peptide
FEKSGT

SEQ ID NO: 448 - protein, 6 aa, Artificial Sequence
  synthetic peptide
FEGYKT

SEQ ID NO: 449 - protein, 6 aa, Artificial Sequence
  synthetic peptide
FESGKT

SEQ ID NO: 450 - protein, 6 aa, Artificial Sequence
  synthetic peptide
FGYEKT

SEQ ID NO: 451 - protein, 6 aa, Artificial Sequence
  synthetic peptide
FSGEKT

SEQ ID NO: 452 - protein, 6 aa, Artificial Sequence
  synthetic peptide
FGEKYT

SEQ ID NO: 453 - protein, 6 aa, Artificial Sequence
  synthetic peptide
FSEKGT

SEQ ID NO: 454 - protein, 6 aa, Artificial Sequence
  synthetic peptide
FEDGYH

SEQ ID NO: 455 - protein, 6 aa, Artificial Sequence
  synthetic peptide
FEDSGH

SEQ ID NO: 456 - protein, 6 aa, Artificial Sequence
  synthetic peptide
FEGYDH

SEQ ID NO: 457 - protein, 6 aa, Artificial Sequence
  synthetic peptide
FESGDH

SEQ ID NO: 458 - protein, 6 aa, Artificial Sequence
  synthetic peptide
FGYEDH

SEQ ID NO: 459 - protein, 6 aa, Artificial Sequence
  synthetic peptide
FSGEDH

SEQ ID NO: 460 - protein, 6 aa, Artificial Sequence
  synthetic peptide
FGEDYH

SEQ ID NO: 461 - protein, 6 aa, Artificial Sequence
  synthetic peptide
FSEDGH

SEQ ID NO: 462 - protein, 6 aa, Artificial Sequence
  synthetic peptide
FPNGYE

SEQ ID NO: 463 - protein, 6 aa, Artificial Sequence
  synthetic peptide
FPNSGE

SEQ ID NO: 464 - protein, 6 aa, Artificial Sequence
  synthetic peptide
FPGYNE

SEQ ID NO: 465 - protein, 6 aa, Artificial Sequence
  synthetic peptide
FPSGNE

SEQ ID NO: 466 - protein, 6 aa, Artificial Sequence
  synthetic peptide
FGYPNE

SEQ ID NO: 467 - protein, 6 aa, Artificial Sequence
  synthetic peptide
FSGPNE

SEQ ID NO: 468 - protein, 6 aa, Artificial Sequence
  synthetic peptide
FGPNYE

SEQ ID NO: 469 - protein, 6 aa, Artificial Sequence
  synthetic peptide
FSPNGE

SEQ ID NO: 470 - protein, 6 aa, Artificial Sequence
  synthetic peptide
FPKGYL

SEQ ID NO: 471 - protein, 6 aa, Artificial Sequence
  synthetic peptide
FPKSGL

SEQ ID NO: 472 - protein, 6 aa, Artificial Sequence
  synthetic peptide
FPGYKL

SEQ ID NO: 473 - protein, 6 aa, Artificial Sequence
  synthetic peptide
FPSGKL

SEQ ID NO: 474 - protein, 6 aa, Artificial Sequence
  synthetic peptide
FGYPKL

SEQ ID NO: 475 - protein, 6 aa, Artificial Sequence
  synthetic peptide
FSGPKL

SEQ ID NO: 476 - protein, 6 aa, Artificial Sequence
  synthetic peptide
FGPKYL

SEQ ID NO: 477 - protein, 6 aa, Artificial Sequence
  synthetic peptide
FSPKGL

SEQ ID NO: 478 - protein, 6 aa, Artificial Sequence
  synthetic peptide
FQNGYK

SEQ ID NO: 479 - protein, 6 aa, Artificial Sequence
  synthetic peptide
FQNSGK

SEQ ID NO: 480 - protein, 6 aa, Artificial Sequence
  synthetic peptide
FQGYNK

SEQ ID NO: 481 - protein, 6 aa, Artificial Sequence
  synthetic peptide
FQSGNK

SEQ ID NO: 482 - protein, 6 aa, Artificial Sequence
  synthetic peptide
FGYQNK

SEQ ID NO: 483 - protein, 6 aa, Artificial Sequence
  synthetic peptide
FSGQNK

SEQ ID NO: 484 - protein, 6 aa, Artificial Sequence
  synthetic peptide
FGQNYK

SEQ ID NO: 485 - protein, 6 aa, Artificial Sequence
  synthetic peptide
FSQNGK

SEQ ID NO: 486 - protein, 6 aa, Artificial Sequence
  synthetic peptide
FNPGYE

SEQ ID NO: 487 - protein, 6 aa, Artificial Sequence
  synthetic peptide
FNPSGE

SEQ ID NO: 488 - protein, 6 aa, Artificial Sequence
  synthetic peptide
FNGYPE

SEQ ID NO: 489 - protein, 6 aa, Artificial Sequence
  synthetic peptide
FNSGPE

SEQ ID NO: 490 - protein, 6 aa, Artificial Sequence
  synthetic peptide
FGYNPE

SEQ ID NO: 491 - protein, 6 aa, Artificial Sequence
  synthetic peptide
FSGNPE

SEQ ID NO: 492 - protein, 6 aa, Artificial Sequence
  synthetic peptide
FGNPYE

SEQ ID NO: 493 - protein, 6 aa, Artificial Sequence
  synthetic peptide
FSNPGE

SEQ ID NO: 494 - protein, 6 aa, Artificial Sequence
  synthetic peptide
FHEGYP

SEQ ID NO: 495 - protein, 6 aa, Artificial Sequence
  synthetic peptide
FHESGP

SEQ ID NO: 496 - protein, 6 aa, Artificial Sequence
  synthetic peptide
FHGYEP

SEQ ID NO: 497 - protein, 6 aa, Artificial Sequence
  synthetic peptide
FHSGEP

SEQ ID NO: 498 - protein, 6 aa, Artificial Sequence
  synthetic peptide
FGYHEP

SEQ ID NO: 499 - protein, 6 aa, Artificial Sequence
  synthetic peptide
FSGHEP

SEQ ID NO: 500 - protein, 6 aa, Artificial Sequence
  synthetic peptide
FGHEYP

SEQ ID NO: 501 - protein, 6 aa, Artificial Sequence
  synthetic peptide
FSHEGP

SEQ ID NO: 502 - protein, 6 aa, Artificial Sequence
  synthetic peptide
FHKGYE

SEQ ID NO: 503 - protein, 6 aa, Artificial Sequence
  synthetic peptide
FHKSGE

SEQ ID NO: 504 - protein, 6 aa, Artificial Sequence
  synthetic peptide
FHGYKE

SEQ ID NO: 505 - protein, 6 aa, Artificial Sequence
  synthetic peptide
FHSGKE

SEQ ID NO: 506 - protein, 6 aa, Artificial Sequence
  synthetic peptide
FGYHKE

SEQ ID NO: 507 - protein, 6 aa, Artificial Sequence
  synthetic peptide
FSGHKE

SEQ ID NO: 508 - protein, 6 aa, Artificial Sequence
  synthetic peptide
FGHKYE

SEQ ID NO: 509 - protein, 6 aa, Artificial Sequence
  synthetic peptide
FSHKGE

SEQ ID NO: 510 - protein, 6 aa, Artificial Sequence
  synthetic peptide
FTHGYN

SEQ ID NO: 511 - protein, 6 aa, Artificial Sequence
  synthetic peptide
FTHSGN

SEQ ID NO: 512 - protein, 6 aa, Artificial Sequence
  synthetic peptide
FTGYHN

SEQ ID NO: 513 - protein, 6 aa, Artificial Sequence
  synthetic peptide
FTSGHN

SEQ ID NO: 514 - protein, 6 aa, Artificial Sequence
  synthetic peptide
FGYTHN

SEQ ID NO: 515 - protein, 6 aa, Artificial Sequence
  synthetic peptide
FSGTHN

SEQ ID NO: 516 - protein, 6 aa, Artificial Sequence
  synthetic peptide
FGTHYN

SEQ ID NO: 517 - protein, 6 aa, Artificial Sequence
  synthetic peptide
FSTHGN

SEQ ID NO: 518 - protein, 6 aa, Artificial Sequence
  synthetic peptide
FTLGYQ

SEQ ID NO: 519 - protein, 6 aa, Artificial Sequence
  synthetic peptide
FTLSGQ

SEQ ID NO: 520 - protein, 6 aa, Artificial Sequence
  synthetic peptide
FTGYLQ

SEQ ID NO: 521 - protein, 6 aa, Artificial Sequence
  synthetic peptide
FTSGLQ

SEQ ID NO: 522 - protein, 6 aa, Artificial Sequence
  synthetic peptide
FGYTLQ

SEQ ID NO: 523 - protein, 6 aa, Artificial Sequence
  synthetic peptide
FSGTLQ

SEQ ID NO: 524 - protein, 6 aa, Artificial Sequence
  synthetic peptide
FGTLYQ

SEQ ID NO: 525 - protein, 6 aa, Artificial Sequence
  synthetic peptide
FSTLGQ

SEQ ID NO: 526 - protein, 6 aa, Artificial Sequence
  synthetic peptide
FKQGYH

SEQ ID NO: 527 - protein, 6 aa, Artificial Sequence
  synthetic peptide
FKQSGH

SEQ ID NO: 528 - protein, 6 aa, Artificial Sequence
  synthetic peptide
FKGYQH

SEQ ID NO: 529 - protein, 6 aa, Artificial Sequence
  synthetic peptide
FKSGQH

SEQ ID NO: 530 - protein, 6 aa, Artificial Sequence
  synthetic peptide
FGYKQH

SEQ ID NO: 531 - protein, 6 aa, Artificial Sequence
  synthetic peptide
FSGKQH

SEQ ID NO: 532 - protein, 6 aa, Artificial Sequence
  synthetic peptide
FGKQYH

SEQ ID NO: 533 - protein, 6 aa, Artificial Sequence
  synthetic peptide
FSKQGH

SEQ ID NO: 534 - protein, 6 aa, Artificial Sequence
  synthetic peptide
FKLGYP

SEQ ID NO: 535 - protein, 6 aa, Artificial Sequence
  synthetic peptide
FKLSGP

SEQ ID NO: 536 - protein, 6 aa, Artificial Sequence
  synthetic peptide
FKGYLP

SEQ ID NO: 537 - protein, 6 aa, Artificial Sequence
  synthetic peptide
FKSGLP

SEQ ID NO: 538 - protein, 6 aa, Artificial Sequence
  synthetic peptide
FGYKLP

SEQ ID NO: 539 - protein, 6 aa, Artificial Sequence
  synthetic peptide
FSGKLP

SEQ ID NO: 540 - protein, 6 aa, Artificial Sequence
  synthetic peptide
FGKLYP

SEQ ID NO: 541 - protein, 6 aa, Artificial Sequence
  synthetic peptide
FSKLGP

SEQ ID NO: 542 - protein, 6 aa, Artificial Sequence
  synthetic peptide
FLEGYD

SEQ ID NO: 543 - protein, 6 aa, Artificial Sequence
  synthetic peptide
FLESGD

SEQ ID NO: 544 - protein, 6 aa, Artificial Sequence
  synthetic peptide
FLGYED

SEQ ID NO: 545 - protein, 6 aa, Artificial Sequence
  synthetic peptide
FLSGED

SEQ ID NO: 546 - protein, 6 aa, Artificial Sequence
  synthetic peptide
FGYLED

SEQ ID NO: 547 - protein, 6 aa, Artificial Sequence
  synthetic peptide
FSGLED

SEQ ID NO: 548 - protein, 6 aa, Artificial Sequence
  synthetic peptide
FGLEYD

SEQ ID NO: 549 - protein, 6 aa, Artificial Sequence
  synthetic peptide
FSLEGD

SEQ ID NO: 550 - protein, 6 aa, Artificial Sequence
  synthetic peptide
FLHGYQ

SEQ ID NO: 551 - protein, 6 aa, Artificial Sequence
  synthetic peptide
FLHSGQ

SEQ ID NO: 552 - protein, 6 aa, Artificial Sequence
  synthetic peptide
FLGYHQ

SEQ ID NO: 553 - protein, 6 aa, Artificial Sequence
  synthetic peptide
FLSGHQ

SEQ ID NO: 554 - protein, 6 aa, Artificial Sequence
  synthetic peptide
FGYLHQ

SEQ ID NO: 555 - protein, 6 aa, Artificial Sequence
  synthetic peptide
FSGLHQ

SEQ ID NO: 556 - protein, 6 aa, Artificial Sequence
  synthetic peptide
FGLHYQ

SEQ ID NO: 557 - protein, 6 aa, Artificial Sequence
  synthetic peptide
FSLHGQ

SEQ ID NO: 558 - protein, 6 aa, Artificial Sequence
  synthetic peptide
FDTGYE

SEQ ID NO: 559 - protein, 6 aa, Artificial Sequence
  synthetic peptide
FDTSGE

SEQ ID NO: 560 - protein, 6 aa, Artificial Sequence
  synthetic peptide
FDGYTE

SEQ ID NO: 561 - protein, 6 aa, Artificial Sequence
  synthetic peptide
FDSGTE

SEQ ID NO: 562 - protein, 6 aa, Artificial Sequence
  synthetic peptide
FGYDTE

SEQ ID NO: 563 - protein, 6 aa, Artificial Sequence
  synthetic peptide
FSGDTE

SEQ ID NO: 564 - protein, 6 aa, Artificial Sequence
  synthetic peptide
FGDTYE

SEQ ID NO: 565 - protein, 6 aa, Artificial Sequence
  synthetic peptide
FSDTGE

SEQ ID NO: 566 - protein, 6 aa, Artificial Sequence
  synthetic peptide
HEQGYF

SEQ ID NO: 567 - protein, 6 aa, Artificial Sequence
  synthetic peptide
HEQSGF

SEQ ID NO: 568 - protein, 6 aa, Artificial Sequence
  synthetic peptide
HEGYQF

SEQ ID NO: 569 - protein, 6 aa, Artificial Sequence
  synthetic peptide
HESGQF

SEQ ID NO: 570 - protein, 6 aa, Artificial Sequence
  synthetic peptide
HGYEQF

SEQ ID NO: 571 - protein, 6 aa, Artificial Sequence
  synthetic peptide
HSGEQF

SEQ ID NO: 572 - protein, 6 aa, Artificial Sequence
  synthetic peptide
HGEQYF

SEQ ID NO: 573 - protein, 6 aa, Artificial Sequence
  synthetic peptide
HSEQGF

SEQ ID NO: 574 - protein, 6 aa, Artificial Sequence
  synthetic peptide
HEKGYP

SEQ ID NO: 575 - protein, 6 aa, Artificial Sequence
  synthetic peptide
HEKSGP

SEQ ID NO: 576 - protein, 6 aa, Artificial Sequence
  synthetic peptide
HEGYKP

SEQ ID NO: 577 - protein, 6 aa, Artificial Sequence
  synthetic peptide
HESGKP

SEQ ID NO: 578 - protein, 6 aa, Artificial Sequence
  synthetic peptide
HGYEKP

SEQ ID NO: 579 - protein, 6 aa, Artificial Sequence
  synthetic peptide
HSGEKP

SEQ ID NO: 580 - protein, 6 aa, Artificial Sequence
  synthetic peptide
HGEKYP

SEQ ID NO: 581 - protein, 6 aa, Artificial Sequence
  synthetic peptide
HSEKGP

SEQ ID NO: 582 - protein, 6 aa, Artificial Sequence
  synthetic peptide
HPEGYD

SEQ ID NO: 583 - protein, 6 aa, Artificial Sequence
  synthetic peptide
HPESGD

SEQ ID NO: 584 - protein, 6 aa, Artificial Sequence
  synthetic peptide
HPGYED

SEQ ID NO: 585 - protein, 6 aa, Artificial Sequence
  synthetic peptide
HPSGED

SEQ ID NO: 586 - protein, 6 aa, Artificial Sequence
  synthetic peptide
HGYPED

SEQ ID NO: 587 - protein, 6 aa, Artificial Sequence
  synthetic peptide
HSGPED

SEQ ID NO: 588 - protein, 6 aa, Artificial Sequence
  synthetic peptide
HGPEYD

SEQ ID NO: 589 - protein, 6 aa, Artificial Sequence
  synthetic peptide
HSPEGD

SEQ ID NO: 590 - protein, 6 aa, Artificial Sequence
  synthetic peptide
HPFGYL

SEQ ID NO: 591 - protein, 6 aa, Artificial Sequence
  synthetic peptide
HPFSGL

SEQ ID NO: 592 - protein, 6 aa, Artificial Sequence
  synthetic peptide
HPGYFL

SEQ ID NO: 593 - protein, 6 aa, Artificial Sequence
  synthetic peptide
HPSGFL

SEQ ID NO: 594 - protein, 6 aa, Artificial Sequence
  synthetic peptide
HGYPFL

SEQ ID NO: 595 - protein, 6 aa, Artificial Sequence
  synthetic peptide
HSGPFL

SEQ ID NO: 596 - protein, 6 aa, Artificial Sequence
  synthetic peptide
HGPFYL

SEQ ID NO: 597 - protein, 6 aa, Artificial Sequence
  synthetic peptide
HSPFGL

SEQ ID NO: 598 - protein, 6 aa, Artificial Sequence
  synthetic peptide
HQEGYL

SEQ ID NO: 599 - protein, 6 aa, Artificial Sequence
  synthetic peptide
HQESGL

SEQ ID NO: 600 - protein, 6 aa, Artificial Sequence
  synthetic peptide
HQGYEL

SEQ ID NO: 601 - protein, 6 aa, Artificial Sequence
  synthetic peptide
HQSGEL

SEQ ID NO: 602 - protein, 6 aa, Artificial Sequence
  synthetic peptide
HGYQEL

SEQ ID NO: 603 - protein, 6 aa, Artificial Sequence
  synthetic peptide
HSGQEL

SEQ ID NO: 604 - protein, 6 aa, Artificial Sequence
  synthetic peptide
HGQEYL

SEQ ID NO: 605 - protein, 6 aa, Artificial Sequence
  synthetic peptide
HSQEGL

SEQ ID NO: 606 - protein, 6 aa, Artificial Sequence
  synthetic peptide
HQTGYN

SEQ ID NO: 607 - protein, 6 aa, Artificial Sequence
  synthetic peptide
HQTSGN

SEQ ID NO: 608 - protein, 6 aa, Artificial Sequence
  synthetic peptide
HQGYTN

SEQ ID NO: 609 - protein, 6 aa, Artificial Sequence
  synthetic peptide
HQSGTN

SEQ ID NO: 610 - protein, 6 aa, Artificial Sequence
  synthetic peptide
HGYQTN

SEQ ID NO: 611 - protein, 6 aa, Artificial Sequence
  synthetic peptide
HSGQTN

SEQ ID NO: 612 - protein, 6 aa, Artificial Sequence
  synthetic peptide
HGQTYN

SEQ ID NO: 613 - protein, 6 aa, Artificial Sequence
  synthetic peptide
HSQTGN

SEQ ID NO: 614 - protein, 6 aa, Artificial Sequence
  synthetic peptide
HNKGYD

SEQ ID NO: 615 - protein, 6 aa, Artificial Sequence
  synthetic peptide
HNKSGD

SEQ ID NO: 616 - protein, 6 aa, Artificial Sequence
  synthetic peptide
HNGYKD

SEQ ID NO: 617 - protein, 6 aa, Artificial Sequence
  synthetic peptide
HNSGKD

SEQ ID NO: 618 - protein, 6 aa, Artificial Sequence
  synthetic peptide
HGYNKD

SEQ ID NO: 619 - protein, 6 aa, Artificial Sequence
  synthetic peptide
HSGNKD

SEQ ID NO: 620 - protein, 6 aa, Artificial Sequence
  synthetic peptide
HGNKYD

SEQ ID NO: 621 - protein, 6 aa, Artificial Sequence
  synthetic peptide
HSNKGD

SEQ ID NO: 622 - protein, 6 aa, Artificial Sequence
  synthetic peptide
HNDGYT

SEQ ID NO: 623 - protein, 6 aa, Artificial Sequence
  synthetic peptide
HNDSGT

SEQ ID NO: 624 - protein, 6 aa, Artificial Sequence
  synthetic peptide
HNGYDT

SEQ ID NO: 625 - protein, 6 aa, Artificial Sequence
  synthetic peptide
HNSGDT

SEQ ID NO: 626 - protein, 6 aa, Artificial Sequence
  synthetic peptide
HGYNDT

SEQ ID NO: 627 - protein, 6 aa, Artificial Sequence
  synthetic peptide
HSGNDT

SEQ ID NO: 628 - protein, 6 aa, Artificial Sequence
  synthetic peptide
HGNDYT

SEQ ID NO: 629 - protein, 6 aa, Artificial Sequence
  synthetic peptide
HSNDGT

SEQ ID NO: 630 - protein, 6 aa, Artificial Sequence
  synthetic peptide
HFTGYK

SEQ ID NO: 631 - protein, 6 aa, Artificial Sequence
  synthetic peptide
HFTSGK

SEQ ID NO: 632 - protein, 6 aa, Artificial Sequence
  synthetic peptide
HFGYTK

SEQ ID NO: 633 - protein, 6 aa, Artificial Sequence
  synthetic peptide
HFSGTK

SEQ ID NO: 634 - protein, 6 aa, Artificial Sequence
  synthetic peptide
HGYFTK

SEQ ID NO: 635 - protein, 6 aa, Artificial Sequence
  synthetic peptide
HSGFTK

SEQ ID NO: 636 - protein, 6 aa, Artificial Sequence
  synthetic peptide
HGFTYK

SEQ ID NO: 637 - protein, 6 aa, Artificial Sequence
  synthetic peptide
HSFTGK

SEQ ID NO: 638 - protein, 6 aa, Artificial Sequence
  synthetic peptide
HTPGYN

SEQ ID NO: 639 - protein, 6 aa, Artificial Sequence
  synthetic peptide
HTPSGN

SEQ ID NO: 640 - protein, 6 aa, Artificial Sequence
  synthetic peptide
HTGYPN

SEQ ID NO: 641 - protein, 6 aa, Artificial Sequence
  synthetic peptide
HTSGPN

SEQ ID NO: 642 - protein, 6 aa, Artificial Sequence
  synthetic peptide
HGYTPN

SEQ ID NO: 643 - protein, 6 aa, Artificial Sequence
  synthetic peptide
HSGTPN

SEQ ID NO: 644 - protein, 6 aa, Artificial Sequence
  synthetic peptide
HGTPYN

SEQ ID NO: 645 - protein, 6 aa, Artificial Sequence
  synthetic peptide
HSTPGN

SEQ ID NO: 646 - protein, 6 aa, Artificial Sequence
  synthetic peptide
HTFGYQ

SEQ ID NO: 647 - protein, 6 aa, Artificial Sequence
  synthetic peptide
HTFSGQ

SEQ ID NO: 648 - protein, 6 aa, Artificial Sequence
  synthetic peptide
HTGYFQ

SEQ ID NO: 649 - protein, 6 aa, Artificial Sequence
  synthetic peptide
HTSGFQ

SEQ ID NO: 650 - protein, 6 aa, Artificial Sequence
  synthetic peptide
HGYTFQ

SEQ ID NO: 651 - protein, 6 aa, Artificial Sequence
  synthetic peptide
HSGTFQ

SEQ ID NO: 652 - protein, 6 aa, Artificial Sequence
  synthetic peptide
HGTFYQ

SEQ ID NO: 653 - protein, 6 aa, Artificial Sequence
  synthetic peptide
HSTFGQ

SEQ ID NO: 654 - protein, 6 aa, Artificial Sequence
  synthetic peptide
HKPGYE

SEQ ID NO: 655 - protein, 6 aa, Artificial Sequence
  synthetic peptide
HKPSGE

SEQ ID NO: 656 - protein, 6 aa, Artificial Sequence
  synthetic peptide
HKGYPE

SEQ ID NO: 657 - protein, 6 aa, Artificial Sequence
  synthetic peptide
HKSGPE

SEQ ID NO: 658 - protein, 6 aa, Artificial Sequence
  synthetic peptide
HGYKPE

SEQ ID NO: 659 - protein, 6 aa, Artificial Sequence
  synthetic peptide
HSGKPE

SEQ ID NO: 660 - protein, 6 aa, Artificial Sequence
  synthetic peptide
HGKPYE

SEQ ID NO: 661 - protein, 6 aa, Artificial Sequence
  synthetic peptide
HSKPGE

SEQ ID NO: 662 - protein, 6 aa, Artificial Sequence
  synthetic peptide
HLEGYF

SEQ ID NO: 663 - protein, 6 aa, Artificial Sequence
  synthetic peptide
HLESGF

SEQ ID NO: 664 - protein, 6 aa, Artificial Sequence
  synthetic peptide
HLGYEF

SEQ ID NO: 665 - protein, 6 aa, Artificial Sequence
  synthetic peptide
HLSGEF

SEQ ID NO: 666 - protein, 6 aa, Artificial Sequence
  synthetic peptide
HGYLEF

SEQ ID NO: 667 - protein, 6 aa, Artificial Sequence
  synthetic peptide
HSGLEF

SEQ ID NO: 668 - protein, 6 aa, Artificial Sequence
  synthetic peptide
HGLEYF

SEQ ID NO: 669 - protein, 6 aa, Artificial Sequence
  synthetic peptide
HSLEGF

SEQ ID NO: 670 - protein, 6 aa, Artificial Sequence
  synthetic peptide
HDTGYL

SEQ ID NO: 671 - protein, 6 aa, Artificial Sequence
  synthetic peptide
HDTSGL

SEQ ID NO: 672 - protein, 6 aa, Artificial Sequence
  synthetic peptide
HDGYTL

SEQ ID NO: 673 - protein, 6 aa, Artificial Sequence
  synthetic peptide
HDSGTL

SEQ ID NO: 674 - protein, 6 aa, Artificial Sequence
  synthetic peptide
HGYDTL

SEQ ID NO: 675 - protein, 6 aa, Artificial Sequence
  synthetic peptide
HSGDTL

SEQ ID NO: 676 - protein, 6 aa, Artificial Sequence
  synthetic peptide
HGDTYL

SEQ ID NO: 677 - protein, 6 aa, Artificial Sequence
  synthetic peptide
HSDTGL

SEQ ID NO: 678 - protein, 6 aa, Artificial Sequence
  synthetic peptide
TEFGYL

SEQ ID NO: 679 - protein, 6 aa, Artificial Sequence
  synthetic peptide
TEFSGL

SEQ ID NO: 680 - protein, 6 aa, Artificial Sequence
  synthetic peptide
TEGYFL

SEQ ID NO: 681 - protein, 6 aa, Artificial Sequence
  synthetic peptide
TESGFL

SEQ ID NO: 682 - protein, 6 aa, Artificial Sequence
  synthetic peptide
TGYEFL

SEQ ID NO: 683 - protein, 6 aa, Artificial Sequence
  synthetic peptide
TSGEFL

SEQ ID NO: 684 - protein, 6 aa, Artificial Sequence
  synthetic peptide
TGEFYL

SEQ ID NO: 685 - protein, 6 aa, Artificial Sequence
  synthetic peptide
TSEFGL

SEQ ID NO: 686 - protein, 6 aa, Artificial Sequence
  synthetic peptide
TPDGYK

SEQ ID NO: 687 - protein, 6 aa, Artificial Sequence
  synthetic peptide
TPDSGK

SEQ ID NO: 688 - protein, 6 aa, Artificial Sequence
  synthetic peptide
TPGYDK

SEQ ID NO: 689 - protein, 6 aa, Artificial Sequence
  synthetic peptide
TPSGDK

SEQ ID NO: 690 - protein, 6 aa, Artificial Sequence
  synthetic peptide
TGYPDK

SEQ ID NO: 691 - protein, 6 aa, Artificial Sequence
  synthetic peptide
TSGPDK

SEQ ID NO: 692 - protein, 6 aa, Artificial Sequence
  synthetic peptide
TGPDYK

SEQ ID NO: 693 - protein, 6 aa, Artificial Sequence
  synthetic peptide
TSPDGK

SEQ ID NO: 694 - protein, 6 aa, Artificial Sequence
  synthetic peptide
TQLGYE

SEQ ID NO: 695 - protein, 6 aa, Artificial Sequence
  synthetic peptide
TQLSGE

SEQ ID NO: 696 - protein, 6 aa, Artificial Sequence
  synthetic peptide
TQGYLE

SEQ ID NO: 697 - protein, 6 aa, Artificial Sequence
  synthetic peptide
TQSGLE

SEQ ID NO: 698 - protein, 6 aa, Artificial Sequence
  synthetic peptide
TGYQLE

SEQ ID NO: 699 - protein, 6 aa, Artificial Sequence
  synthetic peptide
TSGQLE

SEQ ID NO: 700 - protein, 6 aa, Artificial Sequence
  synthetic peptide
TGQLYE

SEQ ID NO: 701 - protein, 6 aa, Artificial Sequence
  synthetic peptide
TSQLGE

SEQ ID NO: 702 - protein, 6 aa, Artificial Sequence
  synthetic peptide
TNDGYL

SEQ ID NO: 703 - protein, 6 aa, Artificial Sequence
  synthetic peptide
TNDSGL

SEQ ID NO: 704 - protein, 5 aa, Artificial Sequence
  synthetic peptide
TNGYD

SEQ ID NO: 705 - protein, 6 aa, Artificial Sequence
  synthetic peptide
TNSGDL

SEQ ID NO: 706 - protein, 6 aa, Artificial Sequence
  synthetic peptide
TGYNDL

SEQ ID NO: 707 - protein, 6 aa, Artificial Sequence
  synthetic peptide
TSGNDL

SEQ ID NO: 708 - protein, 6 aa, Artificial Sequence
  synthetic peptide
TGNDYL

SEQ ID NO: 709 - protein, 6 aa, Artificial Sequence
  synthetic peptide
TSNDGL

SEQ ID NO: 710 - protein, 6 aa, Artificial Sequence
  synthetic peptide
TFHGYE

SEQ ID NO: 711 - protein, 6 aa, Artificial Sequence
  synthetic peptide
TFHSGE

SEQ ID NO: 712 - protein, 6 aa, Artificial Sequence
  synthetic peptide
TFGYHE

SEQ ID NO: 713 - protein, 6 aa, Artificial Sequence
  synthetic peptide
TFSGHE

SEQ ID NO: 714 - protein, 6 aa, Artificial Sequence
  synthetic peptide
TGYFHE

SEQ ID NO: 715 - protein, 6 aa, Artificial Sequence
  synthetic peptide
TSGFHE

SEQ ID NO: 716 - protein, 6 aa, Artificial Sequence
  synthetic peptide
TGFHYE

SEQ ID NO: 717 - protein, 6 aa, Artificial Sequence
  synthetic peptide
TSFHGE

SEQ ID NO: 718 - protein, 6 aa, Artificial Sequence
  synthetic peptide
THLGYK

SEQ ID NO: 719 - protein, 6 aa, Artificial Sequence
  synthetic peptide
THLSGK

SEQ ID NO: 720 - protein, 6 aa, Artificial Sequence
  synthetic peptide
THGYLK

SEQ ID NO: 721 - protein, 6 aa, Artificial Sequence
  synthetic peptide
THSGLK

SEQ ID NO: 722 - protein, 6 aa, Artificial Sequence
  synthetic peptide
TGYHLK

SEQ ID NO: 723 - protein, 6 aa, Artificial Sequence
  synthetic peptide
TSGHLK

SEQ ID NO: 724 - protein, 6 aa, Artificial Sequence
  synthetic peptide
TGHLYK

SEQ ID NO: 725 - protein, 6 aa, Artificial Sequence
  synthetic peptide
TSHLGK

SEQ ID NO: 726 - protein, 6 aa, Artificial Sequence
  synthetic peptide
TLNGYF

SEQ ID NO: 727 - protein, 6 aa, Artificial Sequence
  synthetic peptide
TLNSGF

SEQ ID NO: 728 - protein, 6 aa, Artificial Sequence
  synthetic peptide
TLGYNF

SEQ ID NO: 729 - protein, 6 aa, Artificial Sequence
  synthetic peptide
TLSGNF

SEQ ID NO: 730 - protein, 6 aa, Artificial Sequence
  synthetic peptide
TGYLNF

SEQ ID NO: 731 - protein, 6 aa, Artificial Sequence
  synthetic peptide
TSGLNF

SEQ ID NO: 732 - protein, 6 aa, Artificial Sequence
  synthetic peptide
TGLNYF

SEQ ID NO: 733 - protein, 6 aa, Artificial Sequence
  synthetic peptide
TSLNGF

SEQ ID NO: 734 - protein, 6 aa, Artificial Sequence
  synthetic peptide
TDEGYQ

SEQ ID NO: 735 - protein, 6 aa, Artificial Sequence
  synthetic peptide
TDESGQ

SEQ ID NO: 736 - protein, 6 aa, Artificial Sequence
  synthetic peptide
TDGYEQ

SEQ ID NO: 737 - protein, 6 aa, Artificial Sequence
  synthetic peptide
TDSGEQ

SEQ ID NO: 738 - protein, 6 aa, Artificial Sequence
  synthetic peptide
TGYDEQ

SEQ ID NO: 739 - protein, 6 aa, Artificial Sequence
  synthetic peptide
TSGDEQ

SEQ ID NO: 740 - protein, 6 aa, Artificial Sequence
  synthetic peptide
TGDEYQ

SEQ ID NO: 741 - protein, 6 aa, Artificial Sequence
  synthetic peptide
TSDEGQ

SEQ ID NO: 742 - protein, 6 aa, Artificial Sequence
  synthetic peptide
KEPGYH

SEQ ID NO: 743 - protein, 6 aa, Artificial Sequence
  synthetic peptide
KEPSGH

SEQ ID NO: 744 - protein, 6 aa, Artificial Sequence
  synthetic peptide
KEGYPH

SEQ ID NO: 745 - protein, 6 aa, Artificial Sequence
  synthetic peptide
KESGPH

SEQ ID NO: 746 - protein, 6 aa, Artificial Sequence
  synthetic peptide
KGYEPH

SEQ ID NO: 747 - protein, 6 aa, Artificial Sequence
  synthetic peptide
KSGEPH

SEQ ID NO: 748 - protein, 6 aa, Artificial Sequence
  synthetic peptide
KGEPYH

SEQ ID NO: 749 - protein, 6 aa, Artificial Sequence
  synthetic peptide
KSEPGH

SEQ ID NO: 750 - protein, 6 aa, Artificial Sequence
  synthetic peptide
KEDGYF

SEQ ID NO: 751 - protein, 6 aa, Artificial Sequence
  synthetic peptide
KEDSGF

SEQ ID NO: 752 - protein, 6 aa, Artificial Sequence
  synthetic peptide
KEGYDF

SEQ ID NO: 753 - protein, 6 aa, Artificial Sequence
  synthetic peptide
KESGDF

SEQ ID NO: 754 - protein, 6 aa, Artificial Sequence
  synthetic peptide
KGYEDF

SEQ ID NO: 755 - protein, 6 aa, Artificial Sequence
  synthetic peptide
KSGEDF

SEQ ID NO: 756 - protein, 6 aa, Artificial Sequence
  synthetic peptide
KGEDYF

SEQ ID NO: 757 - protein, 6 aa, Artificial Sequence
  synthetic peptide
KSEDGF

SEQ ID NO: 758 - protein, 6 aa, Artificial Sequence
  synthetic peptide
KPHGYN

SEQ ID NO: 759 - protein, 6 aa, Artificial Sequence
  synthetic peptide
KPHSGN

SEQ ID NO: 760 - protein, 6 aa, Artificial Sequence
  synthetic peptide
KPGYHN

SEQ ID NO: 761 - protein, 6 aa, Artificial Sequence
  synthetic peptide
KPSGHN

SEQ ID NO: 762 - protein, 6 aa, Artificial Sequence
  synthetic peptide
KGYPHN

SEQ ID NO: 763 - protein, 6 aa, Artificial Sequence
  synthetic peptide
KSGPHN

SEQ ID NO: 764 - protein, 6 aa, Artificial Sequence
  synthetic peptide
KGPHYN

SEQ ID NO: 765 - protein, 6 aa, Artificial Sequence
  synthetic peptide
KSPHGN

SEQ ID NO: 766 - protein, 6 aa, Artificial Sequence
  synthetic peptide
KQNGYT

SEQ ID NO: 767 - protein, 6 aa, Artificial Sequence
  synthetic peptide
KQNSGT

SEQ ID NO: 768 - protein, 6 aa, Artificial Sequence
  synthetic peptide
KQGYNT

SEQ ID NO: 769 - protein, 6 aa, Artificial Sequence
  synthetic peptide
KQSGNT

SEQ ID NO: 770 - protein, 6 aa, Artificial Sequence
  synthetic peptide
KGYQNT

SEQ ID NO: 771 - protein, 6 aa, Artificial Sequence
  synthetic peptide
KSGQNT

SEQ ID NO: 772 - protein, 6 aa, Artificial Sequence
  synthetic peptide
KGQNYT

SEQ ID NO: 773 - protein, 6 aa, Artificial Sequence
  synthetic peptide
KSQNGT

SEQ ID NO: 774 - protein, 6 aa, Artificial Sequence
  synthetic peptide
KNPGYL

SEQ ID NO: 775 - protein, 6 aa, Artificial Sequence
  synthetic peptide
KNPSGL

SEQ ID NO: 776 - protein, 6 aa, Artificial Sequence
  synthetic peptide
KNGYPL

SEQ ID NO: 777 - protein, 6 aa, Artificial Sequence
  synthetic peptide
KNSGPL

SEQ ID NO: 778 - protein, 6 aa, Artificial Sequence
  synthetic peptide
KGYNPL

SEQ ID NO: 779 - protein, 6 aa, Artificial Sequence
  synthetic peptide
KSGNPL

SEQ ID NO: 780 - protein, 6 aa, Artificial Sequence
  synthetic peptide
KGNPYL

SEQ ID NO: 781 - protein, 6 aa, Artificial Sequence
  synthetic peptide
KSNPGL

SEQ ID NO: 782 - protein, 6 aa, Artificial Sequence
  synthetic peptide
KNDGYQ

SEQ ID NO: 783 - protein, 6 aa, Artificial Sequence
  synthetic peptide
KNDSGQ

SEQ ID NO: 784 - protein, 6 aa, Artificial Sequence
  synthetic peptide
KNGYDQ

SEQ ID NO: 785 - protein, 6 aa, Artificial Sequence
  synthetic peptide
KNSGDQ

SEQ ID NO: 786 - protein, 6 aa, Artificial Sequence
  synthetic peptide
KGYNDQ

SEQ ID NO: 787 - protein, 6 aa, Artificial Sequence
  synthetic peptide
KSGNDQ

SEQ ID NO: 788 - protein, 6 aa, Artificial Sequence
  synthetic peptide
KGNDYQ

SEQ ID NO: 789 - protein, 6 aa, Artificial Sequence
  synthetic peptide
KSNDGQ

SEQ ID NO: 790 - protein, 6 aa, Artificial Sequence
  synthetic peptide
KFHGYP

SEQ ID NO: 791 - protein, 6 aa, Artificial Sequence
  synthetic peptide
KFHSGP

SEQ ID NO: 792 - protein, 6 aa, Artificial Sequence
  synthetic peptide
KFGYHP

SEQ ID NO: 793 - protein, 6 aa, Artificial Sequence
  synthetic peptide
KFSGHP

SEQ ID NO: 794 - protein, 6 aa, Artificial Sequence
  synthetic peptide
KGYFHP

SEQ ID NO: 795 - protein, 6 aa, Artificial Sequence
  synthetic peptide
KSGFHP

SEQ ID NO: 796 - protein, 6 aa, Artificial Sequence
  synthetic peptide
KGFHYP

SEQ ID NO: 797 - protein, 6 aa, Artificial Sequence
  synthetic peptide
KSFHGP

SEQ ID NO: 798 - protein, 6 aa, Artificial Sequence
  synthetic peptide
KFLGYH

SEQ ID NO: 799 - protein, 6 aa, Artificial Sequence
  synthetic peptide
KFLSGH

SEQ ID NO: 800 - protein, 6 aa, Artificial Sequence
  synthetic peptide
KFGYLH

SEQ ID NO: 801 - protein, 6 aa, Artificial Sequence
  synthetic peptide
KFSGLH

SEQ ID NO: 802 - protein, 6 aa, Artificial Sequence
  synthetic peptide
KGYFLH

SEQ ID NO: 803 - protein, 6 aa, Artificial Sequence
  synthetic peptide
KSGFLH

SEQ ID NO: 804 - protein, 6 aa, Artificial Sequence
  synthetic peptide
KGFLYH

SEQ ID NO: 805 - protein, 6 aa, Artificial Sequence
  synthetic peptide
KSFLGH

SEQ ID NO: 806 - protein, 6 aa, Artificial Sequence
  synthetic peptide
KHPGYD

SEQ ID NO: 807 - protein, 6 aa, Artificial Sequence
  synthetic peptide
KHPSGD

SEQ ID NO: 808 - protein, 6 aa, Artificial Sequence
  synthetic peptide
KHGYPD

SEQ ID NO: 809 - protein, 6 aa, Artificial Sequence
  synthetic peptide
KHSGPD

SEQ ID NO: 810 - protein, 6 aa, Artificial Sequence
  synthetic peptide
KGYHPD

SEQ ID NO: 811 - protein, 6 aa, Artificial Sequence
  synthetic peptide
KSGHPD

SEQ ID NO: 812 - protein, 6 aa, Artificial Sequence
  synthetic peptide
KGHPYD

SEQ ID NO: 813 - protein, 6 aa, Artificial Sequence
  synthetic peptide
KSHPGD

SEQ ID NO: 814 - protein, 6 aa, Artificial Sequence
  synthetic peptide
KTNGYD

SEQ ID NO: 815 - protein, 6 aa, Artificial Sequence
  synthetic peptide
KTNSGD

SEQ ID NO: 816 - protein, 6 aa, Artificial Sequence
  synthetic peptide
KTGYND

SEQ ID NO: 817 - protein, 6 aa, Artificial Sequence
  synthetic peptide
KTSGND

SEQ ID NO: 818 - protein, 6 aa, Artificial Sequence
  synthetic peptide
KGYTND

SEQ ID NO: 819 - protein, 6 aa, Artificial Sequence
  synthetic peptide
KSGTND

SEQ ID NO: 820 - protein, 6 aa, Artificial Sequence
  synthetic peptide
KGTNYD

SEQ ID NO: 821 - protein, 6 aa, Artificial Sequence
  synthetic peptide
KSTNGD

SEQ ID NO: 822 - protein, 6 aa, Artificial Sequence
  synthetic peptide
KDNGYL

SEQ ID NO: 823 - protein, 6 aa, Artificial Sequence
  synthetic peptide
KDNSGL

SEQ ID NO: 824 - protein, 6 aa, Artificial Sequence
  synthetic peptide
KDGYNL

SEQ ID NO: 825 - protein, 6 aa, Artificial Sequence
  synthetic peptide
KDSGNL

SEQ ID NO: 826 - protein, 6 aa, Artificial Sequence
  synthetic peptide
KGYDNL

SEQ ID NO: 827 - protein, 6 aa, Artificial Sequence
  synthetic peptide
KSGDNL

SEQ ID NO: 828 - protein, 6 aa, Artificial Sequence
  synthetic peptide
KGDNYL

SEQ ID NO: 829 - protein, 6 aa, Artificial Sequence
  synthetic peptide
KSDNGL

SEQ ID NO: 830 - protein, 6 aa, Artificial Sequence
  synthetic peptide
KDHGYE

SEQ ID NO: 831 - protein, 6 aa, Artificial Sequence
  synthetic peptide
KDHSGE

SEQ ID NO: 832 - protein, 6 aa, Artificial Sequence
  synthetic peptide
KDGYHE

SEQ ID NO: 833 - protein, 6 aa, Artificial Sequence
  synthetic peptide
KDSGHE

SEQ ID NO: 834 - protein, 6 aa, Artificial Sequence
  synthetic peptide
KGYDHE

SEQ ID NO: 835 - protein, 6 aa, Artificial Sequence
  synthetic peptide
KSGDHE

SEQ ID NO: 836 - protein, 6 aa, Artificial Sequence
  synthetic peptide
KGDHYE

SEQ ID NO: 837 - protein, 6 aa, Artificial Sequence
  synthetic peptide
KSDHGE

SEQ ID NO: 838 - protein, 6 aa, Artificial Sequence
  synthetic peptide
LEFGYK

SEQ ID NO: 839 - protein, 6 aa, Artificial Sequence
  synthetic peptide
LEFSGK

SEQ ID NO: 840 - protein, 6 aa, Artificial Sequence
  synthetic peptide
LEGYFK

SEQ ID NO: 841 - protein, 6 aa, Artificial Sequence
  synthetic peptide
LESGFK

SEQ ID NO: 842 - protein, 6 aa, Artificial Sequence
  synthetic peptide
LGYEFK

SEQ ID NO: 843 - protein, 6 aa, Artificial Sequence
  synthetic peptide
LSGEFK

SEQ ID NO: 844 - protein, 6 aa, Artificial Sequence
  synthetic peptide
LGEFYK

SEQ ID NO: 845 - protein, 6 aa, Artificial Sequence
  synthetic peptide
LSEFGK

SEQ ID NO: 846 - protein, 6 aa, Artificial Sequence
  synthetic peptide
LQEGYN

SEQ ID NO: 847 - protein, 6 aa, Artificial Sequence
  synthetic peptide
LQESGN

SEQ ID NO: 848 - protein, 6 aa, Artificial Sequence
  synthetic peptide
LQGYEN

SEQ ID NO: 849 - protein, 6 aa, Artificial Sequence
  synthetic peptide
LQSGEN

SEQ ID NO: 850 - protein, 6 aa, Artificial Sequence
  synthetic peptide
LGYQEN

SEQ ID NO: 851 - protein, 6 aa, Artificial Sequence
  synthetic peptide
LSGQEN

SEQ ID NO: 852 - protein, 6 aa, Artificial Sequence
  synthetic peptide
LGQEYN

SEQ ID NO: 853 - protein, 6 aa, Artificial Sequence
  synthetic peptide
LSQEGN

SEQ ID NO: 854 - protein, 6 aa, Artificial Sequence
  synthetic peptide
LNQGYT

SEQ ID NO: 855 - protein, 6 aa, Artificial Sequence
  synthetic peptide
LNQSGT

SEQ ID NO: 856 - protein, 6 aa, Artificial Sequence
  synthetic peptide
LNGYQT

SEQ ID NO: 857 - protein, 6 aa, Artificial Sequence
  synthetic peptide
LNSGQT

SEQ ID NO: 858 - protein, 6 aa, Artificial Sequence
  synthetic peptide
LGYNQT

SEQ ID NO: 859 - protein, 6 aa, Artificial Sequence
  synthetic peptide
LSGNQT

SEQ ID NO: 860 - protein, 6 aa, Artificial Sequence
  synthetic peptide
LGNQYT

SEQ ID NO: 861 - protein, 6 aa, Artificial Sequence
  synthetic peptide
LSNQGT

SEQ ID NO: 862 - protein, 6 aa, Artificial Sequence
  synthetic peptide
LFHGYK

SEQ ID NO: 863 - protein, 6 aa, Artificial Sequence
  synthetic peptide
LFHSGK

SEQ ID NO: 864 - protein, 6 aa, Artificial Sequence
  synthetic peptide
LFGYHK

SEQ ID NO: 865 - protein, 6 aa, Artificial Sequence
  synthetic peptide
LFSGHK

SEQ ID NO: 866 - protein, 6 aa, Artificial Sequence
  synthetic peptide
LGYFHK

SEQ ID NO: 867 - protein, 6 aa, Artificial Sequence
  synthetic peptide
LSGFHK

SEQ ID NO: 868 - protein, 6 aa, Artificial Sequence
  synthetic peptide
LGFHYK

SEQ ID NO: 869 - protein, 6 aa, Artificial Sequence
  synthetic peptide
LSFHGK

SEQ ID NO: 870 - protein, 6 aa, Artificial Sequence
  synthetic peptide
LFKGYD

SEQ ID NO: 871 - protein, 6 aa, Artificial Sequence
  synthetic peptide
LFKSGD

SEQ ID NO: 872 - protein, 6 aa, Artificial Sequence
  synthetic peptide
LFGYKD

SEQ ID NO: 873 - protein, 6 aa, Artificial Sequence
  synthetic peptide
LFSGKD

SEQ ID NO: 874 - protein, 6 aa, Artificial Sequence
  synthetic peptide
LGYFKD

SEQ ID NO: 875 - protein, 6 aa, Artificial Sequence
  synthetic peptide
LSGFKD

SEQ ID NO: 876 - protein, 6 aa, Artificial Sequence
  synthetic peptide
LGFKYD

SEQ ID NO: 877 - protein, 6 aa, Artificial Sequence
  synthetic peptide
LSFKGD

SEQ ID NO: 878 - protein, 6 aa, Artificial Sequence
  synthetic peptide
LHDGYF

SEQ ID NO: 879 - protein, 6 aa, Artificial Sequence
  synthetic peptide
LHDSGF

SEQ ID NO: 880 - protein, 6 aa, Artificial Sequence
  synthetic peptide
LHGYDF

SEQ ID NO: 881 - protein, 6 aa, Artificial Sequence
  synthetic peptide
LHSGDF

SEQ ID NO: 882 - protein, 6 aa, Artificial Sequence
  synthetic peptide
LGYHDF

SEQ ID NO: 883 - protein, 6 aa, Artificial Sequence
  synthetic peptide
LSGHDF

SEQ ID NO: 884 - protein, 6 aa, Artificial Sequence
  synthetic peptide
LGHDYF

SEQ ID NO: 885 - protein, 6 aa, Artificial Sequence
  synthetic peptide
LSHDGF

SEQ ID NO: 886 - protein, 6 aa, Artificial Sequence
  synthetic peptide
LTDGYK

SEQ ID NO: 887 - protein, 6 aa, Artificial Sequence
  synthetic peptide
LTDSGK

SEQ ID NO: 888 - protein, 6 aa, Artificial Sequence
  synthetic peptide
LTGYDK

SEQ ID NO: 889 - protein, 6 aa, Artificial Sequence
  synthetic peptide
LTSGDK

SEQ ID NO: 890 - protein, 6 aa, Artificial Sequence
  synthetic peptide
LGYTDK

SEQ ID NO: 891 - protein, 6 aa, Artificial Sequence
  synthetic peptide
LSGTDK

SEQ ID NO: 892 - protein, 6 aa, Artificial Sequence
  synthetic peptide
LGTDYK

SEQ ID NO: 893 - protein, 6 aa, Artificial Sequence
  synthetic peptide
LSTDGK

SEQ ID NO: 894 - protein, 6 aa, Artificial Sequence
  synthetic peptide
LDEGYH

SEQ ID NO: 895 - protein, 6 aa, Artificial Sequence
  synthetic peptide
LDESGH

SEQ ID NO: 896 - protein, 6 aa, Artificial Sequence
  synthetic peptide
LDGYEH

SEQ ID NO: 897 - protein, 6 aa, Artificial Sequence
  synthetic peptide
LDSGEH

SEQ ID NO: 898 - protein, 6 aa, Artificial Sequence
  synthetic peptide
LGYDEH

SEQ ID NO: 899 - protein, 6 aa, Artificial Sequence
  synthetic peptide
LSGDEH

SEQ ID NO: 900 - protein, 6 aa, Artificial Sequence
  synthetic peptide
LGDEYH

SEQ ID NO: 901 - protein, 6 aa, Artificial Sequence
  synthetic peptide
LSDEGH

SEQ ID NO: 902 - protein, 6 aa, Artificial Sequence
  synthetic peptide
DEPGYK

SEQ ID NO: 903 - protein, 6 aa, Artificial Sequence
  synthetic peptide
DEPSGK

SEQ ID NO: 904 - protein, 6 aa, Artificial Sequence
  synthetic peptide
DEGYPK

SEQ ID NO: 905 - protein, 6 aa, Artificial Sequence
  synthetic peptide
DESGPK

SEQ ID NO: 906 - protein, 6 aa, Artificial Sequence
  synthetic peptide
DGYEPK

SEQ ID NO: 907 - protein, 6 aa, Artificial Sequence
  synthetic peptide
DSGEPK

SEQ ID NO: 908 - protein, 6 aa, Artificial Sequence
  synthetic peptide
DGEPYK

SEQ ID NO: 909 - protein, 6 aa, Artificial Sequence
  synthetic peptide
DSEPGK

SEQ ID NO: 910 - protein, 6 aa, Artificial Sequence
  synthetic peptide
DELGYT

SEQ ID NO: 911 - protein, 6 aa, Artificial Sequence
  synthetic peptide
DELSGT

SEQ ID NO: 912 - protein, 6 aa, Artificial Sequence
  synthetic peptide
DEGYLT

SEQ ID NO: 913 - protein, 6 aa, Artificial Sequence
  synthetic peptide
DESGLT

SEQ ID NO: 914 - protein, 6 aa, Artificial Sequence
  synthetic peptide
DGYELT

SEQ ID NO: 915 - protein, 6 aa, Artificial Sequence
  synthetic peptide
DSGELT

SEQ ID NO: 916 - protein, 6 aa, Artificial Sequence
  synthetic peptide
DGELYT

SEQ ID NO: 917 - protein, 6 aa, Artificial Sequence
  synthetic peptide
DSELGT

SEQ ID NO: 918 - protein, 6 aa, Artificial Sequence
  synthetic peptide
DNKGYQ

SEQ ID NO: 919 - protein, 6 aa, Artificial Sequence
  synthetic peptide
DNKSGQ

SEQ ID NO: 920 - protein, 6 aa, Artificial Sequence
  synthetic peptide
DNGYKQ

SEQ ID NO: 921 - protein, 6 aa, Artificial Sequence
  synthetic peptide
DNSGKQ

SEQ ID NO: 922 - protein, 6 aa, Artificial Sequence
  synthetic peptide
DGYNKQ

SEQ ID NO: 923 - protein, 6 aa, Artificial Sequence
  synthetic peptide
DSGNKQ

SEQ ID NO: 924 - protein, 6 aa, Artificial Sequence
  synthetic peptide
DGNKYQ

SEQ ID NO: 925 - protein, 6 aa, Artificial Sequence
  synthetic peptide
DSNKGQ

SEQ ID NO: 926 - protein, 6 aa, Artificial Sequence
  synthetic peptide
DTEGYQ

SEQ ID NO: 927 - protein, 6 aa, Artificial Sequence
  synthetic peptide
DTESGQ

SEQ ID NO: 928 - protein, 6 aa, Artificial Sequence
  synthetic peptide
DTGYEQ

SEQ ID NO: 929 - protein, 6 aa, Artificial Sequence
  synthetic peptide
DTSGEQ

SEQ ID NO: 930 - protein, 6 aa, Artificial Sequence
  synthetic peptide
DGYTEQ

SEQ ID NO: 931 - protein, 6 aa, Artificial Sequence
  synthetic peptide
DSGTEQ

SEQ ID NO: 932 - protein, 6 aa, Artificial Sequence
  synthetic peptide
DGTEYQ

SEQ ID NO: 933 - protein, 6 aa, Artificial Sequence
  synthetic peptide
DSTEGQ

SEQ ID NO: 934 - protein, 6 aa, Artificial Sequence
  synthetic peptide
DKHGYP

SEQ ID NO: 935 - protein, 6 aa, Artificial Sequence
  synthetic peptide
DKHSGP

SEQ ID NO: 936 - protein, 6 aa, Artificial Sequence
  synthetic peptide
DKGYHP

SEQ ID NO: 937 - protein, 6 aa, Artificial Sequence
  synthetic peptide
DKSGHP

SEQ ID NO: 938 - protein, 6 aa, Artificial Sequence
  synthetic peptide
DGYKHP

SEQ ID NO: 939 - protein, 6 aa, Artificial Sequence
  synthetic peptide
DSGKHP

SEQ ID NO: 940 - protein, 6 aa, Artificial Sequence
  synthetic peptide
DGKHYP

SEQ ID NO: 941 - protein, 6 aa, Artificial Sequence
  synthetic peptide
DSKHGP

SEQ ID NO: 942 - protein, 6 aa, Artificial Sequence
  synthetic peptide
DLTGYF

SEQ ID NO: 943 - protein, 6 aa, Artificial Sequence
  synthetic peptide
DLTSGF

SEQ ID NO: 944 - protein, 6 aa, Artificial Sequence
  synthetic peptide
DLGYTF

SEQ ID NO: 945 - protein, 6 aa, Artificial Sequence
  synthetic peptide
DLSGTF

SEQ ID NO: 946 - protein, 6 aa, Artificial Sequence
  synthetic peptide
DGYLTF

SEQ ID NO: 947 - protein, 6 aa, Artificial Sequence
  synthetic peptide
DSGLTF

SEQ ID NO: 948 - protein, 6 aa, Artificial Sequence
  synthetic peptide
DGLTYF